(19) United States
(12) Patent Application Publication
(10) Pub. No.: US 2026/0356836 A1
(43) Pub. Date: Nov. 12, 2026

(54) SUBSTITUTED PYRIDOTRIAZINE COMPOUNDS AND USES THEREOF

(71) Applicant: Gilead Sciences, Inc., Foster City, CA (US)

(72) Inventors: Ana Z. Gonzalez Buenrostro, San Mateo, CA (US); Xiaochun Han, San Jose, CA (US); Gregg M. Schwarzwalder, San Carlos, CA (US); Suzanne M. Szewczyk, San Mateo, CA (US)

(21) Appl. No.: 19/555,019

(22) Filed: Mar. 3, 2026

Related U.S. Application Data
(60) Provisional application No. 63/767,331, filed on Mar. 5, 2025.

(51) Int. Cl. C07D 471/18; A61K 31/551

(52) U.S. Cl. CPC C07D 471/18; A61K 31/551

(57) ABSTRACT

The present disclosure relates generally to compounds, of Formula I:

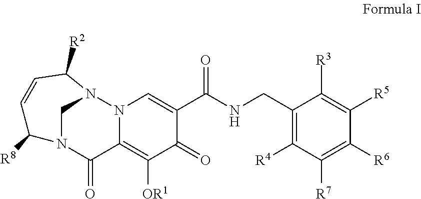

Also disclosed are pharmaceutical compositions comprising said compounds and methods of making said compounds. The compounds of the disclosure are useful in treating or preventing human immunodeficiency virus (HIV) infection.

### CROSS REFERENCE

This application claims priority to U.S. Provisional Application No. 63/767,331, filed on Mar. 5, 2025, the entire contents of which application is hereby incorporated by references in its entirety for all purposes.

### FIELD

Compounds, compositions, and methods that may be used for treating or preventing human immunodeficiency virus (HIV) infection are disclosed. In particular, 5-hydroxy-2,3-dihydro-1H-pyrido[2,1-f][1,2,4]triazine-4,6-dione compounds and methods for their preparation and use as therapeutic or prophylactic agents are disclosed.

### BACKGROUND

Human immunodeficiency virus infection and related diseases are a major public health problem worldwide. Human immunodeficiency virus encodes three enzymes which are required for viral replication: reverse transcriptase, protease, and integrase. Although drugs targeting reverse transcriptase and protease are in wide use and have shown effectiveness, particularly when employed in combination, toxicity and development of resistant strains may limit their usefulness (Palella, et al. N. Engl. J Med. (1998) 338:853-860; Richman, D. D. Nature (2001) 410:995-1001). Accordingly, there is a need for new agents that inhibit the replication of HIV.

A goal of antiretroviral therapy is to achieve viral suppression in the HIV infected patient. Current treatment guidelines published by the United States Department of Health and Human Services provide that achievement of viral suppression requires the use of combination therapies, i.e., several drugs from at least two or more drug classes (Panel on Antiretroviral Guidelines for Adults and Adolescents. Guidelines for the Use of Antiretroviral Agents in Adults and Adolescents Living with HIV. Department of Health and Human Services. Available at http://www.aidsinfo.nih.gov/ContentFiles/AdultandAdolescentGL.pdf. Accessed Feb. 12, 2019). In addition, decisions regarding the treatment of HIV infected patients are complicated when the patient requires treatment for other medical conditions (Id. at F-8). Because the standard of care requires the use of multiple different drugs to suppress HIV, as well as to treat other conditions the patient may be experiencing, the potential for drug interaction is a criterion for selection of a drug regimen. As such, there is a need for antiretroviral therapies having a decreased potential for drug interactions.

In addition, the HIV virus is known to mutate in infected subjects (Tang, et al. Drugs (2012) 72 (9) e1-e25). Because of the proclivity of the HIV virus to mutate, there is a need for anti-HIV drugs to be effective against a range of known HIV variants (Hurt, et al. HIV/AIDS CID (2014) 58, 423-431).

Drugs that offer favorable pharmaceutical properties (for example, improved potency, long-acting pharmacokinetics, low clearance, and/or other properties) are amenable to less frequent administration and provide for better patient compliance. Such improvements can, in turn, optimize drug exposure and limit the emergence of drug resistance.

### SUMMARY

The present disclosure is directed to novel compounds having antiviral activity and pharmaceutically acceptable salts thereof. In some embodiments, the compounds may be used to treat HIV infections, to inhibit the activity of HIV integrase and/or to reduce HIV replication. In some embodiments, compounds disclosed herein may be effective against a range of known drug-resistant HIV mutants. In some embodiments, compounds disclosed herein may have a decreased propensity to cause drug-drug interactions when co-administered with other drugs. In some embodiments, compounds disclosed herein may be administered with less than daily frequency, for example, at weekly, monthly, once every three months, once every six months, or longer intervals.

In some embodiments, the disclosure provides a compound of Formula I:

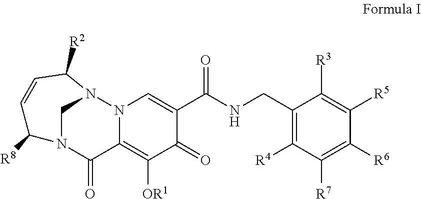

or a pharmaceutically acceptable salt thereof, wherein:
R¹ is —(CR^{1A}R^{1B}O)ₘCO(O—CR^{1C}R^{1D}—CR^{1E}R^{1F})ₙ—(O)ₚR^{1G};
   wherein m is 0 or 1;
   n is an integer between 0 and 10, inclusive;
   p is 0 or 1;
   R^{1A} is H or C₁₋₃alkyl;
   R^{1B} is H or C₁₋₃alkyl;
   each R^{1C} is independently H or C₁₋₃alkyl;
   each R^{1D} is independently H or C₁₋₃alkyl;
   each R^{1E} is independently H or C₁₋₃alkyl;
   each R^{1F} is independently H or C₁₋₃alkyl;
   R^{1G} is C₃₋₆cycloalkyl, C₁₋₂₀alkyl, C₂₋₂₀alkenyl, or a 3 to 8 membered heterocyclyl comprising 1, 2, or 3 heteroatoms selected independently from N, O, and S; wherein the C₃₋₆cycloalkyl, C₁₋₂₀alkyl, C₂₋₂₀alkenyl, or 3 to 8 membered heterocyclyl is optionally substituted with a COOH, C₃₋₆cycloalkyl, C₁₋₃alkoxy, or —NHCOOR^{1H};
   wherein R^{1H} is C₁₋₃alkyl;
R² is H or C₁₋₃alkyl;
each R³, R⁴, R⁵, R⁶ and R⁷ is independently H or halo; and
R⁸ is H or C₁₋₃alkyl.

In some embodiments, the disclosure provides a compound of Formula I, or a pharmaceutically acceptable salt thereof, wherein:
R¹ is —(CR^{1A}R^{1B}O)ₘCO(—CR^{1C}R^{1D}—CR^{1E}R^{1F})ₙ—(O)ₚR^{1G};
   wherein m is 0 or 1;
   n is 0, 1, 2, or 3;
   p is 0 or 1;
   R^{1A} is H or C₁₋₃alkyl;
   R^{1B} is H or C₁₋₃alkyl;
   each R^{1C} is independently H or C₁₋₃alkyl;
   each R^{1D} is independently H or C₁₋₃alkyl;
   each R^{1E} is independently H or C₁₋₃alkyl;
   each R^{1F} is independently H or C₁₋₃alkyl;
   R^{1G} is C₃₋₆cycloalkyl, C₁₋₂₀alkyl, C₂₋₂₀alkenyl, or a 3 to 8 membered heterocyclyl comprising 1, 2, or 3 heteroatoms selected independently from N, O, and S; wherein the C₃₋₆cycloalkyl, C₁₋₂₀alkyl, C₂₋₂₀alkenyl, or 3 to 8 membered heterocyclyl is optionally substituted with a COOH, C₃₋₆cycloalkyl, or C₁₋₃alkoxy;
R² is H or C₁₋₃alkyl;
each R³, R⁴, R⁵, R⁶ and R⁷ is independently H or halo; and
R⁸ is H or C₁₋₃alkyl.

In some embodiments, the disclosure provides a compound of Formula Ia:

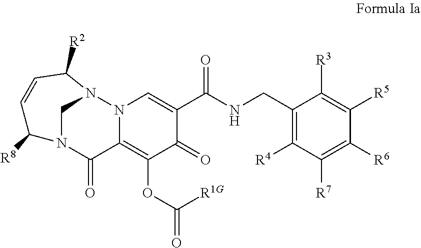

or a pharmaceutically acceptable salt thereof, wherein variables R^{1G}, R², R³, R⁴, R⁵, R⁶, R⁷, and R⁸ are defined according to the definitions provided herein for compounds of Formula I.

In some embodiments, the disclosure provides a compound of Formula Ib:

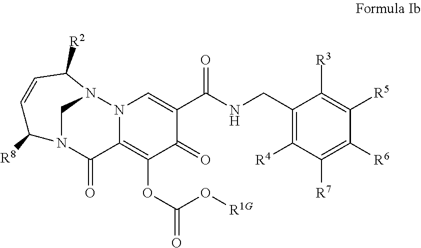

or a pharmaceutically acceptable salt thereof, wherein variables R^{1G}, R², R³, R⁴, R⁵, R⁶, R⁷, and R⁸ are defined according to the definitions provided herein for compounds of Formula I.

In some embodiments, the disclosure provides a compound of Formula Ic:

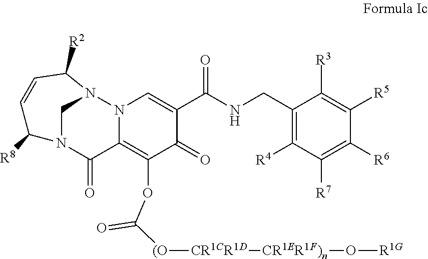

or a pharmaceutically acceptable salt thereof, wherein n is an integer between 1 and 7, inclusive; and variables R^{1C}, R^{1D}, R^{1E}, R^{1F}, R^{1G}, R², R³, R⁴, R⁵, R⁶, R⁷, and R⁸ are defined according to the definitions provided herein for compounds of Formula I.

In some embodiments, the disclosure provides a compound of Formula Id:

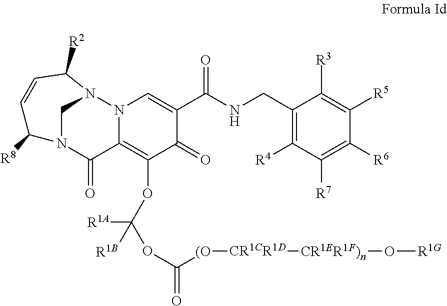

or a pharmaceutically acceptable salt thereof, wherein n is 0, 1, 2 or 3; and variables R^{1A}, R^{1B}, R^{1C}, R^{1D}, R^{1E}, R^{1F}, R^{1G}, R², R³, R⁴, R⁵, R⁶, R⁷, and R⁸ are defined according to the definitions provided herein for compounds of Formula I.

In some embodiments, the disclosure provides a compound of Formula Ie:

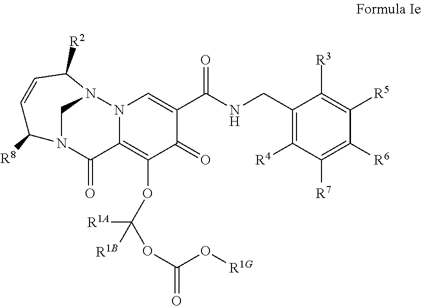

or a pharmaceutically acceptable salt thereof, wherein variables R^{1A}, R^{1B}, R^{1G}, R², R³, R⁴, R⁵, R⁶, R⁷, and R⁸ are defined according to the definitions provided herein for compounds of Formula I.

In some embodiments, the disclosure provides a compound of Formula If:

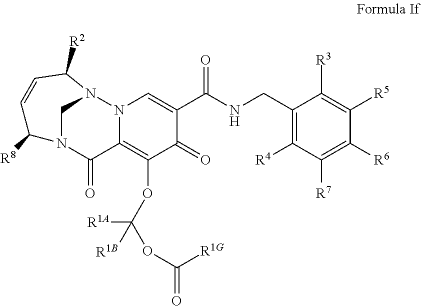

or a pharmaceutically acceptable salt thereof, wherein variables R^{1A}, R^{1B}, R^{1G}, R², R³, R⁴, R⁵, R⁶, R⁷, and R⁸ are defined according to the definitions provided herein for compounds of Formula I.

In some embodiments, a pharmaceutical composition is provided comprising a therapeutically effective amount of a compound of Formula I or a pharmaceutically acceptable salt thereof, and a pharmaceutically acceptable excipient.

In some embodiments, a kit or an article of manufacture is provided comprising a compound of Formula I, or a pharmaceutically acceptable salt thereof, and instructions for use.

In some embodiments, a method of treating an HIV infection in a human having or at risk of having the infection, by administering to the human a therapeutically effective amount of a compound of Formula I or a pharmaceutically acceptable salt thereof, or a pharmaceutical composition of a compound of Formula I, or a pharmaceutically acceptable salt thereof, is provided.

In some embodiments, use of a compound of Formula I, or a pharmaceutically acceptable salt thereof, or a pharmaceutical composition of a compound of Formula I or a pharmaceutically acceptable salt thereof, for treating an HIV infection in a human having or at risk of having the infection is provided.

In some embodiments, use of a compound of Formula I, or a pharmaceutically acceptable salt thereof, or a pharmaceutical composition of a compound of Formula I or a pharmaceutically acceptable salt thereof, in the manufacture of a medicament for treating an HIV infection in a human having or at risk of having the infection is provided.

In some embodiments, a compound of Formula I or a pharmaceutically acceptable salt thereof, or a pharmaceutical composition of a compound of formula I or a pharmaceutically acceptable salt thereof, for use in medical therapy is provided.

In some embodiments, a compound of Formula I or a pharmaceutically acceptable salt thereof, or a pharmaceutical composition of a compound of formula I or a pharmaceutically acceptable salt thereof, for use in treating an HIV infection is provided.

Other embodiments, objects, features, and advantages may be set forth in the detailed description of the embodiments that follows, and in part may be apparent from the description, or may be learned by practice, of the claimed embodiments. These objects and advantages may be realized and attained by the processes and compositions particularly pointed out in the description and claims thereof. The foregoing Summary has been made with the understanding that it is to be considered as a brief and general synopsis of some of the embodiments disclosed herein, is provided for the benefit and convenience of the reader, and is not intended to limit in any manner the scope, or range of equivalents, to which the appended claims are lawfully entitled.

### DETAILED DESCRIPTION

In the following description, certain specific details are set forth in order to provide a thorough understanding of various embodiments disclosed herein. However, one skilled in the art will understand that the embodiments disclosed herein may be practiced without these details. The description below of several embodiments is made with the understanding that the present disclosure is to be considered as an exemplification of the claimed subject matter, and is not intended to limit the appended claims to the specific embodiments illustrated. The headings used throughout this disclosure are provided for convenience only and are not to be construed to limit the claims in any way. Embodiments illustrated under any heading may be combined with embodiments illustrated under any other heading.

### I. DEFINITIONS

Unless the context requires otherwise, throughout the present disclosure and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment disclosed herein. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

“Amino” refers to the —NH₂ radical.

“Hydroxy” or “hydroxyl” refers to the —OH radical.

The term “C₁₋ₙalkyl” as used herein, wherein n is an integer, either alone or in combination with another radical, is intended to mean acyclic, straight or branched chain alkyl radicals containing from 1 to n carbon atoms. “C₁₋₆alkyl” includes, but is not limited to, methyl, ethyl, propyl (n-propyl), butyl (n-butyl), 1methylethyl (isopropyl), 1methylpropyl (sec-butyl), 2methylpropyl (iso-butyl), 1,1dimethylethyl (tertbutyl), pentyl and hexyl. The abbreviation Me denotes a methyl group; Et denotes an ethyl group, Pr denotes a propyl group, iPr denotes a 1-methylethyl group, Bu denotes a butyl group and tBu denotes a 1,1-dimethylethyl group.

“Alkyl” is hydrocarbon containing normal, secondary or tertiary atoms. For example, an alkyl group can have 1 to 20 carbon atoms (i.e., C₁₋₂₀alkyl), 1 to 10 carbon atoms (i.e., C₁₋₁₀alkyl), 1 to 8 carbon atoms (i.e., C₁₋₈alkyl)or 1 to 6 carbon atoms (i.e., C₁₋₆alkyl). Examples of suitable alkyl groups include, but are not limited to, methyl (Me, —CH₃), ethyl (Et, CH₂CH₃), 1-propyl (n-Pr, n-propyl, —CH₂CH₂CH₃), 2-propyl (i-Pr, ipropyl, CH(CH₃)₂), 1-butyl (n-Bu, n-butyl, —CH₂CH₂CH₂CH₃), 2methyl1-propyl (i-Bu, ibutyl, —CH₂CH(CH₃)₂), 2butyl (s-Bu, s-butyl, CH(CH₃)CH₂CH₃), 2-methyl-2-propyl (t-Bu, tbutyl, —C(CH₃)₃), 1-pentyl (n-pentyl, CH₂CH₂CH₂CH₂CH₃), 2-pentyl (CH(CH₃)CH₂CH₂CH₃), 3pentyl (CH(CH₂CH₃)₂), 2-methyl-2-butyl (C(CH₃)₂CH₂CH₃), 3methyl2-butyl (CH(CH₃)CH(CH₃)₂), 3methyl1butyl (CH₂CH₂CH(CH₃)₂), 2-methyl-1-butyl (CH₂CH(CH₃)CH₂CH₃), 1-hexyl (CH₂CH₂CH₂CH₂CH₂CH₃), 2-hexyl (CH(CH₃)CH₂CH₂CH₂CH₃), 3hexyl (CH(CH₂CH₃)(CH₂CH₂CH₃)), 2methyl-2-pentyl (C(CH₃)₂CH₂CH₂CH₃), 3methyl2pentyl (CH(CH₃)CH(CH₃)CH₂CH₃), 4-methyl-2-pentyl (CH(CH₃)CH₂CH(CH₃)₂), 3-methyl-3-pentyl (—C(CH₃)(CH₂CH₃)₂), 2methyl-3-pentyl (—CH(CH₂CH₃)CH(CH₃)₂), 2,3-dimethyl-2-butyl (C(CH₃)₂CH(CH₃)₂), 3,3dimethyl2butyl (—CH(CH₃)C(CH₃)₃, and octyl ((CH₂)₇CH₃). “Alkyl” also refers to a saturated, branched or straight chain hydrocarbon radical having two monovalent radical centers derived by the removal of two hydrogen atoms from the same or two different carbon atoms of a parent alkane. For example, an alkyl group can have 1 to 10 carbon atoms (i.e., C₁₋₁₀alkyl), or 1 to 6 carbon atoms (i.e., C₁₋₆alkyl) or 1-3 carbon atoms (i.e., C₁₋₃alkyl). Typical alkyl radicals include, but are not limited to, methylene (CH₂), 1,1ethyl (CH(CH₃)), 1,2ethyl (CH₂CH₂), 1,1-propyl (CH(CH₂CH₃)), 1,2-propyl (CH₂CH(CH₃)), 1,3propyl (CH₂CH₂CH₂), 1,4-butyl (CH₂CH₂CH₂CH₂), and the like.

The term “alkenyl” as used herein refers to a straight or branched hydrocarbon containing normal, secondary or tertiary carbon atoms with at least one site of unsaturation, i.e., a carbon-carbon, sp² double bond. For example, an alkenyl group can have 2 to 20 carbon atoms (i.e., C₂-C₂₀alkenyl, or C₂₋₂₀alkenyl), 2 to 8 carbon atoms (i.e., C₂-C₈alkenyl or C₂₋₈), or 2 to 6 carbon atoms (i.e., C₂-C₆alkenyl, or C₂₋₆alkenyl). Examples of suitable alkenyl groups include, but are not limited to, ethylene or vinyl (CH═CH₂), allyl (CH₂CH═CH₂), cyclopentenyl (C₅H₇), and 5-hexenyl (CH₂CH₂CH₂CH₂CH═CH₂).

The term “C₂₋ₙalkenyl”, as used herein, wherein n is an integer, either alone or in combination with another radical, is intended to mean an unsaturated, acyclic straight or branched chain radical containing two to n carbon atoms, at least two of which are bonded to each other by a double bond. Examples of such radicals include, but are not limited to, ethenyl (vinyl), 1propenyl, 2propenyl, and 1butenyl. Unless specified otherwise, the term “C₂₋ₙalkenyl” is understood to encompass individual stereoisomers where possible, including but not limited to (E) and (Z) isomers, and mixtures thereof. When a C₂₋ₙalkenyl group is substituted, it is understood to be substituted on any carbon atom thereof which would otherwise bear a hydrogen atom, unless specified otherwise, such that the substitution would give rise to a chemically stable compound, such as are recognized by those skilled in the art.

The term “halo” or “halogen” as used herein refers to fluoro, chloro, bromo and iodo.

The term “C₃₋ₘcycloalkyl” as used herein, wherein m is an integer, either alone or in combination with another radical, is intended to mean a cycloalkyl substituent containing from 3 to m carbon atoms and includes, but is not limited to, cyclopropyl, cyclobutyl, cyclopentyl, cyclohexyl and cycloheptyl. The term includes fully saturated as well as partially unsaturated rings.

It is to be understood that when a variable is substituted, for example, as described by the phrase “C₁₋₆alkyl, either alone or as part of a group, is optionally substituted”, the phrase means that the variable C₁₋₆alkyl can be substituted when it is alone and that it can also be substituted when the variable “C₁₋₆alkyl” is part of a larger group. Similarly, when stated, other variables can also be substituted “either alone or as part of a group.”

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers or axes of chirality and whose molecules are not mirror images of one another. Diastereomers typically have different physical properties, e.g., melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

The term “treatment” or “treating,” to the extent it relates to a disease or condition includes preventing the disease or condition from occurring, inhibiting or ameliorating the disease or condition (e.g., arresting or slowing its development), eliminating the disease or condition (e.g., causing regression or cure of the disease or condition), and/or relieving one or more symptoms of the disease or condition. In the case of HIV, treatment includes reducing the level of HIV viral load in a patient.

In some embodiments, the term “treatment” refers to the administration of a compound or composition according to the present invention to alleviate or eliminate symptoms of HIV infection and/or to reduce viral load in a patient. The term “treatment” also encompasses the administration of a compound or composition according to the present invention before the exposure of the individual to the virus, postexposure of the individual to the virus but before the appearance of symptoms of the disease, and/or prior to the detection of the virus in the blood, to prevent the appearance of symptoms of the disease and/or to prevent the virus from reaching detectible levels in the blood, and the administration of a compound or composition according to the present invention to prevent perinatal transmission of HIV from mother to baby, by administration to the mother before giving birth and to the child within the first days of life.

The term “solvate” refers to a crystalline solid containing amounts of a solvent incorporated within the crystal structure. As used herein, the term “solvate” includes hydrates.

The term “pharmaceutically acceptable” with respect to a substance as used herein means that substance which is, within the scope of sound medical judgment, suitable for use in contact with the tissues of humans and lower animals without undue toxicity, irritation, allergic response, and the like, commensurate with a reasonable benefit/risk ratio, and effective for the intended use when the substance is used in a pharmaceutical composition.

The term “pharmaceutically acceptable salt” as used herein is intended to mean a salt of a compound according to the invention which is, within the scope of sound medical judgment, suitable for use in contact with the tissues of humans and lower animals without undue toxicity, irritation, allergic response, and the like, commensurate with a reasonable benefit/risk ratio, generally water or oil-soluble or dispersible, and effective for their intended use. The term includes pharmaceutically-acceptable acid addition salts and pharmaceutically-acceptable base addition salts. Lists of suitable salts are found in, for example, S. M. Birge et al., J. Pharm. Sci., 1977, 66, pp. 1-19.

The term “pharmaceutically-acceptable acid addition salt” as used herein is intended to mean those salts which retain the biological effectiveness and properties of the free bases and which are not biologically or otherwise undesirable, formed with inorganic acids including but not limited to hydrochloric acid, hydrobromic acid, sulfuric acid, sulfamic acid, nitric acid, phosphoric acid and the like, and organic acids including but not limited to acetic acid, trifluoroacetic acid, adipic acid, ascorbic acid, aspartic acid, benzenesulfonic acid, benzoic acid, butyric acid, camphoric acid, camphorsulfonic acid, cinnamic acid, citric acid, digluconic acid, ethanesulfonic acid, glutamic acid, glycolic acid, glycerophosphoric acid, hemisulfic acid, hexanoic acid, formic acid, fumaric acid, 2-hydroxyethanesulfonic acid (isethionic acid), lactic acid, hydroxymaleic acid, malic acid, malonic acid, mandelic acid, mesitylenesulfonic acid, methanesulfonic acid, naphthalenesulfonic acid, nicotinic acid, 2-naphthalenesulfonic acid, oxalic acid, pamoic acid, pectinic acid, phenylacetic acid, 3-phenylpropionic acid, pivalic acid, propionic acid, pyruvic acid, salicylic acid, stearic acid, succinic acid, sulfanilic acid, tartaric acid, p-toluenesulfonic acid, undecanoic acid and the like.

The term “pharmaceutically-acceptable base addition salt” as used herein is intended to mean those salts which retain the biological effectiveness and properties of the free acids and which are not biologically or otherwise undesirable, formed with inorganic bases including but not limited to ammonia or the hydroxide, carbonate, or bicarbonate of ammonium or a metal cation such as sodium, potassium, lithium, calcium, magnesium, iron, zinc, copper, manganese, aluminum and the like. Particularly preferred are the ammonium, potassium, sodium, calcium, and magnesium salts. Salts derived from pharmaceutically-acceptable organic nontoxic bases include but are not limited to salts of primary, secondary, and tertiary amines, quaternary amine compounds, substituted amines including naturally occurring substituted amines, cyclic amines and basic ion-exchange resins, such as methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purines, piperazine, piperidine, N-ethylpiperidine, tetramethylammonium compounds, tetraethylammonium compounds, pyridine, N,N-dimethylaniline, N-methylpiperidine, N-methylmorpholine, dicyclohexylamine, dibenzylamine, N,N-dibenzylphenethylamine, 1-ephenamine, N,N′-dibenzylethylenediamine, polyamine resins and the like. Particularly preferred organic nontoxic bases are isopropylamine, diethylamine, ethanolamine, trimethylamine, dicyclohexylamine, choline, and caffeine.

The term “antiviral agent” as used herein is intended to mean an agent that is effective to inhibit the formation and/or replication of a virus in a mammal, including but not limited to agents that interfere with either host or viral mechanisms necessary for the formation and/or replication of a virus in a mammal. The term “antiviral agent” includes, for example, an HIV integrase catalytic site inhibitor selected from the group consisting: raltegravir (ISENTRESS®; Merck); elvitegravir (Gilead); soltegravir (GSK; ViiV); GSK 1265744 (GSK; ViiV) and dolutegravir; an HIV nucleoside reverse transcriptase inhibitor selected from the group consisting of: abacavir (ZIAGEN®; GSK); didanosine (VIDEX®; BMS); tenofovir (VIREAD®; Gilead); emtricitabine (EMTRIVA®; Gilead); lamivudine (EPIVIR®; GSK/Shire); stavudine (ZERIT®; BMS); zidovudine (RETROVIR®; GSK); elvucitabine (Achillion); and festinavir (Oncolys); an HIV non-nucleoside reverse transcriptase inhibitor selected from the group consisting of: nevirapine (VIRAMUNE®; BI); efavirenz (SUSTIVA®; BMS); etravirine (INTELENCE®; J&J); rilpivirine (TMC278, R278474; J&J); fosdevirine (GSK/ViiV); and lersivirine (Pfizer/ViiV); an HIV protease inhibitor selected from the group consisting of: atazanavir (REYATAZ®; BMS); darunavir (PREZISTA®; J&J); indinavir (CRIXIVAN®; Merck); lopinavir (KELETRA®; Abbott); nelfinavir (VIRACEPT®; Pfizer); saquinavir (INVIRASE®; Hoffmann-LaRoche); tipranavir (APTIVUS®; BI); ritonavir (NORVIR®; Abbott); and fosamprenavir (LEXIVA®; GSK/Vertex); an HIV entry inhibitor selected from: maraviroc (SELZENTRY®; Pfizer); enfuvirtide (FUZEON®; Trimeris); and BMS-663068 (BMS); and an HIV maturation inhibitor selected from: bevirimat (Myriad Genetics).

The term “inhibitor of HIV replication” as used herein is intended to mean an agent capable of reducing or eliminating the ability of HIV to replicate in a host cell, whether in vitro, ex vivo or in vivo.

The term “substituent”, as used herein and unless specified otherwise, is intended to mean an atom, radical or group which may be bonded to a carbon atom, a heteroatom or any other atom which may form part of a molecule or fragment thereof, which would otherwise be bonded to at least one hydrogen atom. Substituents contemplated in the context of a specific molecule or fragment thereof are those which give rise to chemically stable compounds, such as are recognized by those skilled in the art.

The term “heteroatom” as used herein is intended to mean O, S or N.

The terms “O—C₁₋ₙalkyl” or “C₁₋ₙ alkoxy” as used herein interchangeably, wherein n is an integer, either alone or in combination with another radical, is intended to mean an oxygen atom further bonded to an alkyl radical having 1 to n carbon atoms as defined above. Examples of C₁₋ₙalkoxy include but are not limited to methoxy (CH₃O—), ethoxy (CH₃CH₂O—), propoxy (CH₃CH₂CH₂O—), 1-methylethoxy (iso-propoxy; (CH₃)₂CHO) and 1,1-dimethylethoxy (tert-butoxy; (CH₃)₃CO). When an C₁₋ₙalkoxy is substituted, it is understood to be substituted on the alkyl portion thereof, such that the substitution would give rise to a chemically stable compound, such as are recognized by those skilled in the art.

The term “mammal” as used herein is intended to encompass humans, as well as non-human mammals which are susceptible to infection by HIV. Non-human mammals include but are not limited to domestic animals, such as cows, pigs, horses, dogs, cats, rabbits, rats and mice, and non-domestic animals.

The embodiments disclosed herein are also meant to encompass all pharmaceutically acceptable compounds of Formula I being isotopically-labeled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. In certain embodiments, these radiolabeled compounds are useful to help determine or measure the effectiveness of the compounds, by characterizing, for example, the site or mode of action, or binding affinity to pharmacologically important site of action. Certain isotopically-labeled compounds of Formula I, for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

In certain embodiments, substitution with heavier isotopes such as deuterium, i.e., ²H, may afford certain therapeutic advantages resulting from greater metabolic stability. For example, in vivo half-life may increase or dosage requirements may be reduced. Thus, heavier isotopes may be preferred in some circumstances.

Substitution with positron emitting isotopes, such as ¹¹C, ¹⁸F, ¹⁵O, and ¹³N, can be useful in Positron Emission Topography (PET) studies for examining substrate receptor occupancy. Isotopically-labeled compounds of the compounds disclosed herein can be prepared by techniques known to those skilled in the art or by processes analogous to those described in the Examples as set out below using an appropriate isotopically-labeled reagent in place of the non-labeled reagent previously employed.

The methods, compositions, kits and articles of manufacture provided herein use or include compounds (e.g., a compound of Formula I) or pharmaceutically acceptable salts thereof, in which from 1 to n hydrogen atoms attached to a carbon atom may be replaced by a deuterium atom or D, in which n is the number of hydrogen atoms in the molecule. As known in the art, the deuterium atom is a non-radioactive isotope of the hydrogen atom. Such compounds increase resistance to metabolism, and thus are useful for increasing the half-life of compounds or pharmaceutically acceptable salts thereof, when administered to a mammal. See, e.g., Foster, “Deuterium Isotope Effects in Studies of Drug Metabolism”, Trends Pharmacol. Sci., 5(12):524-527 (1984). Such compounds can be synthesized by means known in the art, for example by employing starting materials in which one or more hydrogen atoms have been replaced by deuterium.

The embodiments disclosed herein are also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the embodiments disclosed herein include compounds produced by a process comprising administering a compound according to the embodiments disclosed herein to a mammal for a period of time sufficient to yield a metabolic product thereof. Such products are typically identified by administering a radiolabeled compound according to the embodiments disclosed herein in a detectable dose to an animal, such as rat, mouse, guinea pig, monkey, or to human, allowing sufficient time for metabolism to occur, and isolating its conversion products from the urine, blood or other biological samples.

The compounds disclosed herein, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic, scalemic, and optically pure forms. Optically active (+) and (−), (R)- and (S)-, or (D)- and (L)-isomers may be prepared using chiral synthons or chiral reagents, or resolved using methods such as chromatography and fractional crystallization. Techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

Optional” or “optionally” means that the subsequently described event or circumstances may or may not occur, and that the description includes instances where said event or circumstance occurs and instances in which it does not. For example, “optionally substituted heterocyclyl” means that the heterocyclyl radical may or may not be substituted and that the description includes both substituted heterocyclyl radicals and heterocyclyl radicals having no substitution.

### II. COMPOUNDS

In some embodiments, the disclosure provides a compound of Formula I:

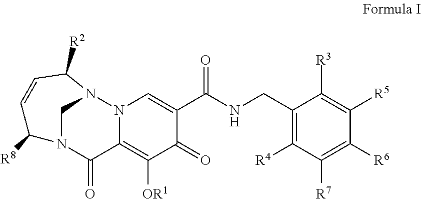

or a pharmaceutically acceptable salt thereof, wherein:
R¹ is —(CR^{1A}R^{1B}O)ₘCO(O—CR^{1C}R^{1D}—CR^{1E}R^{1F})ₙ—(O)ₚR^{1G};
   wherein m is 0 or 1;
   n is an integer between 0 and 10, inclusive;
   p is 0 or 1;
   R^{1A} is H or C₁₋₃alkyl;
   R^{1B} is H or C₁₋₃alkyl;
   each R^{1C} is independently H or C₁₋₃alkyl;
   each R^{1D} is independently H or C₁₋₃alkyl;
   each R^{1E} is independently H or C₁₋₃alkyl;
   each R^{1F} is independently H or C₁₋₃alkyl;
   R^{1G} is C₃₋₆cycloalkyl, C₁₋₂₀alkyl, C₂₋₂₀alkenyl, or a 3 to 8 membered heterocyclyl comprising 1, 2, or 3 heteroatoms selected independently from N, O, and S; wherein the C₃₋₆cycloalkyl, C₁₋₂₀alkyl, C₂₋₂₀alkenyl, or 3 to 8 membered heterocyclyl is optionally substituted with a COOH, C₃₋₆cycloalkyl, C₁₋₃alkoxy, or —NHCOOR^{1H};
   wherein R^{1H} is C₁₋₃alkyl;
R² is H or C₁₋₃alkyl;
each R³, R⁴, R⁵, R⁶ and R⁷ is independently H or halo; and
R⁸ is H or C₁₋₃alkyl.

In some embodiments, of the compound of Formula I, or the pharmaceutically acceptable salt thereof, wherein
R¹ is —(CR^{1A}R^{1B}O)ₘCO(O—CR^{1C}R^{1D}—CR^{1E}R^{1F})ₙ—(O)ₚR^{1G};
   wherein m is 0 or 1;
   n is an integer between 0 and 10, inclusive;
   p is 0 or 1;
   R^{1A} is H or C₁₋₃alkyl;
   R^{1B} is H or C₁₋₃alkyl;
   each R^{1C} is independently H or C₁₋₃alkyl;
   each R^{1D} is independently H or C₁₋₃alkyl;
   each R^{1E} is independently H or C₁₋₃alkyl;
   each R^{1F} is independently H or C₁₋₃alkyl;
   R^{1G} is C₃₋₆cycloalkyl, C₁₋₂₀alkyl, C₂₋₂₀alkenyl, or a 3 to 8 membered heterocyclyl comprising 1, 2, or 3 heteroatoms selected independently from N, O, and S; wherein the C₃₋₆cycloalkyl, C₁₋₂₀alkyl, C₂₋₂₀alkenyl, or 3 to 8 membered heterocyclyl is optionally substituted with a COOH, C₃₋₆cycloalkyl, or C₁₋₃alkoxy;
R² is H or C₁₋₃alkyl;
each R³, R⁴, R⁵, R⁶ and R⁷ is independently H or halo; and
R⁸ is H or C₁₋₃alkyl.

In some embodiments, of the compound of Formula I, or a pharmaceutically acceptable salt thereof, wherein:
R¹ is —(CR^{1A}R^{1B}O)ₘCO(O—CR^{1C}R^{1D}—CR^{1E}R^{1F})ₙ—(O)ₚR^{1G};
   wherein m is 0 or 1;
   n is 0, 1, 2, or 3;
   p is 0 or 1;
   R^{1A} is H or C₁₋₃alkyl;
   R^{1B} is H or C₁₋₃alkyl;
   each R^{1C} is independently H or C₁₋₃alkyl;
   each R^{1D} is independently H or C₁₋₃alkyl;
   each R^{1E} is independently H or C₁₋₃alkyl;
   each R^{1F} is independently H or C₁₋₃alkyl;
   R^{1G} is C₃₋₆cycloalkyl, C₁₋₂₀alkyl, C₂₋₂₀alkenyl, or a 3 to 8 membered heterocyclyl comprising 1, 2, or 3 heteroatoms selected independently from N, O, and S; wherein the C₃₋₆cycloalkyl, C₁₋₂₀alkyl, C₂₋₂₀alkenyl, or 3 to 8 membered heterocyclyl is optionally substituted with a COOH, C₃₋₆cycloalkyl, C₁₋₃alkoxy, or —NHCOOR^{1H};
   wherein R^{1H} is C₁₋₃alkyl;
R² is H or C₁₋₃alkyl;
each R³, R⁴, R⁵, R⁶ and R⁷ is independently H or halo; and
R⁸ is H or C₁₋₃alkyl.

In some embodiments, of the compound of Formula I, or a pharmaceutically acceptable salt thereof, wherein:
R¹ is —(CR^{1A}R^{1B}O)ₘCO(O—CR^{1C}R^{1D}—CR^{1E}R^{1F})ₙ—(O)ₚR^{1G};
   wherein m is 0 or 1;
   n is 0, 1, 2, or 3;
   p is 0 or 1;
   R^{1A} is H or C₁₋₃alkyl;
   R^{1B} is H or C₁₋₃alkyl;
   each R^{1C} is independently H or C₁₋₃alkyl;
   each R^{1D} is independently H or C₁₋₃alkyl;
   each R^{1E} is independently H or C₁₋₃alkyl;
   each R^{1F} is independently H or C₁₋₃alkyl;
   R^{1G} is C₃₋₆cycloalkyl, C₁₋₂₀alkyl, C₂₋₂₀alkenyl, or a 3 to 8 membered heterocyclyl comprising 1, 2, or 3 heteroatoms selected independently from N, O, and S; wherein the C₃₋₆cycloalkyl, C₁₋₂₀alkyl, C₂₋₂₀alkenyl, or 3 to 8 membered heterocyclyl is optionally substituted with a COOH, C₃₋₆cycloalkyl, or C₁₋₃alkoxy;
R² is H or C₁₋₃alkyl;
each R³, R⁴, R⁵, R⁶ and R⁷ is independently H or halo; and
R⁸ is H or C₁₋₃alkyl.

In some embodiments of the compound of Formula I, or the pharmaceutically acceptable salt thereof, m is 0. In some embodiments, m is 1.

In some embodiments of the compound of Formula I, or the pharmaceutically acceptable salt thereof, n is an integer between 0 and 10, inclusive (i.e., n is 0, 1, 2, 3, 4, 5, 6, 7, 8, 9, or 10). In some embodiments, n is an integer between 0 and 7, inclusive (i.e., n is 0, 1, 2, 3, 4, 5, 6, or 7). In some embodiments, n is an integer between 1 and 7, inclusive (i.e., n is 1, 2, 3, 4, 5, 6, or 7). In some embodiments, n is 1, 2, or 3.

In some embodiments of the compound of Formula I, or the pharmaceutically acceptable salt thereof, n is 0, 1, 2, or 3. In some embodiments, n is 0. In some embodiments, n is 1. In some embodiments, n is 2. In some embodiments, n is 3.

In some embodiments of the compound of Formula I, or the pharmaceutically acceptable salt thereof, p is 0. In some embodiments, p is 1.

In some embodiments of the compound of Formula I, or the pharmaceutically acceptable salt thereof, m is 0, n is 0, and p is 0. In some embodiments, m is 0, n is 1, and p is 0. In some embodiments, m is 0, n is 0, and p is 1. In some embodiments, m is 0, n is 1, and p is 1. In some embodiments, m is 0, n is 2, and p is 0. In some embodiments, m is 0, n is 2, and p is 1.

In some embodiments of the compound of Formula I, or the pharmaceutically acceptable salt thereof, m is 1, n is 0, and p is 0. In some embodiments, m is 1, n is 1, and p is 0. In some embodiments, m is 1, n is 0, and p is 1. In some embodiments, m is 1, n is 1, and p is 1. In some embodiments, m is 1, n is 2, and p is 0. In some embodiments, m is 1, n is 2, and p is 1.

In some embodiment of the compound of Formula I, or the pharmaceutically acceptable salt thereof, R¹ is —COR^{1G}. In some embodiments, R¹ is selected from the group consisting of —COCH₃, —COCH₂CH₃, —CO(CH₂)₂CH₃, —CO(CH₂)₃CH₃, —CO(CH₂)₄CH₃, —CO(CH₂)₅CH₃, —CO(CH₂)₆CH₃, —CO(CH₂)₇CH₃, —CO(CH₂)₈CH₃, —CO(CH₂)₉CH₃, —CO(CH₂)₁₀CH₃, —CO(CH₂)₁₁CH₃, —CO(CH₂)₁₂CH₃, —CO(CH₂)₁₃CH₃, —CO(CH₂)₁₄CH₃, —CO(CH₂)₁₅CH₃, —CO(CH₂)₁₆CH₃, —CO(CH₂)₁₇CH₃, —COCH(CH₃)₂, —COCH₂CH(CH₃)₂, —CO(CH₂)₂CH(CH₃)₂, —CO(CH₂)₃CH(CH₃)₂, —CO(CH₂)₄CH(CH₃)₂, —CO(CH₂)₅CH(CH₃)₂, —CO(CH₂)₆CH(CH₃)₂, —CO(CH₂)₇CH(CH₃)₂, —CO(CH₂)₇CH═CH(CH₂)₃CH₃, —CO(CH₂)₃(CH═CHCH₂)₅CH₃, —CO(CH₂)₇(CH═CHCH₂)₂CH₃CH₃, —COCH₂CH₂-cyclopentyl, —CO-cyclopentyl, —CO(CH₂)₅OCH₃, —CO(CH₂)₃COOH, —CO(CH₂)₅COOH, —CO(CH₂)₁₆COOH, and

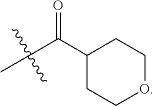 [SMILES: *C(=O)C1CCOCC1]

In some embodiments, R¹ is selected from the group consisting of —COCH₃, —COCH₂CH₃, —CO(CH₂)₂CH₃, —CO(CH₂)₃CH₃, —CO(CH₂)₄CH₃, —CO(CH₂)₅CH₃, —CO(CH₂)₆CH₃, —CO(CH₂)₇CH₃, —CO(CH₂)₈CH₃, —CO(CH₂)₁₀CH₃, —CO(CH₂)₁₂CH₃, —CO(CH₂)₁₄CH₃, —CO(CH₂)₁₆CH₃, —COCH(CH₃)₂, —COCH₂CH(CH₃)₂, —CO(CH₂)₂CH(CH₃)₂, —CO(CH₂)₃CH(CH₃)₂, —CO(CH₂)₄CH(CH₃)₂, —CO(CH₂)₅CH(CH₃)₂, —CO(CH₂)₆CH(CH₃)₂, —CO(CH₂)₇CH═CH(CH₂)₃CH₃, —CO(CH₂)₃(CH═CHCH₂)₅CH₃, —CO(CH₂)₇(CH═CHCH₂)₂CH₃CH₃, —COCH₂CH₂-cyclopentyl, —CO-cyclopentyl, —CO(CH₂)₅OCH₃, —CO(CH₂)₃COOH, —CO(CH₂)₅COOH, and —CO(CH₂)₁₆COOH.

In some embodiments of the compound of Formula I, or the pharmaceutically acceptable salt thereof, R¹ is —CH₂OCOR^{1G}. In some embodiments, R¹ is selected from the group consisting of —CH₂OCOCH₃, —CH₂OCOCH₂CH₃, —CH₂OCO(CH₂)₂CH₃, —CH₂OCO(CH₂)₃CH₃, —CH₂OCO(CH₂)₄CH₃, —CH₂OCO(CH₂)₅CH₃, —CH₂OCO(CH₂)₆CH₃, —CH₂OCO(CH₂)₇CH₃, —CH₂OCO(CH₂)₈CH₃, —CH₂OCO(CH₂)₁₀CH₃, —CH₂OCO(CH₂)₁₂CH₃, —CH₂OCO(CH₂)₁₄CH₃, —CH₂OCO(CH₂)₁₆CH₃, —CH₂OCOCH(CH₃)₂, —CH₂OCOCH₂CH(CH₃)₂, —CH₂OCO(CH₂)₂CH(CH₃)₂, —CH₂OCO(CH₂)₃CH(CH₃)₂, —CH₂OCO(CH₂)₄CH(CH₃)₂, —CH₂OCO(CH₂)₅CH(CH₃)₂, —CH₂OCO(CH₂)₆CH(CH₃)₂, —CH₂OCO(CH₂)₇CH(CH₃)₂, —CH₂OCO(CH₂)₇CH═CH(CH₂)₃CH₃, —CH₂OCO(CH₂)₃(CH═CHCH₂)₅CH₃, —CH₂OCO(CH₂)₇(CH═CHCH₂)₂CH₃CH₃, —CH₂OCOCH₂CH₂-cyclopentyl, —CH₂OCO-cyclopentyl, —CH₂OCO(CH₂)₅OCH₃, —CH₂OCO(CH₂)₃COOH, —CH₂OCO(CH₂)₅COOH, —CH₂OCO(CH₂)₁₆COOH, and

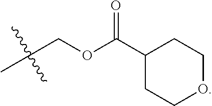 [SMILES: *COC(=O)C1CCOCC1]

In some embodiments, R¹ is selected from the group consisting of —CH₂OCOCH₃, —CH₂OCOCH₂CH₃, —CH₂OCO(CH₂)₂CH₃, —CH₂OCO(CH₂)₃CH₃, —CH₂OCO(CH₂)₄CH₃, —CH₂OCO(CH₂)₅CH₃, —CH₂OCO(CH₂)₆CH₃, —CH₂OCO(CH₂)₇CH₃, —CH₂OCO(CH₂)₈CH₃, —CH₂OCO(CH₂)₁₀CH₃, —CH₂OCO(CH₂)₁₂CH₃, —CH₂OCO(CH₂)₁₄CH₃, —CH₂OCO(CH₂)₁₆CH₃, —CH₂OCOCH(CH₃)₂, —CH₂OCOCH₂CH(CH₃)₂, —CH₂OCO(CH₂)₂CH(CH₃)₂, —CH₂OCO(CH₂)₃CH(CH₃)₂, —CH₂OCO(CH₂)₄CH(CH₃)₂, —CH₂OCO(CH₂)₅CH(CH₃)₂, and —CH₂OCO(CH₂)₆CH(CH₃)₂.

In some embodiments of the compound of Formula I, or the pharmaceutically acceptable salt thereof, R¹ is —COOR^{1G}. In some embodiments, R¹ is selected from the group consisting of —COOCH₃, —COOCH₂CH₃, —COO(CH₂)₂CH₃, —COO(CH₂)₃CH₃, —COO(CH₂)₄CH₃, —COO(CH₂)₅CH₃, —COO(CH₂)₆CH₃, —COO(CH₂)₇CH₃, —COO(CH₂)₈CH₃, —COO(CH₂)₉CH₃, —COO(CH₂)₁₀CH₃, —COO(CH₂)₁₁CH₃, —COO(CH₂)₁₂CH₃, —COO(CH₂)₁₃CH₃, —COO(CH₂)₁₄CH₃, —COO(CH₂)₁₅CH₃, —COO(CH₂)₁₆CH₃, —COO(CH₂)₁₇CH₃, —COOCH(CH₃)₂, —COOCH₂CH(CH₃)₂, —COO(CH₂)₂CH(CH₃)₂, —COO(CH₂)₃CH(CH₃)₂, —COO(CH₂)₄CH(CH₃)₂, —COO(CH₂)₅CH(CH₃)₂, —COO(CH₂)₆CH(CH₃)₂, —COO(CH₂)₇CH(CH₃)₂, —COO(CH₂CH₂O)₂CH₃, —COO(CH₂)₇CH═CH(CH₂)₃CH₃, —COO(CH₂)₃(CH═CHCH₂)₅CH₃, —COO(CH₂)₇(CH═CHCH₂)₂CH₃CH₃, —COOCH₂CH₂-cyclopentyl, —COO-cyclopentyl, —COO(CH₂)₅OCH₃, —COO(CH₂)₃COOH, —COO(CH₂)₅COOH, —COO(CH₂)₁₆COOH, and

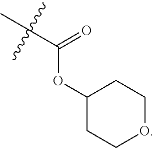 [SMILES: *C(=O)OC1CCOCC1]

In some embodiments, R¹ is selected from the group consisting of —COOCH₃, —COOCH₂CH₃, —COO(CH₂)₂CH₃, —COO(CH₂)₃CH₃, —COO(CH₂)₄CH₃, —COO(CH₂)₅CH₃, —COO(CH₂)₆CH₃, —COO(CH₂)₇CH₃, —COO(CH₂)₈CH₃, —COO(CH₂)₉CH₃, —COO(CH₂)₁₁CH₃, —COO(CH₂)₁₃CH₃, —COO(CH₂)₁₅CH₃, —COO(CH₂)₁₇CH₃, —COOCH(CH₃)₂, —COOCH₂CH(CH₃)₂, —COO(CH₂)₂CH(CH₃)₂, —COO(CH₂)₃CH(CH₃)₂, —COO(CH₂)₄CH(CH₃)₂, —COO(CH₂)₅CH(CH₃)₂, —COO(CH₂)₆CH(CH₃)₂, —COO(CH₂)₇CH(CH₃)₂, —COO(CH₂CH₂O)₂CH₃, and

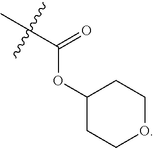 [SMILES: *C(=O)OC1CCOCC1]

In some embodiments of the compound of Formula I, or the pharmaceutically acceptable salt thereof, R¹ is —CH₂OCOOR^{1G} In some embodiments, R¹ is selected from the group consisting of —CH₂OCOOCH₃, —CH₂OCOOCH₂CH₃, —CH₂OCOO(CH₂)₂CH₃, —CH₂OCOO(CH₂)₃CH₃, —CH₂OCOO(CH₂)₄CH₃, —CH₂OCOO(CH₂)₅CH₃, —CH₂OCOO(CH₂)₆CH₃, —CH₂OCOO(CH₂)₇CH₃, —CH₂OCOO(CH₂)₈CH₃, —CH₂OCOO(CH₂)₉CH₃, —CH₂OCOO(CH₂)₁₀CH₃, —CH₂OCOO(CH₂)₁₁CH₃, —CH₂OCOO(CH₂)₁₂CH₃, —CH₂OCOO(CH₂)₁₃CH₃, —CH₂OCOO(CH₂)₁₄CH₃, —CH₂OCOO(CH₂)₁₅CH₃, —CH₂OCOO(CH₂)₁₆CH₃, —CH₂OCOO(CH₂)₁₇CH₃, —CH₂OCOOCH(CH₃)₂, —CH₂OCOOCH₂CH(CH₃)₂, —CH₂OCOO(CH₂)₂CH(CH₃)₂, —CH₂OCOO(CH₂)₃CH(CH₃)₂, —CH₂OCOO(CH₂)₄CH(CH₃)₂, —CH₂OCOO(CH₂)₅CH(CH₃)₂, —CH₂OCOO(CH₂)₆CH(CH₃)₂, —CH₂OCOO(CH₂)₇CH(CH₃)₂, —CH₂OCOO(CH₂)₂OCH₃, —CH₂OCOO(CH₂CH₂O)₂CH₃, —CH(CH₃)OCOOCH(CH₃)₂, —CH₂OCOO(CH₂)₇CH═CH(CH₂)₃CH₃, —CH₂OCOO(CH₂)₃(CH═CHCH₂)₅CH₃, —CH₂OCOO(CH₂)₇(CH═CHCH₂)₂CH₃CH₃, —CH₂OCOOCH₂CH₂-cyclopentyl, —CH₂OCOO-cyclopentyl, —CH₂OCOO(CH₂)₅OCH₃, —CH₂OCOO(CH₂)₃COOH, —CH₂OCOO(CH₂)₅COOH, —CH₂OCOO(CH₂)₁₆COOH, and

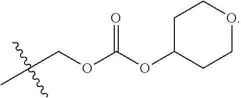 [SMILES: *COC(=O)OC1CCOCC1]

In some embodiments, R¹ is selected from the group consisting of —CH₂OCOOCH₃, —CH₂OCOOCH₂CH₃, —CH₂OCOO(CH₂)₂CH₃, —CH₂OCOO(CH₂)₃CH₃, —CH₂OCOO(CH₂)₄CH₃, —CH₂OCOO(CH₂)₅CH₃, —CH₂OCOO(CH₂)₆CH₃, —CH₂OCOO(CH₂)₇CH₃, —CH₂OCOO(CH₂)₈CH₃, —CH₂OCOO(CH₂)₉CH₃, —CH₂OCOO(CH₂)₁₁CH₃, —CH₂OCOO(CH₂)₁₃CH₃, —CH₂OCOO(CH₂)₁₅CH₃, —CH₂OCOO(CH₂)₁₇CH₃, —CH₂OCOOCH(CH₃)₂, —CH₂OCOOCH₂CH(CH₃)₂, —CH₂OCOO(CH₂)₂CH(CH₃)₂, —CH₂OCOO(CH₂)₃CH(CH₃)₂, —CH₂OCOO(CH₂)₄CH(CH₃)₂, —CH₂OCOO(CH₂)₅CH(CH₃)₂, —CH₂OCOO(CH₂)₆CH(CH₃)₂, —CH₂OCOO(CH₂)₇CH(CH₃)₂, —CH₂OCOO(CH₂)₂OCH₃, —CH₂OCOO(CH₂CH₂O)₂CH₃, and —CH(CH₃)OCOOCH(CH₃)₂.

In some embodiments of the compound of Formula I, or the pharmaceutically acceptable salt thereof, the compound has a Formula Ia:

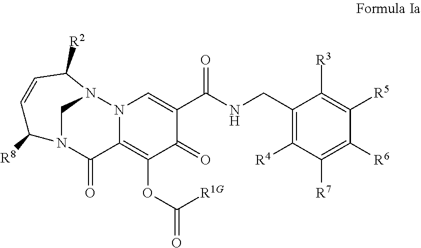

wherein variables R^{1G}, R², R³, R⁴, R⁵, R⁶, R⁷, and R⁸ are defined according to the definitions provided herein for compounds of Formula I.

In some embodiments of the compound of Formula I, or the pharmaceutically acceptable salt thereof, the compound has a Formula Ib:

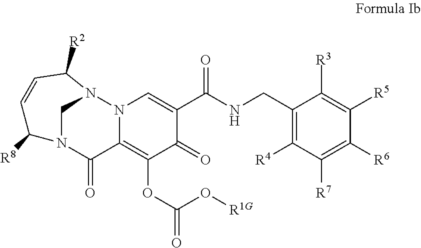

wherein variables R^{1G}, R², R³, R⁴, R⁵, R⁶, R⁷, and R⁸ are defined according to the definitions provided herein for compounds of Formula I.

In some embodiments of the compound of Formula I, or the pharmaceutically acceptable salt thereof, the compound has a Formula Ic:

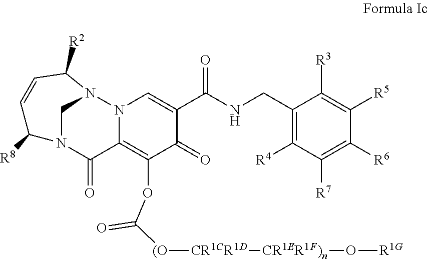

wherein n is an integer between 1 and 7, inclusive; and variables R^{1C}, R^{1D}, R^{1E}, R^{1F}, R^{1G}, R², R³, R⁴, R⁵, R⁶, R⁷, and R⁸ are defined according to the definitions provided herein for compounds of Formula I.

In some embodiments, of the compound of Formula Ic, or the pharmaceutically acceptable salt thereof, n is 1, 2, or 3.

In some embodiments of the compound of Formula I, or the pharmaceutically acceptable salt thereof, the compound has a Formula Id:

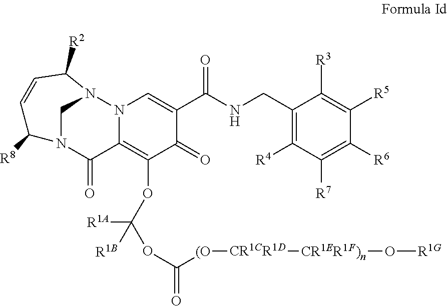

wherein n is 0, 1, 2 or 3; and variables R^{1A}, R^{1B}, R^{1C}, R^{1D}, R^{1E}, R^{1F}, R^{1G}, R², R³, R, R⁶, R⁷, and R⁸ are defined according to the definitions provided herein for compounds of Formula I.

In some embodiments of the compound of Formula Id, or the pharmaceutically acceptable salt thereof, n is 0, 1, 2, or 3. In some embodiments of the compound of Formula Id, or the pharmaceutically acceptable salt thereof, n is 1, 2, or 3.

In some embodiments of the compound of Formula I, or the pharmaceutically acceptable salt thereof, the compound has a Formula Ie:

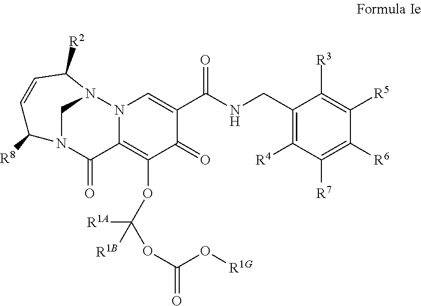

wherein variables R^{1A}, R^{1B}, R^{1G}, R², R³, R⁴, R⁵, R⁶, R⁷, and R⁸ are defined according to the definitions provided herein for compounds of Formula I.

In some embodiments of the compound of Formula I, or the pharmaceutically acceptable salt thereof, the compound has a Formula If:

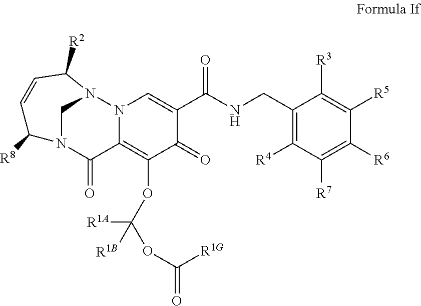

wherein variables R^{1A}, R^{1B}, R^{1G}, R², R³, R⁴, R⁵, R⁶, R⁷, and R⁸ are defined according to the definitions provided herein for compounds of Formula I.

In some embodiments of the compounds of Formula I, Id, Ie, and If, R^{1A} is H. In some embodiments, R^{1B} is H. In some embodiments, R^{1A} is H and R^{1B} is H. In some embodiments, R^{1A} is H and R^{1B} is C₁₋₃alkyl. In some embodiments, R^{1A} is H and R^{1B} is methyl.

In some embodiments of the compounds of Formula I, Id, Ie, and If, R^{1A} is C₁₋₃alkyl. In some embodiments, R^{1A} is methyl. In some embodiments, each R^{1A} is methyl and R^{1B} is H.

In some embodiments of the compounds of Formula I, Id, Ie, and If, R^{1A} is C₁₋₃alkyl and R^{1B} is C₁₋₃alkyl. In some embodiments, R^{1A} is methyl and R^{1B} is methyl.

In some embodiments of the compounds of Formula I, Ic, and Id, each Ric and each R^{1D} is H. In some embodiments, each R^{1C} is methyl and each R^{1D} is H. In some embodiments, each R^{1E} and each R^{1F} is H. In some embodiments, each R^{1E} is methyl and each R^{1F} is H.

In some embodiments of the compounds of Formula I, Ic, and Id, each R^{1C} is H. In some embodiments, each R^{1D} is H. In some embodiments, each R^{1E} is H. In some embodiments, each R^{1F} is H. In some embodiments, each R^{1C} is H, each R^{1D} is H, each R^{1E} is H, and each R^{1F} is H. In some embodiments, each R^{1C} is methyl, each R^{1D} is H, each R^{1E} is H, and each R^{1F} is H. In some embodiments, each Ric is methyl, each R^{1D} is H, each R^{1E} is methyl, and each R^{1F} is H.

In some embodiments of the compounds of Formula I, Ia, Ib, Ic, Id, Ie, and If, or the pharmaceutically acceptable salt thereof, R^{1G} is C₃₋₆cycloalkyl, C₁₋₂₀alkyl, C₂₋₂₀alkenyl, or a 3 to 8 membered heterocyclyl comprising 1, 2, or 3 heteroatoms selected independently from N, O, and S; wherein the C₃₋₆cycloalkyl, C₁₋₂₀alkyl, C₂₋₂₀alkenyl, or 3 to 8 membered heterocyclyl is optionally substituted with a COOH, C₃₋₆cycloalkyl, C₁₋₃alkoxy, or —NHCOOR^{1H}; wherein R^{1H} is C₁₋₃alkyl. In some embodiments, R^{1G} is C₃₋₆cycloalkyl, C₁₋₂₀alkyl, C₂₋₂₀alkenyl, or a 3 to 8 membered heterocyclyl comprising 1, 2, or 3 heteroatoms selected independently from N, O, and S; wherein the C₃₋₆cycloalkyl, C₁₋₂₀alkyl, C₂₋₂₀alkenyl, or 3 to 8 membered heterocyclyl is optionally substituted with a COOH, C₃₋₆cycloalkyl, C₁₋₃alkoxy, or —NHCOOR^{1H}; wherein R^{1H} is methyl.

In some embodiments of the compounds of Formula I, Ia, Ib, Ic, Id, Ie, and If, or the pharmaceutically acceptable salt thereof, R^{1G} is C₃₋₆cycloalkyl, C₁₋₂₀alkyl, C₂₋₂₀alkenyl, or a 3 to 8 membered heterocyclyl comprising 1, 2, or 3 heteroatoms selected independently from N, O, and S; wherein the C₃₋₆cycloalkyl, C₁₋₂₀alkyl, C₂₋₂₀alkenyl, or 3 to 8 membered heterocyclyl is optionally substituted with a COOH, C₃₋₆cycloalkyl, or C₁₋₃alkoxy.

In some embodiments of the compounds of Formula I, Ia, Ib, Ic, Id, Ie, and If, or the pharmaceutically acceptable salt thereof, R^{1G} is C₅₋₆cycloalkyl, C₁₋₂₀alkyl or C₂₋₂₀alkenyl; wherein the C₅₋₆cycloalkyl, C₁₋₂₀alkyl or C₂₋₂₀alkenyl is optionally substituted with COOH, C₅₋₆cycloalkyl, C₁₋₃alkoxy, or —NHCOOR^{1H}. In some embodiments, R^{1G} is C₅₋₆cycloalkyl, C₁₋₂₀alkyl or C₂₋₂₀alkenyl; wherein the C₅₋₆cycloalkyl, C₁₋₂₀alkyl or C₂₋₂₀alkenyl is optionally substituted with COOH, C₅₋₆cycloalkyl, or C₁₋₃alkoxy.

In some embodiments of the compounds of Formula I, Ia, Ib, Ic, Id, Ie, and If, or the pharmaceutically acceptable salt thereof, R^{1G} is C₁₋₂₀alkyl or C₂₋₂₀alkenyl; wherein the C₁₋₂₀alkyl or C₂₋₂₀alkenyl is optionally substituted with a COOH, C₅₋₆cycloalkyl or C₁₋₃alkoxy. In some embodiments, R^{1G} is C₁₋₂₀alkyl or C₂₋₂₀alkenyl; wherein the C₂₋₂₀alkyl or C₂₋₂₀alkenyl is optionally substituted with a COOH. In some embodiments, R^{1G} is C₁₋₂₀alkyl or C₂₋₂₀alkenyl; wherein the C₁₋₂₀alkyl or C₂₋₂₀alkenyl is optionally substituted with a COOH.

In some embodiments of the compounds of Formula I, Ia, Ib, Ic, Id, Ie, and If, or the pharmaceutically acceptable salt thereof, R^{1G} is C₁₋₂₀alkyl optionally substituted with a COOH, C₃₋₆cycloalkyl, C₁₋₃alkoxy, or —NHCOOR^{1H}. In some embodiments, R^{1G} is C₁₋₂₀alkyl optionally substituted with a COOH, C₃₋₆cycloalkyl, or C₁₋₃alkoxy. In some embodiments, R^{1G} is C₁₋₂₀alkyl optionally substituted with a COOH, cyclopentyl, methoxy, or —NHCOOCH₃. In some embodiments, R^{1G} is C₁₋₂₀alkyl optionally substituted with a COOH, or cyclopentyl, methoxy. In some embodiments, R^{1G} is C₁₋₂₀alkyl optionally substituted with a COOH, C₃₋₆cycloalkyl, or C₁₋₃alkoxy. In some embodiments, R^{1G} is C₁₋₈alkyl optionally substituted with a COOH, cyclopentyl, or methoxy. In some embodiments, R^{1G} is C₁₋₈alkyl optionally substituted with a COOH. In some embodiments, R^{1G} is C₁₋₈alkyl optionally substituted with a cyclopentyl.

In some embodiments of the compounds of Formula I, Ia, Ib, Ic, Id, Ie, and If, or the pharmaceutically acceptable salt thereof, R^{1G} is C₂₋₂₀alkenyl optionally substituted with a COOH.

In some embodiments of the compounds of Formula I, Ia, Ib, Ic, Id, Ie, and If, or the pharmaceutically acceptable salt thereof, R^{1G} is C₃₋₆cycloalkyl. In some embodiments, R^{1G} is C₅₋₆cycloalkyl. In some embodiments, R^{1G} is cyclopentyl.

In some embodiments of the compounds of Formula I, Ia, Ib, Ic, Id, Ie, and If, or the pharmaceutically acceptable salt thereof, R^{1G} is C₁₋₂₀alkyl or C₂₋₂₀alkenyl. In some embodiments, R^{1G} is C₁₋₂₀alkyl. In some embodiments, R^{1G} is C₂₋₂₀alkenyl.

In some embodiments of the compounds of Formula I, Ia, Ib, Ic, Id, Ie, and If, or the pharmaceutically acceptable salt thereof, R^{1G} is a 3 to 8 membered heterocyclyl comprising 1, 2, or 3 heteroatoms selected independently from N, O, and S; wherein the 3 to 8 membered heterocyclyl is optionally substituted with a COOH, C₃₋₆cycloalkyl, C₁₋₃alkoxy, or —NHCOOR^{1H}; wherein R^{1H} is C₁₋₃alkyl. In some embodiments, R^{1G} is a 3 to 8 membered heterocyclyl comprising 1, 2, or 3 heteroatoms selected independently from N, O, and S; wherein the 3 to 8 membered heterocyclyl is optionally substituted with a COOH, C₃₋₆cycloalkyl, or C₁₋₃alkoxy.

In some embodiments of the compounds of Formula I, Ia, Ib, Ic, Id, Ie, and If, or the pharmaceutically acceptable salt thereof, R^{1G} is selected from the group consisting of —CH₃, —CH₂CH₃, —(CH₂)₂CH₃, —(CH₂)₃CH₃, —(CH₂)₄CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, —(CH₂)₁₄CH₃, —(CH₂)₁₅CH₃, —(CH₂)₁₆CH₃, —(CH₂)₁₇CH₃, —CH(CH₃)₂, —CH₂CH(CH₃)₂, —(CH₂)₂CH(CH₃)₂, —(CH₂)₃CH(CH₃)₂, —(CH₂)₄CH(CH₃)₂, —(CH₂)₅CH(CH₃)₂, —(CH₂)₆CH(CH₃)₂, —(CH₂)₇CH(CH₃)₂, —(CH₂)₇CH═CH(CH₂)₃CH₃, —(CH₂)₃(CH═CHCH₂)₅CH₃, —(CH₂)₇(CH═CHCH₂)₂(CH₂)₃CH₃, —CH₂CH₂-cyclopentyl, cyclopentyl, —(CH₂)₅OCH₃, —(CH₂)₃COOH, —(CH₂)₅COOH, —(CH₂)₁₆COOH, and

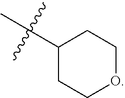 [SMILES: *C1CCOCC1]

In some embodiments of the compounds of Formula I, Ia, Ib, Ic, Id, Ie, and If, or the pharmaceutically acceptable salt thereof, R² is H or C₁₋₃alkyl. In some embodiments, R² is H. In some embodiments, R² is C₁₋₃alkyl. In some embodiments, R² is methyl.

In some embodiments of the compounds of Formula I, Ia, Ib, Ic, Id, Ie, and If, or the pharmaceutically acceptable salt thereof, R⁸ is H or C₁₋₃alkyl. In some embodiments, R⁸ is H. In some embodiments, R⁸ is C₁₋₃alkyl. In some embodiments, R⁸ is methyl.

In some embodiments of the compounds of Formula I, Ia, Ib, Ic, Id, Ie, and If, or the pharmaceutically acceptable salt thereof, R² is methyl and R⁸ is H or C₁₋₃alkyl. In some embodiments, R² methyl and R⁸ is C₁₋₃alkyl. In some embodiments, R² is methyl and R⁸ is methyl.

In some embodiments of the compounds of Formula I, Ia, Ib, Ic, Id, Ie, and If, or the pharmaceutically acceptable salt thereof, R⁸ is methyl and R² is H or C₁₋₃alkyl. In some embodiments, R⁸ is methyl and R² is C₁₋₃alkyl.

In some embodiments of the compounds of Formula I, Ia, Ib, Ic, Id, Ie, and If, or the pharmaceutically acceptable salt thereof, R³ is H or halo. In some embodiments, R³ is halo. In some embodiments, R³ is Cl or F. In some embodiments, R³ is Cl. In some embodiments, R³ is F. In some embodiments, R² is methyl, R³ is F, and R⁸ is methyl. In some embodiments, R³ is H.

In some embodiments of the compounds of Formula I, Ia, Ib, Ic, Id, Ie, and If, or the pharmaceutically acceptable salt thereof, R⁴ is H or halo. In some embodiments, R⁴ is halo. In some embodiments, R⁴ is Cl or F. In some embodiments, R⁴ is F. In some embodiments, R⁴ is Cl. In some embodiments, R² is methyl, R³ is F, R⁴ is F, and R⁸ is methyl. In some embodiments, R⁴ is H.

In some embodiments of the compounds of Formula I, Ia, Ib, Ic, Id, Ie, and If, or the pharmaceutically acceptable salt thereof, R⁶ is H or halo. In some embodiments, R⁶ is halo. In some embodiments, R⁶ is Cl or F. In some embodiments, R⁶ is F. In some embodiments, R⁶ is Cl. In some embodiments, R² is methyl, R³ is F, R⁴ is F, R⁶ is F, and R⁸ is methyl. In some embodiments, R⁶ is H.

In some embodiments of the compounds of Formula I, Ia, Ib, Ic, Id, Ie, and If, or the pharmaceutically acceptable salt thereof, R³, R⁴, and R⁶ are each independently a halo. In some embodiments, R³, R⁴, and R⁶ are each independently Cl or F. In some embodiments, R³, R⁴, and R⁶ are each F. In some embodiments, R³, R⁴ and R⁶ are each independently halo, R² is methyl and R⁸ is methyl. In some embodiments, R³, R⁴ and R⁶ are each independently Cl or F, R² is methyl, and R⁸ is methyl.

In some embodiments of the compounds of Formula I, Ia, Ib, Ic, Id, Ie, and If, or the pharmaceutically acceptable salt thereof, R³ and R⁴ are each F, and R² and R⁸ are each methyl. In some embodiments, R³ and R⁶ are each F, and R² and R⁸ are each methyl. In some embodiments, R⁴ and R⁶ are each F, and R² and R⁸ are each methyl.

In some embodiments of the compounds of Formula I, Ia, Ib, Ic, Id, Ie, and If, or the pharmaceutically acceptable salt thereof, R⁵ is H or halo. In some embodiments, R⁵ is halo. In some embodiments, R⁵ is Cl or F. In some embodiments, R⁵ is H. In some embodiments, R² is methyl, R⁵ is H, and R⁸ is methyl. In some embodiments, R³, R⁴ and R⁶ are each F, R⁵ is H, and R² is methyl and R⁸ is methyl.

In some embodiments of the compounds of Formula I, Ia, Ib, Ic, Id, Ie, and If, or the pharmaceutically acceptable salt thereof, R⁷ is H or halo. In some embodiments, R⁷ is halo. In some embodiments, R⁷ is Cl or F. In some embodiments, R⁷ is H. In some embodiments, R² is methyl, R⁷ is H, and R⁸ is methyl. In some embodiments, R³, R⁴ and R⁶ are each F, R⁷ is H, and R² is methyl and R⁸ is methyl.

In some embodiments of the compounds of Formula I, Ia, Ib, Ic, Id, Ie, and If, or the pharmaceutically acceptable salt thereof, R⁵ and R⁷ are each H. In some embodiments, R⁵ is H and R⁷ is halo. In some embodiments R⁵ is halo and R⁷ is H. In some embodiments R⁵ is halo and R⁷ is halo. In some embodiments, R⁵ is H and R⁷ is Cl or F. In some embodiments R⁵ is Cl or F and R⁷ is H. In some embodiments R⁵ is Cl or F and R⁷ is Cl or F.

In some embodiments of the compounds of Formula I, Ia, Ib, Ic, Id, Ie, and If, or the pharmaceutically acceptable salt thereof, R² is methyl, R⁵ and R⁷ are each H, and R⁸ is methyl. In some embodiments, R² is methyl, R³, R⁴ and R⁶ are each F, R⁵ and R⁷ are each H, and R⁸ is methyl.

In some embodiments of the compounds of Formula I and Ia, or the pharmaceutically acceptable salt thereof, the compound is selected from the group consisting of the compounds in Table 1:

TABLE 1

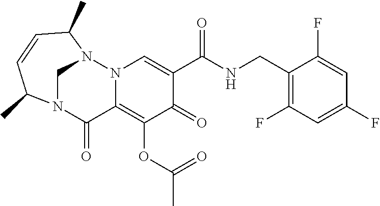

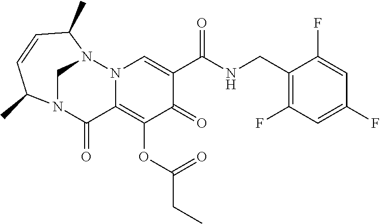

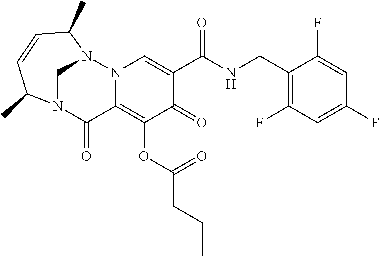

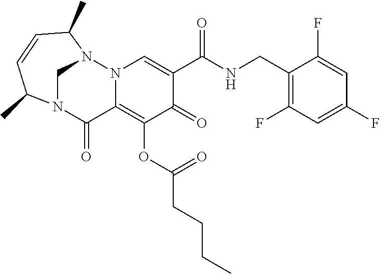

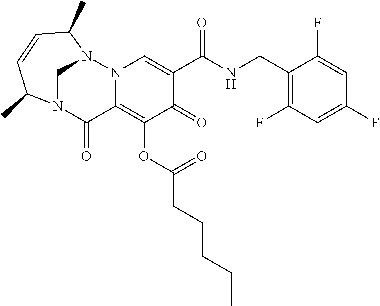

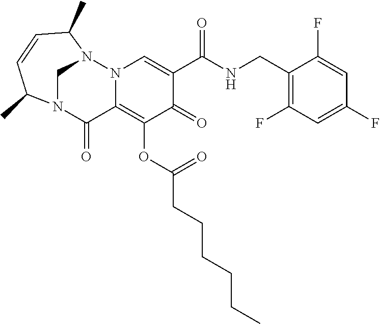

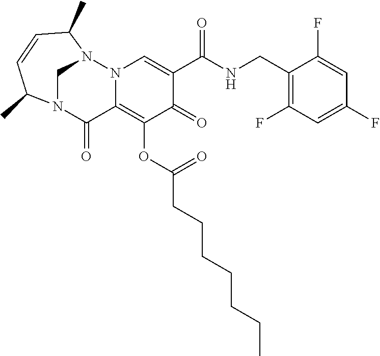

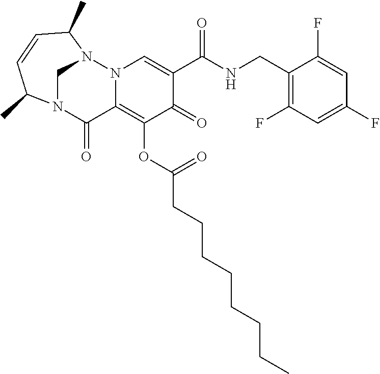

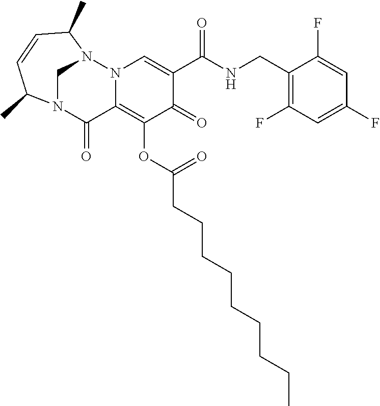

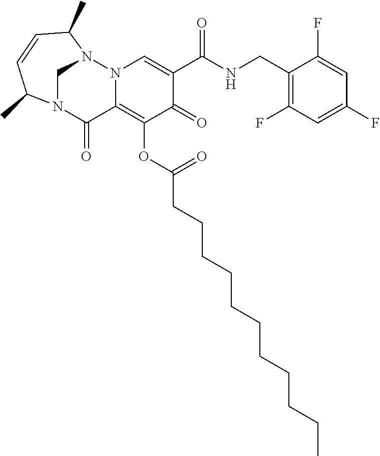

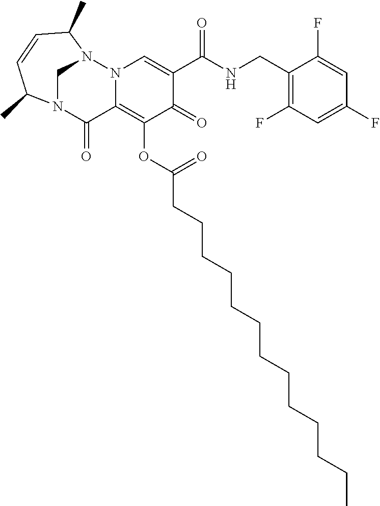

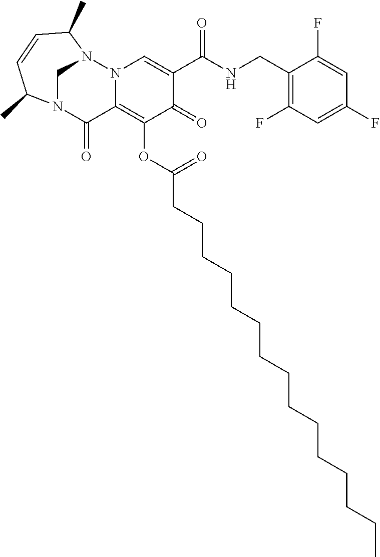

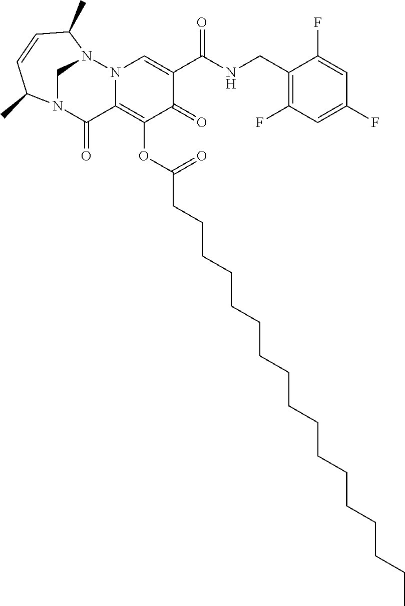

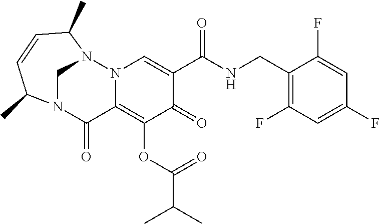

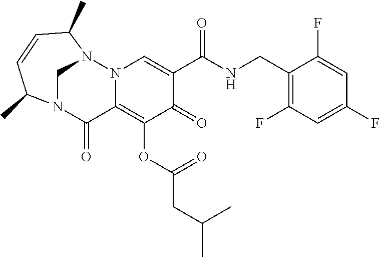

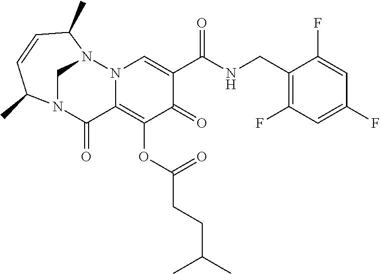

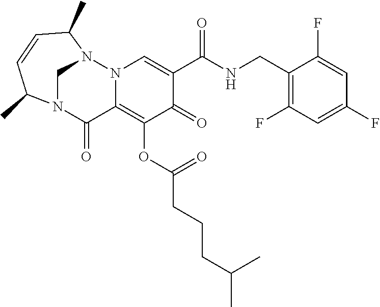

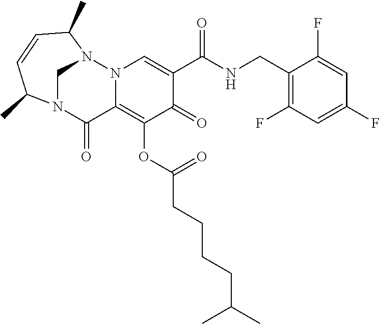

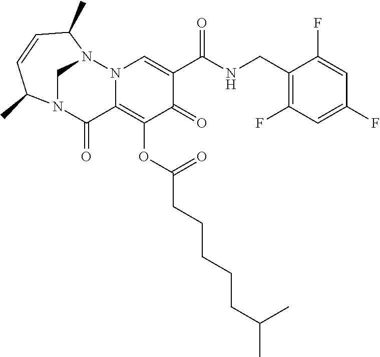

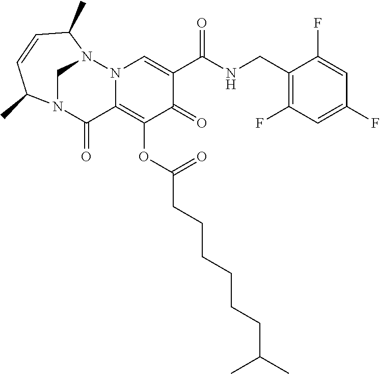

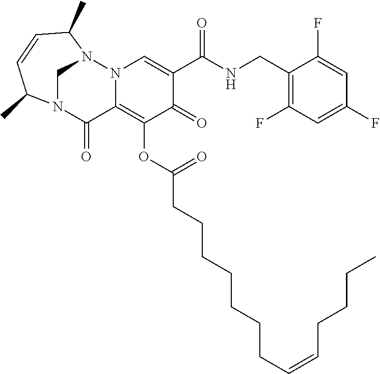

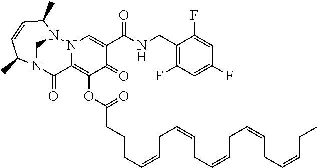

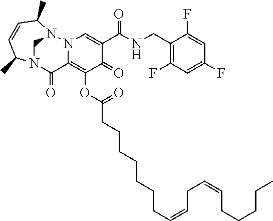

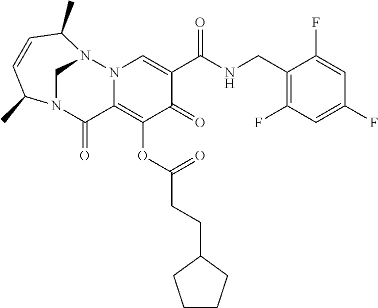

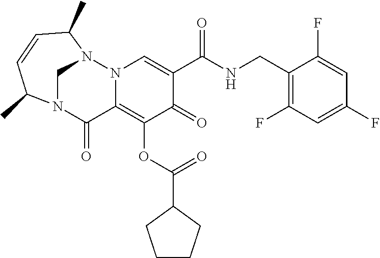

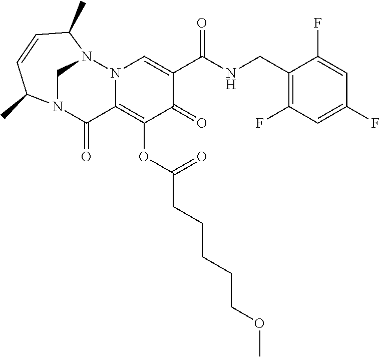

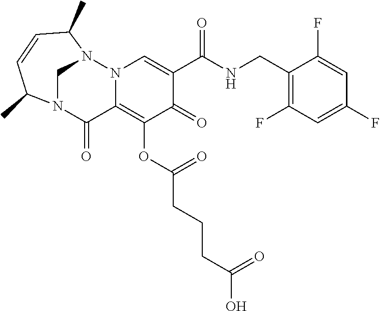

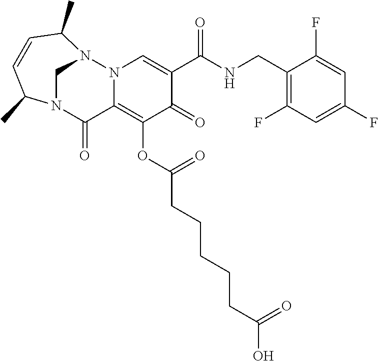

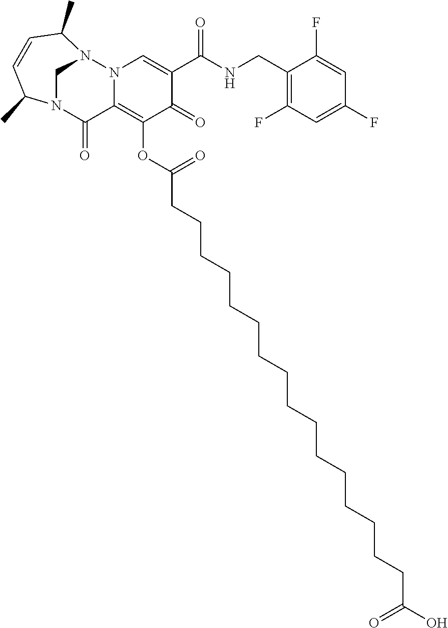

In some embodiments of the compounds of Formula I and If, or the pharmaceutically acceptable salt thereof, the compound is selected from the group consisting of the compounds in Table 2:

TABLE 2

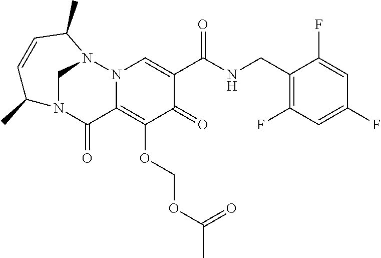

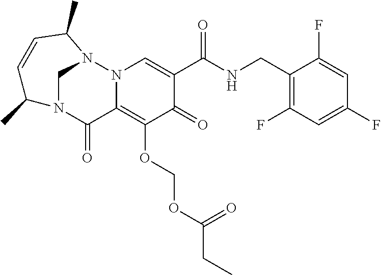

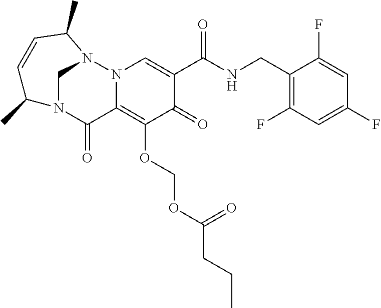

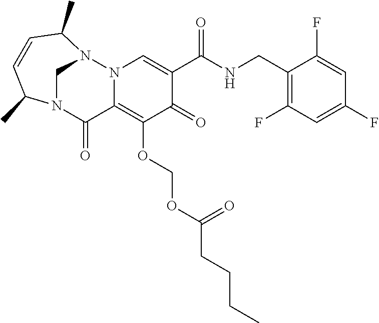

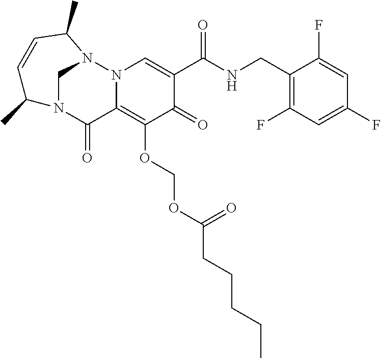

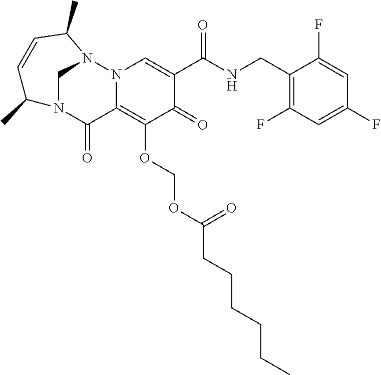

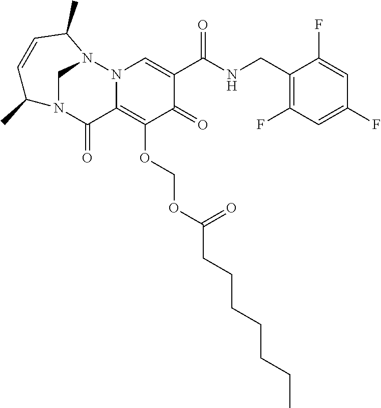

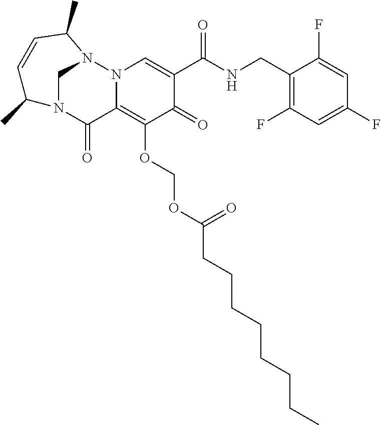

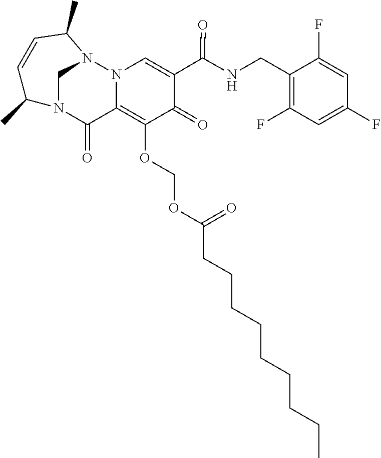

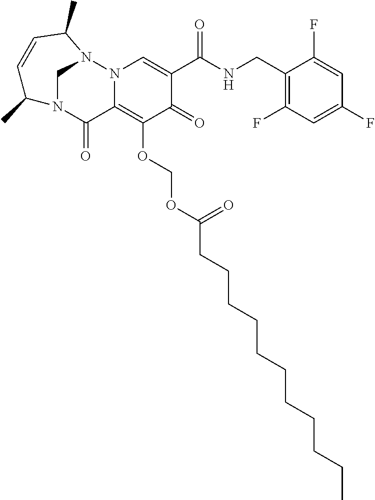

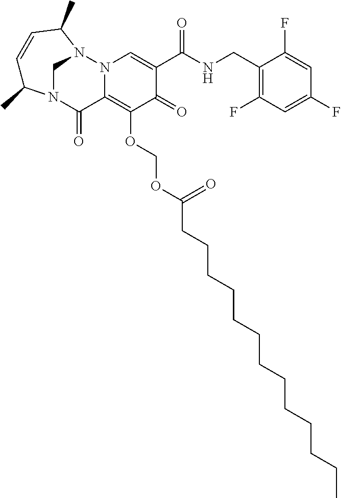

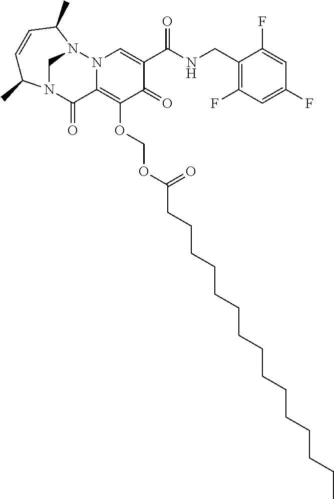

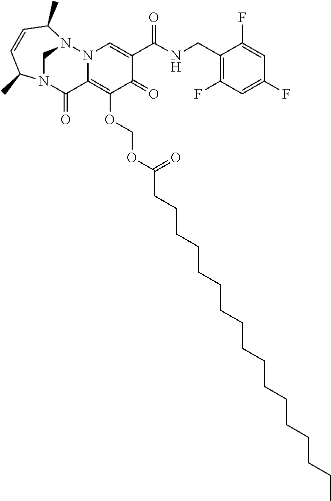

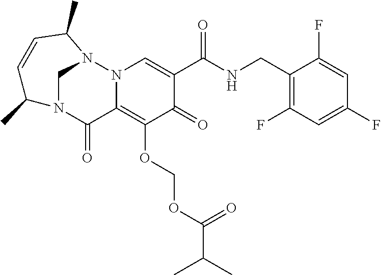

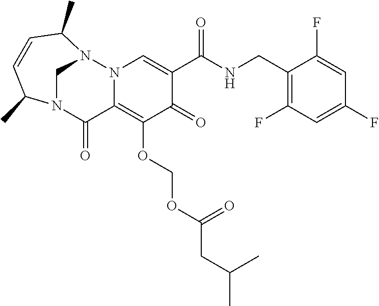

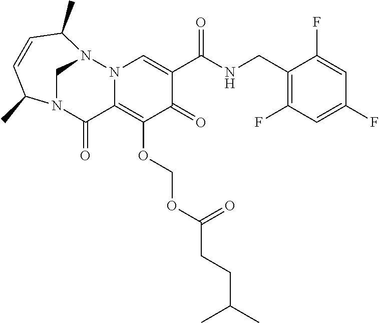

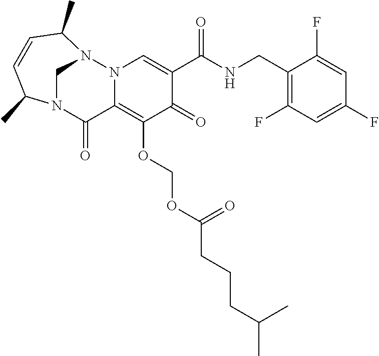

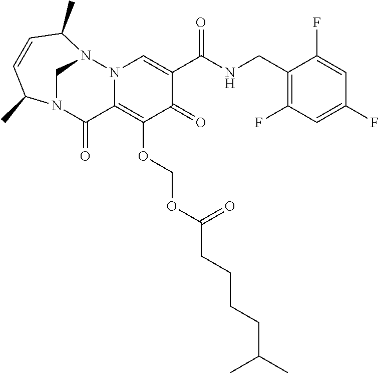

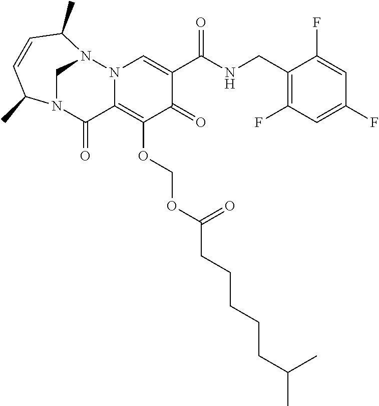

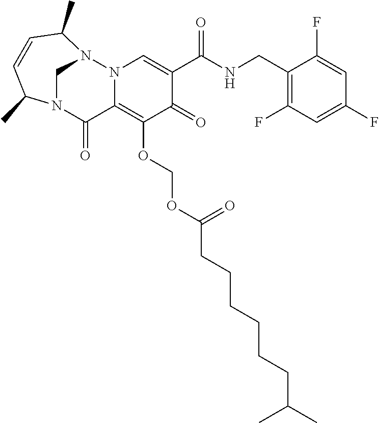

or a pharmaceutically acceptable salt thereof.

In some embodiments of the compounds of Formula I, Ib, and Ic, or the pharmaceutically acceptable salt thereof, the compound is selected from the group consisting of the compounds in Table 3:

TABLE 3

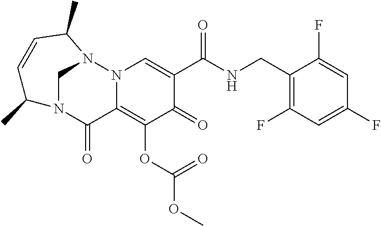

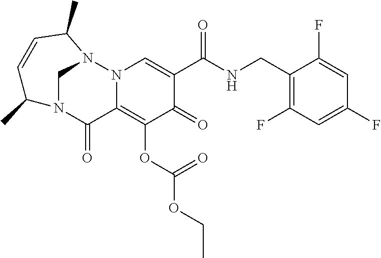

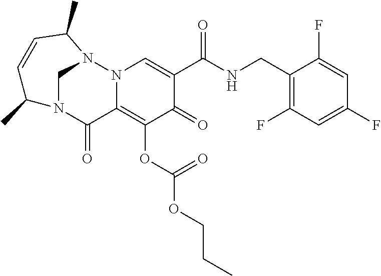

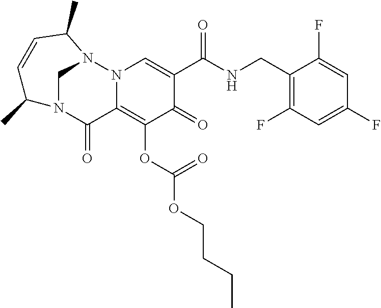

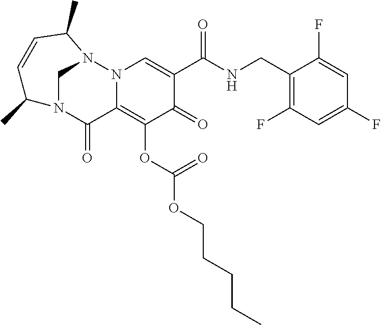

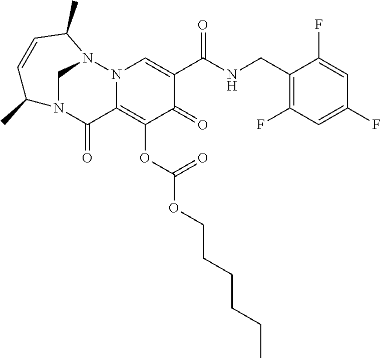

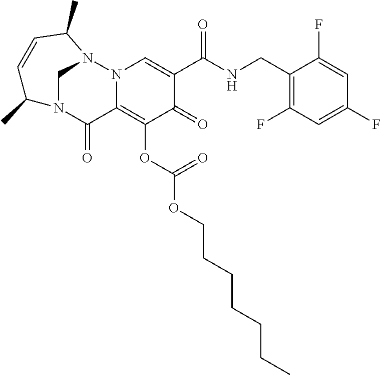

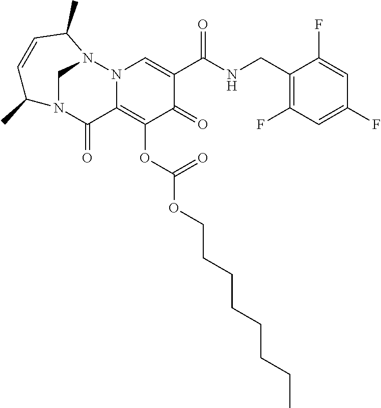

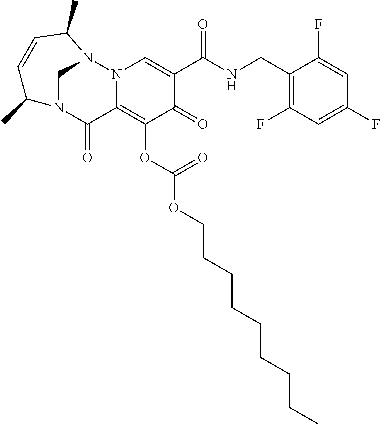

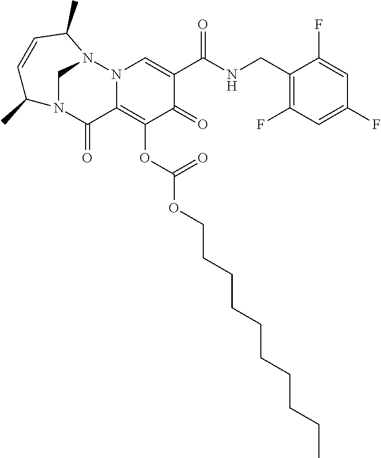

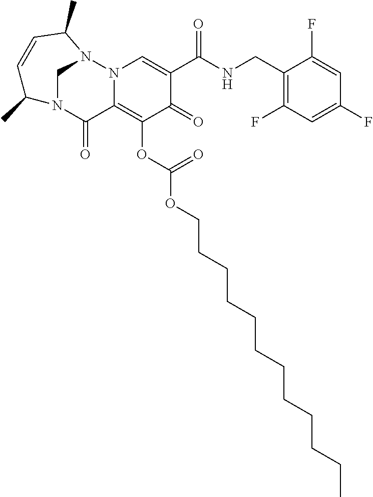

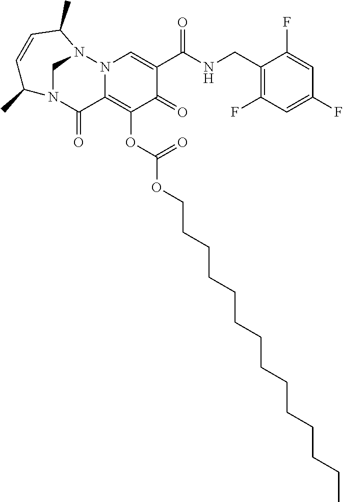

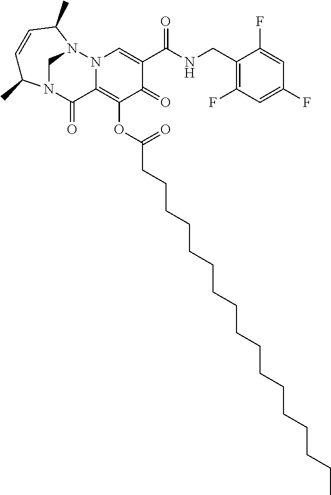

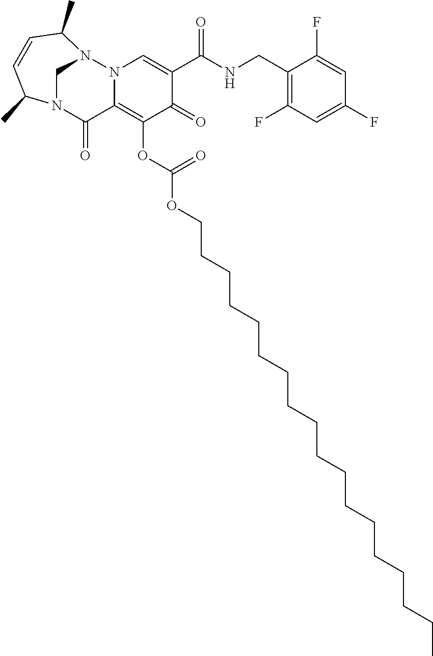

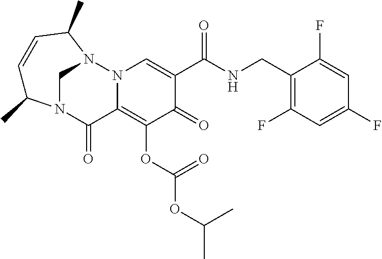

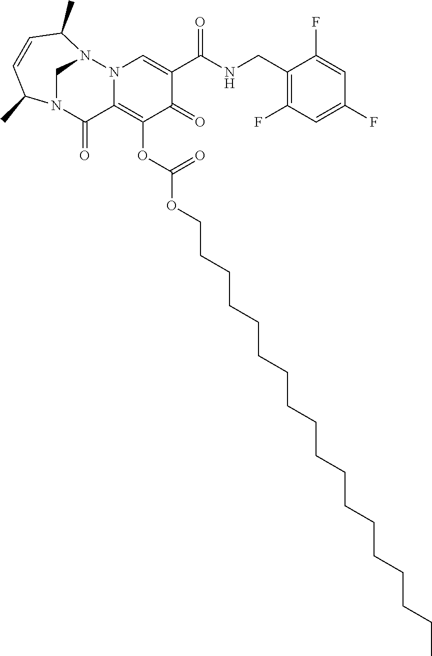

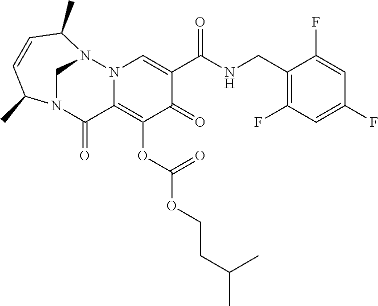

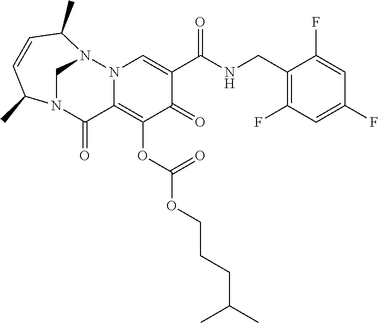

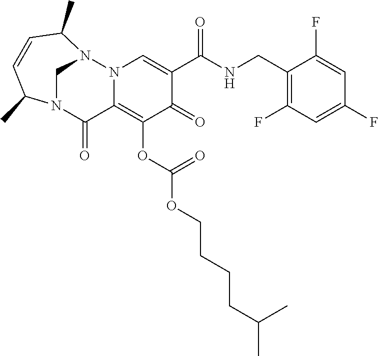

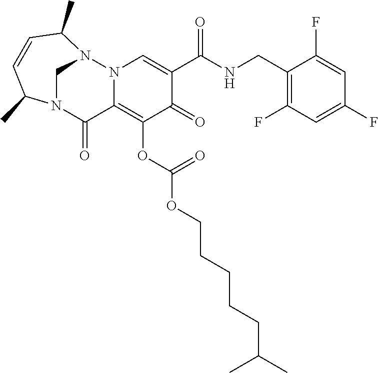

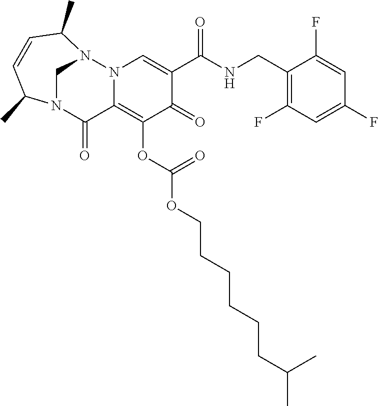

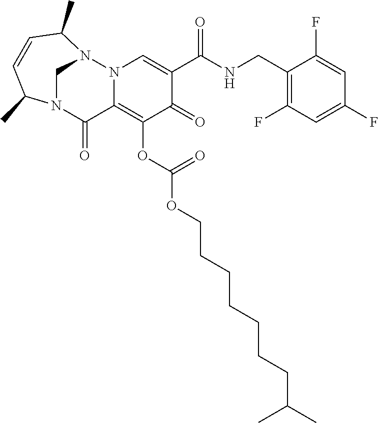

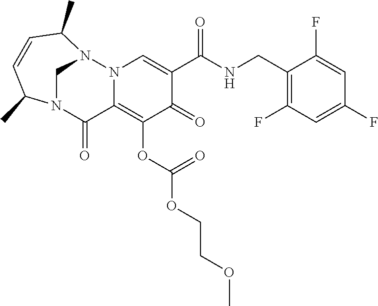

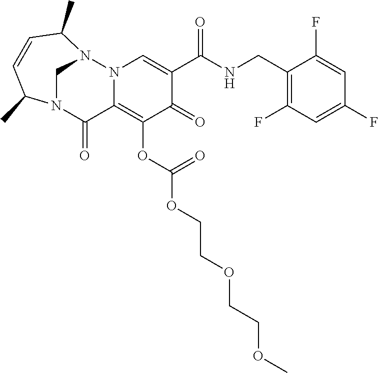

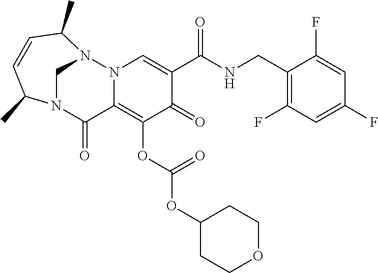

In some embodiments of the compounds of Formula I, Id, and Ie, or the pharmaceutically acceptable salt thereof, the compound is selected from the group consisting of the compounds in Table 4:

TABLE 4

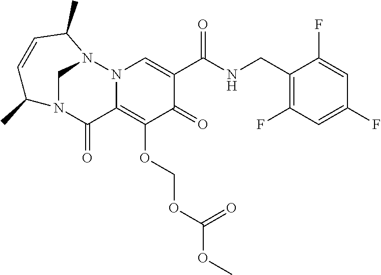

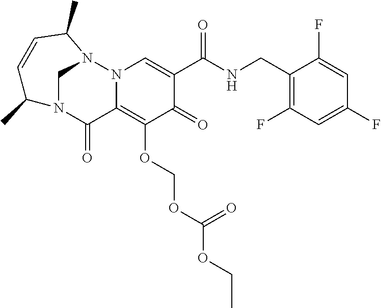

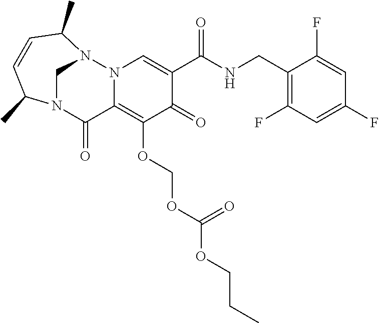

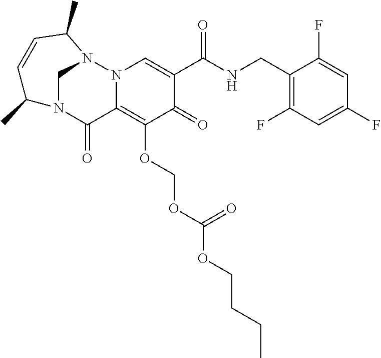

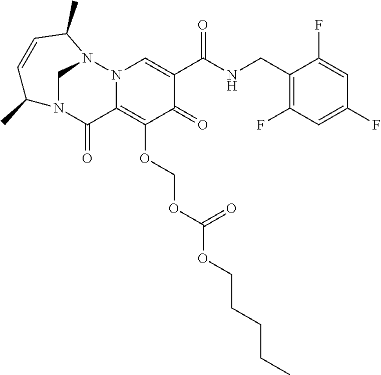

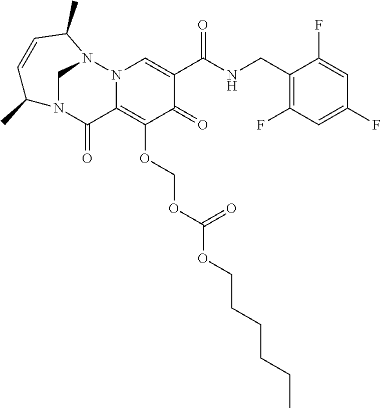

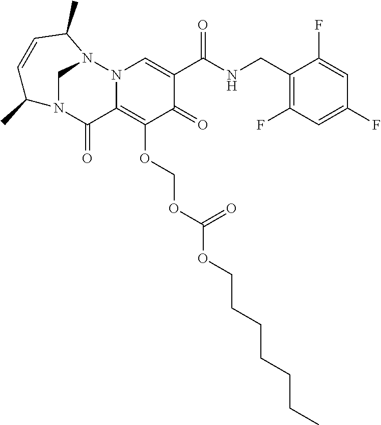

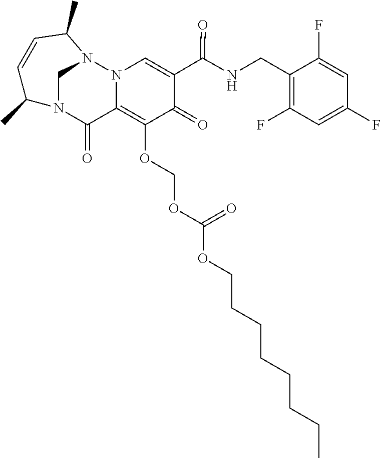

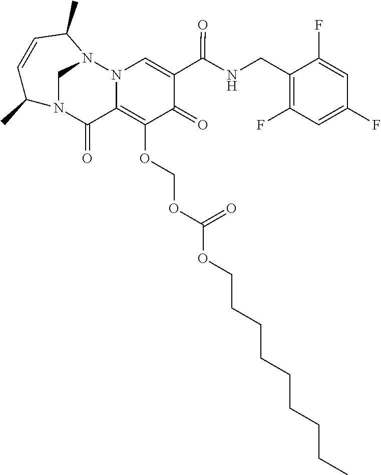

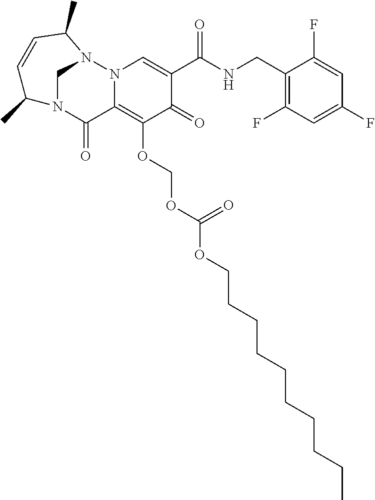

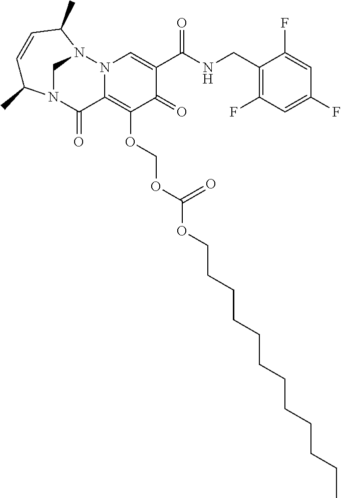

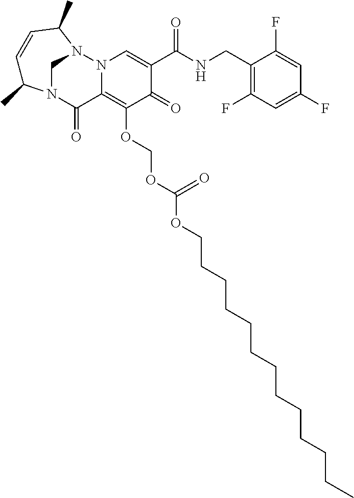

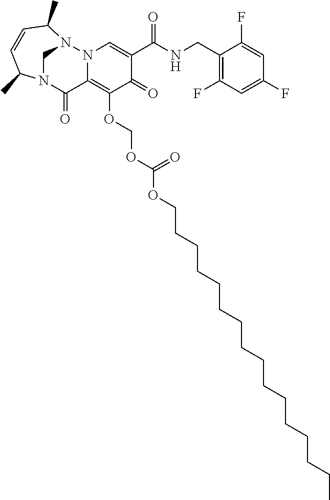

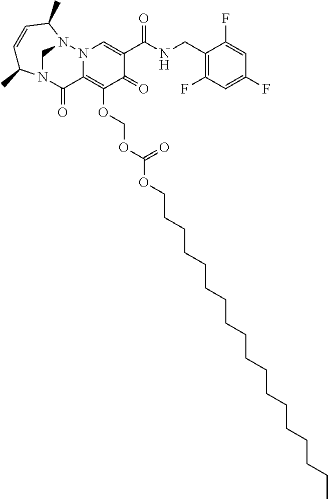

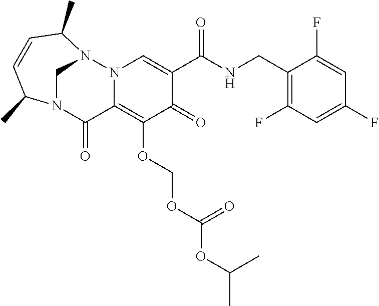

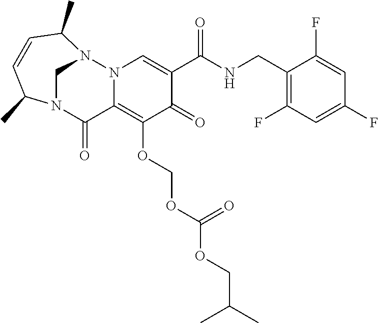

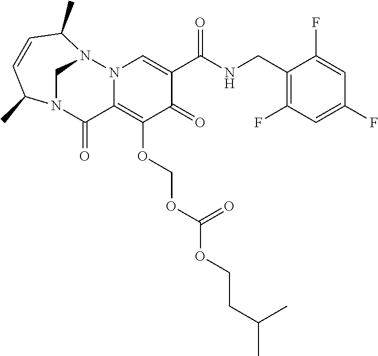

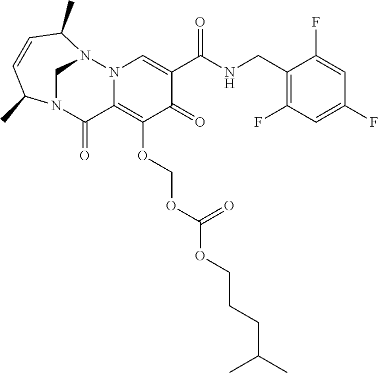

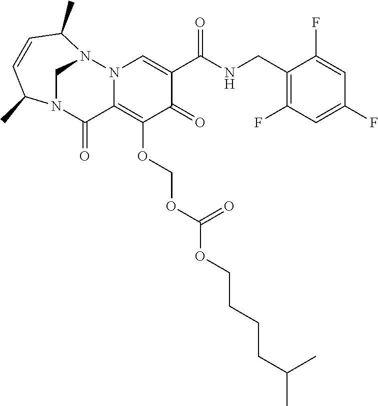

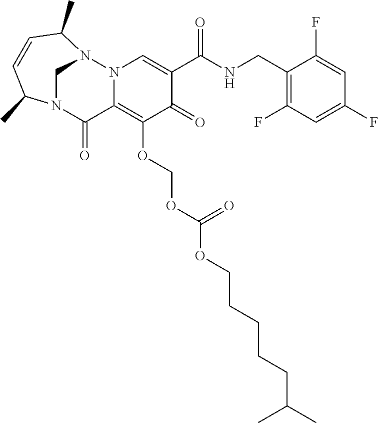

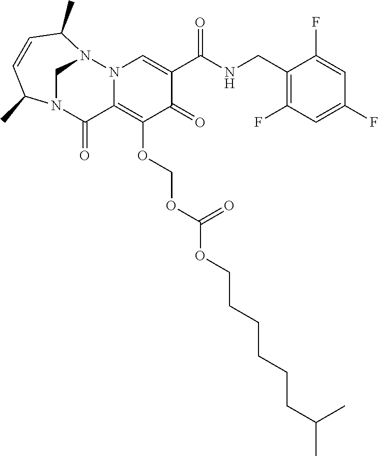

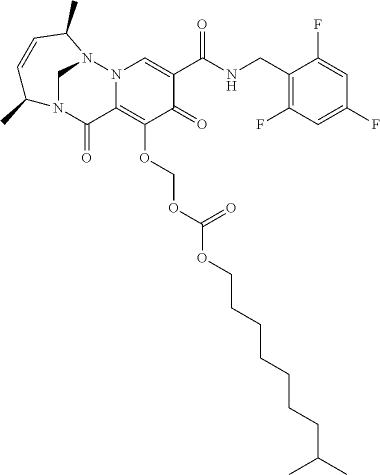

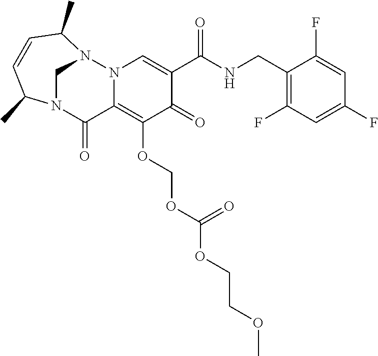

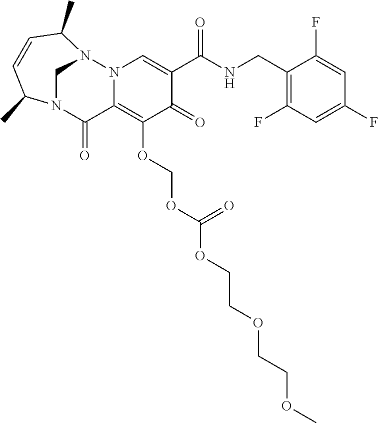

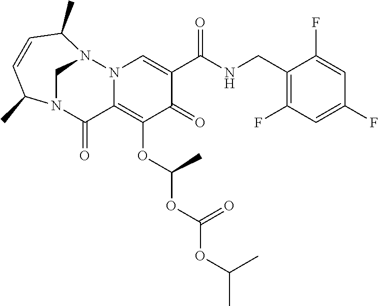

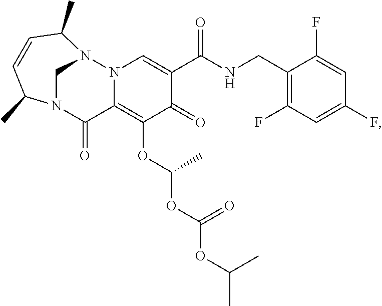

or a pharmaceutically acceptable salt thereof.

In some embodiments of the compounds of Formula I, Ia, Ib, Ic, Id, Ie, or If, or the pharmaceutically acceptable salt thereof, the compound is selected from the group consisting of the compounds in Table 1, Table 2, Table 3, and Table 4.

In some embodiments, the compounds disclosed herein have therapeutic activity. In some embodiments, the compounds disclosed herein are prodrugs, which upon administration to the human body can be converted to compounds having therapeutic activity. In some embodiments, the compounds disclosed herein are prodrugs of certain compounds disclosed in U.S. application Ser. No. 17/578,020, and PCT Application No. PCT/US2022/012773, the disclosures of which are incorporated herein by reference in their entireties. For example, the compounds disclosed herein can be converted to compounds of Formula II:

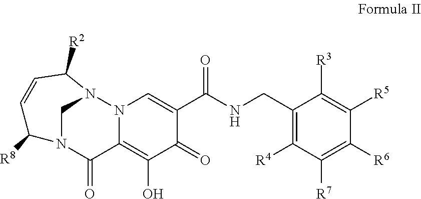

or pharmaceutically acceptable salts thereof, wherein R², R³, R⁴, R⁵, R⁶, R⁷, and R⁸ are as defined according to any other embodiment described herein. Accordingly, the present invention includes a method of converting a compound of Formula I, or a pharmaceutically acceptable salt thereof, to a compound of Formula II, or a pharmaceutically acceptable salt thereof, by (1) contacting the compound of Formula I, or a pharmaceutically acceptable salt thereof, with cell-containing aqueous media capable of converting —OR¹ to —OH (e.g., either enzymatically or chemically through acid or base hydrolysis); or (2) administering the compound to a patient whereby the compound of Formula I, or pharmaceutically acceptable salt thereof, is converted to a compound of Formula II, or a pharmaceutically acceptable salt thereof, through biologic pathways (e.g., enzymes) or through contact with biologic fluids and/or tissues conducive to converting —OR¹ to —OH (e.g., through acid or base hydrolysis).

The present invention further provides the treatment or prophylaxis of HIV infection in a patient in need thereof by contacting the patient with a compound of Formula II, or a pharmaceutically acceptable salt thereof, whereby the compound of Formula II, or a pharmaceutically acceptable salt thereof, is generated within the patient upon administration to the patient of a compound of Formula I, or a pharmaceutically acceptable salt thereof. In some embodiments, the compound of Formula II that is generated within the patient is Intermediate A, as described below in the Example section.

### III. PHARMACEUTICAL COMPOSITIONS

Compounds provided herein are usually administered in the form of pharmaceutical compositions. Thus, provided herein are also pharmaceutical compositions that comprise one or more of the compounds provided herein or pharmaceutically acceptable salts, isomer, or a mixture thereof and one or more pharmaceutically acceptable vehicles selected from carriers, adjuvants and excipients. The compounds provided herein may be the sole active ingredient or one of the active ingredients of the pharmaceutical compositions. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

In one aspect, provided herein are pharmaceutical compositions comprising a compound provided herein (e.g., a compound of Formula I, Ia, Ib, Ic, Id, Ie, or If), or a pharmaceutically acceptable salt thereof, and a pharmaceutically acceptable excipient or carrier. In some embodiments, the pharmaceutical compositions comprise a therapeutically effective amount of a compound provided herein, or a pharmaceutically acceptable salt thereof, and a pharmaceutically acceptable excipient or carrier.

In some embodiments, the pharmaceutical compositions provided herein further comprise one or more (e.g., one, two, three, four, one or two, one to three, or one to four) additional therapeutic agents, or a pharmaceutically acceptable salt thereof. In some embodiments, the pharmaceutical compositions further comprise a therapeutically effective amount of the one or more (e.g., one, two, three, four, one or two, one to three, or one to four) additional therapeutic agents, or a pharmaceutically acceptable salt thereof. In some embodiments, the pharmaceutical compositions further comprise one, tow, three, or four additional therapeutic agents.

The pharmaceutical compositions may be administered in either single or multiple doses. The pharmaceutical compositions may be administered by various methods including, for example, rectal, buccal, intranasal and transdermal routes. In some embodiments, the pharmaceutical compositions may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds provided herein. Administration may be via, for example, capsule or enteric coated tablets. In making the pharmaceutical compositions that include at least one compound provided herein or pharmaceutically acceptable salts, isomer, or a mixture thereof, the active ingredient (such as a compound provided herein) is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the pharmaceutical compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

The pharmaceutical compositions that include at least one compound described herein or pharmaceutically acceptable salts, isomer, or a mixture thereof can be formulated so as to provide quick, sustained or delayed release of the active ingredient (such as a compound provided herein) after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present disclosure employs transdermal delivery devices (“patches”). Such transdermal patches may be used to provide continuous or discontinuous infusion of the compounds provided herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or pharmaceutically acceptable salts, isomer, or a mixture thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with materials such as shellac, cetyl alcohol, and cellulose acetate.

Pharmaceutical compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### IV. METHODS OF TREATMENT

In one embodiment, methods of treating an HIV (e.g., HIV-1 and/or HIV-2) infection in a human having or at risk of having the infection comprising administering to the human a therapeutically effective amount of a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof, are provided.

In some embodiments, the methods further comprise administering to the human a therapeutically effective amount of one, two, three, or four additional therapeutic agents. In certain embodiments, the additional therapeutic agent or agents are anti-HIV agents. In particular embodiments, the additional therapeutic agent or agents are HIV protease inhibitors, HIV non-nucleoside or non-nucleotide inhibitors of reverse transcriptase, HIV nucleoside or nucleotide inhibitors of reverse transcriptase, HIV capsid inhibitors, gp41 inhibitors, CXCR4 inhibitors, gpl20 inhibitors, CCR5 inhibitors, latency reversing agents, capsid polymerization inhibitors, HIV bNAbs (broadly neutralizing HIV antibodies), TLR7 agonists, pharmacokinetic enhancers, other drugs for treating HIV, or combinations thereof.

In some embodiments, the additional thereapeutic agent or agents are abacavir, tenofovir alafenamide, tenofovir disoproxil, N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide, or a pharmaceutically acceptable salt thereof. In one embodiment, the additional therapeutic agent or agents are abacavir, tenofovir alafenamide, tenofovir disoproxil, lenacapavir, or a pharmaceutically acceptable salt thereof. In one embodiment, the additional therapeutic agent or agents are abacavir, tenofovir alafenamide, tenofovir disoproxil, lenacapavir, GS-5894, islatravir, or a pharmaceutically acceptable salt thereof. In some embodiments, the additional therapeutic agent or agents are lenacapavir, islatravir. In some embodiments, the additional therapeutic agent is lenacapavir. In some embodiments, the additional therapeutic agent is islatravir.

In some embodiments, the additional therapeutic agent or agents are abacavir, tenofovir alafenamide, tenofovir disoproxil, N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide, or a prodrug thereof. In one embodiment, the additional therapeutic agent or agents are abacavir, tenofovir alafenamide, tenofovir disoproxil, lenacapavir, or a prodrug thereof. In one embodiment, the additional therapeutic agent or agents are abacavir, tenofovir alafenamide, tenofovir disoproxil, lenacapavir, GS-5894, islatravir, or a prodrug thereof. In some embodiments, the additional therapeutic agent or agents are lenacapavir or islatravir, or a prodrug thereof. In some embodiments, the additional therapeutic agent is lenacapavir or a prodrug thereof. In some embodiments, the additional therapeutic agent is islatravir or a prodrug thereof.

In another embodiment, a use of a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof, for treating an HIV (e.g., HIV-1 and/or HIV-2) infection in a human having or at risk of having the infection is provided.

In another embodiment, a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof, for use in medical therapy is provided.

In another embodiment, a compound of Formula I, Ia, Ib, Ic, Id, Ie, or If, or a pharmaceutically acceptable salt thereof, or a pharmaceutical composition of Formula I, Ia, Ib, Ic, Id, Ie, or If, or pharmaceutically acceptable salt thereof, for use in treating an HIV infection is provided.

In another embodiment, a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof for use in a method of treating an HIV infection in a human having or at risk of having the infection, is provided.

In another embodiment, a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof for use in a method of treating an HIV infection in a human having or at risk of having the infection, is provided wherein said method further comprises administering to the human one, two, three, or four additional therapeutic agents.

In another embodiment, a compound of I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof for use in a method of treating an HIV infection in a human having or at risk of having the infection, is provided wherein said method further comprises administering to the human one, two, three, or four additional therapeutic agents selected from the group consisting of HIV protease inhibitors, HIV non-nucleoside or non-nucleotide inhibitors of reverse transcriptase, HIV nucleoside or nucleotide inhibitors of reverse transcriptase, HIV capsid inhibitors, gp41 inhibitors, CXCR4 inhibitors, gp120 inhibitors, CCR5 inhibitors, latency reversing agents, capsid polymerization inhibitors, HIV bNAbs, TLR7 agonists, pharmacokinetic enhancers, other drugs for treating HIV, or combinations thereof. In one embodiment, the one, two, three, or four additional therapeutic agents are selected from HIV protease inhibitors, HIV non-nucleoside inhibitors of reverse transcriptase, HIV nucleoside or nucleotide inhibitors of reverse transcriptase, latency reversing agents, HIV capsid inhibitors, HIV bNAbs, TLR7 agonists, and combinations thereof.

In another embodiment, a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof for use in a method of treating an HIV infection in a human having or at risk of having the infection, is provided wherein said method further comprises administering to the human a therapeutically effective amount of tenofovir disoproxil and emtricitabine.

In another embodiment, a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof for use in a method of treating an HIV infection in a human having or at risk of having the infection, is provided wherein said method further comprises administering to the human a therapeutically effective amount of tenofovir alafenamide and emtricitabine.

In another embodiment, a compound of Formula I, Ia, Ib, Ic, Id, Ie, or If, or a pharmaceutically acceptable salt thereof, or a pharmaceutical composition of a compound of I, Ia, Ib, Ic, Id, Ie, or If, or a pharmaceutically acceptable salt thereof for use in a method of treating an HIV infection in a human having or at risk of having the infection, is provided wherein said method further comprises administering to the human a therapeutically effective amount of tenofovir disoproxil.

In another embodiment, a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof for use in a method of treating an HIV infection in a human having or at risk of having the infection, is provided wherein said method further comprises administering to the human a therapeutically effective amount of tenofovir alafenamide.

In another embodiment, a method of using a compound of Formula I, Ia, Ib, Ic, Id, Ie, or If, or a pharmaceutically acceptable salt thereof in therapy is provided. In particular, a method of treating the proliferation of the HIV virus, treating AIDS, or delaying the onset of AIDS or ARC symptoms in a mammal (e.g., a human) is provided, comprising administering to the mammal a compound of Formula I, Ia, Ib, Ic, Id, Ie, or If, or a pharmaceutically acceptable salt thereof, and a pharmaceutically acceptable excipient.

In another embodiment, a composition comprising a compound of Formula I, Ia, Ib, Ic, Id, Ie, or If, or a pharmaceutically acceptable salt thereof, and a pharmaceutically acceptable excipient, for use in a method of treating the proliferation of the HIV virus, treating AIDS, or delaying the onset of AIDS or ARC symptoms in a mammal (e.g., a human) is provided.

In one embodiment, a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof, is provided for use in preventing HIV infection.

For example, in one embodiment, a compound of Formula I, Ia, Ib, Ic, Id, Ie, or If, or a pharmaceutically acceptable salt thereof, or a pharmaceutical composition of a compound of Formula I, Ia, Ib, Ic, Id, Ie, or If, or a pharmaceutically acceptable salt thereof, is provided for use in pre-exposure prophylaxis (PrEP), i.e., before the exposure of the individual to the HIV virus to prevent HIV infection from taking hold if the individual is exposed to the virus and/or to keep the virus from establishing a permanent infection and/or to prevent the appearance of symptoms of the disease and/or to prevent the virus from reaching detectable levels in the blood.

In another embodiment, the use of a compound of Formula I, Ia, Ib, Ic, Id, Ie, or If, or a pharmaceutically acceptable salt thereof, for the manufacture of a medicament for treating an HIV infection in a human being having or at risk of having the infection is disclosed.

In another embodiment, the use of a compound of Formula I, Ia, Ib, Ic, Id, Ie, or If, or a pharmaceutically acceptable salt thereof, as a research tool is disclosed.

In another embodiment, an article of manufacture comprising a composition effective to treat an HIV infection; and packaging material comprising a label which indicates that the composition can be used to treat infection by HIV is disclosed. Exemplary compositions comprise a compound of Formula I, Ia, Ib, Ic, Id, Ie, or If, or a pharmaceutically acceptable salt thereof.

In still another embodiment, a method of inhibiting the replication of HIV is disclosed. The method comprises exposing the virus to an effective amount of the compound of Formula I, Ia, Ib, Ic, Id, Ie, or If, or a pharmaceutically acceptable salt thereof, under conditions where replication of HIV is inhibited.

In another embodiment, the use of a compound of Formula I, Ia, Ib, Ic, Id, Ie, or If, or a pharmaceutically acceptable salt thereof, to inhibit the activity of the HIV integrase enzyme is disclosed.

In another embodiment, the use of a compound of Formula I, Ia, Ib, Ic, Id, Ie, or If, or a pharmaceutically acceptable salt thereof, to inhibit the replication of HIV is disclosed.

### V. ADMINISTRATION

The compounds of the present disclosure (for example, a compound of Formula I, Ia, Ib, Ic, Id, Ie, or If, or a pharmaceutically acceptable salt thereof, can be administered by any route appropriate to the condition to be treated. Suitable routes include oral, rectal, nasal, topical (including buccal and sublingual), transdermal, vaginal and parenteral (including subcutaneous, intramuscular, intravenous, intradermal, intrathecal and epidural), and the like. In some embodiments, the administration is oral, intravenous, subcutaneous, or intramuscular. It will be appreciated that the preferred route may vary with, for example, the condition of the recipient. An advantage of certain compounds disclosed herein is that they are orally bioavailable and can be dosed orally.

A compound of the present disclosure may be administered to an individual in accordance with an effective dosing regimen for a desired period of time or duration, such as at least about one month, at least about 2 months, at least about 3 months, at least about 6 months, or at least about 12 months or longer. In some embodiments, the compound is administered for the duration of the individual's life.

The specific dose level of a compound of the present disclosure for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The dosage may also be described as a total amount of a compound described herein administered per dose. Dosage of a compound of Formula I, Ia, Ib, Ic, Id, Ie, or If, or a pharmaceutically acceptable salt or pharmaceutically acceptable tautomer thereof, may be between about 1 mg and 4,000 mg, between about 2,000 to 4,000 mg, between about 1 to 2,000 mg, between about 1 to 1,000 mg, between about 10 to 500 mg, between about 20 to 500 mg, between about 50 to 300 mg, between about 75 to 200 mg, or between about 15 to 150 mg.

The dosage or dosing frequency of a compound of the present disclosure may be adjusted over the course of the treatment, based on the judgment of the administering physician.

The compounds of the present disclosure may be administered to an individual (e.g., a human) in a therapeutically effective amount.

In some embodiments, the compound is administered once every month, once every two months, once every three months, once every four months, once every five months, once every six months, once every seven months, once every eight months, once every nine months, once every ten months, once every eleven months, once every twelve months, or longer.

In some embodiments, the compound is administered once every month. In some embodiments, the compound is administered every two months. In some embodiments, the compound is administered every three months. In some embodiments, the compound is administered every four months. In some embodiments, the compound is administered every five months. In some embodiments, the compound is administered every six months. In some embodiments, the compound is administered every seven months. In some embodiments, the compound is administered every eight months. In some embodiments, the compound is administered every nine months. In some embodiments, the compound is administered every ten months. In some embodiments, the compound is administered every eleven months. In some embodiments, the compound is administered every year.

The compounds provided herein can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day. In some embodiments, a therapeutically effective amount of the compounds provided herein include from about 0.3 mg to about 30 mg per dose, or from about 30 mg to about 300 mg per dose, or from about 0.3 g to about 30 mg per dose, or from about g to about 300 g per dose.

A compound of the present disclosure may be combined with one or more additional therapeutic agents in any dosage amount of the compound of the present disclosure (e.g., from 1 mg to 1000 mg of compound). Therapeutically effective amounts may include from about 0.1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose, or such as from about 0.01 mg per dose to about 1000 mg per dose, or such as from about 0.01 mg per dose to about 100 mg per dose, or such as from about 0.1 mg per dose to about 100 mg per dose, or such as from about 1 mg per dose to about 100 mg per dose, or such as from about 1 mg per dose to about 10 mg per dose, or such as from about 1 mg per dose to about 1000 mg per dose.

A single dose can be administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month. In some embodiments, a compound disclosed herein is administered once daily in a method disclosed herein. In some embodiments, a compound disclosed herein is administered twice daily in a method disclosed herein.

In some embodiments, a compound disclosed herein is administered once every 10 days. In some embodiments, a compound disclosed herein is administered once every 15 days. In some embodiments, a compound disclosed herein is administered once every 20 days. In some embodiments, a compound disclosed herein is administered once every 10-15 days. In some embodiments, a compound disclosed herein is administered once every 15-20 days. In some embodiments, a compound disclosed herein is administered once every 10-20 days. In some embodiments, a compound disclosed herein is administered once every month. In some embodiments, a compound disclosed herein is administered once every 2 months. In some embodiments, a compound disclosed herein is administered once every 3 months. In some embodiments, a compound disclosed herein is administered once every 4 months. In some embodiments, a compound disclosed herein is administered once every 5 months. In some embodiments, a compound disclosed herein is administered once every 6 months. In some embodiments, a compound disclosed herein is administered once every 8 months. In some embodiments, a compound disclosed herein is administered once every 10 months. In some embodiments, a compound disclosed herein is administered once every year.

The frequency of dosage of the compound of the present disclosure will be determined by the needs of the individual patient and can be, for example, once per day, once per week, once per two weeks, once per month, once per two months, once per three months, once per four months, once per six months, or less. Administration of the compound continues for as long as necessary to treat the HIV infection.

### VI. KITS AND ARTICLES OF MANUFACTURE

In one aspect, provided herein are kits that comprise a compound provided herein, (e.g., a compound of Formula I, Ia, Ib, Ic, Id, Ie, or If,), or a pharmaceutically acceptable salt, stereoisomer, or solvate thereof, and suitable packaging. In some embodiments, the kit further comprises instructions for use. In some embodiments, the kit comprises a compound provided herein (e.g., a compound of Formula I, Ia, Ib, Ic, Id, Ie, or If,), or a pharmaceutically acceptable salt, stereoisomer, or solvate thereof, and a label and/or instructions for use of the compounds in the treatment of the indications, including the diseases or conditions, described herein.

In some embodiments, the kits further comprise one or more (e.g., one, two, three, four, one or two, one to three, or one to four) additional therapeutic agents, or a pharmaceutically acceptable salt thereof.

In one aspect, provided herein are articles of manufacture that comprise a compound described herein or pharmaceutically acceptable salts, isomer, or a mixture thereof in a suitable container. In some embodiments, the container may be a vial, jar, ampoule, preloaded syringe, or intravenous bag.

### VII. COMBINATION THERAPY

In certain embodiments, a method for treating an HIV infection is provided, comprising administering to the human a therapeutically effective amount of a compound disclosed herein, or a pharmaceutically acceptable salt thereof, in combination with a therapeutically effective amount of one, two, three, or four additional therapeutic agents. In one embodiment, a method for treating an HIV infection is provided, comprising administering to the human a therapeutically effective amount of a compound disclosed herein, or a pharmaceutically acceptable salt thereof, in combination with a therapeutically effective amount of one, two, three, or four additional therapeutic agents.

In one embodiment, pharmaceutical compositions comprising a compound disclosed herein, or a pharmaceutically acceptable salt thereof, in combination with one, two, three, or four additional therapeutic agents, and a pharmaceutically acceptable carrier, diluent, or excipient are provided.

In certain embodiments, the present disclosure provides a method for treating an HIV infection, comprising administering to a subject in need thereof a therapeutically effective amount of a compound disclosed herein, or a pharmaceutically acceptable salt thereof, in combination with a therapeutically effective amount of one, two, three, or four additional therapeutic agents which are suitable for treating an HIV infection.

In certain embodiments, a compound disclosed herein, or a pharmaceutically acceptable salt thereof, is combined with one, two, three, four, or more additional therapeutic agents. In certain embodiments, a compound disclosed herein, or a pharmaceutically acceptable salt thereof, is combined with one, two, three, or four additional therapeutic agents. In certain embodiments, a compound disclosed herein, or a pharmaceutically acceptable salt thereof, is combined with two additional therapeutic agents. In other embodiments, a compound disclosed herein, or a pharmaceutically acceptable salt thereof, is combined with three additional therapeutic agents. In further embodiments, a compound disclosed herein, or a pharmaceutically acceptable salt thereof, is combined with four additional therapeutic agents. The one, two, three, four, or more additional therapeutic agents can be different therapeutic agents selected from the same class of therapeutic agents, and/or they can be selected from different classes of therapeutic agents.

### Administration of HIV Combination Therapy

In certain embodiments, a compound disclosed herein is administered with one, two, three, or four additional therapeutic agents. Co-administration of a compound disclosed herein with one, two, three, or four additional therapeutic agents generally refers to simultaneous or sequential administration of a compound disclosed herein and one, two, three, or four additional therapeutic agents, such that therapeutically effective amounts of the compound disclosed herein and the one, two, three, or four additional therapeutic agents are both present in the body of the patient. When administered sequentially, the combination may be administered in two or more administrations.

Co-administration includes administration of unit dosages of the compounds disclosed herein before or after administration of unit dosages of one, two, three, or four additional therapeutic agents. For example, the compound disclosed herein may be administered within seconds, minutes, or hours of the administration of the one, two, three, or four additional therapeutic agents. In some embodiments, a unit dose of a compound disclosed herein is administered first, followed within seconds or minutes by administration of a unit dose of one, two, three, or four additional therapeutic agents. Alternatively, a unit dose of one, two, three, or four additional therapeutic agents is administered first, followed by administration of a unit dose of a compound disclosed herein within seconds or minutes. In other embodiments, a unit dose of a compound disclosed herein is administered first, followed, after a period of hours (e.g., 1-12 hours), by administration of a unit dose of one, two, three, or four additional therapeutic agents. In yet other embodiments, a unit dose of one, two, three, or four additional therapeutic agents is administered first, followed, after a period of hours (e.g., 1-12 hours), by administration of a unit dose of a compound disclosed herein.

In certain embodiments, a kit comprising a compound disclosed herein (e.g., a compound of Formula I, Ia, Ib, Ic, Id, Ie, or If, or a pharmaceutically acceptable salt thereof, and one or more (e.g., one, two, three, or four) additional therapeutic agents is provided.

In a specific embodiment, the kit includes a compound disclosed herein, or a pharmaceutically acceptable salt thereof, an HIV nucleoside or nucleotide inhibitor of reverse transcriptase and an HIV capsid inhibitor or an HIV capsid polymerization inhibitor.

### HIV Combination Therapy

In the above embodiments, the additional therapeutic agent or agents may be an anti-HIV agent. In some instances, the additional therapeutic agent can be HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, HIV capsid inhibitors, nucleocapsid protein 7 (NCp7) inhibitors, HIV Tat or Rev inhibitors, inhibitors of Tat-TAR-P-TEFb, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T-cell receptors, TCR-T, autologous T-cell therapies, engineered B cells, NK cells), latency reversing agents, immune-based therapies, phosphatidylinositol 3-kinase (PI3K) inhibitors, HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, Fatty acid synthase inhibitor, HIV vif gene modulators, Vif dimerization antagonists, HIV-1 viral infectivity factor inhibitors, HIV-1 Nef modulators, TNF alpha ligand inhibitors, HIV Nef inhibitors, Hck tyrosine kinase modulators, mixed lineage kinase-3 (MLK-3) inhibitors, HIV-1 splicing inhibitors, integrin antagonists, nucleoprotein inhibitors, splicing factor modulators, COMM domain containing protein 1 modulators, HIV ribonuclease H inhibitors, IFN antagonists, retrocyclin modulators, CD3 antagonists, CDK-4 inhibitors, CDK-6 inhibitors, CDK-9 inhibitors, Cytochrome P450 3 inhibitors, CXCR4 modulators, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, HPK1 (MAP4K1) inhibitors, proprotein convertase PC9 stimulators, ATP dependent RNA helicase DDX3X inhibitors, reverse transcriptase priming complex inhibitors, G6PD and NADH-oxidase inhibitors, mTOR complex 1 inhibitors, mTOR complex 2 inhibitors, P-Glycoprotein modulators, RNA polymerase modulators, TAT protein inhibitors, Prolyl endopeptidase inhibitors, Phospholipase A2 inhibitors, pharmacokinetic enhancers, HIV gene therapy, HIV vaccines, anti-HIV peptides, and combinations thereof.

In some embodiments, the additional therapeutic agent or agents are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent or agents are chosen from HIV protease inhibitors, HIV non-nucleoside or non-nucleotide inhibitors of reverse transcriptase, HIV nucleoside or nucleotide inhibitors of reverse transcriptase, HIV integrase inhibitors, HIV capsid inhibitors, gp41 inhibitors, CXCR4 inhibitors, gp120 inhibitors, CCR5 inhibitors, Nef inhibitors, latency reversing agents, HIV bNAbs, agonists of TLR7, TLR8, and TLR9, HIV vaccines, cytokines, immune checkpoint inhibitors, FLT3 ligands, T cell and NK cell recruiting bispecific antibodies, chimeric T cell receptors targeting HIV antigens, pharmacokinetic enhancers, and other drugs for treating HIV, and combinations thereof.

In some embodiments, the additional therapeutic agent or agents are chosen from dolutegravir, cabotegravir, islatravir, darunavir, bictegravir, elsulfavirine, rilpivirine, and lenacapavir, and combinations thereof.

### HIV Combination Drugs

Examples of combination drugs include, but are not limited to, ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); darunavir, tenofovir alafenamide hemifumarate, emtricitabine, and cobicistat; efavirenz, lamivudine, and tenofovir disoproxil fumarate; lamivudine and tenofovir disoproxil fumarate; tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; tenofovir analog; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); BIKTARVY® (bictegravir+emtricitabine+tenofovir alafenamide), DOVATO® (dolutegravir+lamivudine), TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dolutegravir+lamivudine, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine, lopinavir+ritonavir+abacavir+lamivudine, lamivudine, cabotegravir+rilpivirine, 3-BNC117+albuvirtide, elpida (elsulfavirine, VM-1500), and VM-1500A, lenacapavir+islatravir (oral, injectable), and dual-target HIV-1 reverse transcriptase/nucleocapsid protein 7 inhibitors.

### Other HIV Drugs

Examples of other drugs for treating HIV include, but are not limited to, aspernigrin C, acemannan, alisporivir, BanLec, deferiprone, Gamimune, metenkefalin, naltrexone, Prolastin, REP 9, RPI-MN, VSSP, HIviral, SB-728-T, 1,5-dicaffeoylquinic acid, rHIV7-shl-TAR-CCR5RZ, AAV-eCD4-Ig gene therapy, MazF gene therapy, BlockAide, bevirimat derivatives, ABBV-382, ABX-464, AG-1105, APH-0812, APH0202, bryostatin-1, bryostatin analogs, BIT-225, BRII-732, BRII-778, CYT-107, CS-TATI-1, fluoro-beta-D-arabinose nucleic acid (FANA)-modified antisense oligonucleotides, FX-101, griffithsin, GSK-3739937, GSK-3739937 (long-acting), HGTV-43, HPH-116, HS-10234, hydroxychloroquine, IMB-10035, IMO-3100, IND-02, JL-18008, LADAVRU, MK-1376, MK-2048, MK-4250, MK-8507, MK-8558, MK-8591 (islatravir), NOV-205, OB-002H, ODE-Bn-TFV, PA-1050040 (PA-040), PC-707, PGN-007, QF-036, S-648414, SCY-635, SB-9200, SCB-719, TR-452, TEV-90110, TEV-90112, TEV-90111, TEV-90113, RN-18, DIACC-1010, Fasnall, Immuglo, 2-CLIPS peptide, HRF-4467, thrombospondin analogs, TBL-1004HI, VG-1177, xl-081, AVI-CO-004, rfhSP-D, [18F]-MC-225, URMC-099-C, RES-529, Verdinexor, IMC-M113V, IML-106, antiviral fc conjugate (AVC), WP-1096, WP-1097, Gammora, ISR-CO48, ISR-48, ISR-49, MK-8527, cannabinoids, ENOB-HV-32, HiviCide-I, T-1144, VIR-576, nipamovir, Covimro, and ABBV-1882.

### HIV Protease Inhibitors

Examples of HIV protease inhibitors include, but are not limited to, amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, ASC-09+ritonavir, AEBL-2, DG-17, GS-1156, TMB-657 (PPL-100), T-169, BL-008, MK-8122, TMB-607, GRL-02031, and TMC-310911.

Additional examples of HIV protease inhibitors are described, e.g., in U.S. Pat. No. 10,294,234, and U.S. Patent Application Publication Nos. US2020030327 and US2019210978.

### HIV Gag Protein Inhibitors

Examples of HIV Gag protein inhibitors include, but are not limited to, HRF-10071.

### HIV Ribonuclease H Inhibitors

Examples of HIV ribonuclease H inhibitors include, but are not limited to, NSC-727447.

### HIV Nef Inhibitors

Examples of HIV Nef inhibitors include, but are not limited to, FP-1.

### HIV Reverse Transcriptase Inhibitors

Examples of HIV non-nucleoside or non-nucleotide inhibitors of reverse transcriptase include, but are not limited to, dapivirine, delavirdine, delavirdine mesylate, doravirine, efavirenz, etravirine, lentinan, nevirapine, rilpivirine, ACC-007, ACC-008, AIC-292, F-18, KM-023, PC-1005, M1-TFV, M2-TFV, VM-1500A-LAI, PF-3450074, elsulfavirine (sustained release oral, HIV infection), doravirine+islatravir (fixed dose combination/oral tablet formulation, HIV-1 infection), elsulfavirine (long acting injectable nanosuspension, HIV infection), and elsulfavirine (VM-1500).

Examples of HIV nucleoside or nucleotide inhibitors of reverse transcriptase include, but are not limited to, adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir octadecyloxyethyl ester (AGX-1009), tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, lamivudine, phosphazid, stavudine, zalcitabine, zidovudine, rovafovir etalafenamide (GS-9131), GS-9148, MK-8504, islatravir, MK-8583, VM-2500, and KP-1461.

Additional examples of HIV nucleoside or nucleotide inhibitors of reverse transcriptase include, but are not limited to, those described in patent publications US2007049754, US2016250215, US2016237062, US2016251347, US2002119443, US2013065856, US2013090473, US2014221356, and WO04096286.

### HIV Integrase Inhibitors

Examples of HIV integrase inhibitors include, but are not limited to, elvitegravir, elvitegravir (extended-release microcapsules), curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, PEGylated raltegravir, dolutegravir, JTK-351, bictegravir, AVX-15567, cabotegravir (long acting injectable), diketo quinolin-4-1 derivatives, integrase-LEDGF inhibitor, ledgins, M-522, M-532, MK-0536, NSC-310217, NSC-371056, NSC-48240, NSC-642710, NSC-699171, NSC-699172, NSC-699173, NSC-699174, stilbenedisulfonic acid, T169, STP-0404, VM-3500, XVIR-110, and ACC-017.

Examples of HIV non-catalytic site, or allosteric, integrase inhibitors (NCINI) include, but are not limited to, CX-05045, CX-05168, and CX-14442.

Additional examples of HIV capsid inhibitors include, but are not limited to, those described in U.S. Patent Application Publication Nos. US20200317689, US20210284642, US2014221356 and US2016016973.

### HIV Viral Infectivity Factor Inhibitors

Examples of HIV viral infectivity factor inhibitors include, but are not limited to, 2-amino-N-(2-methoxyphenyl)-6-((4-nitrophenyl)thio)benzamide derivatives, and Irino-L.

### HIV Entry Inhibitors

Examples of HIV entry (fusion) inhibitors include, but are not limited to, AAR-501, LBT-5001, cenicriviroc, CCR5 inhibitors, gp41 inhibitors, CD4 attachment inhibitors, gp120 inhibitors, gp160 inhibitors, and CXCR4 inhibitors.

Examples of CCR5 inhibitors include, but are not limited to, aplaviroc, vicriviroc, maraviroc, maraviroc (long acting injectable nanoemulsion), cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, thioraviroc and vMIP (Haimipu).

Examples of gp41 inhibitors include, but are not limited to, albuvirtide, enfuvirtide, griffithsin (gp41/gp120/gp160 inhibitor), BMS-986197, enfuvirtide biobetter, enfuvirtide biosimilar, HIV-1 fusion inhibitors (P26-Bapc), ITV-1, ITV-2, ITV-3, ITV-4, CPT-31, Cl3hmAb, lipuvirtide, PIE-12 trimer and sifuvirtide.

Examples of CD4 attachment inhibitors include, but are not limited to, ibalizumab and CADA analogs.

Examples of gp120 inhibitors include, but are not limited to, anti-HIV microbicide, Radha-108 (receptol) 3B3-PE38, BMS818251, BanLec, bentonite-based nanomedicine, fostemsavir tromethamine, IQP-0831, VVX-004, and BMS-663068.

Examples of gp160 inhibitors include, but are not limited to, fangchinoline.

Examples of CXCR4 inhibitors include, but are not limited to, plerixafor, ALT-1188, N15 peptide, and vMIP (Haimipu).

### HIV Maturation Inhibitors

Examples of HIV maturation inhibitors include, but are not limited to, BMS-955176, GSK-3640254 and GSK-2838232.

### Latency Reversing Agents

Examples of latency reversing agents include, but are not limited to, toll-like receptor (TLR) agonists (including TLR7 agonists, e.g., GS-9620, TLR8 agonists, and TLR9 agonists), histone deacetylase (HDAC) inhibitors, proteasome inhibitors such as velcade, protein kinase C (PKC) activators, Smyd2 inhibitors, BET-bromodomain 4 (BRD4) inhibitors (such as ZL-0580, apabetalone), ionomycin, IAP antagonists (inhibitor of apoptosis proteins, such as APG-1387, LBW-242), SMAC mimetics (including TL32711, LCL161, GDC-0917, HGS1029, AT-406, Debio-1143), PMA, SAHA (suberanilohydroxamic acid, or suberoyl, anilide, and hydroxamic acid), NIZ-985, IL-15 modulating antibodies (including IL-15, IL-15 fusion proteins, and IL-15 receptor agonists), JQl, disulfiram, amphotericin B, and ubiquitin inhibitors such as largazole analogs, APH-0812, and GSK-343. Examples of PKC activators include, but are not limited to, indolactam, prostratin, ingenol B, and DAG-lactones.

Additional examples of TLR7 agonists include, but are not limited to, those described in U.S. Patent Application Publication No. US2010143301.

Additional examples of TLR8 agonists include, but are not limited to, those described in U.S. Patent Application Publication No. US2017071944.

### Histone Deacetylase (HDAC) Inhibitors

In some embodiments, the agents as described herein are combined with an inhibitor of a histone deacetylase, e.g., histone deacetylase 1, histone deacetylase 9 (HDAC9, HD7, HD7b, HD9, HDAC, HDAC7, HDAC7B, HDAC9B, HDAC9FL, HDRP, MITR; Gene ID: 9734). Examples of HDAC inhibitors include without limitation, abexinostat, ACY-241, AR-42, BEBT-908, belinostat, CKD-581, CS-055 (HBI-8000), CT-101, CUDC-907 (fimepinostat), entinostat, givinostat, mocetinostat, panobinostat, pracinostat, quisinostat (JNJ-26481585), resminostat, ricolinostat, romidepsin, SHP-141, TMB-ADC, valproic acid (VAL-001), vorinostat, tinostamustine, remetinostat, and entinostat.

### Capsid Inhibitors

Examples of capsid inhibitors include, but are not limited to, capsid polymerization inhibitors or capsid disrupting compounds, HIV nucleocapsid p7 (NCp7) inhibitors such as azodicarbonamide, HIV p24 capsid protein inhibitors, lenacapavir (GS-6207), GS-CA1, AVI-621, AVI-101, AVI-201, AVI-301, and AVI-CAN1-15 series, PF-3450074, HIV-1 capsid inhibitors (HIV-1 infection, Shandong University), and compounds described in (GSK WO2019/087016).

Additional examples of capsid inhibitors include, but not limited to, those described in U.S. Patent Application Publication Nos. US2018051005 and US2016108030.

### Cytochrome P450 3 Inhibitors

Examples of Cytochrome P450 3 inhibitors include, but are not limited to, those described in U.S. Pat. No. 7,939,553.

### RNA Polymerase Modulators

Examples of RNA polymerase modulators include, but are not limited to, those described in U.S. Pat. Nos. 10,065,958 and 8,008,264.

### Immune Checkpoint Modulators

In various embodiments, the agents as described herein, are combined with one or more blockers or inhibitors of inhibitory immune checkpoint proteins or receptors and/or with one or more stimulators, activators or agonists of one or more stimulatory immune checkpoint proteins or receptors. Blockade or inhibition of inhibitory immune checkpoints can positively regulate T-cell or NK cell activation and prevent immune escape of infected cells. Activation or stimulation of stimulatory immune check points can augment the effect of immune checkpoint inhibitors in infective therapeutics. In various embodiments, the immune checkpoint proteins or receptors regulate T cell responses (e.g., reviewed in Xu et al., J Exp Clin Cancer Res. (2018) 37:110). In various embodiments, the immune checkpoint proteins or receptors regulate NK cell responses (e.g., reviewed in Davis et al., Semin Immunol. (2017) 31:64-75 and Chiossone et al., Nat Rev Immunol. (2018) 18(11):671-688).

Examples of immune checkpoint proteins or receptors include without limitation CD27, CD70; CD40, CD40LG; CD47, CD48 (SLAMF2), transmembrane and immunoglobulin domain containing 2 (TMIGD2, CD28H), CD84 (LY9B, SLAMF5), CD96, CD160, MS4A1 (CD20), CD244 (SLAMF4); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); natural killer cell cytotoxicity receptor 3 ligand 1 (NCR3LG1, B7H6); HERV-H LTR-associating 2 (HHLA2, B7H7); inducible T cell co-stimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF8 (CD30), TNFSF8 (CD30L); TNFRSF10A (CD261, DR4, TRAILR1), TNFRSF9 (CD137), TNFSF9 (CD137L); TNFRSF10B (CD262, DR5, TRAILR2), TNFRSF10 (TRAIL); TNFRSF14 (HVEM, CD270), TNFSF14 (HVEML); CD272 (B and T lymphocyte associated (BTLA)); TNFRSF17 (BCMA, CD269), TNFSF13B (BAFF); TNFRSF18 (GITR), TNFSF18 (GITRL); MHC class I polypeptide-related sequence A (MICA); MHC class I polypeptide-related sequence B (MICB); CD274 (CD274, PDL1, PD-L1); programmed cell death 1 (PDCD1, PD1, PD-1); cytotoxic T-lymphocyte associated protein 4 (CTLA4, CD152); CD80 (B7-1), CD28; nectin cell adhesion molecule 2 (NECTIN2, CD112); CD226 (DNAM-1); Poliovirus receptor (PVR) cell adhesion molecule (PVR, CD155); PVR related immunoglobulin domain containing (PVRIG, CDI12R); T cell immunoreceptor with Ig and ITIM domains (TIGIT); T cell immunoglobulin and mucin domain containing 4 (TIMD4; TIM4); hepatitis A virus cellular receptor 2 (HAVCR2, TIMD3, TIM3); galectin 9 (LGALS9); lymphocyte activating 3 (LAG3, CD223); signaling lymphocytic activation molecule family member 1 (SLAMF1, SLAM, CD150); lymphocyte antigen 9 (LY9, CD229, SLAMF3); SLAM family member 6 (SLAMF6, CD352); SLAM family member 7 (SLAMF7, CD319); UL16 binding protein 1 (ULBP1); UL16 binding protein 2 (ULBP2); UL16 binding protein 3 (ULBP3); retinoic acid early transcript 1E (RAETIE; ULBP4); retinoic acid early transcript 1G (RAETIG; ULBP5); retinoic acid early transcript 1L (RAETIL; ULBP6); lymphocyte activating 3 (CD223); killer cell immunoglobulin like receptor, three Ig domains and long cytoplasmic tail 1 (KIR, CD158E1); killer cell lectin like receptor C1 (KLRC1, NKG2A, CD159A); killer cell lectin like receptor K1 (KLRK1, NKG2D, CD314); killer cell lectin like receptor C2 (KLRC2, CD159c, NKG2C); killer cell lectin like receptor C3 (KLRC3, NKG2E); killer cell lectin like receptor C4 (KLRC4, NKG2F); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor D1 (KLRD1); SLAM family member 7 (SLAMF7); and Hematopoietic Progenitor Kinase 1 (HPK1, MAP4K1).

In various embodiments, the agents described herein are combined with one or more blockers or inhibitors of one or more T-cell inhibitory immune checkpoint proteins or receptors. Illustrative T-cell inhibitory immune checkpoint proteins or receptors include without limitation CD274 (CD274, PDL1, PD-L1); programmed cell death 1 ligand 2 (PDCD1LG2, PD-L2, CD273); programmed cell death 1 (PDCD1, PD1, PD-1); cytotoxic T-lymphocyte associated protein 4 (CTLA4, CD152); CD276 (B7H3); V-set domain containing T cell activation inhibitor 1 (VTCN1, B7H4); V-set immunoregulatory receptor (VSIR, B7H5, VISTA); immunoglobulin superfamily member 11 (IGSF11, VSIG3); TNFRSF14 (HVEM, CD270), TNFSF14 (HVEML); CD272 (B and T lymphocyte associated (BTLA)); PVR related immunoglobulin domain containing (PVRIG, CD112R); T cell immunoreceptor with Ig and ITIM domains (TIGIT); lymphocyte activating 3 (LAG3, CD223); hepatitis A virus cellular receptor 2 (HAVCR2, TIMD3, TIM3); galectin 9 (LGALS9); killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); and killer cell immunoglobulin like receptor, three Ig domains and long cytoplasmic tail 1 (KIR3DL1). In various embodiments, the agents, as described herein, are combined with one or more agonist or activators of one or more T-cell stimulatory immune checkpoint proteins or receptors. Illustrative T-cell stimulatory immune checkpoint proteins or receptors include without limitation CD27, CD70; CD40, CD40LG; inducible T cell costimulator (ICOS, CD278); inducible T cell costimulator ligand (ICOSLG, B7H2); TNF receptor superfamily member 4 (TNFRSF4, OX40); TNF superfamily member 4 (TNFSF4, OX40L); TNFRSF9 (CD137), TNFSF9 (CD137L); TNFRSF18 (GITR), TNFSF18 (GITRL); CD80 (B7-1), CD28; nectin cell adhesion molecule 2 (NECTIN2, CD112); CD226 (DNAM-1); CD244 (2B4, SLAMF4), Poliovirus receptor (PVR) cell adhesion molecule (PVR, CD155). See, e.g., Xu et al., J Exp Clin Cancer Res. (2018) 37:110.

In various embodiments, the agents as described herein, are combined with one or more blockers or inhibitors of one or more NK-cell inhibitory immune checkpoint proteins or receptors. Illustrative NK-cell inhibitory immune checkpoint proteins or receptors include without limitation killer cell immunoglobulin like receptor, three Ig domains and long cytoplasmic tail 1 (KIR, CD158E1); killer cell immunoglobulin like receptor, two Ig domains and long cytoplasmic tail 1 (KIR2DL1); killer cell immunoglobulin like receptor, two Ig domains and long cytoplasmic tail 2 (KIR2DL2); killer cell immunoglobulin like receptor, two Ig domains and long cytoplasmic tail 3 (KIR2DL3); killer cell immunoglobulin like receptor, three Ig domains and long cytoplasmic tail 1 (KIR3DL1); killer cell lectin like receptor C1 (KLRC1, NKG2A, CD159A); and killer cell lectin like receptor D1 (KLRD1, CD94). In various embodiments, the agents as described herein, are combined with one or more agonist or activators of one or more NK-cell stimulatory immune checkpoint proteins or receptors. Illustrative NK-cell stimulatory immune checkpoint proteins or receptors include without limitation CD16, CD226 (DNAM-1); CD244 (2B4, SLAMF4); killer cell lectin like receptor K1 (KLRK1, NKG2D, CD314); SLAM family member 7 (SLAMF7). See, e.g., Davis et al., Semin Immunol. (2017) 31:64-75; Fang et al., Semin Immunol. (2017) 31:37-54; and Chiossone et al., Nat Rev Immunol. (2018) 18(11):671-688.

In some embodiments, the one or more immune checkpoint inhibitors comprises a proteinaceous (e.g., antibody or fragment thereof, or antibody mimetic) inhibitor of PD-L1 (CD274), PD-1 (PDCD1) or CTLA4. In some embodiments, the one or more immune checkpoint inhibitors comprises a small organic molecule inhibitor of PD-L1 (CD274), PD-1 (PDCD1) or CTLA4. In some embodiments, the small molecule inhibitor of CD274 or PDCD1 is selected from the group consisting of GS-4224, GS-4416, INCB086550 and MAX10181. In some embodiments, the small molecule inhibitor of CTLA4 comprises BPI-002.

Examples of inhibitors of CTLA4 that can be co-administered include without limitation ipilimumab, tremelimumab, BMS-986218, AGEN1181, AGEN1884, BMS-986249, MK-1308, REGN-4659, ADU-1604, CS-1002, BCD-145, APL-509, JS-007, BA-3071, ONC-392, AGEN-2041, JHL-1155, KN-044, CG-0161, ATOR-1144, PBI-5D3H5, BPI-002, as well as multi-specific inhibitors FPT-155 (CTLA4/PD-L1/CD28), PF-06936308 (PD-1/CTLA4), MGD-019 (PD-1/CTLA4), KN-046 (PD-1/CTLA4), MEDI-5752 (CTLA4/PD-1), XmAb-20717 (PD-1/CTLA4), and AK-104 (CTLA4/PD-1).

Examples of inhibitors of PD-L1 (CD274) or PD-1 (PDCD1) that can be co-administered include without limitation pembrolizumab, nivolumab, cemiplimab, pidilizumab, AMP-224, MEDI0680 (AMP-514), spartalizumab, atezolizumab, avelumab, durvalumab, BMS-936559, CK-301, PF-06801591, BGB-A317 (tislelizumab), GLS-010 (WBP-3055), AK-103 (HX-008), AK-105, CS-1003, HLX-10, MGA-012, BI-754091, AGEN-2034, JS-001 (toripalimab), JNJ-63723283, genolimzumab (CBT-501), LZM-009, BCD-100, LY-3300054, SHR-1201, SHR-1210 (camrelizumab), Sym-021, ABBV-181(budigalimab), PD1-PIK, BAT-1306, (MSB0010718C), CX-072, CBT-502, TSR-042 (dostarlimab), MSB-2311, JTX-4014, BGB-A333, SHR-1316, CS-1001 (WBP-3155, KN-035, IBI-308 (sintilimab), HLX-20, KL-A167, STI-A1014, STI-A1015 (IMC-001), BCD-135, FAZ-053, TQB-2450, MDX1105-01, GS-4224, GS-4416, INCB086550, MAX10181, as well as multi-specific inhibitors FPT-155 (CTLA4/PD-L1/CD28), PF-06936308 (PD-1/CTLA4), MGD-013 (PD-1/LAG-3), FS-118 (LAG-3/PD-L1) MGD-019 (PD-1/CTLA4), KN-046 (PD-1/CTLA4), MEDI-5752 (CTLA4/PD-1), RO-7121661 (PD-1/TIM-3), XmAb-20717 (PD-1/CTLA4), AK-104 (CTLA4/PD-1), M7824 (PD-L1/TGFβ-EC domain), CA-170 (PD-L1/VISTA), CDX-527 (CD27/PD-L1), LY-3415244 (TIM3/PDL1), and INBRX-105 (4-1BB/PDL1).

In various embodiments, the agents as described herein are combined with anti-TIGIT antibodies, such as BMS-986207, RG-6058, and AGEN-1307.

### TNF Receptor Superfamily (TNFRSF) Member Agonists or Activators

In various embodiments, the agents as described herein are combined with an agonist of one or more TNF receptor superfamily (TNFRSF) members, e.g., an agonist of one or more of TNFRSF1A (NCBI Gene ID: 7132), TNFRSF1B (NCBI Gene ID: 7133), TNFRSF4 (OX40, CD134; NCBI Gene ID: 7293), TNFRSF5 (CD40; NCBI Gene ID: 958), TNFRSF6 (FAS, NCBI Gene ID: 355), TNFRSF7 (CD27, NCBI Gene ID: 939), TNFRSF8 (CD30, NCBI Gene ID: 943), TNFRSF9 (4-1BB, CD137, NCBI Gene ID: 3604), TNFRSF10A (CD261, DR4, TRAILR1, NCBI Gene ID: 8797), TNFRSF10B (CD262, DR5, TRAILR2, NCBI Gene ID: 8795), TNFRSF10C (CD263, TRAILR3, NCBI Gene ID: 8794), TNFRSF10D (CD264, TRAILR4, NCBI Gene ID: 8793), TNFRSF11A (CD265, RANK, NCBI Gene ID: 8792), TNFRSF11B (NCBI Gene ID: 4982), TNFRSF12A (CD266, NCBI Gene ID: 51330), TNFRSF13B (CD267, NCBI Gene ID: 23495), TNFRSF13C (CD268, NCBI Gene ID: 115650), TNFRSF16 (NGFR, CD271, NCBI Gene ID: 4804), TNFRSF17 (BCMA, CD269, NCBI Gene ID: 608), TNFRSF18 (GITR, CD357, NCBI Gene ID: 8784), TNFRSF19 (NCBI Gene ID: 55504), TNFRSF21 (CD358, DR6, NCBI Gene ID: 27242), and TNFRSF25 (DR3, NCBI Gene ID: 8718).

Examples of anti-TNFRSF4 (OX40) antibodies that can be co-administered include without limitation, MEDI6469, MEDI6383, MEDI0562 (tavolixizumab), MOXR0916, PF-04518600, RG-7888, GSK-3174998, INCAGN1949, BMS-986178, GBR-8383, ABBV-368, and those described in WO2016179517, WO2017096179, WO2017096182, WO2017096281, and WO2018089628.

Examples of anti-TNFRSF5 (CD40) antibodies that can be co-administered include without limitation RG7876, SEA-CD40, APX-005M and ABBV-428.

In some embodiments, the anti-TNFRSF7 (CD27) antibody varlilumab (CDX-1127) is co-administered.

Examples of anti-TNFRSF9 (4-1BB, CD137) antibodies that can be co-administered include without limitation urelumab, utomilumab (PF-05082566), AGEN2373 and ADG-106.

Examples of anti-TNFRSF18 (GITR) antibodies that can be co-administered include without limitation, MEDI1873, FPA-154, INCAGN-1876, TRX-518, BMS-986156, MK-1248, GWN-323, and those described in WO2017096179, WO2017096276, WO2017096189, and WO2018089628. In some embodiments, an antibody, or fragment thereof, co-targeting TNFRSF4 (OX40) and TNFRSF18 (GITR) is co-administered. Such antibodies are described, e.g., in WO2017096179 and WO2018089628.

### Bi-and Tri-Specific Natural Killer (NK)-Cell Engagers

In various embodiments, the agents as described herein, are combined with a bi-specific NK-cell engager (BiKE) or a tri-specific NK-cell engager (TriKE) (e.g., not having an Fc) or bi-specific antibody (e.g., having an Fc) against an NK cell activating receptor, e.g., CD16A, C-type lectin receptors (CD94/NKG2C, NKG2D, NKG2E/H and NKG2F), natural cytotoxicity receptors (NKp30, NKp44 and NKp46), killer cell C-type lectin-like receptor (NKp65, NKp80), Fc receptor FcγR (which mediates antibody-dependent cell cytotoxicity), SLAM family receptors (e.g., 2B4, SLAM6 and SLAM7), killer cell immunoglobulin-like receptors (KIR) (KIR-2DS and KIR-3DS), DNAM-1 and CD137 (41BB). As appropriate, the anti-CD16 binding bi-specific molecules may or may not have an Fc. Illustrative bi-specific NK-cell engagers that can be co-administered target CD16 and one or more HIV-associated antigens as described herein. BiKEs and TriKEs are described, e.g., in Felices et al., Methods Mol Biol. (2016) 1441:333-346; Fang et al., Semin Immunol. (2017) 31:37-54. Examples of trispecific NK cell engagers (TRiKE) include, but are not limited to, OXS-3550, HIV-TriKE, and CD16-IL-15-B7H3 TriKe.

### Indoleamine-Pyrrole-2,3-Dioxygenase (IDO1) Inhibitors

In various embodiments, the agents as described herein are combined with an inhibitor of indoleamine 2,3-dioxygenase 1 (IDO1; NCBI Gene ID: 3620). Examples of IDO1 inhibitors include without limitation, BLV-0801, epacadostat, F-001287, GBV-1012, GBV-1028, GDC-0919, indoximod, NKTR-218, NLG-919-based vaccine, PF-06840003, pyranonaphthoquinone derivatives (SN-35837), resminostat, SBLK-200802, BMS-986205, shIDO-ST, EOS-200271, KHK-2455, and LY-3381916.

### Toll-Like Receptor (TLR) Agonists

In various embodiments, the agents as described herein are combined with an agonist of a toll-like receptor (TLR), e.g., an agonist of TLR1 (NCBI Gene ID: 7096), TLR2 (NCBI Gene ID: 7097), TLR3 (NCBI Gene ID: 7098), TLR4 (NCBI Gene ID: 7099), TLR5 (NCBI Gene ID: 7100), TLR6 (NCBI Gene ID: 10333), TLR7 (NCBI Gene ID: 51284), TLR8 (NCBI Gene ID: 51311), TLR9 (NCBI Gene ID: 54106), and/or TLR10 (NCBI Gene ID: 81793). Example TLR7 agonists that can be co-administered include without limitation AL-034, DSP-0509, GS-9620 (vesatolimod), vesatolimod analog, LHC-165, TMX-101 (imiquimod), GSK-2245035, resiquimod, DSR-6434, DSP-3025, IMO-4200, MCT-465, MEDI-9197, 3M-051, SB-9922, 3M-052, Limtop, TMX-30X, TMX-202, RG-7863, RG-7854, RG-7795, and the compounds disclosed in US20100143301 (Gilead Sciences), US20110098248 (Gilead Sciences), and US20090047249 (Gilead Sciences), US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR7/TLR8 agonists include without limitation NKTR-262, telratolimod and BDB-001. TLR8 agonists include without limitation E-6887, IMO-4200, IMO-8400, IMO-9200, MCT-465, MEDI-9197, motolimod, resiquimod, GS-9688, VTX-1463, VTX-763, 3M-051, 3M-052, and the compounds disclosed in US20140045849 (Janssen), US20140073642 (Janssen), WO2014/056953 (Janssen), WO2014/076221 (Janssen), WO2014/128189 (Janssen), US20140350031 (Janssen), WO2014/023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics). TLR9 agonists include without limitation AST-008, cobitolimod, CMP-001, IMO-2055, IMO-2125, S-540956, litenimod, MGN-1601, BB-001, BB-006, IMO-3100, IMO-8400, IR-103, IMO-9200, agatolimod, DIMS-9054, DV-1079, DV-1179, AZD-1419, lefitolimod (MGN-1703), CYT-003, CYT-003-QbG10, tilsotolimod and PUL-042. Examples of TLR3 agonist include rintatolimod, poly-ICLC, RIBOXXON®, Apoxxim, RIBOXXIM®, IPH-33, MCT-465, MCT-475, and ND-1.1. TLR4 agonists include, but are not limited to, G-100 and GSK-1795091.

### CDK Inhibitors or Antagonists

In some embodiments, the agents described herein are combined with an inhibitor or antagonist of CDK. In some embodiments, the CDK inhibitor or antagonist is selected from the group consisting of VS2-370.

### STING Agonists, RIG-I and NOD2 Modulators

In some embodiments, the agents described herein are combined with a stimulator of interferon genes (STING). In some embodiments, the STING receptor agonist or activator is selected from the group consisting of ADU-5100 (MIW-815), SB-11285, MK-1454, SR-8291, AdVCA0848, GSK-532, SYN-STING, MSA-1, SR-8291, STING agonist (latent HIV), 5,6-dimethylxanthenone-4-acetic acid (DMXAA), cyclic-GAMP (cGAMP) and cyclic-di-AMP. In some embodiments, the agents described herein are combined with a RIG-I modulator such as RGT-100, or NOD2 modulator, such as SB-9200, and IR-103.

### LAG-3 and TIM-3 Inhibitors

In certain embodiments, the agents as described herein are combined with an anti-TIM-3 antibody, such as TSR-022, LY-3321367, MBG-453, INCAGN-2390.

In certain embodiments, the antibodies or antigen-binding fragments described herein are combined with an anti LAG-3 (Lymphocyte-activation) antibody, such as relatlimab (ONO-4482), LAG-525, MK-4280, REGN-3767, INCAGN2385.

### Interleukin Agonists

In certain embodiments, the agents described herein are combined with an interleukin agonist, such as IL-2, IL-7, IL-15, IL-10, IL-12 agonists; examples of IL-2 agonists such as proleukin (aldesleukin, IL-2); BC-IL (Cel-Sci), pegylated IL-2 (e.g., NKTR-214); modified variants of IL-2 (e.g., THOR-707), bempegaldesleukin, AIC-284, ALKS-4230, CUI-101, Neo-2/15; examples of IL-15 agonists, such as ALT-803, NKTR-255, and hetIL-15, interleukin-15/Fc fusion protein, AM-0015, NIZ-985, SO-C101, IL-15 Synthorin (pegylated Il-15), P-22339, and a IL-15 -PD-1 fusion protein N-809; examples of IL-7 include without limitation CYT-107.

Examples of additional immune-based therapies that can be combined with an agent of this disclosure include, but are not limited to, interferon alfa, interferon alfa-2b, interferon alfa-n3, pegylated interferon alfa, interferon gamma; FLT3 agonists such as CDX-301, GS-3583, gepon, normferon, peginterferon alfa-2a, peginterferon alfa-2b, and RPI-MN.

### Phosphatidylinositol 3-Kinase (PI3K) Inhibitors

Examples of PI3K inhibitors include, but are not limited to, idelalisib, alpelisib, buparlisib, CAI orotate, copanlisib, duvelisib, gedatolisib, neratinib, panulisib, perifosine, pictilisib, pilaralisib, puquitinib mesylate, rigosertib, rigosertib sodium, sonolisib, taselisib, AMG-319, AZD-8186, BAY-1082439, CLR-1401, CLR-457, CUDC-907, DS-7423, EN-3342, GSK-2126458, GSK-2269577, GSK-2636771, INCB-040093, LY-3023414, MLN-1117, PQR-309, RG-7666, RP-6530, RV-1729, SAR-245409, SAR-260301, SF-1126, TGR-1202, UCB-5857, VS-5584, XL-765, and ZSTK-474.

### Alpha-4/Beta-7 Antagonists

Examples of Integrin alpha-4/beta-7 antagonists include, but are not limited to, PTG-100, TRK-170, abrilumab, etrolizumab, carotegrast methyl, and vedolizumab.

### HPK1 Inhibitors

Examples of HPK1 inhibitors include, but are not limited to, ZYF-0272, and ZYF-0057.

### HIV Targeting Antibodies

Examples of HIV antibodies, bispecific antibodies, and “antibody-like” therapeutic proteins include, but are not limited to, DARTs®, DUOBODIES®, BITES®, XmAbs®, TandAbs®, Fab derivatives, bNAbs (broadly neutralizing HIV-1 antibodies), TMB-360, TMB-370, and those targeting HIV gp120 or gp41, antibody-Recruiting Molecules targeting HIV, anti-CD63 monoclonal antibodies, anti-GB virus C antibodies, anti-GP120/CD4, gp120 bispecific monoclonal antibody, CCR5 bispecific antibodies, anti-Nef single domain antibodies, anti-Rev antibody, camelid derived anti-CD18 antibodies, camelid-derived anti-ICAM-1 antibodies, DCVax-001, gp140 targeted antibodies, gp41-based HIV therapeutic antibodies, human recombinant mAbs (PGT-121), PGT121.414.LS, ibalizumab, ibalizumab (second generation), Immuglo, MB-66, clone 3 human monoclonal antibody targeting KLIC (HIV infection), GS-9721, BG-HIV, VRC-HIVMAB091-00-AB.

Various bNAbs may be used. Examples include, but are not limited to, those described in U.S. Pat. Nos. 8,673,307, 9,493,549, 9,783,594, 10,239,935, US2018371086, US2020223907, WO2014/063059, WO2012/158948, WO2015/117008, and PCT/US2015/41272, and WO2017/096221, including antibodies 12A12, 12A21, NIH45-46, bANC131, 8ANC134, 1B2530, INC9, 8ANC195. 8ANC196, 10-259, 10-303, 10-410, 10-847, 10-996, 10-1074, 10-1121, 10-1130, 10-1146, 10-1341, 10-1369, and 10-1074GM. Additional examples include those described in Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al, Nucleic Acids Res., 42 (Database issue):DI 133-9 (2014), Mascola et al., Immunol Rev., 254(1):225-44 (2013), such as 2F5, 4E10, M66.6, CAP206-CH12, 10E81 (all of which bind the MPER of gp41); PG9, PG16, CH01-04 (all of which bind V1V2-glycan), 2G12 (which binds to outer domain glycan); b12, HJ16, CH103-106, VRC01-03, VRC-PG04, 04b, VRC-CH30-34, 3BNC62, 3BNC89, 3BNC91, 3BNC95, 3BNC104, 3BNC176, and 8ANC131 (all of which bind to the CD4 binding site).

Additional broadly neutralizing antibodies that can be used as a second therapeutic agent in a combination therapy are described, e.g., in U.S. Pat. Nos. 8,673,307; 9,493,549; 9,783,594; and WO 2012/154312; WO2012/158948; WO 2013/086533; WO 2013/142324; WO2014/063059; WO 2014/089152, WO 2015/048462; WO 2015/103549; WO 2015/117008; WO2016/014484; WO 2016/154003; WO 2016/196975; WO 2016/149710; WO2017/096221; WO 2017/133639; WO 2017/133640, which are hereby incorporated herein by reference in their entireties for all purposes. Additional examples include, but are not limited to, those described in Sajadi et al., Cell. (2018) 173(7):1783-1795; Sajadi et al., J Infect Dis. (2016) 213(1):156-64; Klein et al., Nature, 492(7427): 118-22 (2012), Horwitz et al., Proc Natl Acad Sci USA, 110(41): 16538-43 (2013), Scheid et al., Science, 333: 1633-1637 (2011), Scheid et al., Nature, 458:636-640 (2009), Eroshkin et al., Nucleic Acids Res., 42 (Database issue):DI 133-9 (2014), Mascola et al., Immunol Rev., 254(1):225-44 (2013), such as 2F5, 4E10, M66.6, CAP206-CH12, 10E8, 10E8v4, 10E8-5R-100cF, DH511.11P, 7b2, 10-1074, and LN01 (all of which bind the MPER of gp41).

Examples of additional antibodies include, but are not limited to, bavituximab, UB-421, BF520.1, BiIA-SG, CH01, CH59, C2F5, C4E10, C2F5+C2G12+C4E10, CAP256V2LS, 3BNC117, 3BNC117-LS, 3BNC60, DH270.1, DH270.6, D1D2, 10-1074-LS, Cl3hmAb, GS-9722 (elipovimab), DH411-2, BG18, GS-9721, GS-9723, PGT145, PGT121, PGT-121.60, PGT-121.66, PGT122, PGT-123, PGT-124, PGT-125, PGT-126, PGT-151, PGT-130, PGT-133, PGT-134, PGT-135, PGT-128, PGT-136, PGT-137, PGT-138, PGT-139, MDX010 (ipilimumab), DH511, DH511-2, N6, N6LS, N49P6, N49P7, N49P7.1, N49P9, N49P11, N60P1.1, N60P25.1, N60P2.1, N60P31.1, N60P22, NIH 45-46, PGC14, PGG14, PGT-142, PGT-143, PGT-144, PGDM1400, PGDM12, PGDM21, PCDN-33A, 2Dm2m, 4Dm2m, 6Dm2m, PGDM1400, MDXO10 (ipilimumab), VRCO1, VRC-01-LS, A32, 7B2, 10E8, VRC-07-523, VRC07-523LS, VRC24, VRC41.01, 10E8VLS, 3810109, 10E8v4, IMC-HIV, iMabm36, eCD4-Ig, IOMA, CAP256-VRC26.25, DRVIA7,VRC-HIVMAB080-00-AB, VRC-HIVMABO60-00-AB, P2G12, VRC07, 354BG8, 354BG18, 354BG42, 354BG33, 354BG129, 354BG188, 354BG411, 354BG426, VRC29.03, CAP256, CAP256-VRC26.08, CAP256-VRC26.09, CAP256-VRC26.25, PCT64-24E and VRC38.01, PGT-151, CAP248-2B, 35022, ACS202, VRC34 and VRC34.01, 10E8, 10E8v4, 10E8-5R-100cF, 4E10, DH511.11P, 2F5, 7b2, and LNO1.

Examples of HIV bispecific and trispecific antibodies include without limitation MGD014, B12BiTe, BiIA-SG, TMB-bispecific, SAR-441236, VRC-01/PGDM-1400/10E8v4, 10E8.4/iMab, 10E8v4/PGT121-VRC01.

Examples of in vivo delivered bNAbs include without limitation AAV8-VRC07; mRNA encoding anti-HIV antibody VRC01; and engineered B-cells encoding 3BNC117 (Hartweger et al., J. Exp. Med. 2019, 1301).

### Pharmacokinetic Enhancers

Examples of pharmacokinetic enhancers include, but are not limited to, cobicistat and ritonavir.

### Additional Therapeutic Agents

Examples of additional therapeutic agents include, but are not limited to, the compounds disclosed in WO 2004/096286 (Gilead Sciences), WO 2006/015261 (Gilead Sciences), WO 2006/110157 (Gilead Sciences), WO 2012/003497 (Gilead Sciences), WO 2012/003498 (Gilead Sciences), WO 2012/145728 (Gilead Sciences), WO 2013/006738 (Gilead Sciences), WO 2013/159064 (Gilead Sciences), WO 2014/100323 (Gilead Sciences), US 2013/0165489 (University of Pennsylvania), US 2014/0221378 (Japan Tobacco), US 2014/0221380 (Japan Tobacco), WO 2009/062285 (Boehringer Ingelheim), WO 2010/130034 (Boehringer Ingelheim), WO 2013/006792 (Pharma Resources), US 20140221356 (Gilead Sciences), US 20100143301 (Gilead Sciences) and WO 2013/091096 (Boehringer Ingelheim).

### HIV Vaccines

Examples of HIV vaccines include, but are not limited to, peptide vaccines, recombinant subunit protein vaccines, live vector vaccines, DNA vaccines, HIV MAG DNA vaccine, CD4-derived peptide vaccines, vaccine combinations, adenoviral vector vaccines (an adenoviral vector such as Ad5, Ad26 or Ad35), simian adenovirus (chimpanzee, gorilla, rhesus i.e., rhAd), adeno-associated virus vector vaccines, Chimpanzee adenoviral vaccines (e.g., ChAdOX1, ChAd68, ChAd3, ChAd63, ChAd83, ChAd155, ChAd157, Pan5, Pan6, Pan7, Pan9), Coxsackieviruses based vaccines, enteric virus based vaccines, Gorilla adenovirus vaccines, lentiviral vector based vaccine, arenavirus vaccines (such as LCMV, Pichinde), bi-segmented or tri-segmented arenavirus based vaccine, trimer-based HIV-1 vaccine, measles virus based vaccine, flavivirus vector based vaccines, tobacco mosaic virus vector based vaccine, Varicella-zoster virus based vaccine, Human parainfluenza virus 3 (PIV3) based vaccines, poxvirus based vaccine (modified vaccinia virus Ankara (MVA), orthopoxvirus-derived NYVAC, and avipoxvirus-derived ALVAC (canarypox virus) strains); fowlpox virus based vaccine, rhabdovirus-based vaccines, such as VSV and marabavirus; recombinant human CMV (rhCMV) based vaccine, alphavirus-based vaccines, such as semliki forest virus, venezuelan equine encephalitis virus and sindbis virus; (see Lauer, Clinical and Vaccine Immunology, 2017, DOI: 10.1128/CVI.00298-16); LNP formulated mRNA based therapeutic vaccines; LNP-formulated self-replicating RNA/self-amplifying RNA vaccines.

Examples of vaccines include: AAVLP-HIV vaccine, AE-298p, anti-CD40.Env-gp140 vaccine, Ad4-EnvC150, BG505 SOSIP.664 gp140 adjuvanted vaccine, BG505 SOSIP.GT1.1 gp140 adjuvanted vaccine, ChAdOx1.tHIVconsv1 vaccine, CMV-MVA triplex vaccine, ChAdOx1.HTI, Chimigen HIV vaccine, ConM SOSIP.v7 gp140, ALVAC HIV (vCP1521), AIDSVAX B/E (gp120), monomeric gp120 HIV-1 subtype C vaccine, MPER-656 liposome subunit vaccine, Remune, ITV-1, Contre Vir, Ad5-ENVA-48, DCVax-001 (CDX-2401), Vacc-4x, Vacc-C5, VAC-3S, multiclade DNA recombinant adenovirus-5 (rAd5), rAd5 gag-pol env A/B/C vaccine, Pennvax-G, Pennvax-GP, Pennvax-G/MVA-CMDR, HIV-TriMix-mRNA vaccine, HIV-LAMP-vax, Ad35, Ad35-GRIN, NAcGM3/VSSP ISA-51, poly-ICLC adjuvanted vaccines, TatImmune, GTU-multiHIV (FIT-06), ChAdV63.HIVconsv, gp140[delta]V2.TV1+MF-59, rVSVIN HIV-1 gag vaccine, SeV-EnvF, SeV-Gag vaccine, AT-20, DNK-4, ad35-Grin/ENV, TBC-M4, HIVAX, HIVAX-2, N123-VRC-34.01 inducing epitope-based HIV vaccine, NYVAC-HIV-PT1, NYVAC-HIV-PT4, DNA-HIV-PT123, rAAV1-PG9DP, GOVX-B11, GOVX-B21, GOVX-C55, TVI-HIV-1, Ad-4 (Ad4-env Clade C+Ad4-mGag), Paxvax, EN41-UGR7C, EN41-FPA2, ENOB-HV-11, ENOB-HV-12, PreVaxTat, AE-H, MYM-V101, CombiHIVvac, ADVAX, MYM-V201, MVA-CMDR, MagaVax, DNA-Ad5 gag/pol/nef/nev (HVTN505), MVATG-17401, ETV-01, CDX-1401, DNA and Sev vectors vaccine expressing SCaVII, rcAD26.MOS1.HIV-Env, Ad26.Mod.HIV vaccine, Ad26.Mod.HIV+MVA mosaic vaccine+gp140, AGS-004, AVX-101, AVX-201, PEP-6409, SAV-001, ThV-01, TL-01, TUTI-16, VGX-3300, VIR-1111, IHV-001, and virus-like particle vaccines such as pseudovirion vaccine, CombiVICHvac, LFn-p24 B/C fusion vaccine, GTU-based DNA vaccine, HIV gag/pol/nef/env DNA vaccine, anti-TAT HIV vaccine, conjugate polypeptides vaccine, dendritic-cell vaccines (such as DermaVir), gag-based DNA vaccine, GI-2010, gp41 HIV-1 vaccine, HIV vaccine (PIKA adjuvant), i-key/MHC class II epitope hybrid peptide vaccines, ITV-2, ITV-3, ITV-4, LIPO-5, multiclade Env vaccine, MVA vaccine, Pennvax-GP, pp71-deficient HCMV vector HIV gag vaccine, rgp160 HIV vaccine, RNActive HIV vaccine, SCB-703, Tat Oyi vaccine, TBC-M4, UBI HIV gp120, Vacc-4x+romidepsin, variant gp120 polypeptide vaccine, rAd5 gag-pol env A/B/C vaccine, DNA.HTI and MVA.HTI, VRC-HIVDNA016-00-VP+VRC-HIVADV014-00-VP, INO-6145, JNJ-9220, gp145 C.6980; eOD-GT8 60mer based vaccine, PD-201401, env (A, B, C, A/E)/gag (C) DNA Vaccine, gp120 (A,B,C,A/E) protein vaccine, PDPHV-201401, Ad4-EnvCN54, EnvSeq-1 Envs HIV-1 vaccine (GLA-SE adjuvanted), HIV p24gag prime-boost plasmid DNA vaccine, HIV-1 iglb12 neutralizing VRC-01 antibody-stimulating anti-CD4 vaccine, arenavirus vector-based vaccines (Vaxwave, TheraT), MVA-BN HIV-1 vaccine regimen, mRNA based prophylactic vaccines, VPI-211, multimeric HIV gp120 vaccine (Fred Hutchinson cancer center), TBL-1203HI, CH505 TF chTrimer, CD40.HIVRI.Env vaccine, Drep-HIV-PT-1, mRNA-1644, and mRNA-1574.

### Birth Control (Contraceptive) Combination Therapy

In certain embodiments, the agents described herein are combined with a birth control or contraceptive regimen. Therapeutic agents used for birth control (contraceptive) that can be combined with an agent of this disclosure include without limitation cyproterone acetate, desogestrel, dienogest, drospirenone, estradiol valerate, ethinyl Estradiol, ethynodiol, etonogestrel, levomefolate, levonorgestrel, lynestrenol, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

In a particular embodiment, a compound disclosed herein, or a pharmaceutically acceptable salt thereof, is combined with one, two, three, or four additional therapeutic agents selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA@(tenofovir alafenamide, emtricitabine, cobicistat, and elvitegravir); BIKTARVY® (bictegravir+emtricitabine+tenofovir alafenamide), adefovir; adefovir dipivoxil; cobicistat; emtricitabine; tenofovir; tenofovir alafenamide and elvitegravir; tenofovir alafenamide+elvitegravir (rectal formulation, HIV infection); tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; PEGylated raltegravir; raltegravir and lamivudine; lamivudine+lopinavir+ritonavir+abacavir; maraviroc; tenofovir+emtricitabine+maraviroc, enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, an agent disclosed herein, or a pharmaceutical composition thereof, is combined with an HIV nucleoside or nucleotide inhibitor of reverse transcriptase and an HIV non-nucleoside inhibitor of reverse transcriptase. In another specific embodiment, an agent disclosed herein, or a pharmaceutical composition thereof, is combined with an HIV nucleoside or nucleotide inhibitor of reverse transcriptase, and an HIV protease inhibiting compound. In an additional embodiment, an agent disclosed herein, or a pharmaceutical composition thereof, is combined with an HIV nucleoside or nucleotide inhibitor of reverse transcriptase, an HIV non-nucleoside inhibitor of reverse transcriptase, and a pharmacokinetic enhancer. In certain embodiments, an agent disclosed herein, or a pharmaceutical composition thereof, is combined with at least one HIV nucleoside inhibitor of reverse transcriptase, an integrase inhibitor, and a pharmacokinetic enhancer. In another embodiment, an agent disclosed herein, or a pharmaceutical composition thereof, is combined with two HIV nucleoside or nucleotide inhibitors of reverse transcriptase.

In another embodiment, an agent disclosed herein, or a pharmaceutical composition thereof, is combined with a first additional therapeutic agent chosen from dolutegravir, cabotegravir, islatravir, darunavir, bictegravir, elsulfavirine, rilpivirine, and lenacapavir and a second additional therapeutic agent chosen from emtricitabine and lamivudine.

In some embodiments, an agent disclosed herein, or a pharmaceutical composition thereof, is combined with a first additional therapeutic agent (a contraceptive) selected from the group consisting of cyproterone acetate, desogestrel, dienogest, drospirenone, estradiol valerate, ethinyl Estradiol, ethynodiol, etonogestrel, levomefolate, levonorgestrel, lynestreno, medroxyprogesterone acetate, mestranol, mifepristone, misoprostol, nomegestrol acetate, norelgestromin, norethindrone, noretynodrel, norgestimate, ormeloxifene, segestersone acetate, ulipristal acetate, and any combinations thereof.

### Gene Therapy and Cell Therapy

In certain embodiments, the agents described herein are combined with a gene or cell therapy regimen. Gene therapy and cell therapy include without limitation the genetic modification to silence a gene; genetic approaches to directly kill the infected cells; the infusion of immune cells designed to replace most of the patient's own immune system to enhance the immune response to infected cells, or activate the patient's own immune system to kill infected cells, or find and kill the infected cells; genetic approaches to modify cellular activity to further alter endogenous immune responsiveness against the infection. Examples of cell therapy include without limitation LB-1903, ENOB-HV-01, ENOB-HV-21, ENOB-HV-31, GOVX-BO1, HSPCs overexpressing ALDH1 (LV-800, HIV infection), AGT103-T, and SupTI cell based therapy. Examples of dendritic cell therapy include without limitation AGS-004. CCR5 gene editing agents include without limitation SB-728T, SB-728-HSPC. CCR5 gene inhibitors include without limitation Cal-1, and lentivirus vector CCR5 shRNA/TRIM5alpha/TAR decoy-transduced autologous CD34-positive hematopoietic progenitor cells (HIV infection/HIV-related lymphoma). In some embodiments, C34-CCR5/C34-CXCR4 expressing CD4-positive T-cells are co-administered with one or more multi-specific antigen binding molecules. In some embodiments, the agents described herein are co-administered with AGT-103-transduced autologous T-cell therapy or AAV-eCD4-Ig gene therapy.

### Gene Editors

In certain embodiments, the agents described herein are combined with a gene editor, e.g., an HIV targeted gene editor. In various embodiments, the genome editing system can be selected from the group consisting of: a CRISPR/Cas9 complex, a zinc finger nuclease complex, a TALEN complex, a homing endonucleases complex, and a meganuclease complex. An illustrative HIV targeting CRISPR/Cas9 system includes without limitation EBT-101.

### CAR-T Cell Therapy

In some embodiments, the agents described herein can be co-administered with a population of immune effector cells engineered to express a chimeric antigen receptor (CAR), wherein the CAR comprises an HIV antigen binding domain. The HIV antigen include an HIV envelope protein or a portion thereof, gp120 or a portion thereof, a CD4 binding site on gp120, the CD4-induced binding site on gp120, N glycan on gp120, the V2 of gp120, the membrane proximal region on gp41. The immune effector cell is a T-cell or an NK cell. In some embodiments, the T-cell is a CD4+ T-cell, a CD8+ T-cell, or a combination thereof. Cells can be autologous or allogeneic. Examples of HIV CAR-T include A-1801, A-1902, convertible CAR-T, VC-CAR-T, CMV-N6-CART, anti-HIV duoCAR-T, anti-CD4 CART-cell therapy, CD4 CAR+C34-CXCR4+CCR5 ZFN T-cells, dual anti-CD4 CART-T cell therapy (CD4 CAR+C34-CXCR4 T-cells), anti-CD4 MicAbody antibody+anti-MicAbody CAR T-cell therapy (iNKG2D CAR, HIV infection), GP-120 CAR-T therapy, autologous hematopoietic stem cells genetically engineered to express a CD4 CAR and the C46 peptide.

### TCR T-Cell Therapy

In certain embodiments, the agents described herein are combined with a population of TCR-T-cells. TCR-T-cells are engineered to target HIV derived peptides present on the surface of virus-infected cells, for example, ImmTAV.

### B-Cell Therapy

In certain embodiments, the antibodies or antigen-binding fragments described herein are combined with a population of B cells genetically modified to express broadly neutralizing antibodies, such as 3BNC117 (Hartweger et al., J. Exp. Med. 2019, 1301, Moffett et al., Sci. Immunol. 4, eaax0644 (2019) 17 May 2019.

A compound as disclosed herein (e.g., any compound of Formula I) may be combined with one, two, three, or four additional therapeutic agents in any dosage amount of the compound of Formula I (e.g., from 1 mg to 500 mg of compound).

In one embodiment, kits comprising a compound disclosed herein, or a pharmaceutically acceptable salt thereof, in combination with one or more (e.g., one, two, three, one or two, or one to three) additional therapeutic agents are provided.

In one embodiment, the additional therapeutic agent or agents of the kit is an anti-HIV agent, selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), cell therapies (such as chimeric antigen receptor T-cell, CAR-T, and engineered T cell receptors, TCR-T, autologous T cell therapies), compounds that target the HIV capsid, latency reversing agents, HIV bNAbs, immune-based therapies, phosphatidylinositol 3-kinase (PI3K) inhibitors, HIV antibodies, broadly neutralizing HIV antibodies, bispecific antibodies and “antibody-like” therapeutic proteins, HIV p17 matrix protein inhibitors, IL-13 antagonists, peptidyl-prolyl cis-trans isomerase A modulators, protein disulfide isomerase inhibitors, complement C5a receptor antagonists, DNA methyltransferase inhibitor, HIV vif gene modulators, Vif dimerization antagonists, HIV viral infectivity factor inhibitors, TAT protein inhibitors, HIV Nef modulators, Hck tyrosine kinase modulators, mixed lineage kinase-3 (MLK-3) inhibitors, HIV splicing inhibitors, Rev protein inhibitors, integrin antagonists, nucleoprotein inhibitors, splicing factor modulators, COMM domain containing protein 1 modulators, HIV ribonuclease H inhibitors, retrocyclin modulators, CDK-9 inhibitors, dendritic ICAM-3 grabbing nonintegrin 1 inhibitors, HIV GAG protein inhibitors, HIV POL protein inhibitors, Complement Factor H modulators, ubiquitin ligase inhibitors, deoxycytidine kinase inhibitors, cyclin dependent kinase inhibitors, proprotein convertase PC9 stimulators, ATP dependent RNA helicase DDX3X inhibitors, reverse transcriptase priming complex inhibitors, G6PD and NADH-oxidase inhibitors, pharmacokinetic enhancers, HIV gene therapy, HIV vaccines, and combinations thereof.

In some embodiments, the additional therapeutic agent or agents of the kit are selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In a specific embodiment, the kit includes a compound disclosed herein, or a pharmaceutically acceptable salt thereof, and an HIV nucleoside or nucleotide inhibitor of reverse transcriptase. In a specific embodiment, the kit includes a compound disclosed herein, or a pharmaceutically acceptable salt thereof, and an HIV nucleoside or nucleotide inhibitor of reverse transcriptase and an HIV non-nucleoside inhibitor of reverse transcriptase. In another specific embodiment, the kit includes a compound disclosed herein, or a pharmaceutically acceptable salt thereof, and an HIV nucleoside or nucleotide inhibitor of reverse transcriptase, and an HIV protease inhibiting compound. In an additional embodiment, the kit includes a compound disclosed herein, or a pharmaceutically acceptable salt thereof, an HIV nucleoside or nucleotide inhibitor of reverse transcriptase, an HIV non-nucleoside inhibitor of reverse transcriptase, and a pharmacokinetic enhancer. In certain embodiments, the kit includes a compound disclosed herein, or a pharmaceutically acceptable salt thereof, at least one HIV nucleoside inhibitor of reverse transcriptase, an integrase inhibitor, and a pharmacokinetic enhancer. In another embodiment, the kit includes a compound disclosed herein, or a pharmaceutically acceptable salt thereof, and two HIV nucleoside or nucleotide inhibitors of reverse transcriptase. In a specific embodiment, the kit includes a compound disclosed herein, or a pharmaceutically acceptable salt thereof, an HIV nucleoside or nucleotide inhibitor of reverse transcriptase and an HIV capsid inhibitor. In a specific embodiment, the kit includes a compound disclosed herein, or a pharmaceutically acceptable salt thereof, an HIV nucleoside inhibitor of reverse transcriptase and an HIV capsid inhibitor. In a specific embodiment, the kit includes a compound disclosed herein, or a pharmaceutically acceptable salt thereof, and an HIV capsid inhibitor. In a specific embodiment, the kit includes a compound disclosed herein, or a pharmaceutically acceptable salt thereof, and one, two, three or four HIV bNAbs. In a specific embodiment, the kit includes a compound disclosed herein, or a pharmaceutically acceptable salt thereof, one, two, three or four HIV bNAbs and an HIV capsid inhibitor. In a specific embodiment, the kit includes a compound disclosed herein, or a pharmaceutically acceptable salt thereof, one, two, three or four HIV bNAbs, an HIV capsid inhibitor, and an HIV nucleoside inhibitor of reverse transcriptase.

### HIV Long Acting Therapy

Examples of drugs that are being developed as long acting regimens include, but are not limited to, cabotegravir, rilpivirine, any integrase LA, VM-1500 LAI, maraviroc (LAI), tenofovir implant, islatravir implant, doravirine, raltegravir, and long acting dolutegravir.

### PGP Inhibitors

In some embodiments, the compounds described herein can be used in combination with a P-glycoprotein (PGP) inhibitor. Examples of PGP inhibitors include, but are not limited to, verapamil, dexverapamil, cyclosporine, zosuquidar, laniquidar, elacridar, tariquidar, and encequidar. In some embodiments, the compounds described herein can be used in combination with encequidar. In some embodiments, the compounds described herein can be used in combination with a pharmaceutically acceptable salt of encequidar. In some embodiments, the compounds described herein can be used in combination with a mesylate salt of encequidar.

In some embodiments, the compounds described herein can be co-administered with a P-glycoprotein (PGP) inhibitor. Examples of PGP inhibitors include, but are not limited to, verapamil, dexverapamil, cyclosporine, zosuquidar, laniquidar, elacridar, tariquidar, and encequidar. In some embodiments, the compounds described herein can be co-administered with encequidar. In some embodiments, the compounds described herein can be co-administered with a pharmaceutically acceptable salt of encequidar. In some embodiments, the compounds described herein can be co-administered with a mesylate salt of encequidar.

### VII. COMPOUND PREPARATION

In some embodiments, the present disclosure provides processes and intermediates useful for preparing the compounds disclosed herein or pharmaceutically acceptable salts thereof.

Compounds disclosed herein can be purified by any of the means known in the art, including chromatographic means, including but not limited to high-performance liquid chromatography (HPLC), preparative thin layer chromatography, flash column chromatography, ion exchange chromatography, and supercritical fluid chromatography (SFC). Any suitable stationary phase can be used, including but not limited to, normal and reversed phases as well as ionic resins. In some embodiments, the disclosed compounds are purified via silica gel and/or alumina chromatography.

During any of the processes for preparation of the compounds provided herein, it can be necessary and/or desirable to protect sensitive or reactive groups on any of the molecules concerned. This can be achieved by means of conventional protecting groups as described in standard works, such as T. W. Greene and P. G. M. Wuts, PROTECTIVE GROUPS IN ORGANIC SYNTHESIS, 4th ed., Wiley, New York 2006. The protecting groups can be removed at a convenient subsequent stage using methods known from the art.

Exemplary chemical entities useful in methods of the embodiments will now be described by reference to illustrative synthetic schemes for their general preparation herein and the specific examples that follow. Skilled artisans will recognize that, to obtain the various compounds herein, starting materials can be suitably selected so that the ultimately desired substituents will be carried through the reaction scheme with or without protection as appropriate to yield the desired product. Alternatively, it can be necessary or desirable to employ, in the place of the ultimately desired substituent, a suitable group that can be carried through the reaction scheme and replaced as appropriate with the desired substituent. Furthermore, one of skill in the art will recognize that the transformations shown in the schemes below can be performed in any order that is compatible with the functionality of the particular pendant groups. Additionally, the amounts of reagents, the reaction conditions (e.g. temperature/pressure), and reaction time can be determined/optimized by persons of skill in the art inview of the current disclosure.

Compounds disclosed herein can be prepared from commercially available reagents using the synthetic methods and reaction schemes described herein, or using other reagents and conventional methods known to persons of ordinary skill in the art. For instance, representative syntheses of compounds of the present disclosure are described in the schemes below, and the particular examples that follow.

### IX. EXAMPLES

### Synthesis of Intermediate A

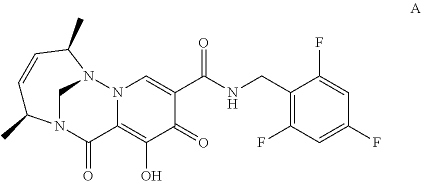

(1S,2R,5S)-8-hydroxy-2,5-dimethyl-7,9-dioxo-N-(2,4,6-trifluorobenzyl)-2,5,7,9-tetrahydro-1,6-methanopyrido[1,2-b][1,2,5]triazonine-10-carboxamide (Intermediate A) was prepared according to Example 24 of WO2022159387.

### Example 1: Synthesis of Esters of Intermediate A

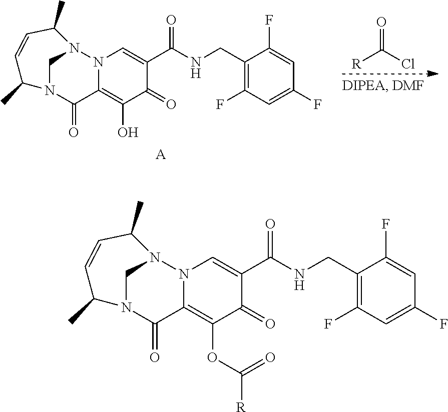

To a solution of Intermediate A in DMF is added DIPEA and the mixture is cooled to 0 °C. The acyl chloride (RCOCl) is added and the reaction mixture is allowed to warm to rt and stir for 48 h. The reaction mixture is diluted with EtOAc and is washed with water and brine. The organic phase is dried over Na₂SO₄, filtered, and concentrated. The residue is purified by silica gel column chromatography (0-100% EtOAc/hexane) and fractions containing product are pooled, concentrated, and lyophilized to afford the desired compound.

### Example 1a: Alternative Synthesis of Esters of Intermediate a Starting from Corresponding Acids

### Step 1: Synthesis of Acyl Chloride

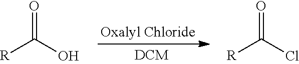

To a solution of corresponding acid (1 eq) in DCM (10 vol), was added oxalyl dichloride (1.8 eq) at 0 °C. Then the reaction mixture was allowed to warm to ambient temperature and stir for 2 h. The reaction mixture was diluted with DCM and washed with water followed by brine solution. The organic phase was dried over Na₂SO₄, filtered, and concentrated to afford the corresponding acyl chloride.

### Step 2: Synthesis of Esters

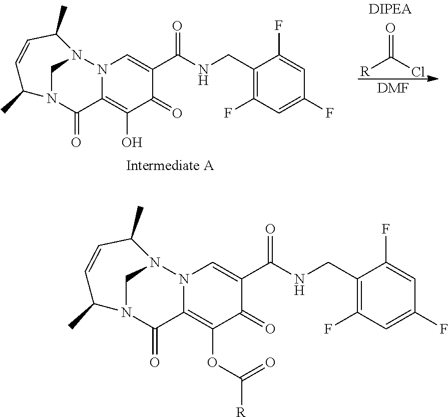

To a solution of Intermediate A (0.2 g, 0.44 mmol, 1 eq) in DMF (2 mL) was added DIPEA (0.3 mL, 1.76 mmol, 4 eq) and the mixture is cooled to 0 °C. The acyl chloride (0.1 mL, 1.1 mmol, 2.5 eq) was added and the reaction mixture was allowed to warm to rt and stir for 48 h. The reaction mixture was diluted with EtOAc and was washed with water and brine. The organic phase was dried over Na₂SO₄, filtered, and concentrated. The residue was purified by silica gel column chromatography (0-100% EtOAc/hexane) and fractions containing product were pooled, concentrated, and lyophilized from 1:1 MeCN/water to afford the desired compound.

The following compounds are prepared following the procedure of Example 1 using the acyl chlorides indicated below.

Alternatively, the following compounds were prepared following the procedure of Step 2 of Example 1a using the acyl chlorides indicated below. The acyl chlorides for compounds C19-C23 and C26 were prepared following the procedure of Step 1 of Example 1a using the corresponding acids indicated below.

| Compound | | |
|---|---|---|
| No. | Structure | Acyl Chloride* |
| Cl | 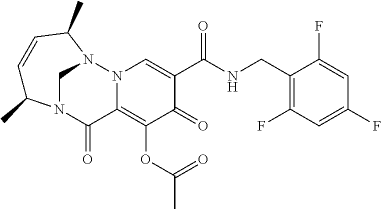 | CH₃COCl |
| C2 | 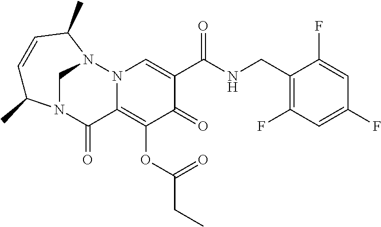 | CH₃CH₂COCl |
| C3 | 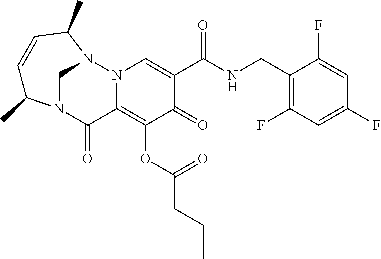 | CH₃(CH₂)₂COCl |
| C4 | 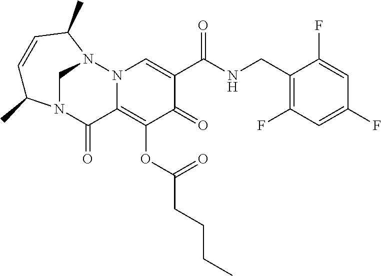 | CH₃(CH₂)₃COCl |
| C5 | 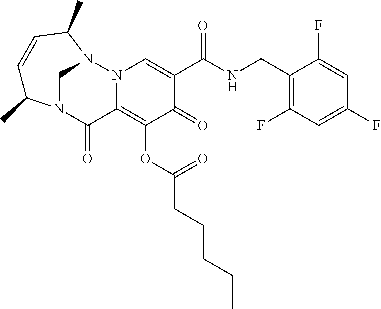 | CH₃(CH₂)₄COCl |
| C6 | 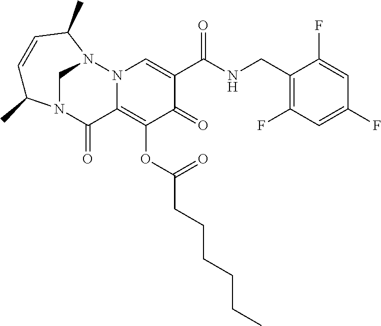 | CH₃(CH₂)₅COCl |
| C7 | 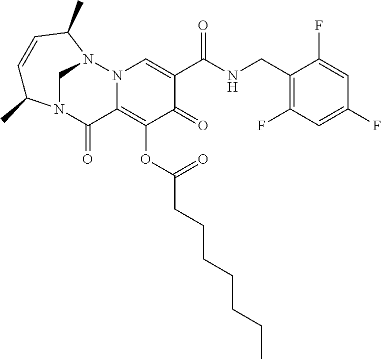 | CH₃(CH₂)₆COCl |
| C8 | 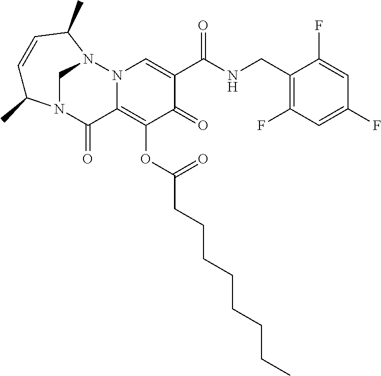 | CH₃(CH₂)₇COCl |
| C9 | 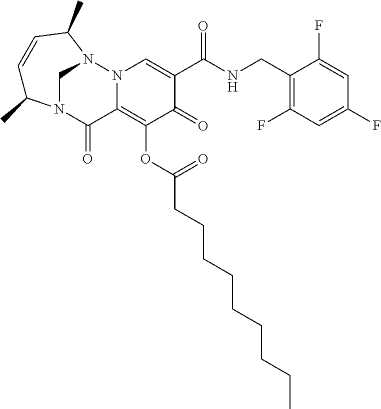 | CH₃(CH₂)₈COCl |
| C10 | 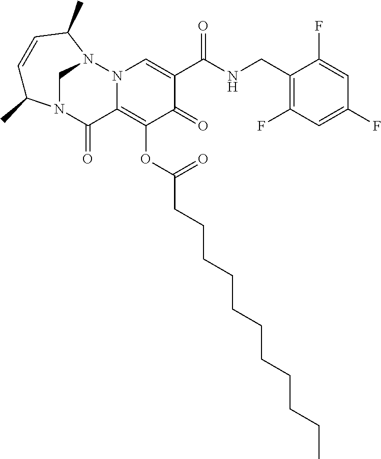 | CH₃(CH₂)₁₀COCl |
| C11 | 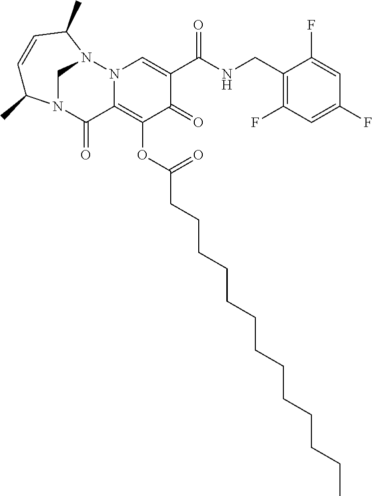 | CH₃(CH₂)₁₂COCl |
| C12 | 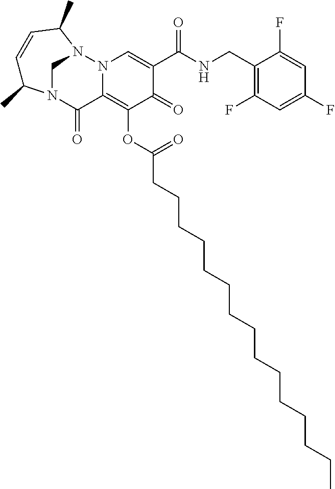 | CH₃(CH₂)₁₄COCl |
| C13 | 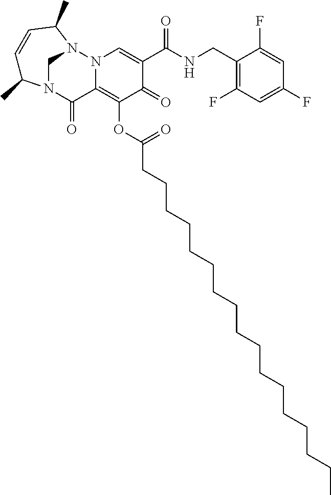 | CH₃(CH₂)₁₆COCl |
| C14 | 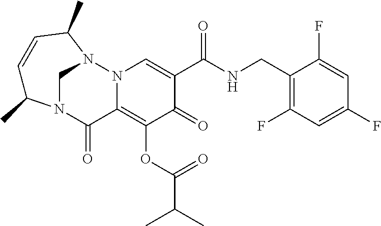 | (CH₃)₂CHCOCl |
| C15 | 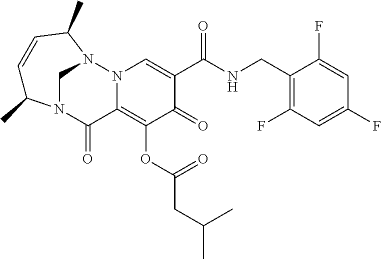 | (CH₃)₂CHCH₂COCl |
| C16 | 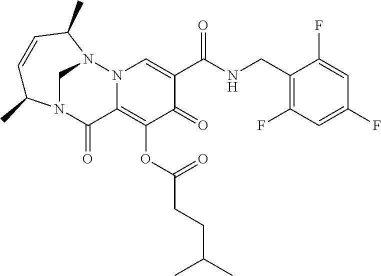 | (CH₃)₂CH(CH₂)₂COCl |
| C17 | 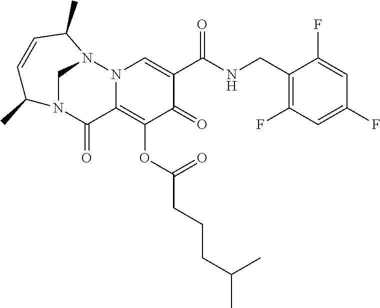 | (CH₃)₂CH(CH₂)₃COCl |
| C18 | 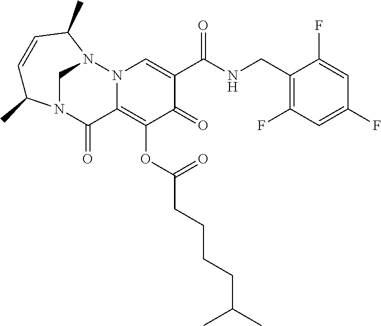 | (CH₃)₂CH(CH₂)₄COCl |
| C19 | 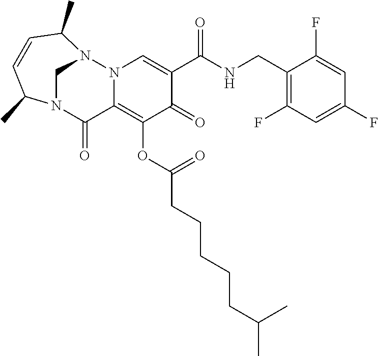 | (CH₃)₂CH(CH₂)₅COCl Prepared starting from corresponding acid (CH₃)₂CH(CH₂)₅COOH |
| C20 | 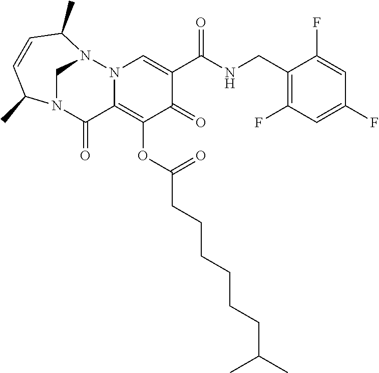 | (CH₃)₂CH(CH₂)₆COCl Prepared starting from corresponding acid (CH₃)₂CH(CH₂)₆COOH |
| C21 | 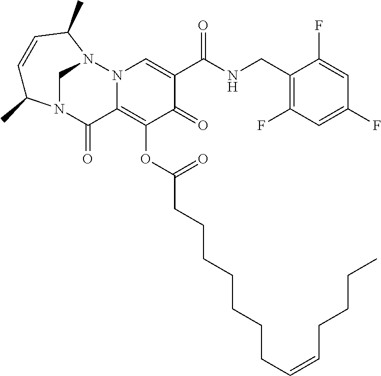 | Prepared starting from corresponding acid CH₃(CH₂)₃CH═CH(CH₂)₇COOH |
| C22 | 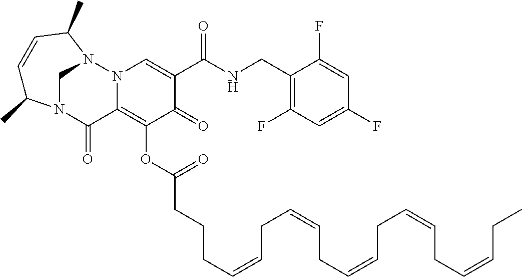 | CH₃(CH₂CH═CH)₅(CH₂)₃COCl Prepared starting from corresponding acid CH₃(CH₂CH═CH)₅(CH₂)₃COOH |
| C23 | 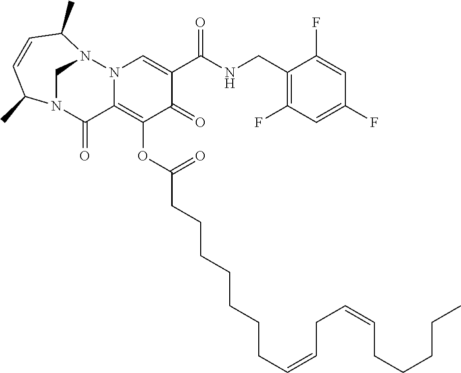 | CH₃(CH₂)₃(CH₂CH═CH)₂(CH₂)₇COCl Prepared starting from corresponding acid CH₃(CH₂)₃(CH₂CH═CH)₂(CH₂)₇COOH |
| C24 | 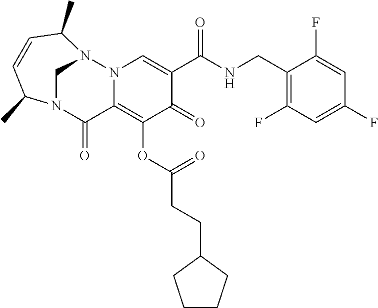 | cyclopentyl-CH₂CH₂COCl |
| C25 | 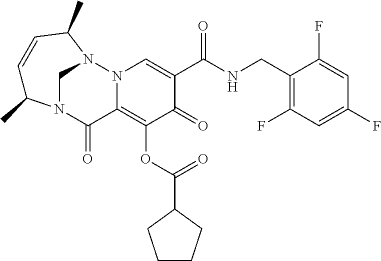 | cyclopentyl-COCl |
| C26 | 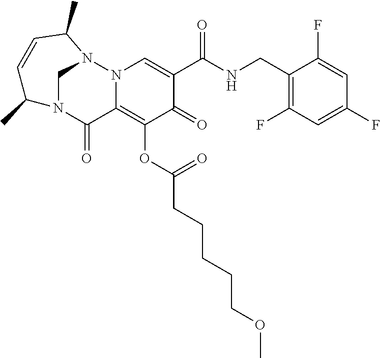 | CH₃O(CH₂)₅COCl Prepared starting from corresponding acid CH₃O(CH₂)₅COOH |

*Commercially available or prepared according to literature.

### Example 2: Synthesis of Carboxylic Acid Substituted Esters of Intermediate A

### Step 1: Synthesis of Acyl Chloride

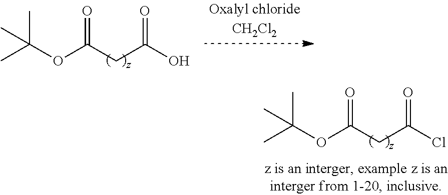

To a solution of the carboxylic acid in CH₂Cl₂ (1.35 M) at 0 °C is added 1 drop of DMF followed by oxalyl chloride. The reaction mixture is stirred at 0 °C until completion and concentrated. The residue is used in the subsequent step without further purification.

### Step 2: Acylation of Intermediate A

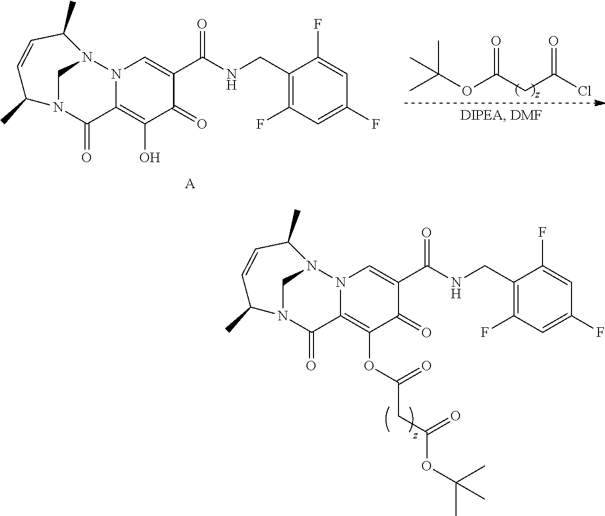

To a solution of Intermediate A in DMF (0.2 M) is added DIPEA and the mixture is cooled to 0 °C. The acyl chloride is added and the reaction mixture is allowed to warm to rt and stir for 48 h. The reaction mixture is diluted with EtOAc and is washed with water and brine. The organic phase is dried over Na₂SO₄, filtered, and concentrated. The residue is purified by silica gel column chromatography (0-100% EtOAc/hexane) and fractions containing product are pooled and concentrated to afford the desired compound.

### Step 3: Deprotection of Tert-Butyl Ester

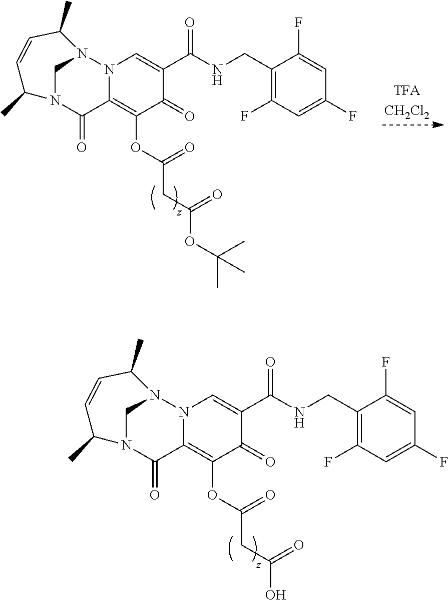

To a solution of the tert-butyl ester in CH₂Cl₂ (0.1 M) at 0 °C is added TFA dropwise. The reaction mixture is allowed to warm to rt. Upon completion, the reaction mixture is concentrated and the residue is purified by reverse phase prep HPLC (5-100% MeCN/H₂O w/0.1% TFA) to afford the desired compound after lyophilization.

The following compounds are prepared following the procedure of Example 2 using the carboxylic acid indicated below.

| Compound | | |
|---|---|---|
| No. | Structure | Carboxylic Acid* |
| C27 | 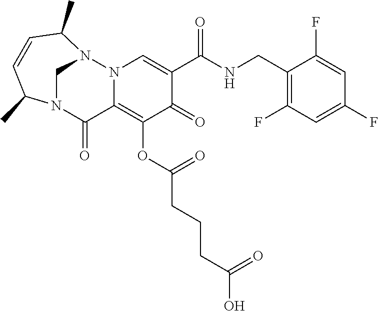 | COOH(CH₂)₃COOC(CH₃)₃ |
| C28 | 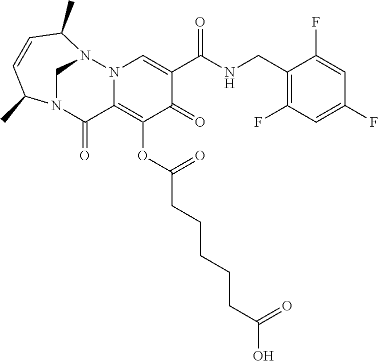 | COOH(CH₂)₅COOC(CH₃)₃ |
| C29 | 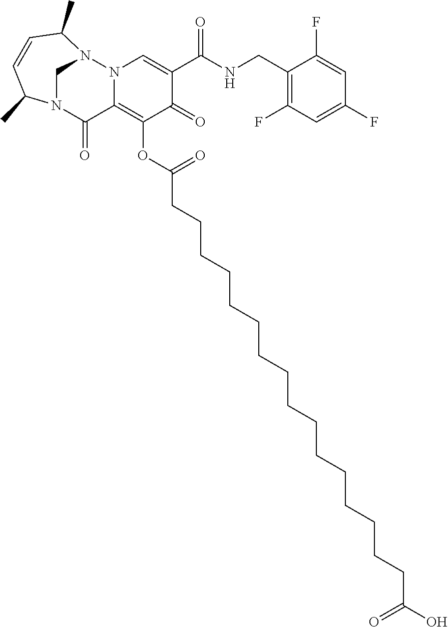 | COOH(CH₂)₁₆COOC(CH₃)₃ |

*Commercially available or prepared according to literature.

### Example 2a: Alternative Synthesis of Compound C27

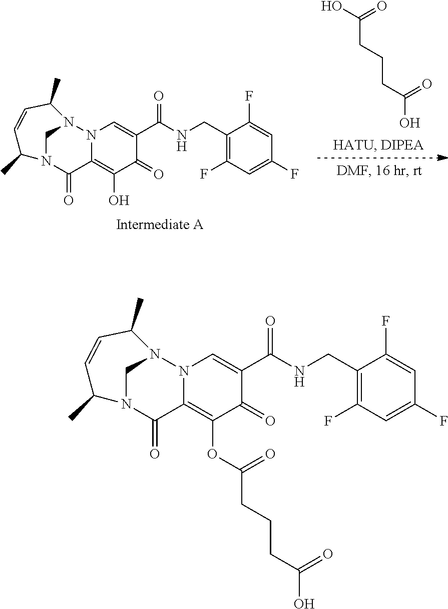

To a solution of Intermediate A (0.25 g, 0.55 mmol) and glutaric acid (0.117 g, 0.89 mmol) in DMF (4.0 mL), were added DIPEA (0.29 mL, 1.67 mmol) and HATU (311.66 mg, 1.11 mmol) at room temperature. The resulting mixture was stirred for 16 hours. Progress of the reaction was monitored by TLC and LCMS. After completion, the reaction mixture was diluted with ethyl acetate (50 mL), washed with water (2×50 mL) and brine solution (2×50 mL). The organic layers were dried over anhydrous sodium sulphate and concentrated under reduced pressure to give crude compound. The crude compound was purified by Prep-HPLC column chromatography with the condition below, and followed by Prep-SFC purification with the condition below. The pure fractions were concentrated and lyophilized to obtain compound C27. LCMS (m/z): 562 [M+H]⁺.

Prep. HPLC Method:

MOBILE PHASE A:0.1% TFA IN WATER MOBILE PHASE B: ACN GRADIENT ELUTION:(T/% B)=0/30,2/40,10/90,15/90,15.1/98,18/98,18.1/30,21/30 SOLUBILITY: ACN-THF-WATER FLOW RATE: 15 ML/min COLUMN: CHIRALPACK-IC(19*150 mm)5μ

### Prep-SFC Method:

Column: CHIRALCEL OX-H (4.6*150 mm) 5 μm, Co-Solvent: 0.5% DEA IN METHANOL

Total Flow: 4 mL/min, % of CO₂: 60, % of Co-Solvent: 40, ABPR: 1500 psi, Column Temp: 30 °C, Diluent: ACN.

### Example 2b: Alternative Synthesis of Compound C28

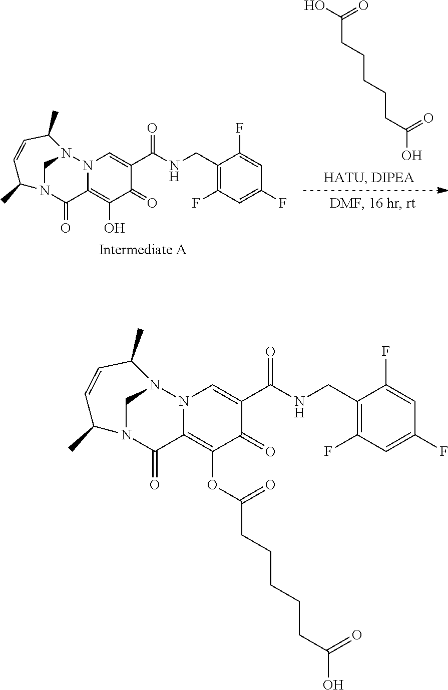

To a solution of Intermediate A (0.25 g, 0.55 mmol) and heptanedioic acid (0.143 g, 0.89 mmol) in DMF (4.0 mL), were added DIPEA (0.29 mL, 1.67 mmol) and HATU (311.66 mg, 1.11 mmol) at room temperature. The resulting mixture was stirred for 16 hours. Progress of the reaction was monitored by TLC and LCMS. After completion, the reaction mixture was diluted with ethyl acetate (50 mL), washed with water (2×50 mL) and brine solution (2×50 mL). The organic layers were dried over anhydrous sodium sulphate and concentrated under reduced pressure to give crude compound. The crude compound was purified Prep-HPLC column chromatography with the condition below in ACN: WATER, and the pure fractions were collected and lyophilized to afford compound C28.

Prep. HPLC Method:

MOBILE PHASE A:0.1% FORMIC ACID IN WATER MOBILE PHASE B: ACN GRADIENT ELUTION:(T/% B)=0/65,2/65,10/80,16.1/99,19/99,19.1/65,21/65 SOLUBILITY: ACN-THF-WATER FLOW RATE: 15 ML/min COLUMN=SUNFIRE-C18(19*150 mm); 5u

### Example 2c: Alternative Synthesis of Compound C29

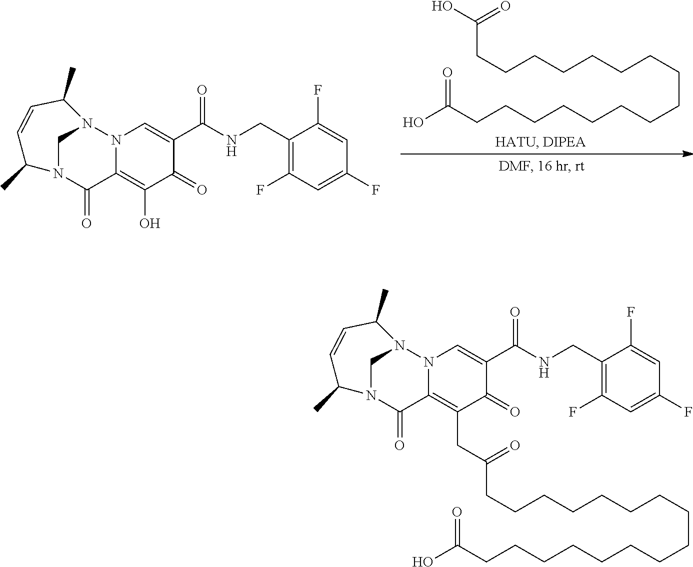

To a solution of Intermediate A (0.25 g, 0.55 mmol) and octadecanedioic acid (0.193 g, 1.1 mmol) in DMF (4.0 mL), were added DIPEA (0.29 mL, 1.67 mmol) and HATU (311.66 mg, 1.11 mmol) at room temperature. The resulting mixture was stirred for 16 hours. Progress of the reaction was monitored by TLC and LCMS. After completion, the reaction mixture was diluted with ethyl acetate (50 mL), washed with water (2×50 mL) and brine solution (2×50 mL). The organic layers were dried over anhydrous sodium sulphate and concentrated under reduced pressure to give crude compound. The crude compound was purified Prep-HPLC column chromatography with the condition below in ACN: WATER, and the pure fractions were collected and lyophilized to afford compound C29.

Prep. HPLC Method:

MOBILE PHASE A:0.1% FORMIC ACID IN WATER MOBILE PHASE B: ACN GRADIENT ELUTION:(T/% B)=0/65,2/65,10/80,16.1/99,19/99,19.1/65,21/65 SOLUBILITY: ACN-THF-WATER FLOW RATE: 15 ML/min COLUMN=SUNFIRE-C18(19*150 mm); 5u

The characterization data of the compounds C1-C29 are shown in the table below.

| | Chemical | |
|---|---|---|
| Compound | Formula | |
| No. | MS (m/z) | ¹H NMR Peak Table |
| C1 | C23H21F3N4O5 | 1H NMR (400 MHz, DMSO) δ 10.17 (t, J = 5.8 Hz, 1H), |
| | 491.34 [M + H] | 8.43 (s, 1H), 7.27-7.14 (m, 2H), 5.64 (dt, J = 11.6, 2.4 Hz, |
| | | 1H), 5.35 (ddd, J = 11.6, 3.6, 2.5 Hz, 1H), 5.31-5.21 (m, |
| | | 1H), 5.05 (d, J = 14.6 Hz, 1H), 4.77 (d, J = 14.5 Hz, 1H), |
| | | 4.55 (d, J = 5.8 Hz, 2H), 3.84 (dd, J = 6.9, 3.7 Hz, 1H), |
| | | 2.26 (s, 3H), 1.34 (d, J = 6.8 Hz, 3H), 1.28 (d, J = 7.3 Hz, 3H). |
| C2 | C24H23F3N4O5 | 1H NMR (400 MHz, DMSO) δ 10.16 (t, J = 5.8 Hz, 1H), |
| | 505.39 [M + H] | 8.43 (s, 1H), 7.27-7.14 (m, 2H), 5.64 (dt, J = 11.6, 2.4 Hz, |
| | | 1H), 5.35 (ddd, J = 11.5, 3.5, 2.5 Hz, 1H), 5.30-5.23 (m, |
| | | 1H), 5.04 (d, J = 14.6 Hz, 1H), 4.77 (d, J = 14.5 Hz, 1H), |
| | | 4.55 (d, J = 5.8 Hz, 2H), 3.84 (dt, J = 6.4, 3.3 Hz, 1H), 2.58 |
| | | (qd, J = 7.5, 4.0 Hz, 2H), 1.34 (d, J = 6.8 Hz, 3H), 1.28 (d, |
| | | J = 7.3 Hz, 3H), 1.14 (t, J = 7.5 Hz, 3H). |
| C3 | C25H25F3N4O5 | 1H NMR (400 MHz, DMSO) δ 10.15 (t, J = 5.8 Hz, 1H), |
| | 519.42 [M + H] | 8.43 (s, 1H), 7.26-7.14 (m, 2H), 5.64 (dt, J = 11.6, 2.4 Hz, |
| | | 1H), 5.35 (ddd, J = 11.6, 3.6, 2.5 Hz, 1H), 5.27 (dq, J = 7.7, |
| | | 2.7 Hz, 1H), 5.04 (d, J = 14.6 Hz, 1H), 4.77 (d, J = 14.5 Hz, |
| | | 1H), 4.55 (d, J = 5.8 Hz, 2H), 3.85 (dt, J = 6.6, 3.2 Hz, 1H), |
| | | 2.63-2.51 (m, 2H), 1.65 (h, J = 7.3 Hz, 2H), 1.34 (d, J = |
| | | 6.8 Hz, 3H), 1.28 (d, J = 7.3 Hz, 3H), 0.99 (t, J = 7.4 Hz, 3H). |
| C4 | C26H27F3N4O5 | 1H NMR (400 MHz, DMSO) δ 10.15 (t, J = 5.8 Hz, 1H), |
| | 533.37 [M + H] | 8.43 (s, 1H), 7.26-7.14 (m, 2H), 5.64 (dt, J = 11.6, 2.4 Hz, |
| | | 1H), 5.35 (ddd, J = 11.5, 3.6, 2.5 Hz, 1H), 5.30-5.23 (m, |
| | | 1H), 5.04 (d, J = 14.6 Hz, 1H), 4.77 (d, J = 14.5 Hz, 1H), |
| | | 4.55 (d, J = 5.8 Hz, 2H), 3.84 (dd, J = 6.5, 3.2 Hz, 1H), |
| | | 2.64-2.51 (m, 2H), 1.68-1.56 (m, 2H), 1.46-1.37 (m, |
| | | 2H), 1.34 (d, J = 6.8 Hz, 3H), 1.28 (d, J = 7.3 Hz, 3H), 0.91 |
| | | (t, J = 7.3 Hz, 3H). |
| C5 | C27H29F3N4O5 | 1H NMR (400 MHz, DMSO) δ 10.15 (t, J = 5.8 Hz, 1H), |
| | 547.41 [M + H] | 8.43 (s, 1H), 7.26-7.14 (m, 2H), 5.64 (dt, J = 11.6, 2.4 Hz, |
| | | 1H), 5.35 (ddd, J = 11.6, 3.6, 2.5 Hz, 1H), 5.30-5.23 (m, |
| | | 1H), 5.04 (d, J = 14.6 Hz, 1H), 4.77 (d, J = 14.5 Hz, 1H), |
| | | 4.55 (d, J = 5.8 Hz, 2H), 3.84 (dd, J = 7.0, 3.6 Hz, 1H), |
| | | 2.64-2.52 (m, 2H), 1.63 (p, J = 7.3 Hz, 2H), 1.41-1.20 |
| | | (m, 10H), 0.88 (t, J = 7.1 Hz, 3H). |
| C6 | C28H31F3N4O5 | 1H NMR (400 MHz, DMSO) δ 10.15 (t, J = 5.8 Hz, 1H), |
| | 561.40 [M + H] | 8.43 (s, 1H), 7.26-7.14 (m, 2H), 5.64 (dt, J = 11.6, 2.4 Hz, |
| | | 1H), 5.35 (ddd, J = 11.6, 3.6, 2.5 Hz, 1H), 5.26 (dq, J = 7.7, |
| | | 2.7 Hz, 1H), 5.04 (d, J = 14.6 Hz, 1H), 4.77 (d, J = 14.5 Hz, |
| | | 1H), 4.55 (d, J = 5.7 Hz, 2H), 3.84 (dq, J = 6.6, 3.3 Hz, |
| | | 1H), 2.64-2.51 (m, 2H), 1.68-1.56 (m, 2H), 1.45-1.18 |
| | | (m, 12H), 0.92-0.81 (m, 3H). |
| C7 | C29H33F3N4O5 | 1H NMR (400 MHz, DMSO) δ 10.15 (t, J = 5.8 Hz, 1H), |
| | 575.44 [M + H] | 8.43 (s, 1H), 7.26-7.14 (m, 2H), 5.64 (dt, J = 11.6, 2.4 Hz, |
| | | 1H), 5.35 (ddd, J = 11.5, 3.6, 2.5 Hz, 1H), 5.26 (dq, J = 7.7, |
| | | 2.7 Hz, 1H), 5.04 (d, J = 14.6 Hz, 1H), 4.77 (d, J = 14.5 Hz, |
| | | 1H), 4.55 (d, J = 5.7 Hz, 2H), 3.88-3.80 (m, 1H), 2.64- |
| | | 2.51 (m, 2H), 1.62 (p, J = 7.2 Hz, 2H), 1.46-1.16 (m, |
| | | 14H), 0.91-0.82 (m, 3H). |
| C8 | C30H35F3N4O5 | 1H NMR (400 MHz, DMSO) δ 10.15 (t, J = 5.8 Hz, 1H), |
| | 589.48 [M + H] | 8.43 (s, 1H), 7.26-7.14 (m, 2H), 5.64 (dt, J = 11.6, 2.4 Hz, |
| | | 1H), 5.35 (ddd, J = 11.6, 3.6, 2.5 Hz, 1H), 5.30-5.21 (m, |
| | | 1H), 5.04 (d, J = 14.6 Hz, 1H), 4.77 (d, J = 14.5 Hz, 1H), |
| | | 4.55 (d, J = 5.7 Hz, 2H), 3.88-3.81 (m, 1H), 2.63-2.51 |
| | | (m, 2H), 1.62 (p, J = 7.3 Hz, 2H), 1.46-1.14 (m, 16H), |
| | | 0.90-0.82 (m, 3H). |
| C9 | C31H37F3N4O5 | 1H NMR (400 MHz, DMSO) δ 10.15 (t, J = 5.8 Hz, 1H), |
| | 603.47 [M + H] | 8.43 (s, 1H), 7.26-7.14 (m, 2H), 5.64 (dt, J = 11.5, 2.4 Hz, |
| | | 1H), 5.35 (ddd, J = 11.5, 3.6, 2.5 Hz, 1H), 5.26 (d, J = 8.2 |
| | | Hz, 1H), 5.04 (d, J = 14.6 Hz, 1H), 4.77 (d, J = 14.5 Hz, |
| | | 1H), 4.54 (d, J = 5.8 Hz, 2H), 3.85 (s, 1H), 2.63-2.51 (m, |
| | | 2H), 1.62 (p, J = 7.2 Hz, 2H), 1.44-1.17 (m, 18H), 0.90- |
| | | 0.82 (m, 3H). |
| C10 | C33H41F3N4O5 | 1H NMR (400 MHz, DMSO) δ 10.15 (t, J = 5.8 Hz, 1H), |
| | 631.49 [M + H] | 8.43 (s, 1H), 7.26-7.13 (m, 2H), 5.64 (dt, J = 11.5, 2.4 Hz, |
| | | 1H), 5.35 (ddd, J = 11.6, 3.6, 2.5 Hz, 1H), 5.31-5.21 (m, |
| | | 1H), 5.04 (d, J = 14.6 Hz, 1H), 4.77 (d, J = 14.5 Hz, 1H), |
| | | 4.55 (d, J = 5.7 Hz, 2H), 3.90-3.80 (m, 1H), 2.63-2.51 (m, |
| | | 2H), 1.62 (p, J = 7.2 Hz, 2H), 1.45-1.16 (m, 22H), 0.89- |
| | | 0.81 (m, 3H). |
| C11 | C35H45F3N4O5 | 1H NMR (400 MHz, DMSO) δ 10.16 (t, J = 5.8 Hz, 1H), |
| | 659.54 [M + H] | 8.43 (s, 1H), 7.24-7.15 (m, 2H), 5.64 (dt, J = 11.6, 2.4 Hz, |
| | | 1H), 5.35 (dt, J = 11.6, 3.0 Hz, 1H), 5.26 (s, 1H), 5.04 (d, J = |
| | | 14.6 Hz, 1H), 4.77 (d, J = 14.5 Hz, 1H), 4.54 (d, J = 5.8 |
| | | Hz, 2H), 3.90-3.80 (m, 1H), 2.61-2.51 (m, 2H), 1.62 (p, J = |
| | | 7.3 Hz, 2H), 1.45-1.15 (m, 26H), 0.89-0.81 (m, 3H). |
| C12 | C37H49F3N4O5 | 1H NMR (401 MHz, DMSO) δ 10.15 (t, J = 5.8 Hz, 1H), |
| | 687.61 [M + H] | 8.43 (s, 1H), 7.25-7.14 (m, 2H), 5.64 (dt, J = 11.6, 2.4 Hz, |
| | | 1H), 5.35 (ddd, J = 11.6, 3.6, 2.5 Hz, 1H), 5.30-5.23 (m, |
| | | 1H), 5.04 (d, J = 14.6 Hz, 1H), 4.77 (d, J = 14.6 Hz, 1H), |
| | | 4.54 (d, J = 5.8 Hz, 2H), 3.88-3.80 (m, 1H), 2.55 (dd, J = |
| | | 7.5, 6.0 Hz, 2H), 1.62 (p, J = 7.3 Hz, 2H), 1.44-1.16 (m, |
| | | 30H), 0.89-0.81 (m, 3H). |
| C13 | C39H53F3N4O5 | 1H NMR (400 MHz, DMSO) δ 10.16 (t, J = 5.8 Hz, 1H), |
| | 715.63 [M + H] | 8.43 (s, 1H), 7.24-7.14 (m, 2H), 5.64 (dt, J = 11.6, 2.4 Hz, |
| | | 1H), 5.35 (ddd, J = 11.6, 3.6, 2.5 Hz, 1H), 5.26 (dd, J = 7.7, |
| | | 2.5 Hz, 1H), 5.04 (d, J = 14.6 Hz, 1H), 4.76 (d, J = 14.6 Hz, |
| | | 1H), 4.54 (d, J = 5.8 Hz, 2H), 3.90-3.80 (m, 1H), 2.59- |
| | | 2.51 (m, 2H), 1.62 (p, J = 7.5 Hz, 2H), 1.42-1.16 (m, |
| | | 34H), 0.89-0.81 (m, 3H). |
| C14 | C25H25F3N4O5 | 1H NMR (400 MHz, DMSO) δ 10.13 (t, J = 5.8 Hz, 1H), |
| | 519.28 [M + H] | 8.43 (s, 1H), 7.26-7.14 (m, 2H), 5.64 (dt, J = 11.6, 2.5 Hz, |
| | | 1H), 5.35 (ddd, J = 11.6, 3.6, 2.5 Hz, 1H), 5.26 (s, 1H), |
| | | 5.04 (d, J = 14.6 Hz, 1H), 4.77 (d, J = 14.6 Hz, 1H), 4.54 |
| | | (d, J = 5.7 Hz, 2H), 3.85 (s, 1H), 2.80 (hept, J = 6.9 Hz, |
| | | 1H), 1.34 (d, J = 6.8 Hz, 3H), 1.28 (d, J = 7.3 Hz, 3H), 1.23 |
| | | (dd, J = 7.0, 5.8 Hz, 6H). |
| C15 | C26H27F3N4O5 | 1H NMR (400 MHz, DMSO) δ 10.15 (t, J = 5.8 Hz, 1H), |
| | 533.41 [M + H] | 8.43 (s, 1H), 7.20 (t, J = 8.6 Hz, 2H), 5.64 (dt, J = 11.5, 2.4 |
| | | Hz, 1H), 5.40-5.31 (m, 1H), 5.26 (s, 1H), 5.04 (d, J = |
| | | 14.6 Hz, 1H), 4.77 (d, J = 14.5 Hz, 1H), 4.55 (d, J = 5.7 Hz, |
| | | 2H), 3.85 (d, J = 3.5 Hz, 1H), 2.47-2.36 (m, 2H), 2.16- |
| | | 2.04 (m, 1H), 1.34 (d, J = 6.8 Hz, 3H), 1.28 (d, J = 7.3 Hz, |
| | | 3H), 1.02 (dd, J = 6.7, 2.2 Hz, 6H). |
| C16 | C27H29F3N4O5 | 1H NMR (400 MHz, DMSO) δ 10.15 (t, J = 5.8 Hz, 1H), |
| | 547.41 [M + H] | 8.43 (s, 1H), 7.26-7.15 (m, 2H), 5.64 (dt, J = 11.6, 2.4 Hz, |
| | | 1H), 5.35 (ddd, J = 11.6, 3.6, 2.5 Hz, 1H), 5.27 (s, 1H), |
| | | 5.04 (d, J = 14.6 Hz, 1H), 4.77 (d, J = 14.6 Hz, 1H), 4.55 |
| | | (d, J = 5.8 Hz, 2H), 3.88-3.81 (m, 1H), 2.64-2.51 (m, |
| | | 2H), 1.67 (dp, J = 13.1, 6.6 Hz, 1H), 1.61-1.47 (m, 2H), |
| | | 1.34 (d, J = 6.7 Hz, 3H), 1.28 (d, J = 7.3 Hz, 3H), 0.91 (d, J = |
| | | 6.5 Hz, 6H). |
| C17 | C28H31F3N4O5 | 1H NMR (400 MHz, DMSO) δ 10.16 (t, J = 5.8 Hz, 1H), |
| | 561.44 [M + H] | 8.43 (s, 1H), 7.26-7.15 (m, 2H), 5.64 (dt, J = 11.5, 2.4 Hz, |
| | | 1H), 5.35 (ddd, J = 11.6, 3.6, 2.5 Hz, 1H), 5.30-5.23 (m, |
| | | 1H), 5.04 (d, J = 14.6 Hz, 1H), 4.77 (d, J = 14.5 Hz, 1H), |
| | | 4.55 (d, J = 5.8 Hz, 2H), 3.84 (dd, J = 7.1, 3.9 Hz, 1H), |
| | | 2.64-2.51 (m, 2H), 1.69-1.47 (m, 3H), 1.37-1.21 (m, |
| | | 8H), 0.88 (d, J = 6.6 Hz, 6H). |
| C18 | C29H33F3N4O5 | H NMR (400 MHz, DMSO) δ 10.15 (t, J = 5.8 Hz, 1H), |
| | 575.48 [M + H] | 8.43 (s, 1H), 7.20 (t, J = 8.6 Hz, 2H), 5.64 (dt, J = 11.6, 2.4 |
| | | Hz, 1H), 5.35 (dt, J = 11.6, 3.1 Hz, 1H), 5.26 (s, 1H), 5.04 |
| | | (d, J = 14.6 Hz, 1H), 4.77 (d, J = 14.5 Hz, 1H), 4.54 (d, J = |
| | | 5.7 Hz, 2H), 3.84 (d, J = 3.8 Hz, 1H), 2.64-2.50 (m, 2H), |
| | | 1.61 (p, J = 7.3 Hz, 2H), 1.52 (dq, J = 13.2, 6.6 Hz, 1H), |
| | | 1.46-1.36 (m, 2H), 1.34 (d, J = 6.8 Hz, 3H), 1.27 (d, J = |
| | | 7.3 Hz, 3H), 1.25-1.13 (m, 2H), 0.86 (d, J = 6.6 Hz, 6H). |
| C19 | C30H35F3N4O5 | 1H NMR (400 MHz, DMSO) δ 10.15 (t, J = 5.8 Hz, 1H), |
| | 589.47 [M + H] | 8.43 (s, 1H), 7.20 (t, J = 8.6 Hz, 2H), 5.64 (dt, J = 11.6, 2.4 |
| | | Hz, 1H), 5.35 (dt, J = 11.9, 3.0 Hz, 1H), 5.29-5.22 (m, |
| | | 1H), 5.04 (d, J = 14.7 Hz, 1H), 4.77 (d, J = 14.6 Hz, 1H), |
| | | 4.55 (d, J = 5.8 Hz, 2H), 3.84 (s, 1H), 2.62-2.51 (m, 2H), |
| | | 1.63 (p, J = 6.8 Hz, 2H), 1.52 (hept, J = 6.7 Hz, 1H), 1.43- |
| | | 1.21 (m, 10H), 1.21-1.11 (m, 2H), 0.86 (d, J = 6.6 Hz, 6H). |
| C20 | C31H37F3N4O5 | 1H NMR (400 MHz, DMSO) δ 10.15 (t, J = 5.8 Hz, 1H), |
| | 603.51 [M + H] | 8.43 (s, 1H), 7.26-7.14 (m, 2H), 5.64 (dt, J = 11.6, 2.4 Hz, |
| | | 1H), 5.35 (ddd, J = 11.6, 3.6, 2.5 Hz, 1H), 5.26 (dt, J = 7.2, |
| | | 2.7 Hz, 1H), 5.04 (d, J = 14.6 Hz, 1H), 4.77 (d, J = 14.5 Hz, |
| | | 1H), 4.54 (d, J = 5.8 Hz, 2H), 3.88-3.81 (m, 1H), 2.63- |
| | | 2.51 (m, 2H), 1.68-1.54 (m, 2H), 1.50 (dq, J = 13.1, 6.6 |
| | | Hz, 1H), 1.44-1.21 (m, 11H), 1.16 (q, J = 7.8 Hz, 3H), |
| | | 0.85 (d, J = 6.6 Hz, 6H). |
| C21 | C35H43F3N4O5 | 1H NMR (400 MHz, DMSO) δ 10.15 (t, J = 5.8 Hz, 1H), |
| | 657.59 [M + H] | 8.43 (s, 1H), 7.25-7.14 (m, 2H), 5.64 (dt, J = 11.6, 2.4 Hz, |
| | | 1H), 5.40-5.23 (m, 4H), 5.04 (d, J = 14.6 Hz, 1H), 4.77 |
| | | (d, J = 14.5 Hz, 1H), 4.54 (d, J = 5.7 Hz, 2H), 3.87-3.81 |
| | | (m, 1H), 2.63-2.51 (m, 2H), 2.03-1.95 (m, 4H), 1.62 (p, |
| | | J = 7.4 Hz, 2H), 1.46-1.17 (m, 18H), 0.90-0.82 (m, 3H). |
| C22 | C41H47F3N4O5 | 1H NMR (401 MHz, DMSO) δ 10.15 (t, J = 5.8 Hz, 1H), |
| | 733.60 [M + H] | 8.43 (s, 1H), 7.19 (t, J = 8.6 Hz, 2H), 5.63 (dt, J = 11.6, 2.4 |
| | | Hz, 1H), 5.44-5.21 (m, 12H), 5.04 (d, J = 14.6 Hz, 1H), |
| | | 4.76 (d, J = 14.5 Hz, 1H), 4.54 (d, J = 5.7 Hz, 2H), 3.88- |
| | | 3.78 (m, br, 1H), 2.84-2.73 (m, 8H), 2.56 (td, J = 7.2, 3.7 |
| | | Hz, 2H), 2.19 (q, J = 7.0 Hz, 2H), 2.10-1.96 (m, 2H), 1.69 |
| | | (p, J = 7.4 Hz, 2H), 1.34 (d, J = 6.8 Hz, 3H), 1.27 (d, J = |
| | | 7.3 Hz, 3H), 0.91 (t, J = 7.5 Hz, 3H). |
| C23 | C39H49F3N4O5 | 1H NMR (400 MHz, DMSO) δ 10.15 (t, J = 5.8 Hz, 1H), |
| | 711.41 [M + H] | 8.43 (s, 1H), 7.19 (t, J = 8.6 Hz, 2H), 5.64 (dt, J = 11.6, 2.4 |
| | | Hz, 1H), 5.40-5.23 (m, 6H), 5.04 (d, J = 14.6 Hz, 1H), |
| | | 4.76 (d, J = 14.6 Hz, 1H), 4.54 (d, J = 5.8 Hz, 2H), 3.86- |
| | | 3.79 (m, 1H), 2.74 (t, J = 6.3 Hz, 2H), 2.63-2.51 (m, 2H), |
| | | 2.01 (q, J = 6.7 Hz, 4H), 1.62 (p, J = 7.4 Hz, 2H), 1.40- |
| | | 1.20 (m, 20H), 0.85 (t, J = 6.8 Hz, 3H). |
| C24 | C29H31F3N4O5 | 1H NMR (400 MHz, DMSO) δ 10.15 (t, J = 5.8 Hz, 1H), |
| | 573.46 [M + H] | 8.43 (s, 1H), 7.20 (t, J = 8.6 Hz, 2H), 5.64 (dt, J = 11.6, 2.4 |
| | | Hz, 1H), 5.35 (dt, J = 11.7, 2.9 Hz, 1H), 5.26 (s, 1H), 5.04 |
| | | (d, J = 14.6 Hz, 1H), 4.77 (d, J = 14.6 Hz, 1H), 4.55 (d, J = |
| | | 5.8 Hz, 2H), 3.84 (dd, J = 7.0, 3.6 Hz, 1H), 2.56 (td, J = |
| | | 7.5, 5.2 Hz, 2H), 1.94-1.41 (m, 9H), 1.34 (d, J = 6.8 Hz, |
| | | 3H), 1.28 (d, J = 7.3 Hz, 3H), 1.16-1.05 (m, 2H). |
| C25 | C27H27F3N4O5 | 1H NMR (400 MHz, DMSO) δ 10.13 (t, J = 5.8 Hz, 1H), |
| | 545.43 [M + H] | 8.43 (s, 1H), 7.26-7.14 (m, 2H), 5.64 (dt, J = 11.6, 2.4 Hz, |
| | | 1H), 5.35 (ddd, J = 11.6, 3.6, 2.5 Hz, 1H), 5.27 (dt, J = 7.4, |
| | | 2.7 Hz, 1H), 5.04 (d, J = 14.6 Hz, 1H), 4.77 (d, J = 14.5 Hz, |
| | | 1H), 4.54 (d, J = 5.7 Hz, 2H), 3.85 (s, 1H), 3.08-2.96 (m, |
| | | 1H), 2.02-1.86 (m, 4H), 1.72-1.52 (m, 4H), 1.34 (d, J = |
| | | 6.8 Hz, 3H), 1.28 (d, J = 7.3 Hz, 3H). |
| C26 | C28H31F3N4O6 | 1H NMR (400 MHz, DMSO) δ 10.15 (t, J = 5.8 Hz, 1H), |
| | 577.43 [M + H] | 8.43 (s, 1H), 7.20 (t, J = 8.6 Hz, 2H), 5.64 (dt, J = 11.5, 2.4 |
| | | Hz, 1H), 5.35 (dt, J = 11.7, 3.0 Hz, 1H), 5.30-5.23 (m, |
| | | 1H), 5.04 (d, J = 14.6 Hz, 1H), 4.77 (d, J = 14.7 Hz, 1H), |
| | | 4.55 (d, J = 5.7 Hz, 2H), 3.89-3.79(m, 1H), 3.31 (d, J = |
| | | 6.4 Hz, 2H), 3.22 (s, 3H), 2.56 (q, J = 7.2 Hz, 2H), 1.64 (p, |
| | | J = 7.4 Hz, 2H), 1.52 (dp, J = 13.6, 6.7 Hz, 2H), 1.41 (qd, J = |
| | | 7.4, 4.1 Hz, 2H), 1.34 (d, J = 6.8 Hz, 3H), 1.28 (d, J = 7.3 |
| | | Hz, 3H). |
| C27 | C26H25F3N4O7 | 1H NMR (400 MHz, DMSO) δ 12.05 (br, 1H), 10.31 (t, J = |
| | 563.21 [M + H] | 5.8 Hz, 1H), 8.21 (s, 1H), 7.28-7.15 (m, 2H), 5.64 (dt, J = |
| | | 11.4, 2.3 Hz, 1H), 5.35 (dt, J = 11.8, 2.5 Hz, 1H), 5.32- |
| | | 5.23 (m, 1H), 5.00 (d, J = 14.4 Hz, 1H), 4.67 (d, J = 14.4 |
| | | Hz, 1H), 4.56 (t, J = 6.3 Hz, 2H), 3.86-3.75 (br, 1H), 2.66- |
| | | 2.29 (m, 4H), 1.94-1.70 (m, 2H), 1.30-1.22 (m, 6H). |
| C28 | C28H29F3N4O7 | 1H NMR (400 MHz, DMSO) δ 12.11-11.90 (br, 1H), |
| | 591.45 [M + H] | 10.15 (t, J = 5.8 Hz, 1H), 8.43 (d, J = 1.4 Hz, 1H), 7.20 (t, J = |
| | | 8.6 Hz, 2H), 5.64 (dt, J = 11.5, 2.4 Hz, 1H), 5.35 (dt, J = |
| | | 11.8, 3.0 Hz, 1H), 5.31-5.23 (m, 1H), 5.04 (d, J = 14.6 Hz, |
| | | 1H), 4.77 (d, J = 14.5 Hz, 1H), 4.55 (d, J = 5.7 Hz, 2H), |
| | | 3.90-3.79 (m, 1H), 2.55 (q, J = 7.2 Hz, 2H), 2.21 (t, J = 7.3 |
| | | Hz, 2H), 1.63 (p, J = 7.4 Hz, 2H), 1.53 (p, J = 7.4 Hz, 2H), |
| | | 1.44-1.30 (m, 5H), 1.28 (d, J = 7.3 Hz, 3H). |
| C29 | C39H51F3N4O7 | 1H NMR (401 MHz, DMSO) δ 12.05-11.85 (br, 1H), 10.15 |
| | 745.67 [M + H] | (t, J = 5.8 Hz, 1H), 8.43 (s, 1H), 7.26-7.14 (m, 2H), 5.64 |
| | | (dt, J = 11.6, 2.4 Hz, 1H), 5.35 (ddd, J = 11.6, 3.6, 2.5 Hz, |
| | | 1H), 5.26 (dt, J = 7.3, 2.7 Hz, 1H), 5.04 (d, J = 14.6 Hz, |
| | | 1H), 4.77 (d, J = 14.6 Hz, 1H), 4.54 (d, J = 5.8 Hz, 2H), |
| | | 3.84 (s, 1H), 2.63-2.51 (m, 2H), 2.17 (t, J = 7.4 Hz, 2H), |
| | | 1.62 (p, J = 7.2 Hz, 2H), 1.47 (t, J = 7.1 Hz, 2H), 1.42- |
| | | 1.15 (m, 30 H). |

### Example 3: Synthesis of Acetal Esters of Intermediate A

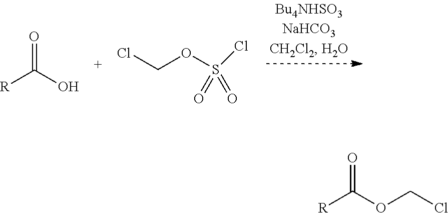

To a mixture of the carboxylic acid (RCOOH) and sodium bicarbonate in water and CH₂Cl₂ is added tetrabutylammonium hydrogen sulfate under vigorous stirring. Chloromethyl chlorosulfate is added and the reaction mixture is stirred at room temperature for 4 h. The reaction mixture is diluted with CH₂Cl₂, washed with brine, dried over Na₂SO₄, filtered, and concentrated to afford the desired product. The product is used directly in the next step.

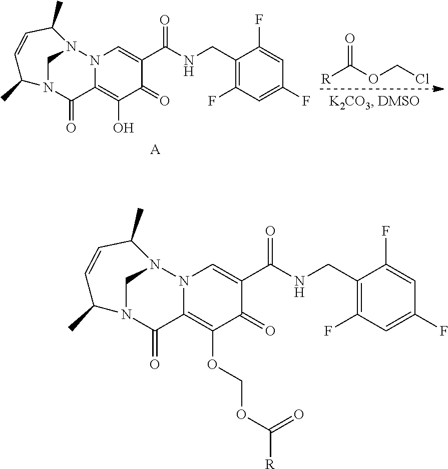

Intermediate A is dissolved in DMSO, followed by the addition K₂CO₃. The chloromethyl ester (RCOOCH₂Cl) is added to the mixture dropwise. The resulting mixture is stirred at room temperature overnight. The mixture is diluted with EtOAc, washed with water and brine, dried over sodium sulfate, filtered and concentrated. The crude material is purified by column chromatography (0-100% EtOAc/Hexane) to afford the desired compound.

### Example 3a: Alternative Synthesis of Acetal Esters of Intermediate A

### Step 1: Synthesis of Chloromethyl Ester

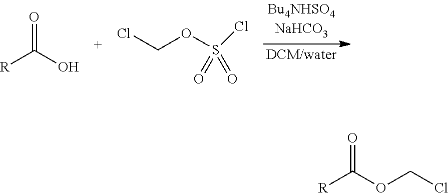

To a mixture of the corresponding acid (1.0 eq) and sodium bicarbonate (6 eq) in water and DCM (20 vol) (1:2 ratio), was added tetrabutylammonium hydrogen sulfate (0.1 eq) under vigorous stirring. Then chloromethyl sulfurochloridate (1.1 eq) was added and the reaction mixture was stirred at room temperature for 18 h. The reaction mixture was diluted with CH₂Cl₂, washed with brine. Organic layer was dried over Na₂SO₄, filtered, and concentrated to afford the corresponding chloromethyl ester.

### Step 2: Alkylation of Intermediate A

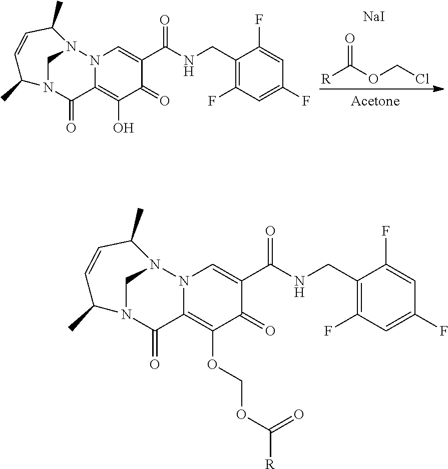

To a stirred solution of Intermediate A (1 eq.) in acetone (20 vol), were added potassium carbonate, (5 eq.) sodium iodide (1 eq.) followed by corresponding chloromethyl ester (3 eq.) at ambient temperature. Then room temperature was allowed to heated to 45 °C and stirred for 18 h. The reaction mixture was partitioned between ethyl acetate and water. The organic layer was washed with brine solution. The organic layer was dried with anhydrous sodium sulfate, filtered and the filtrate was concentrated to afford the corresponding crude product. The crude product was purified by one of the following purification methods:

### Purification Method-1:

The crude product was purified by silica gel column chromatography (50-70% EtOAc/hexane) and fractions containing product were pooled and concentrated. Then resulting product was stirred in diethyl ether (2×30 mL) and decanted. Obtained product was lyophilized from 1:1 MeCN/water to afford the desired compound.

### Purification Method-2:

The crude product was purified by silica gel column chromatography (45-60% EtOAc/hexane) and fractions containing product were pooled and concentrated. The crude compound was stirred in n-pentane (2×30 mL) and decanted. Obtained product was lyophilized from 1:1 MeCN/water to afford the desired compound.

### Purification Method-3:

The crude compound was purified Prep-HPLC column chromatography with below condition in ACN: WATER, the pure fractions were collected and lyophilized to afford the desired compound.

The following compounds are prepared following the procedure of Example 3 using the chloromethyl esters indicated below.

Alternatively, the following compounds were prepared following the procedure of Step 2 of Example 3a using the chloromethyl esters indicated below. The chloromethyl esters of compounds C32-C49 were prepared following the procedure of Step 1 of Example 3a using the carboxylic acids indicated below. Compounds C30-C35 and C43-C47 were purified by Purification Method-1. Compounds C36-C42 and C49 were purified by Purification Method-2. Compounds C48 was purified by Purification Method-2.

| Compound | | Chloromethyl ester | Carboxylic acid |
|---|---|---|---|
| No. | Structure | (RCOOCH₂Cl)* | (RCOOH) |
| C30 | 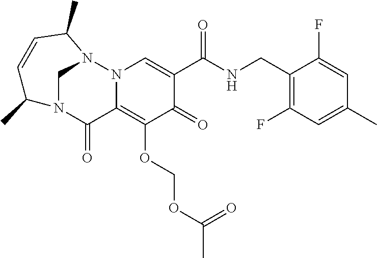 | CH₃COOCH₂Cl | CH₃COOH |
| C31 | 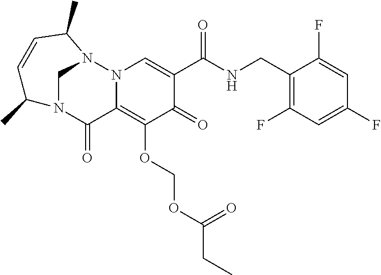 | CH₃CH₂COOCH₂Cl | CH₃CH₂COOH |
| C32 | 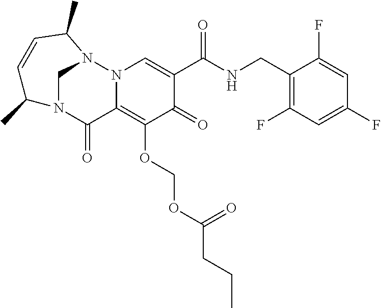 | CH₃(CH₂)₂COOCH₂Cl | CH₃(CH₂)₂COOH |
| C33 | 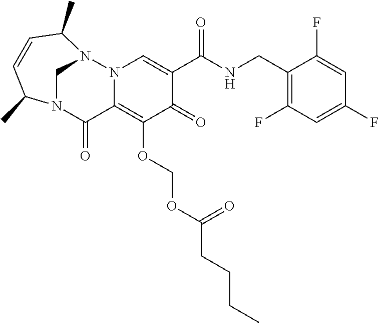 | CH₃(CH₂)₃COOCH₂Cl | CH₃(CH₂)₃COOH |
| C34 | 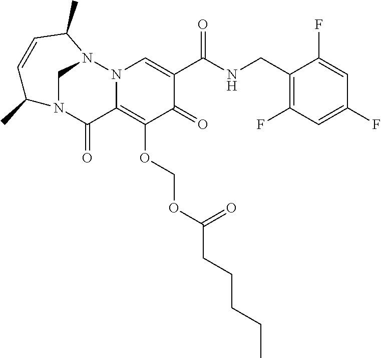 | CH₃(CH₂)₄COOCH₂Cl | CH₃(CH₂)₄COOH |
| C35 | 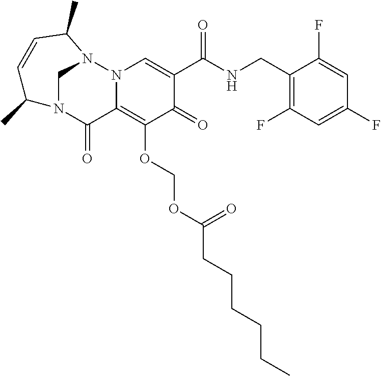 | CH₃(CH₂)₅COOCH₂Cl | CH₃(CH₂)₅COOH |
| C36 | 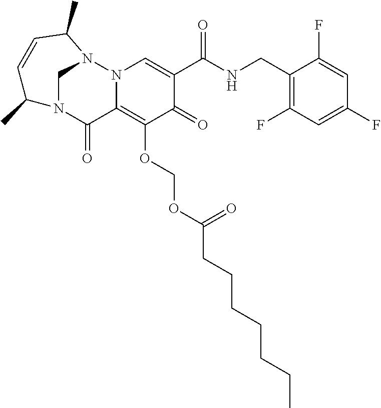 | CH₃(CH₂)₆COOCH₂Cl | CH₃(CH₂)₆COOH |
| C37 | 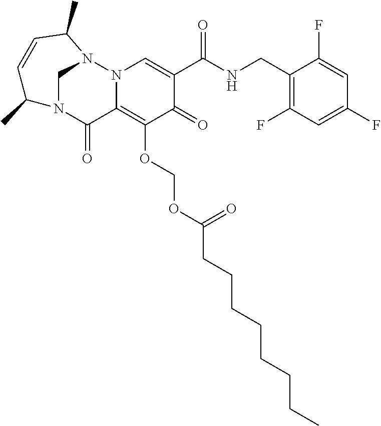 | CH₃(CH₂)₇COOCH₂Cl | CH₃(CH₂)₇COOH |
| C38 | 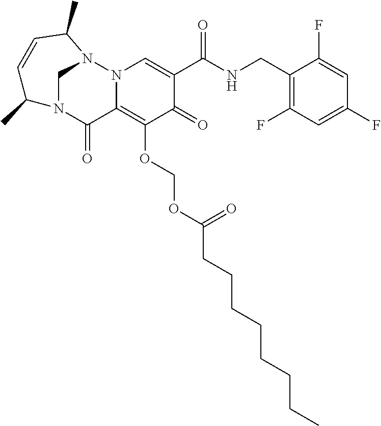 | CH₃(CH₂)₈COOCH₂Cl | CH₃(CH₂)₈COOH |
| C39 | 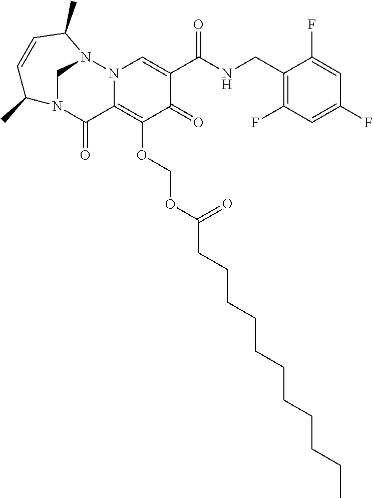 | CH₃(CH₂)₁₀COOCH2 | CH₃(CH₂)₁₀COOH |
| C40 | 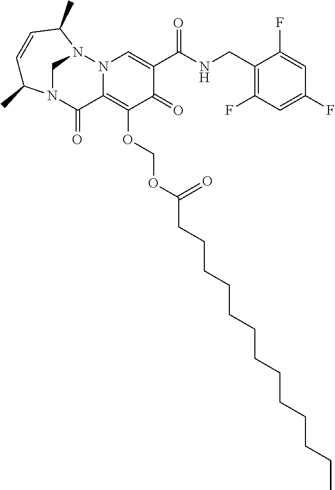 | CH₃(CH₂)₁₂COOCH₂Cl | CH₃(CH₂)₁₂COOH |
| C41 | 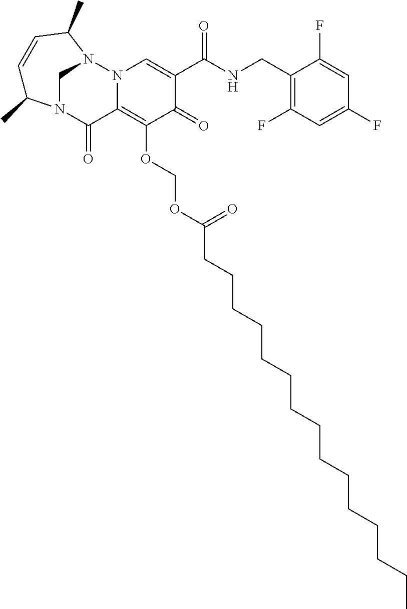 | CH₃(CH₂)₁₄COOCH₂Cl | CH₃(CH₂)₁₄COOH |
| C42 | 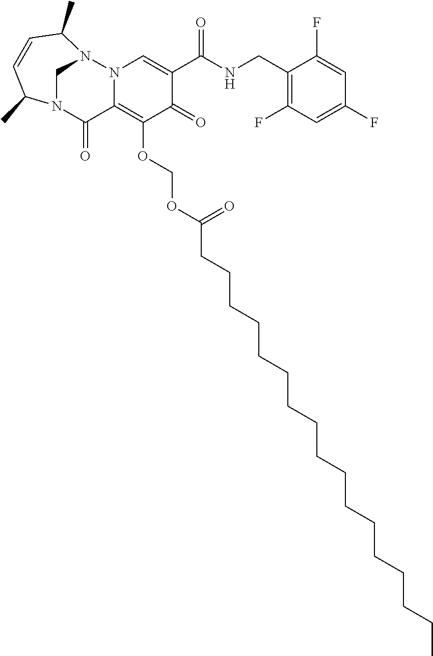 | CH₃(CH₂)₁₆COOCH₂Cl | CH₃(CH₂)₁₆COOH |
| C43 | 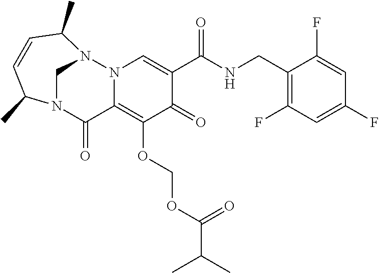 | (CH₃)₂CHCOOCH₂Cl | (CH₃)₂CHCOOH |
| C44 | 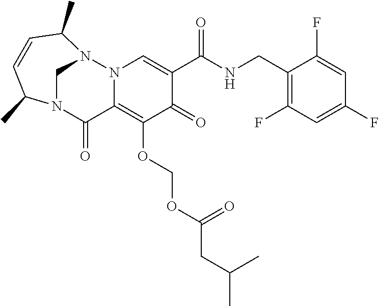 | (CH₃)₂CHCH₂COOC | (CH₃)₂CHCH₂CO |
| C45 | 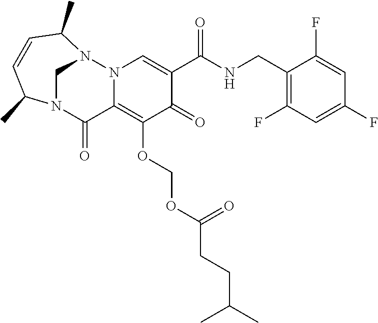 | (CH₃)₂CH(CH₂)₂COOCH₂Cl | (CH₃)₂CH(CH₂)₂COOH |
| C46 | 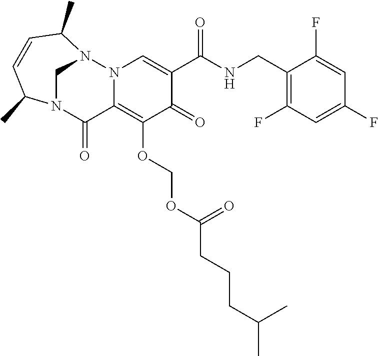 | (CH₃)₂CH(CH₂)₃COOCH₂Cl | (CH₃)₂CH(CH₂)₃COOH |
| C47 | 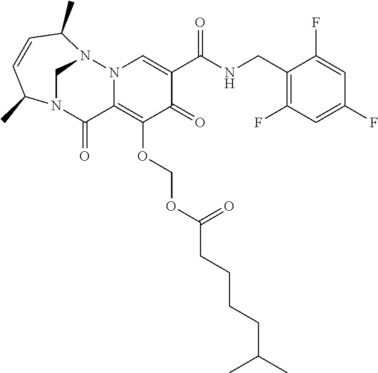 | (CH₃)₂CH(CH₂)₄COOCH₂Cl | (CH₃)₂CH(CH₂)₄COOH |
| C48 | 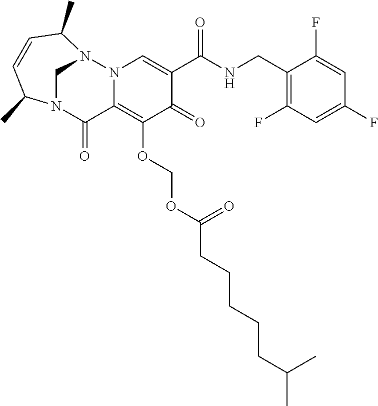 | (CH₃)₂CH(CH₂)₅COOCH₂Cl | (CH₃)₂CH(CH₂)₅COOH |
| C49 | 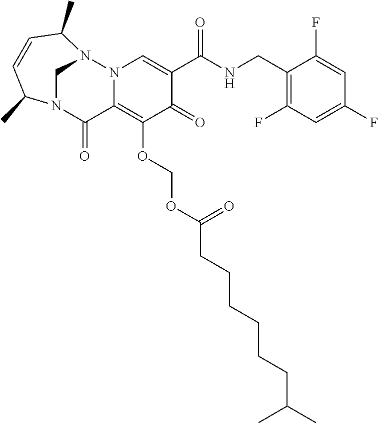 | (CH₃)₂CH(CH₂)₆COOCH₂Cl | (CH₃)₂CH(CH₂)₆COOH |

*Commercially available or prepared from the corresponding carboxylic acid.

The characterization data of the compounds C30-C49 are shown in the table below.

| | Chemical | |
|---|---|---|
| Compound | Formula | |
| No. | MS (m/z) | ¹H NMR Peak Table |
| C30 | C24H23F3N4O6 | 1H NMR (400 MHz, DMSO) δ 10.29 (t, J = 5.8 Hz, 1H), |
| | 521.33 [M + H] | 8.35 (s, 1H), 7.21 (t, J = 8.7 Hz, 2H), 5.82 (d, J = 6.3 Hz, |
| | | 1H), 5.68-5.59 (m, 2H), 5.40-5.27 (m, 2H), 5.01 (d, J = |
| | | 14.6 Hz, 1H), 4.67 (d, J = 14.6 Hz, 1H), 4.55 (d, J = 5.7 |
| | | Hz, 2H), 3.79 (dq, J = 6.6, 3.3 Hz, 1H), 2.01 (s, 3H), 1.29 |
| | | (dd, J = 19.6, 7.0 Hz, 6H). |
| C31 | C25H25F3N4O6 | 1H NMR (400 MHz, DMSO) δ 10.29 (t, J = 5.8 Hz, 1H), |
| | 535.28 [M + H] | 8.35 (s, 1H), 7.27-7.15 (m, 2H), 5.82 (d, J = 6.3 Hz, 1H), |
| | | 5.68-5.59 (m, 2H), 5.40-5.26 (m, 1H), 5.30 (s, 1H), |
| | | 5.01 (d, J = 14.5 Hz, 1H), 4.66 (d, J = 14.2 Hz, 1H), 4.55 |
| | | (d, J = 5.8 Hz, 2H), 3.79 (dq, J = 6.5, 3.2 Hz, 1H), 2.35- |
| | | 2.24 (m, 2H), 1.31 (d, J = 6.7 Hz, 3H), 1.26 (d, J = 7.3 Hz, |
| | | 3H), 0.99 (t, J = 7.5 Hz, 3H). |
| C32 | C26H27F3N4O6 | H NMR (400 MHz, DMSO) δ 10.29 (t, J = 5.8 Hz, 1H), |
| | 549.42 [M + H] | 8.35 (s, 1H), 7.27-7.15 (m, 2H), 5.83 (d, J = 6.3 Hz, 1H), |
| | | 5.68-5.59 (m, 2H), 5.40-5.26 (m, 1H), 5.29 (s, 1H), |
| | | 5.01 (d, J = 14.5 Hz, 1H), 4.65 (d, J = 14.7 Hz, 1H), 4.55 |
| | | (d, J = 5.8 Hz, 2H), 3.79 (dq, J = 6.5, 3.1 Hz, 1H), 2.27 |
| | | (td, J = 7.3, 2.2 Hz, 2H), 1.51 (h, J = 7.4 Hz, 2H), 1.29 |
| | | (dd, J = 20.3, 7.0 Hz, 6H), 0.85 (t, J = 7.4 Hz, 3H). |
| C33 | C27H29F3N4O6 | 1H NMR (400 MHz, DMSO) δ 10.28 (t, J = 5.8 Hz, 1H), |
| | 563.37 [M + H] | 8.35 (s, 1H), 7.27-7.15 (m, 2H), 5.83 (d, J = 6.3 Hz, 1H), |
| | | 5.68-5.59 (m, 2H), 5.40-5.26 (m, 2H), 5.01 (d, J = 14.6 |
| | | Hz, 1H), 4.65 (d, J = 14.6 Hz, 1H), 4.55 (d, J = 5.8 Hz, |
| | | 2H), 3.79 (dq, J = 6.5, 3.1 Hz, 1H), 2.29 (td, J = 7.3, 1.6 |
| | | Hz, 2H), 1.53-1.41 (m, 2H), 1.34-1.19 (m, 8H), 0.83 (t, |
| | | J = 7.3 Hz, 3H). |
| C34 | C28H31F3N4O6 | 1H NMR (400 MHz, DMSO) δ 10.29 (t, J = 5.8 Hz, 1H), |
| | 577.33 [M + H] | 8.35 (s, 1H), 7.27-7.15 (m, 2H), 5.84 (d, J = 6.3 Hz, 1H), |
| | | 5.68-5.59 (m, 2H), 5.40-5.25 (m, 2H), 5.01 (d, J = 14.5 |
| | | Hz, 1H), 4.65 (d, J = 14.5 Hz, 1H), 4.55 (d, J = 5.8 Hz, |
| | | 2H), 3.79 (dq, J = 6.5, 3.2 Hz, 1H), 2.28 (td, J = 7.3, 1.8 |
| | | Hz, 2H), 1.48 (p, J = 7.3 Hz, 2H), 1.34-1.18 (m, 10H), |
| | | 0.87-0.78 (m, 3H). |
| C35 | C29H33F3N4O6 | 1H NMR (400 MHz, DMSO) δ 10.29 (t, J = 5.8 Hz, 1H), |
| | 591.32 | 8.35 (s, 1H), 7.27-7.14 (m, 2H), 5.84 (d, J = 6.3 Hz, 1H), |
| | [M + H] | 5.68-5.59 (m, 2H), 5.40-5.25 (m, 2H), 5.01 (d, J = 14.6 |
| | | Hz, 1H), 4.65 (d, J = 14.5 Hz, 1H), 4.55 (d, J = 5.8 Hz, |
| | | 2H), 3.79 (dq, J = 6.5, 3.1 Hz, 1H), 2.28 (td, J = 7.3, 1.8 |
| | | Hz, 2H), 1.47 (q, J = 7.3 Hz, 2H), 1.34-1.17 (m, 12H), |
| | | 0.88-0.80 (m, 3H). |
| C36 | C30H35F3N4O6 | 1H NMR (400 MHz, DMSO) δ 10.28 (t, J = 5.8 Hz, 1H), |
| | 605.35 [M + H] | 8.35 (s, 1H), 7.20 (t, J = 8.7 Hz, 2H), 5.84 (d, J = 6.3 Hz, |
| | | 1H), 5.68-5.59 (m, 2H), 5.40-5.25 (m, 2H), 5.01 (d, J = |
| | | 14.5 Hz, 1H), 4.65 (d, J = 14.5 Hz, 1H), 4.55 (d, J = 5.8 |
| | | Hz, 2H), 3.78 (dq, J = 6.4, 3.1 Hz, 1H), 2.28 (td, J = 7.3, |
| | | 1.8 Hz, 2H), 1.51-1.42 (m, 2H), 1.34-1.20 (m, 14H), |
| | | 0.88-0.80 (m, 3H). |
| C37 | C31H37F3N4O6 | 1H NMR (400 MHz, DMSO) δ 10.28 (t, J = 5.8 Hz, 1H), |
| | 619.39[M + H] | 8.35 (s, 1H), 7.20 (dd, J = 9.2, 8.0 Hz, 2H), 5.84 (d, J = 6.3 |
| | | Hz, 1H), 5.68-5.59 (m, 2H), 5.40-5.27 (m, 1H), 5.28 (s, |
| | | 1H), 5.01 (d, J = 14.6 Hz, 1H), 4.65 (d, J = 14.5 Hz, 1H), |
| | | 4.55 (d, J = 5.8 Hz, 2H), 3.79 (dt, J = 6.5, 3.3 Hz, 1H), |
| | | 2.28 (td, J = 7.4, 1.9 Hz, 2H), 1.47 (t, J = 7.1 Hz, 2H), 1.34- |
| | | 1.19 (m, 16H), 0.89-0.81 (m, 3H). |
| C38 | C32H39F3N4O6 | 1H NMR (400 MHz, DMSO) δ 10.29 (t, J = 5.8 Hz, 1H), |
| | 633.38 [M + H] | 8.35 (s, 1H), 7.27-7.15 (m, 2H), 5.84 (d, J = 6.3 Hz, 1H), |
| | | 5.68-5.59 (m, 2H), 5.40-5.32 (m, 1H), 5.31-5.25 (m, |
| | | 1H), 5.01 (d, J = 14.5 Hz, 1H), 4.65 (d, J = 14.5 Hz, 1H), |
| | | 4.55 (d, J = 5.8 Hz, 2H), 3.79 (dq, J = 6.5, 3.2 Hz, 1H), |
| | | 2.28 (td, J = 7.3, 1.9 Hz, 2H), 1.47 (t, J = 7.2 Hz, 2H), 1.34- |
| | | 1.15 (m, 18H), 0.89-0.80 (m, 3H). |
| C39 | C34H43F3N4O6 | 1H NMR (400 MHz, DMSO) δ 10.29 (t, J = 5.8 Hz, 1H), |
| | 661.47 [M + H] | 8.35 (s, 1H), 7.20 (dd, J = 9.2, 8.1 Hz, 2H), 5.84 (d, J = 6.3 |
| | | Hz, 1H), 5.68-5.59 (m, 2H), 5.35 (dt, J = 11.5, 3.1 Hz, |
| | | 1H), 5.32-5.25 (m, 1H), 5.01 (d, J = 14.6 Hz, 1H), 4.65 (d, |
| | | J = 14.5 Hz, 1H), 4.55 (d, J = 5.8 Hz, 2H), 3.79 (dq, J = |
| | | 6.4, 3.2 Hz, 1H), 2.28 (td, J = 7.3, 1.8 Hz, 2H), 1.51-1.42 |
| | | (m, 2H), 1.34-1.19 (m, 22H), 0.89-0.81 (m, 3H). |
| C40 | C36H47F3N4O6 | 1H NMR (400 MHz, DMSO) δ 10.29 (t, J = 5.8 Hz, 1H), |
| | 689.72 [M + H] | 8.35 (s, 1H), 7.27-7.14 (m, 2H), 5.84 (d, J = 6.3 Hz, 1H), |
| | | 5.67-5.59 (m, 2H), 5.40-5.25 (m, 2H), 5.01 (d, J = 14.5 |
| | | Hz, 1H), 4.65 (d, J = 14.5 Hz, 1H), 4.55 (d, J = 5.8 Hz, |
| | | 2H), 3.79 (dq, J = 6.4, 3.1 Hz, 1H), 2.28 (td, J = 7.3, 1.8 |
| | | Hz, 2H), 1.51-1.43 (m, 2H), 1.36-1.13 (m, 26H), 0.89- |
| | | 0.81 (m, 3H). |
| C41 | C38H51F3N4O6 | 1H NMR (400 MHz, DMSO) δ 10.29 (t, J = 5.8 Hz, 1H), |
| | 717.71 [M + H] | 8.35 (s, 1H), 7.26-7.14 (m, 2H), 5.84 (d, J = 6.3 Hz, 1H), |
| | | 5.67-5.59 (m, 2H), 5.40-5.25 (m, 2H), 5.01 (d, J = 14.6 |
| | | Hz, 1H), 4.65 (d, J = 14.5 Hz, 1H), 4.55 (d, J = 5.8 Hz, |
| | | 2H), 3.78 (dq, J = 6.5, 3.1 Hz, 1H), 2.28 (td, J = 7.3, 1.8 |
| | | Hz, 2H), 1.47 (t, J = 7.2 Hz, 2H), 1,34-1.15 (m, 30H), 0.89- |
| | | 0.81 (m, 3H). |
| C42 | C40H55F3N4O6 | 1H NMR (400 MHz, DMSO) δ 10.29 (t, J = 5.8 Hz, 1H), |
| | 745.51 [M + H] | 8.35 (s, 1H), 7.26-7.14 (m, 2H), 5.84 (d, J = 6.3 Hz, 1H), |
| | | 5.67-5.59 (m, 2H), 5.40-5.25 (m, 2H), 5.01 (d, J = 14.6 |
| | | Hz, 1H), 4.65 (d, J = 14.5 Hz, 1H), 4.55 (d, J = 5.8 Hz, |
| | | 2H), 3.78 (dq, J = 6.4, 3.2 Hz, 1H), 2.28 (td, J = 7.3, 1.8 |
| | | Hz, 2H), 1.51-1.43 (m, 2H), 1.34-1.17 (m, 34 H), 0.89- |
| | | 0.81 (m, 3H). |
| C43 | C26H27F3N4O6 | 1H NMR (400 MHz, Acetonitrile-d3) δ 10.29 (d, J = 7.1 |
| | 549.18 [M + H] | Hz, 1H), 8.42 (s, 1H), 6.85 (t, J = 8.5 Hz, 2H), 5.85 (d, J = |
| | | 6.2 Hz, 1H), 5.70 (dd, J = 7.0, 1.8 Hz, 1H), 5.62 (dt, J = |
| | | 11.1, 2.4 Hz, 1H), 5.37 (ddd, J = 9.7, 5.1, 2.7 Hz, 2H), |
| | | 4.94 (d, J = 14.5 Hz, 1H), 4.59 (t, J = 5.2 Hz, 2H), 4.48 (d, |
| | | J = 14.5 Hz, 1H), 3.76 (tq, J = 6.7, 3.5 Hz, 1H), 2.53 (tt, J = |
| | | 14.0, 7.0 Hz, 1H), 1.33 (d, J = 6.7 Hz, 3H), 1.27 (d, J = |
| | | 7.4 Hz, 3H), 1.16-1.00 (m, 6H). |
| C44 | C27H29F3N4O6 | 1H NMR (400 MHz, DMSO) δ 10.28 (t, J = 5.8 Hz, 1H), |
| | 563.30 [M + H] | 8.35 (s, 1H), 7.25-7.17 (m, 2H), 5.84 (d, J = 6.3 Hz, 1H), |
| | | 5.68-5.59 (m, 2H), 5.40-5.28 (m, 1H), 5.28 (s, 1H), |
| | | 5.01 (d, J = 14.6 Hz, 1H), 4.65 (d, J = 14.5 Hz, 1H), 4.55 |
| | | (d, J = 5.8 Hz, 2H), 3.79 (dd, J = 6.5, 3.3 Hz, 1H), 2.18 |
| | | (dd, J = 7.0, 2.0 Hz, 2H), 1.94 (dt, J = 13.5, 6.7 Hz, 1H), |
| | | 1.29 (dd, J = 19.2, 7.0 Hz, 6H), 0.87 (dd, J = 6.7, 1.8 Hz, 6H). |
| C45 | C28H31F3N4O6 | 1H NMR (400 MHz, DMSO) δ 10.28 (t, J = 5.8 Hz, 1H), |
| | 577.33 [M + H] | 8.35 (s, 1H), 7.25-7.16 (m, 2H), 5.84 (d, J = 6.3 Hz, 1H), |
| | | 5.68-5.59 (m, 2H), 5.35 (dt, J = 11.4, 3.1 Hz, 1H), 5.30 |
| | | (s, 1H), 5.02 (d, J = 14.5 Hz, 1H), 4.65 (d, J = 14.5 Hz, |
| | | 1H), 4.55 (d, J = 5.7 Hz, 2H), 3.79 (dd, J = 6.6, 3.3 Hz, |
| | | 1H), 2.29 (dd, J = 8.3, 7.0 Hz, 2H), 1.49 (dq, J = 12.9, 6.4 |
| | | Hz, 1H), 1.43-1.23 (m, 8H), 0.82 (d, J = 6.5 Hz, 6H). |
| C46 | C29H33F3N4O6 | 1H NMR (400 MHz, DMSO) δ 10.29 (t, J = 5.8 Hz, 1H), |
| | 591.45[M + H] | 8.35 (s, 1H), 7.27-7.14 (m, 2H), 5.84 (d, J = 6.3 Hz, 1H), |
| | | 5.68-5.59 (m, 2H), 5.40-5.25 (m, 1H), 5.29 (s, 1H), |
| | | 5.01 (d, J = 14.5 Hz, 1H), 4.66 (d, J = 14.5 Hz, 1H), 4.55 |
| | | (d, J = 5.8 Hz, 2H), 3.79 (dq, J = 6.5, 3.2 Hz, 1H), 2.27 |
| | | (td, J = 7.3, 2.0 Hz, 2H), 1.55-1.41 (m, 3H), 1.29 (dd, J = |
| | | 19.5, 7.0 Hz, 6H), 1.18-1.06 (m, 2H), 0.83 (d, J = 6.7 Hz, 6H). |
| C47 | C30H35F3N4O6 | 1H NMR (400 MHz, DMSO) δ 10.29 (t, J = 5.8 Hz, 1H), |
| | 605.49 [M + H] | 8.35 (s, 1H), 7.27-7.14 (m, 2H), 5.84 (d, J = 6.3 Hz, 1H), |
| | | 5.71-5.60 (m, 2H), 5.40-5.25 (m, 2H), 5.01 (d, J = 14.6 |
| | | Hz, 1H), 4.65 (d, J = 14.4 Hz, 1H), 4.55 (d, J = 5.8 Hz, |
| | | 2H), 3.78 (dq, J = 6.4, 3.1 Hz, 1H), 2.37-2.24 (m, 2H), |
| | | 1.55-1.40 (m, 3H), 1.37-1.18 (m, 8H), 1.16-1.06 (m, |
| | | 2H), 0.83 (d, J = 6.6 Hz, 6H). |
| C48 | C31H37F3N4O6 | 1H NMR (400 MHz, DMSO) δ 10.29 (t, J = 5.8 Hz, 1H), |
| | 619.34 [M + H] | 8.35 (s, 1H), 7.27-7.15 (m, 2H), 5.84 (d, J = 6.2 Hz, 1H), |
| | | 5.68-5.59 (m, 2H), 5.40-5.25 (m, 2H), 5.01 (d, J = 14.5 |
| | | Hz, 1H), 4.65 (d, J = 14.4 Hz, 1H), 4.55 (d, J = 5.8 Hz, |
| | | 2H), 3.79 (s, 1H), 2.28 (td, J = 7.3, 1.8 Hz, 2H), 1.47 (h, J = |
| | | 6.6 Hz, 3H), 1.37-1.18 (m, 10H), 1.15-1.08 (m, 2H), |
| | | 0.83 (d, J = 6.6 Hz, 6H). |
| C49 | C32H39F3N4O6 | 1H NMR (401 MHz, DMSO) δ 10.29 (t, J = 5.7 Hz, 1H), |
| | 633.38 [M + H] | 8.35 (s, 1H), 7.20 (t, J = 8.6 Hz, 2H), 5.84 (d, J = 6.3 Hz, |
| | | 1H), 5.68-5.59 (m, 2H), 5.40-5.27 (m, 1H), 5.28 (s, |
| | | 1H), 5.01 (d, J = 14.5 Hz, 1H), 4.65 (d, J = 14.5 Hz, 1H), |
| | | 4.55 (d, J = 5.8 Hz, 2H), 3.79 (dq, J = 6.4, 3.1 Hz, 1H), |
| | | 2.28 (td, J = 7.3, 1.9 Hz, 2H), 1.52- 1.41 (m, 3H), 1.29 |
| | | (dd, J = 20.0, 7.0 Hz, 6H), 1.24-1.17 (m, 6H), 1.16-1.06 |
| | | (m, 2H), 0.83 (d, J = 6.6 Hz, 6H). |

### Example 3b: Alternate Synthesis of (((1S,2R,5S)-2,5-dimethyl-7,9-dioxo-10-((2,4,6-trifluorobenzyl)carbamoyl)-2,5,7,9-tetrahydro-1,6-methanopyrido[1,2-b][1,2,5]triazonin-8-yl)oxy)methyl isobutyrate (C43)

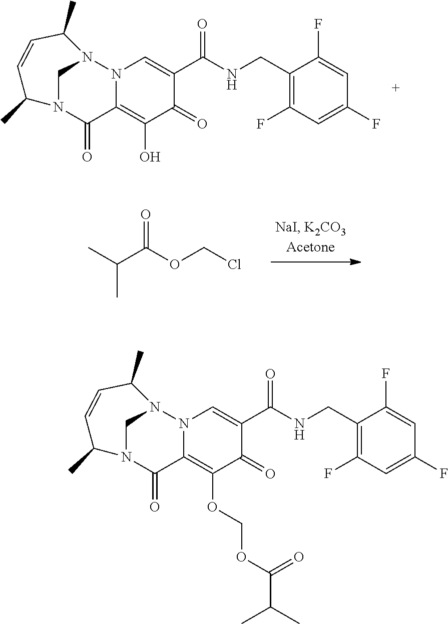

A solution of chloromethyl isobutyrate (0.009 g, 0.067 mmol) and sodium iodide (0.011 g, 0.076 mmol) in acetone (0.1 mL) was stirred at room temperature for 24 h. In a separate vessel, Intermediate A (0.020 g, 0.045 mmol) and potassium carbonate (0.012 g, 0.089 mmol) were mixed with acetone (0.1 mL) and to this mixture was added the solution of the iodomethyl isobutyrate. After stirring at rt for 24 h, water was added, and the mixture was extracted with EtOAc. The organic phase was dried over MgSO₄, filtered, and concentrated. The residue was purified by silica gel column chromatography to afford the title compound. MS (m/z) 549.18 [M+H]+. ¹H NMR (400 MHz, Acetonitrile-d3) δ 10.29 (d, J=7.1 Hz, 1H), 8.42 (s, 1H), 6.85 (t, J=8.5 Hz, 2H), 5.85 (d, J=6.2 Hz, 1H), 5.70 (dd, J=7.0, 1.8 Hz, 1H), 5.62 (dt, J=11.1, 2.4 Hz, 1H), 5.37 (ddd, J=9.7, 5.1, 2.7 Hz, 2H), 4.94 (d, J=14.5 Hz, 1H), 4.59 (t, J=5.2 Hz, 2H), 4.48 (d, J=14.5 Hz, 1H), 3.76 (tq, J=6.7, 3.5 Hz, 1H), 2.53 (tt, J=14.0, 7.0 Hz, 1H), 1.33 (d, J=6.7 Hz, 3H), 1.27 (d, J=7.4 Hz, 3H), 1.16-1.00 (m, 8H).

### Example 4: Synthesis of Carbonates of Intermediate A

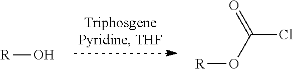

To a solution of bis(trichloromethyl)carbonate in THF at 0 °C is added pyridine followed by a solution of the alcohol (ROH) in THF. The resulting suspension is removed from cooling bath and allowed to warm to room temperature and stir for 3 hours. The reaction mixture is filtered through Celite® and rinsed with CH₂Cl₂. The filtrate is washed with 1N HCl, dried over Na₂SO₄, filtered, and concentrated to afford the desired product. The product is used directly in the next step.

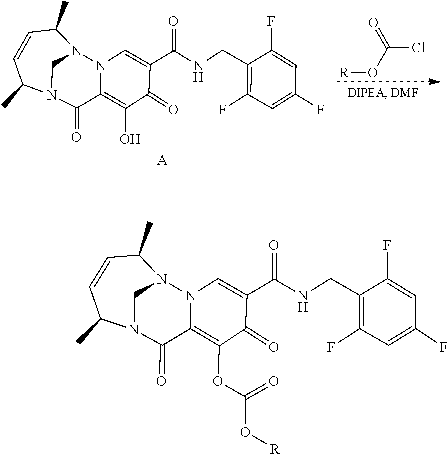

To a mixture of Intermediate A in DMF at 0 °C is added N,N-diisopropylethylamine followed by the chloroformate (ROCOCl). The resulting mixture is removed from cooling bath and allowed to warm to room temperature and stir for 16 hours. The reaction mixture is then diluted with EtOAc, washed with 1 N HCl (2×), water (2×), and brine. The organic phase is dried over Na₂SO₄, filtered, concentrated and purified by silica gel column chromatography to give the desired compound.

### Example 4a: Alternative Synthesis of Carbonates of Intermediate A

### Step 1: Synthesis of Chloroformates

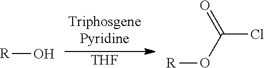

To a solution of bis(trichloromethyl)carbonate (0.5 equiv.) in THF (0.1 M) at 0 °C was added pyridine (1.3 equiv.) followed by a solution of the alcohol (1 equiv.) in THF. The resulting suspension was removed from cooling bath and allowed to warm to room temperature and stir for 3 hours. The reaction mixture was filtered through Celite® and rinsed with CH₂Cl₂. The filtrate was washed with 1N HCl, dried over Na₂SO₄, filtered, and concentrated to afford the desired product. The product was used directly in the next step.

### Step 2: Synthesis of Carbonates

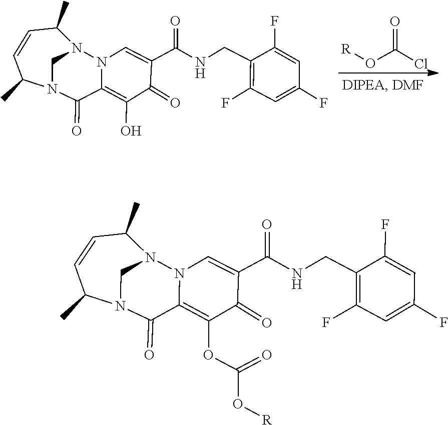

To a solution of Intermediate A (1.0 eq) in DMF (20 vol.) was added DIPEA (2.5 eq.) at 0 °C, stirred for 5 min. Then the corresponding chloroformate (1.5 eq.) was added at same temperature. Then reaction mixture was allowed to ambient temperature and stirred for 3 h. The reaction mixture was diluted with EtOAc and was washed with water and brine solution. The organic phase was dried over Na₂SO₄, filtered, and concentrated to afford corresponding carbonate.

### Purification Method-1:

The crude product was purified by silica gel column chromatography (65-70% EtOAc/hexane) and fractions containing product were pooled and concentrated. The crude compound was stirred in n-pentane (2×30 mL) and decanted. Obtained product was lyophilized from 1:1 MeCN/water to afford the desired compound.

### Purification Method-2:

The crude product was purified by silica gel column chromatography (50-70% EtOAc/hexane) and fractions containing product were pooled and concentrated. Then resulting product was stirred in diethyl ether (2×30 mL) and decanted. Obtained product was lyophilized from 1:1 MeCN/water to afford the desired compound.

The following compounds are prepared following the procedure of Example 4 using the chloroformates indicated below.

Alternatively, the following compounds were prepared following the procedure of Step 2 of Example 4a using the chloroformates indicated below. The chloroformates for compounds C50, C51, and C61-C74 were prepared following the procedure of Step 1 of Example 4a using the alcohols indicated below. Compounds C50-C53, C61-C66 and C74 were purified by Purification Method-1. Compounds C54-C60 and C67-C73 were purified by Purification Method-2.

| Compound | | Chloroformate | |
|---|---|---|---|
| No. | Structure | (ROCOCl)* | Alcohol (R—OH) |
| C50 | 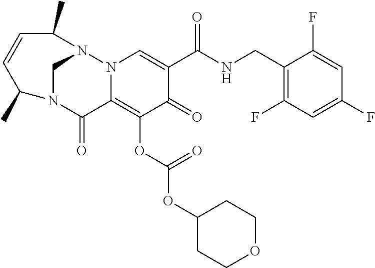 | tetrahydro-2H-pyran-4-yl carbonochloridate | tetrahydro-2H-pyran- 4-ol |
| C51 | 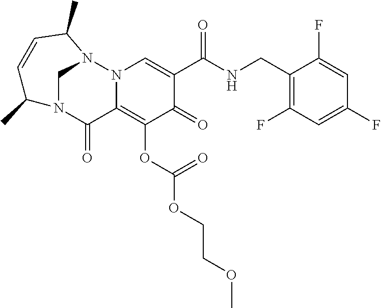 | CH₃OCH₂CH₂OCOCl | CH₃OCH₂CH₂OH |
| C52 | 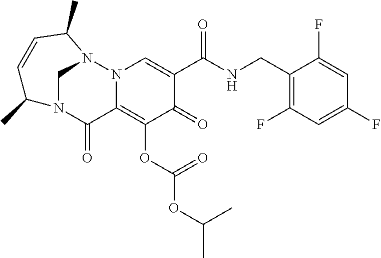 | (CH₃)₂CHOCOCl | (CH₃)₂CHOH |
| C53 | 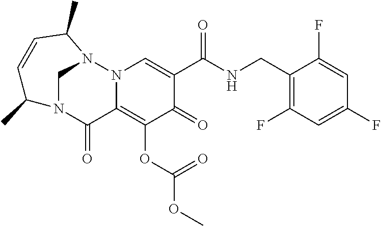 | CH₃OCOCl | CH₃OH |
| C54 | 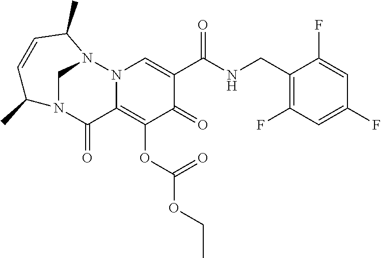 | CH₃CH₂OCOCl | CH₃CH₂OH |
| C55 | 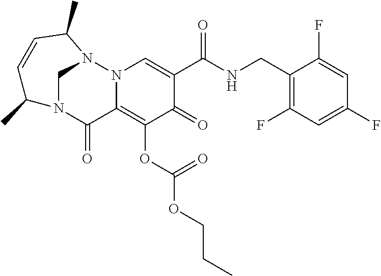 | CH₃(CH₂)₂OCOCl | CH₃(CH₂)₂OH |
| C56 | 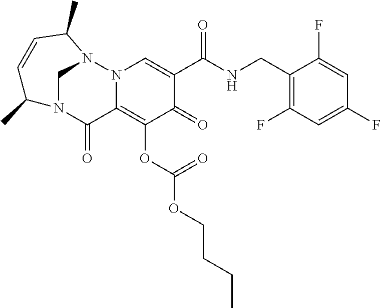 | CH₃(CH₂)₃OCOCl | CH₃(CH₂)₃OH |
| C57 | 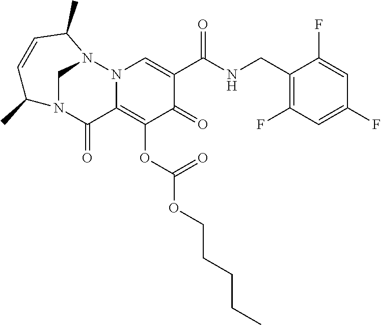 | CH₃(CH₂)₄OCOCl | CH₃(CH₂)₄OH |
| C58 | 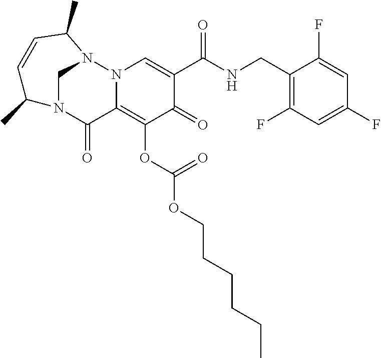 | CH₃(CH₂)₅OCOCl | CH₃(CH₂)₅OH |
| C59 | 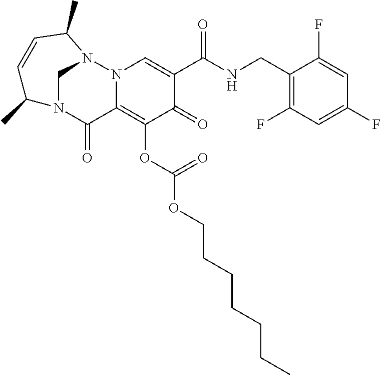 | CH₃(CH₂)₆OCOCl | CH₃(CH₂)₆OH |
| C60 | 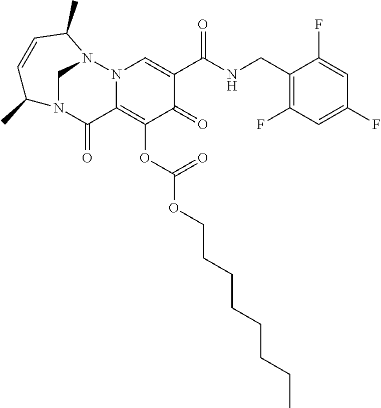 | CH₃(CH₂)₇OCOCl | CH₃(CH₂)₇OH |
| C61 | 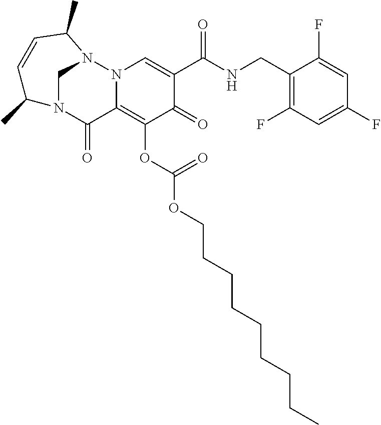 | CH₃(CH₂)₈OCOCl | CH₃(CH₂)₈OH |
| C62 | 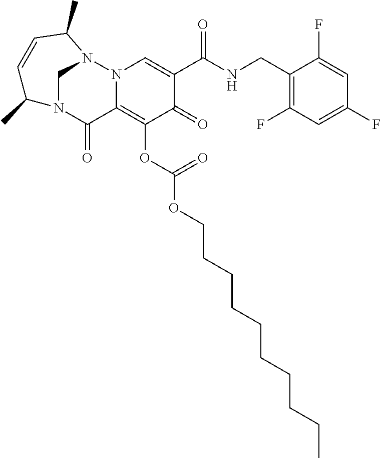 | CH₃(CH₂)₉OCOCl | CH₃(CH₂)₉OH |
| C63 | 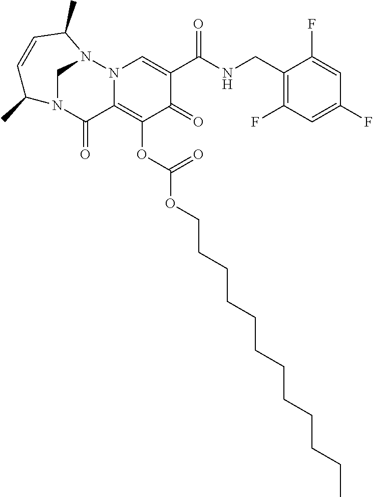 | CH₃(CH₂)₁₁OCOCl | CH₃(CH₂)₁₁OH |
| C64 | 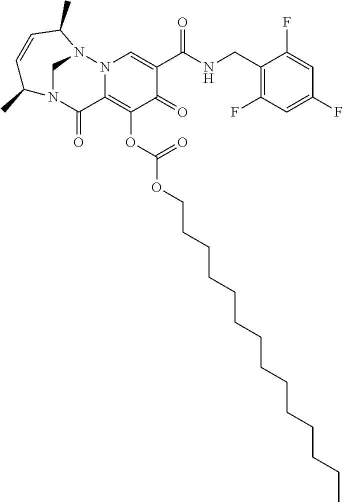 | CH₃(CH₂)₁₃OCOCl | CH₃(CH₂)₁₃OH |
| C65 | 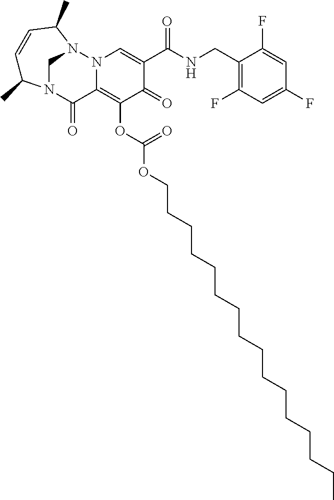 | CH₃(CH₂)₁₅OCOCl | CH₃(CH₂)₁₅OH |
| C66 | 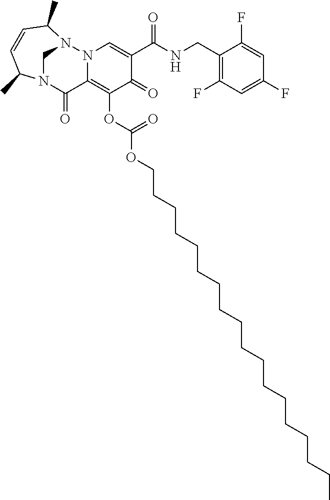 | CH₃(CH₂)₁₇OCOCl | CH₃(CH₂)₁₇OH |
| C67 | 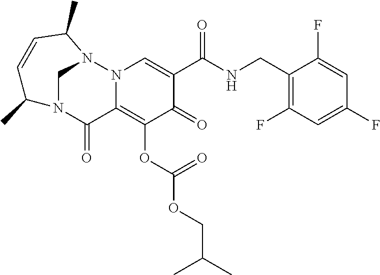 | (CH₃)₂CHCH₂OCOCl | (CH₃)₂CHCH₂OH |
| C68 | 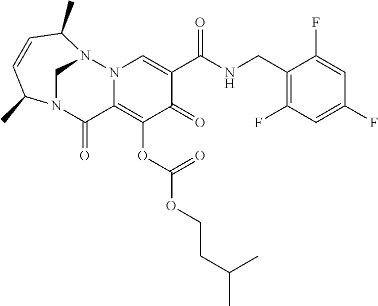 | (CH₃)₂CHCH₂CH₂OCOCl | (CH₃)₂CHCH₂CH₂OH |
| C69 | 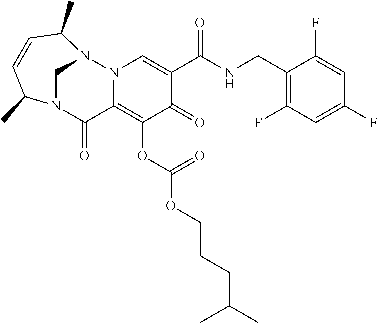 | (CH₃)₂CH(CH₂)₃OCOCl | (CH₃)₂CH(CH₂)₃OH |
| C70 | 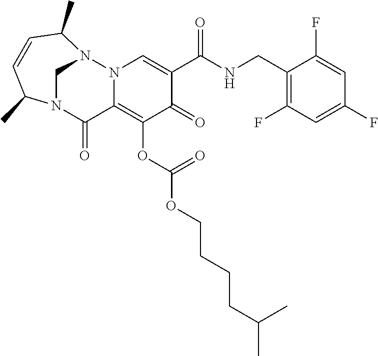 | (CH₃)₂CH(CH₂)₄OCOCl | (CH₃)₂CH(CH₂)₄OH |
| C71 | 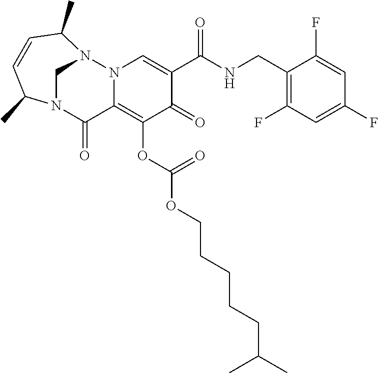 | (CH₃)₂CH(CH₂)₅OCOCl | (CH₃)₂CH(CH₂)₅OH |
| C72 | 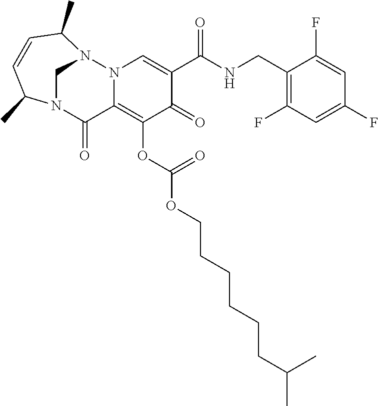 | (CH₃)₂CH(CH₂)₆OCOCl | (CH₃)₂CH(CH₂)₆OH |
| C73 | 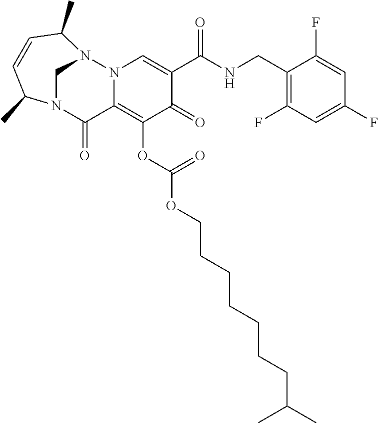 | (CH₃)₂CH(CH₂)₇OCOCl | (CH₃)₂CH(CH₂)₇OH |
| C74 | 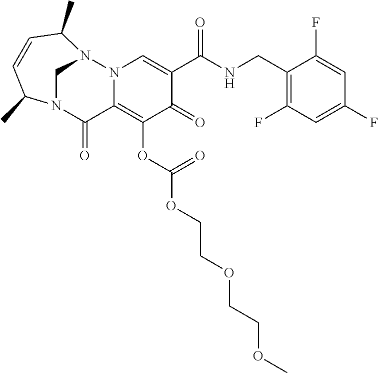 | CH₃(OCH₂CH₂)₂OCOCl | CH₃(OCH₂CH₂)₂OH |

*Commercially available or prepared from the corresponding alcohol.

The characterization data of the compounds C50-C74 are shown in the table below.

| | Chemical | |
|---|---|---|
| Compound | Formula | |
| No. | MS (m/z) | ¹H NMR Peak Table |
| C50 | C27H27F3N4O7 | 1H NMR (400 MHz, Acetonitrile-d3) δ 10.16 (s, 1H), 8.51 |
| | 577.10 [M + H] | (s, 1H), 6.98-6.72 (m, 2H), 5.76-5.52 (m, 1H), 5.52- |
| | | 5.26 (m, 2H), 4.98 (d, J = 14.5 Hz, 1H), 4.93-4.78 (m, |
| | | 1H), 4.68-4.50 (m, 3H), 3.84 (dq, J = 10.4, 6.1, 5.3 Hz, |
| | | 3H), 3.50 (td, J = 11.1, 2.9 Hz, 2H), 1.90-1.85 (m, 2H), |
| | | 1.84-1.61 (m, 2H), 1.36 (d, J = 6.8 Hz, 3H), 1.30 (d, J = |
| | | 7.4 Hz, 3H). |
| C51 | C25H25F3N4O7 | 1H NMR (400 MHz, Acetonitrile-d3) δ 10.16 (s, 1H), 8.50 |
| | 551.01 [M + H] | (s, 1H), 7.00-6.70 (m, 2H), 5.75-5.47 (m, 1H), 5.47- |
| | | 5.22 (m, 2H), 4.98 (d, J = 14.6 Hz, 1H), 4.75-4.44 (m, |
| | | 3H), 4.44-4.21 (m, 2H), 3.83 (dq, J = 6.5, 3.2 Hz, 1H), |
| | | 3.72-3.52 (m, 2H), 3.34 (s, 3H), 1.36 (d, J = 6.8 Hz, 3H), |
| | | 1.30 (d, J = 7.2 Hz, 3H). |
| C52 | C25H25F3N4O6 | 1H NMR (400 MHz, CD3CN) δ 10.20 (s, 1H), 8.53 (s, |
| | 535.10 [M + H] | 1H), 6.94-6.81 (m, 2H), 5.66 (dt, J = 11.1, 2.5 Hz, 1H), |
| | | 5.45-5.35 (m, 2H), 5.04-4.87 (m, 2H), 4.70-4.55 (m, |
| | | 3H), 3.85 (dh, J = 6.6, 3.5 Hz, 1H), 1.37 (dd, J = 10.9, 6.5 |
| | | Hz, 9H), 1.32 (d, J = 7.3 Hz, 3H). |
| C53 | C23H21F3N4O6 | 1H NMR (400 MHz, DMSO) δ 10.11 (t, J = 5.8 Hz, 1H), |
| | 507.39 [M + H] | 8.45 (s, 1H), 7.27-7.15 (m, 2H), 5.64 (dt, J = 11.6, 2.5 |
| | | Hz, 1H), 5.35 (ddd, J = 11.6, 3.6, 2.5 Hz, 1H), 5.27 (dd, J = |
| | | 7.3, 2.7 Hz, 1H), 5.05 (d, J = 14.6 Hz, 1H), 4.79 (d, J = |
| | | 14.6 Hz, 1H), 4.55 (d, J = 5.9 Hz, 2H), 3.87 (dq, J = 6.4, |
| | | 3.0 Hz, 1H), 3.84 (s, 3H), 1.35 (d, J = 6.8 Hz, 3H), 1.28 (d, |
| | | J = 7.3 Hz, 3H). |
| C54 | C24H23F3N4O6 | 1H NMR (400 MHz, DMSO) δ 10.11 (t, J = 5.8 Hz, 1H), |
| | 521.25 [M + H] | 8.45 (s, 1H), 7.27-7.15 (m, 2H), 5.64 (dt, J = 11.5, 2.4 |
| | | Hz, 1H), 5.35 (ddd, J = 11.6, 3.6, 2.5 Hz, 1H), 5.30-5.20 |
| | | (m, 1H), 5.05 (d, J = 14.6 Hz, 1H), 4.78 (d, J = 14.6 Hz, |
| | | 1H), 4.55 (d, J = 5.8 Hz, 2H), 4.25 (q, J = 7.0 Hz, 2H), |
| | | 3.86 (dt, J = 6.4, 3.3 Hz, 1H), 1.38-1.24 (m, 9H). |
| C55 | C25H25F3N4O6 | 1H NMR (400 MHz, DMSO) δ 10.12 (t, J = 5.8 Hz, 1H), |
| | 535.26 [M + H] | 8.45 (s, 1H), 7.27-7.14 (m, 2H), 5.64 (dt, J = 11.6, 2.4 |
| | | Hz, 1H), 5.40-5.23 (m, 2H), 5.05 (d, J = 14.6 Hz, 1H), |
| | | 4.78 (d, J = 14.7 Hz, 1H), 4.62-4.49 (m, 2H), 4.16 (tt, J = |
| | | 6.8, 3.5 Hz, 2H), 3.87 (dq, J = 6.4, 3.0 Hz, 1H), 1.74- |
| | | 1.60 (m, 2H), 1.35 (d, J = 6.8 Hz, 3H), 1.28 (d, J = 7.3 Hz, |
| | | 3H), 0.92 (t, J = 7.4 Hz, 3H). |
| C56 | C26H27F3N4O6 | 1H NMR (400 MHz, DMSO) δ 10.11 (t, J = 5.8 Hz, 1H), |
| | 549.31 [M + H] | 8.45 (s, 1H), 7.27-7.14 (m, 2H), 5.64 (dt, J = 11.6, 2.4 |
| | | Hz, 1H), 5.35 (ddd, J = 11.6, 3.6, 2.5 Hz, 1H), 5.31-5.24 |
| | | (m, 1H), 5.05 (d, J = 14.6 Hz, 1H), 4.78 (d, J = 14.5 Hz, |
| | | 1H), 4.55 (d, J = 5.8 Hz, 2H), 4.20 (td, J = 6.5, 2.4 Hz, |
| | | 2H), 3.86 (dq, J = 6.3, 3.0 Hz, 1H), 1.63 (dq, J = 8.4, 6.6 |
| | | Hz, 2H), 1.41-1.31 (m, 5H), 1.28 (d, J = 7.3 Hz, 3H), 0.90 |
| | | (t, J = 7.4 Hz, 3H). |
| C57 | C27H29F3N4O6 | 1H NMR (400 MHz, DMSO) δ 10.11 (t, J = 5.8 Hz, 1H), |
| | 563.42 [M + H] | 8.45 (s, 1H), 7.27-7.14 (m, 2H), 5.64 (dt, J = 11.6, 2.4 |
| | | Hz, 1H), 5.35 (ddd, J = 11.6, 3.6, 2.5 Hz, 1H), 5.30-5.23 |
| | | (m, 1H), 5.05 (d, J = 14.6 Hz, 1H), 4.78 (d, J = 14.5 Hz, |
| | | 1H), 4.55 (d, J = 5.8 Hz, 2H), 4.24-4.14 (m, 2H), 3.86 |
| | | (dq, J = 6.4, 3.1 Hz, 1H), 1.65 (t, J = 7.0 Hz, 2H), 1.38- |
| | | 1.25 (m, 10H), 0.91-0.83 (m, 3H). |
| C58 | C28H31F3N4O6 | 1H NMR (400 MHz, DMSO) δ 10.11 (t, J = 5.8 Hz, 1H), |
| | 577.33 [M + H] | 8.45 (s, 1H), 7.26-7.14 (m, 2H), 5.64 (dt, J = 11.6, 2.4 |
| | | Hz, 1H), 5.35 (ddd, J = 11.6, 3.6, 2.5 Hz, 1H), 5.27 (dd, J = |
| | | 7.7, 2.6 Hz, 1H), 5.05 (d, J = 14.6 Hz, 1H), 4.78 (d, J = |
| | | 14.5 Hz, 1H), 4.55 (d, J = 5.8 Hz, 2H), 4.19 (t, J = 6.4 Hz, |
| | | 2H), 3.90-3.80 (m, 1H), 1.64 (p, J = 6.6 Hz, 2H), 1.40- |
| | | 1.20 (m, 12H), 0.90-0.82 (m, 3H). |
| C59 | C29H33F3N4O6 | 1H NMR (400 MHz, DMSO) δ 10.11 (t, J = 5.8 Hz, 1H), |
| | 591.36 [M + H] | 8.45 (s, 1H), 7.25-7.16 (m, 2H), 5.65 (dt, J = 11.6, 2.4 |
| | | Hz, 1H), 5.40-5.31 (m, 1H), 5.29-5.26 (m, 1H), 5.05 (d, |
| | | J = 14.6 Hz, 1H), 4.78 (d, J = 14.6 Hz, 1H), 4.55 (d, J = |
| | | 5.8 Hz, 2H), 4.23-4.15 (m, 2H), 3.90-3.81 (M, 1H), 1.63 |
| | | (q, J = 6.8 Hz, 2H), 1.38-1.23 (m, 14H), 0.90-0.82 (m, 3H). |
| C60 | C30H35F3N4O6 | 1H NMR (400 MHz, DMSO) δ 10.11 (t, J = 5.8 Hz, 1H), |
| | 605.40 [M + H] | 8.45 (s, 1H), 7.26-7.14 (m, 2H), 5.65 (dt, J = 11.5, 2.4 |
| | | Hz, 1H), 5.35 (ddd, J = 11.6, 3.6, 2.5 Hz, 1H), 5.30-5.22 |
| | | (m, 1H), 5.05 (d, J = 14.6 Hz, 1H), 4.78 (d, J = 14.5 Hz, |
| | | 1H), 4.55 (d, J = 5.8 Hz, 2H), 4.19 (td, J = 6.5, 1.5 Hz, |
| | | 2H), 3.85 (dq, J = 6.4, 3.1 Hz, 1H), 1.64 (p, J = 6.7 Hz, |
| | | 2H), 1.39-1.18 (m, 16), 0.90-0.82 (m, 3H). |
| C61 | C31H37F3N4O6 | 1H NMR (401 MHz, DMSO) δ 10.11 (t, J = 5.8 Hz, 1H), |
| | 619.43 [M + H] | 8.45 (s, 1H), 7.26-7.14 (m, 2H), 5.64 (dt, J = 11.6, 2.4 |
| | | Hz, 1H), 5.35 (ddd, J = 11.6, 3.6, 2.5 Hz, 1H), 5.26 (dt, J = |
| | | 7.8, 2.7 Hz, 1H), 5.05 (d, J = 14.6 Hz, 1H), 4.78 (d, J = |
| | | 14.5 Hz, 1H), 4.55 (d, J = 5.8 Hz, 2H), 4.19 (td, J = 6.5, |
| | | 1.9 Hz, 2H), 3.85 (dq, J = 6.4, 3.0 Hz, 1H), 1.64 (p, J = 6.6 |
| | | Hz, 2H), 1.38-1.18 (m, 18H), 0.90-0.82 (m, 3H). |
| C62 | C32H39F3N4O6 | 1H NMR (400 MHz, DMSO) δ 10.11 (t, J = 5.8 Hz, 1H), |
| | 633.42 [M + H] | 8.45 (s, 1H), 7.26-7.14 (m, 2H), 5.64 (dt, J = 11.6, 2.4 |
| | | Hz, 1H), 5.35 (ddd, J = 11.6, 3.6, 2.5 Hz, 1H), 5.30-5.23 |
| | | (m, 1H), 5.05 (d, J = 14.6 Hz, 1H), 4.78 (d, J = 14.6 Hz, |
| | | 1H), 4.55 (d, J = 5.8 Hz, 2H), 4.19 (td, J = 6.5, 1.8 Hz, |
| | | 2H), 3.85 (dq, J = 6.3, 3.0 Hz, 1H), 1.64 (p, J = 6.6 Hz, |
| | | 2H), 1.38-1.18 (m, 20H), 0.89-0.81 (m, 3H). |
| C63 | C34H43F3N4O6 | H NMR (400 MHz, DMSO) δ 10.11 (t, J = 5.8 Hz, 1H), |
| | 661.53 [M + H] | 8.45 (s, 1H), 7.20 (t, J = 8.6 Hz, 2H), 5.64 (dt, J = 11.6, |
| | | 2.5 Hz, 1H), 5.35 (dt, J = 11.6, 3.0 Hz, 1H), 5.31-5.22 (m, |
| | | 1H), 5.05 (d, J = 14.6 Hz, 1H), 4.78 (d, J = 14.5 Hz, 1H), |
| | | 4.55 (d, J = 5.9 Hz, 2H), 4.23-4.15 (m, 2H), 3.91-3.80 |
| | | (m, 1H), 1.63 (q, J = 6.7 Hz, 2H), 1.38-1.16 (m, |
| | | 24H), 0.89-0.81 (m, 3H). |
| C64 | C36H47F3N4O6 | 1H NMR (400 MHz, DMSO) δ 10.12 (t, J = 5.8 Hz, 1H), |
| | 689.46 [M + H] | 8.45 (s, 1H), 7.26-7.14 (m, 2H), 5.64 (dt, J = 11.6, 2.4 |
| | | Hz, 1H), 5.35 (ddd, J = 11.6, 3.6, 2.5 Hz, 1H), 5.27 (dd, J = |
| | | 7.4, 2.7 Hz, 1H), 5.05 (d, J = 14.7 Hz, 1H), 4.78 (d, J = |
| | | 14.5 Hz, 1H), 4.55 (d, J = 5.8 Hz, 2H), 4.19 (td, J = 6.5, |
| | | 1.8 Hz, 2H), 3.85 (dq, J = 6.6, 3.0 Hz, 1H), 1.64 (p, J = 6.5 |
| | | Hz, 2H), 1.44-1.16 (m, 28H), 0.89-0.81 (m, 3H). |
| C65 | C38H51F3N4O6 | 1H NMR (400 MHz, DMSO) δ 10.11 (t, J = 5.8 Hz, 1H), |
| | 717.57 [M + H] | 8.45 (s, 1H), 7.20 (dd, J = 9.2, 8.0 Hz, 2H), 5.64 (dt, J = |
| | | 11.6, 2.5 Hz, 1H), 5.35 (ddd, J = 11.6, 3.6, 2.5 Hz, 1H), |
| | | 5.31-5.21(m, 1H), 5.05 (d, J = 14.6 Hz, 1H), 4.78 (d, J = |
| | | 14.5 Hz, 1H), 4.55 (d, J = 5.8 Hz, 2H), 4.24-4.14 (m, |
| | | 2H), 3.85 (dq, J = 6.5, 3.1 Hz, 1H), 1.63 (q, J = 6.8 Hz, |
| | | 2H), 1.341-1.15 (m, 32H), 0.89-0.81 (m, 3H). |
| C66 | C40H55F3N4O6 | 1H NMR (401 MHz, DMSO) δ 10.12 (t, J = 5.8 Hz, 1H), |
| | 745.55 [M + H] | 8.45 (s, 1H), 7.20 (t, J = 8.6 Hz, 2H), 5.64 (dt, J = 11.6, |
| | | 2.4 Hz, 1H), 5.35 (dt, J = 11.7, 3.0 Hz, 1H), 5.31-5.21 (m, |
| | | 1H), 5.05 (d, J = 14.6 Hz, 1H), 4.78 (d, J = 14.6 Hz, 1H), |
| | | 4.55 (d, J = 5.8 Hz, 2H), 4.22-4.15 (m, 2H), 3.90- |
| | | 3.80(m, 1H), 1.70-1.58 (m, 2H), 1.44-1.16(m, 36H), 0.89- |
| | | 0.81 (m, 3H). |
| C67 | C26H27F3N4O6 | H NMR (400 MHz, DMSO) δ 10.11 (t, J = 5.8 Hz, 1H), |
| | 549.34 [M + H] | 8.45 (s, 1H), 7.27-7.15 (m, 2H), 5.64 (dt, J = 11.6, 2.4 |
| | | Hz, 1H), 5.35 (ddd, J = 11.6, 3.6, 2.5 Hz, 1H), 5.31- |
| | | 5.22(m, 1H), 5.05 (d, J = 14.6 Hz, 1H), 4.78 (d, J = 14.5 |
| | | Hz, 1H), 4.58-4.52 (m, 2H), 4.07-3.93 (m, 2H), 3.86 |
| | | (dd, J = 6.8, 3.5 Hz, 1H), 1.96 (hept, J = 6.8 Hz, 1H), 1.35 |
| | | (d, J = 6.8 Hz, 3H), 1.28 (d, J = 7.3 Hz, 3H), 0.95-0.89 |
| | | (m, 6H). |
| C68 | C27H29F3N4O6 | 1H NMR (400 MHz, DMSO) δ 10.11 (t, J = 5.8 Hz, 1H), |
| | 563.30 [M + H] | 8.45 (s, 1H), 7.21 (t, J = 8.6 Hz, 2H), 5.64 (dt, J = 11.6, |
| | | 2.4 Hz, 1H), 5.35 (dt, J = 11.8, 3.0 Hz, 1H), 5.30-5.24 |
| | | (m, 1H), 5.05 (d, J = 14.6 Hz, 1H), 4.78 (d, J = 14.5 Hz, |
| | | 1H), 4.58-4.50 (m, 2H), 4.23 (td, J = 6.7, 2.6 Hz, 2H), |
| | | 3.86 (dq, J = 6.7, 3.2 Hz, 1H), 1.68 (dp, J = 13.4, 6.7 Hz, |
| | | 1H), 1.55 (q, J = 6.7 Hz, 2H), 1.35 (d, J = 6.8 Hz, 3H), |
| | | 1.28 (d, J = 7.3 Hz, 3H), 0.89 (d, J = 6.6 Hz, 6H). |
| C69 | C28H31F3N4O6 | 1H NMR (400 MHz, DMSO) δ 10.11 (t, J = 5.8 Hz, 1H), |
| | 577.37 [M + H] | 8.45 (s, 1H), 7.27-7.14 (m, 2H), 5.65 (dt, J = 11.6, 2.4 |
| | | Hz, 1H), 5.35 (ddd, J = 11.6, 3.6, 2.5 Hz, 1H), 5.31-5.23 |
| | | (m, 1H), 5.05 (d, J = 14.6 Hz, 1H), 4.78 (d, J = 14.9 Hz, |
| | | 1H), 4.62-4.50 (m, 2H), 4.18 (t, J = 6.5 Hz, 2H), 3.86 |
| | | (dq, J = 6.4, 3.0 Hz, 1H), 1.64 (dt, J = 16.1, 6.6 Hz, 2H), |
| | | 1.53 (dp, J = 13.3, 6.6 Hz, 1H), 1.35 (d, J = 6.8 Hz, 3H), |
| | | 1.28 (d, J = 7.3 Hz, 3H), 1.26-1.19 (m, 2H), 0.85 (d, J = |
| | | 6.6 Hz, 6H). |
| C70 | C29H33F3N4O6 | 1H NMR (400 MHz, DMSO) δ 10.11 (t, J = 5.8 Hz, 1H), |
| | 591.36 [M + H] | 8.45 (s, 1H), 7.27-7.14 (m, 2H), 5.65 (dt, J = 11.6, 2.4 |
| | | Hz, 1H), 5.35 (ddd, J = 11.6, 3.6, 2.5 Hz, 1H), 5.26 (dp, J = |
| | | 8.9, 3.1 Hz, 1H), 5.05 (d, J = 14.6 Hz, 1H), 4.78 (d, J = |
| | | 14.5 Hz, 1H), 4.58-4.50 (m, 2H), 4.20 (t, J = 6.4 Hz, |
| | | 2H), 3.85 (dp, J = 10.1, 3.7 Hz, 1H), 1.62 (p, J = 6.8 Hz, |
| | | 2H), 1.50 (dp, J = 13.3, 6.7 Hz, 1H), 1.38-1.23 (m, 5H), |
| | | 1.28 (d, J = 7.3 Hz, 3H), 1.25-1.12 (m, 2H), 0.85 (d, J = |
| | | 6.6 Hz, 6H). |
| C71 | C30H35F3N4O6 | 1H NMR (401 MHz, DMSO) δ 10.11 (t, J = 5.8 Hz, 1H), |
| | 605.31 [M + H] | 8.45 (s, 1H), 7.26-7.14 (m, 2H), 5.65 (dt, J = 11.6, 2.4 |
| | | Hz, 1H), 5.36 (ddd, J = 11.5, 3.6, 2.5 Hz, 1H), 5.27 (dt, J = |
| | | 7.5, 2.7 Hz, 1H), 5.05 (d, J = 14.6 Hz, 1H), 4.78 (d, J = |
| | | 14.5 Hz, 1H), 4.55 (d, J = 5.7 Hz, 2H), 4.19 (td, J = 6.5, |
| | | 1.2 Hz, 2H), 3.85 (dq, J = 6.3, 3.0 Hz, 1H), 1.65 (p, J = 6.7 |
| | | Hz, 2H), 1.51 (dp, J = 13.2, 6.6 Hz, 1H), 1.38-1.22 (m, |
| | | 10H), 1.14 (q, J = 7.4 Hz, 2H), 0.85 (d, J = 6.6 Hz, 6H). |
| C72 | C31H37F3N4O6 | 1H NMR (401 MHz, DMSO) δ 10.11 (t, J = 5.8 Hz, 1H), |
| | 619.53 | 8.45 (s, 1H), 7.26-7.15 (m, 2H), 5.64 (dt, J = 11.5, 2.4 |
| | [ M + H] | Hz, 1H), 5.40-5.31 (m, 1H), 5.30-5.22 (m, 1H), 5.05 (d, |
| | | J = 14.6 Hz, 1H), 4.78 (d, J = 14.5 Hz, 1H), 4.55 (d, J = |
| | | 5.8 Hz, 2H), 4.19 (tt, J = 6.1, 3.1 Hz, 2H), 3.86 (dt, J = |
| | | 6.5, 3.2 Hz, 1H), 1.64 (p, J = 6.7 Hz, 2H), 1.50 (dp, J = |
| | | 13.2, 6.6 Hz, 1H), 1.38-1.22 (m, 12H), 1.14 (d, J = 6.9 |
| | | Hz, 2H), 0.85 (d, J = 6.6 Hz, 6H). |
| C73 | C32H39F3N4O6 | 1H NMR (400 MHz, DMSO) δ 10.12 (t, J = 5.8 Hz, 1H), |
| | 633.38 [M + H] | 8.45 (s, 1H), 7.26-7.14 (m, 2H), 5.64 (dt, J = 11.6, 2.4 |
| | | Hz, 1H), 5.36 (ddd, J = 11.6, 3.6, 2.5 Hz, 1H), 5.27 (dq, J = |
| | | 7.7, 2.7 Hz, 1H), 5.05 (d, J = 14.6 Hz, 1H), 4.78 (d, J = |
| | | 14.5 Hz, 1H), 4.55 (d, J = 5.8 Hz, 2H), 4.19 (tt, J = 6.1, 3.1 |
| | | Hz, 2H), 3.85 (dq, J = 6.4, 3.0 Hz, 1H), 1.64 (p, J = 6.6 |
| | | Hz, 2H), 1.57-1.41 (m, J = 6.6 Hz, 1H), 1.38-1.18(m, |
| | | 14H), 1.14 (q, J = 6.7 Hz, 2H), 0.85 (d, J = 6.6 Hz, 6H). |
| C74 | C27H29F3N4O8 | 1H NMR (400 MHz, DMSO) δ 10.11 (t, J = 5.8 Hz, 1H), |
| | 595.31 [M + H] | 8.45 (s, 1H), 7.27-7.14 (m, 2H), 5.65 (dt, J = 11.6, 2.4 |
| | | Hz, 1H), 5.35 (ddd, J = 11.6, 3.6, 2.5 Hz, 1H), 5.31-5.24 |
| | | (m, 1H), 5.05 (d, J = 14.6 Hz, 1H), 4.79 (d, J = 14.5 Hz, |
| | | 1H), 4.55 (d, J = 5.8 Hz, 2H), 4.30 (td, J = 4.0, 2.4 Hz, |
| | | 2H), 3.87 (dq, J = 6.5, 3.0 Hz, 1H), 3.71-3.64 (m, 2H), |
| | | 3.60-3.53 (m, 2H), 3.49-3.42 (m, 2H), 3.25 (s, 3H), |
| | | 1.32 (dd, J = 24.3, 7.0 Hz, 6H). |

### Example 4b: Alternate synthesis of (1S,2R,5S)-2,5-dimethyl-7,9-dioxo-10-((2,4,6-trifluorobenzyl)carbamoyl)-2,5,7,9-tetrahydro-1,6-methanopyrido[1,2-b][1,2,5]triazonin-8-yl (tetrahydro-2H-pyran-4-yl) carbonate (C50)

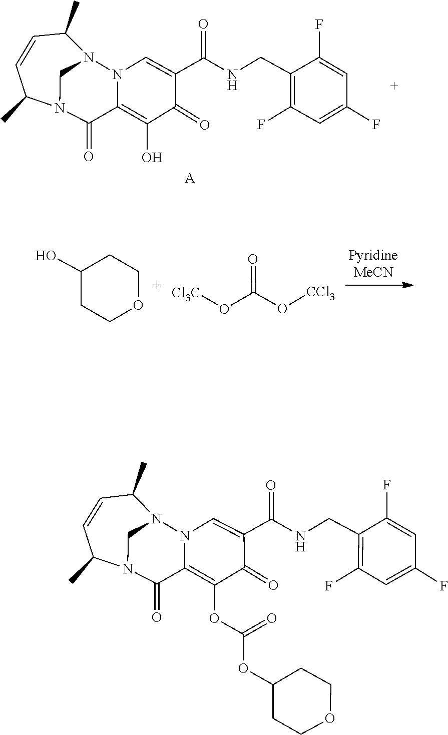

To a solution of triphosgene (0.010 g, 0.034 mmol) in MeCN (1 mL) was added pyridine (0.014 mL, 0.178 mmol). Tetrahydro-2H-pyran-4-ol (0.006 mL, 0.067 mmol) was added followed by a solution of Intermediate A (0.020 g, 0.045 mmol) in MeCN (1 mL). After 27 h, the reaction mixture was quenched with saturated aqueous NaHCO₃ and extracted with EtOAc. The organic phase was dried over Na₂SO₄, filtered, concentrated, and purified by silica gel column chromatography to afford the title compound. MS (m/z) 577.10 [M+H]+. ¹H NMR (400 MHz, Acetonitrile-d3) δ 10.16 (s, 1H), 8.51 (s, 1H), 6.98-6.72 (m, 2H), 5.76-5.52 (m, 1H), 5.52-5.26 (m, 2H), 4.98 (d, J=14.5 Hz, 1H), 4.93-4.78 (m, 1H), 4.68-4.50 (m, 3H), 3.84 (dq, J=10.4, 6.1, 5.3 Hz, 3H), 3.50 (td, J=11.1, 2.9 Hz, 2H), 1.84-1.61 (m, 2H), 1.36 (d, J=6.8 Hz, 3H), 1.30 (d, J=7.4 Hz, 3H).

### Example 4c: Alternate synthesis of (1S,2R,5S)-2,5-dimethyl-7,9-dioxo-10-((2,4,6-trifluorobenzyl)carbamoyl)-2,5,7,9-tetrahydro-1,6-methanopyrido[1,2-b][1,2,5]triazonin-8-yl (2-methoxyethyl) carbonate (C51)

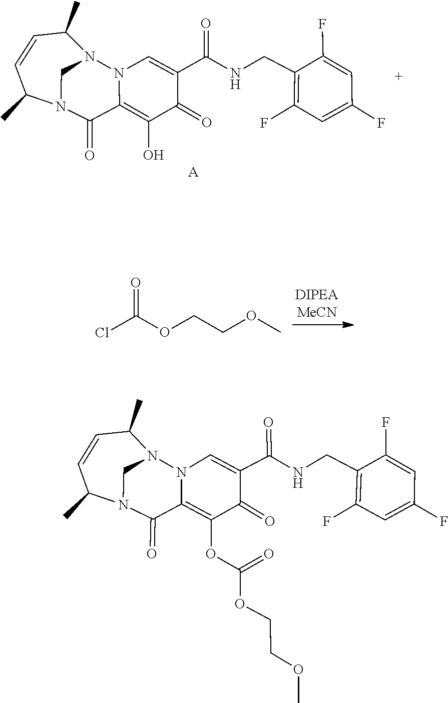

A mixture of Intermediate A (0.015 g, 0.034 mmol), 2-methoxyethyl chloroformate (0.006 mL, 0.050 mmol), and DIPEA (0.016 mL, 0.092 mmol) was stirred at room temperature for 2 h. The reaction mixture was quenched with saturated aqueous NaHCO₃ and extracted with EtOAc. The organic phase was dried over Na2SO4, filtered, and concentrated to afford the title product. MS (m/z) 551.14 [M+H]+. ¹H NMR (400 MHz, Acetonitrile-d3) δ 10.16 (s, 1H), 8.50 (s, 1H), 7.00-6.70 (m, 2H), 5.75-5.47 (m, 1H), 5.47-5.22 (m, 2H), 4.98 (d, J=14.6 Hz, 1H), 4.75-4.44 (m, 3H), 4.44-4.21 (m, 2H), 3.83 (dq, J=6.5, 3.2 Hz, 1H), 3.72-3.52 (m, 2H), 3.34 (s, 3H), 1.36 (d, J=6.8 Hz, 3H), 1.30 (d, J=7.2 Hz, 3H).

### Example 4d: Synthesis of (1S,2R,5S)-2,5-dimethyl-7,9-dioxo-10-((2,4,6-trifluorobenzyl)carbamoyl)-2,5,7,9-tetrahydro-1,6-methanopyrido[1,2-b][1,2,5]triazonin-8-yl isopropyl carbonate (C52)

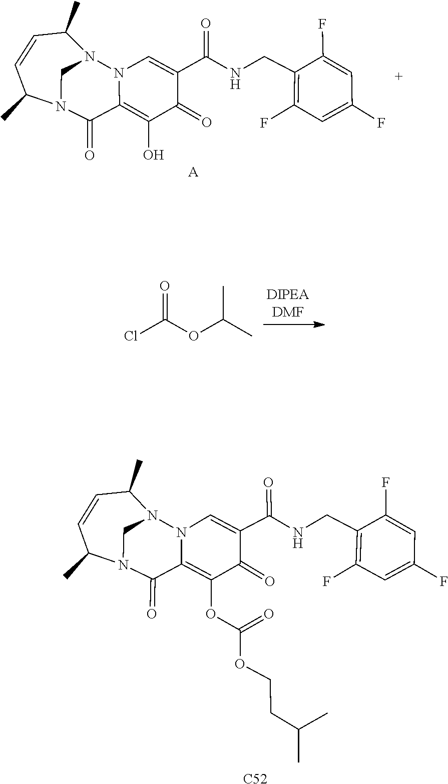

Intermediate A (96 mg, 0.214 mmol) was dissolved in DMF (1.5 ml) and DIPEA (111 mg, 0.856 mmol) was added. The mixture was cooled to 0 °C with ice-water bath. Then isopropyl chloroformate (63 mg, 0.514 mmol) was added slowly. The cold bath was removed, and the reaction mixture was stirred at rt for 17 h. The reaction mixture was diluted with EtOAc (10 ml) and treated with water (10 ml) and brine (10 ml) sequentially. The org anic phase was separated and dried over sodium sulfate, filtered and concentrated. The residue was purified by silica gel chromatography (0-100% EtOAc in hexanes) to afford the title compound. MS (m/z): 535.10 [M+H]+. 1H NMR (400 MHz, CD3CN) δ 10.20 (s, 1H), 8.53 (s, 1H), 6.94-6.81 (m, 2H), 5.66 (dt, J=11.1, 2.5 Hz, 1H), 5.45-5.35 (m, 2H), 5.04-4.87 (m, 2H), 4.70-4.55 (m, 3H), 3.85 (dh, J=6.6, 3.5 Hz, 1H), 1.37 (dd, J=10.9, 6.5 Hz, 9H), 1.32 (d, J=7.3 Hz, 3H).

### Example 5: Synthesis of Acetal Carbonates of Intermediate A

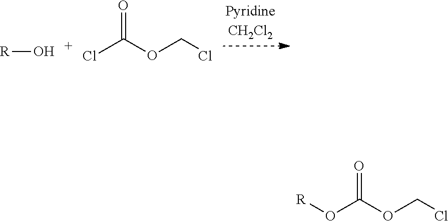

To a mixture of alcohol and chloromethyl chloroformate in CH₂Cl₂ at 0 °C is added pyridine. The reaction mixture is allowed to warm to rt and stir for 2 h. The reaction mixture is washed with 0.5 N HCl (2×) and the organic phase is dried over Na₂SO₄, filtered, and concentrated to afford the desired product. The product is used directly in the next step.

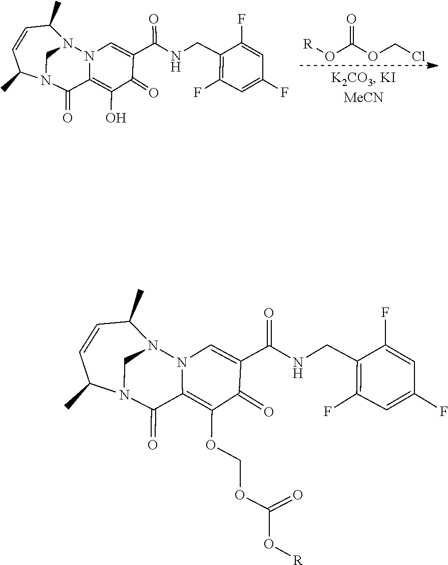

To a mixture of Intermediate A in MeCN is added potassium carbonate, potassium iodide, and the chloromethyl carbonate. The mixture is heated to 60 °C for 16 h, cooled to room temperature, and filtered. The filter cake is rinsed with MeCN and the filtrate is concentrated. The residue is purified by reverse phase prep HPLC (5-100% MeCN/H₂O w/0.1% TFA) to afford the desired compound after lyophilization.

### Example 5a: Alternative Synthesis of Acetal Carbonates of Intermediate A

### Step 1: Synthesis of Chloromethyl Carbonate

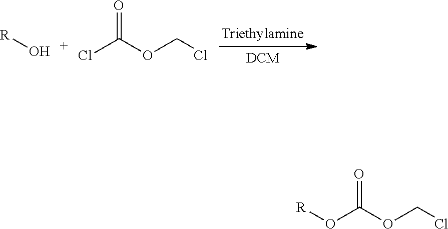

To a mixture of the corresponding alcohol (1.0 eq.) in DCM (20 vol) was added chloromethyl carbonochloridate (1.20 eq.) under vigorous stirring. Then cooled to 0 °C, and Triethylamine (1.50 eq.) and the reaction mixture was stirred at room temperature for 18 h. The reaction mixture was diluted with CH₂Cl₂, washed with water and followed by brine, dried over Na₂SO₄, filtered, and concentrated to afford the desired chloromethyl carbonate.

### Step 2: Alkylation

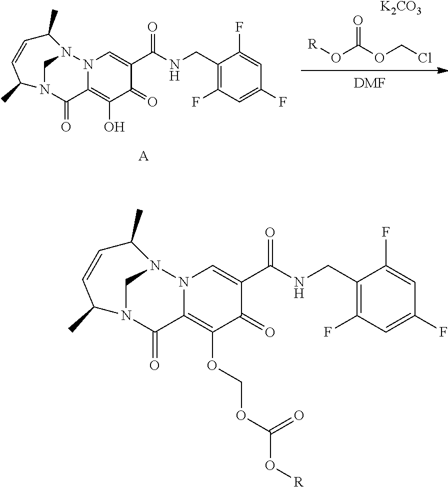

To a stirred solution of Intermediate A (1 eq,) in DMF (20 vol.), was added potassium carbonate (3 eq.) followed by chloromethyl carbonate (6 eq.). Then resulting reaction was stirred for 18 h at ambient temperature. The reaction mixture was diluted with EtOAc, and was washed with water followed by brine solution. The organic phase was dried over Na₂SO₄, filtered, and concentrated to obtained corresponding desired crude product.

### Example 5b: Alternative Synthesis of Acetal Carbonates of Intermediate A

### Step 1: Synthesis of Chloromethyl Carbonate

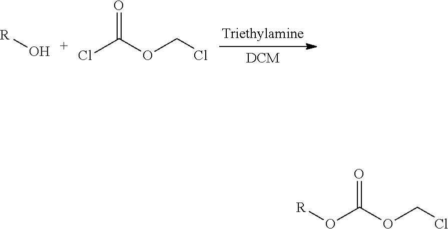

To a mixture of the corresponding alcohol (1.0 eq.) in DCM (20 vol.), was added chloromethyl carbonochloridate (1.2 eq.) under vigorous stirring. Then cooled to 0 °C, and Triethylamine (1.5 eq.) and the reaction mixture was stirred at room temperature for 18 h. The reaction mixture was diluted with CH₂Cl₂ and washed with water, followed by brine, dried over Na₂SO₄, filtered, and concentrated to afford the desired chloromethyl carbonate.

### Step 2: Synthesis of Iodomethyl Carbonate

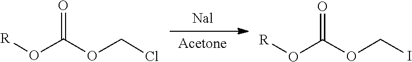

To a stirred solution of corresponding chloromethyl carbonate (1.0 eq.) in Acetone, were added Sodium iodide (1.5 eq.) at ambient temperature and stirred for 18 h at 45 °C. The reaction mixture was diluted with EtOAc and was washed with water and brine. The organic phase was dried over Na₂SO₄, filtered, and concentrated to obtain corresponding iodomethyl carbonate.

### Step 3: Alkylation

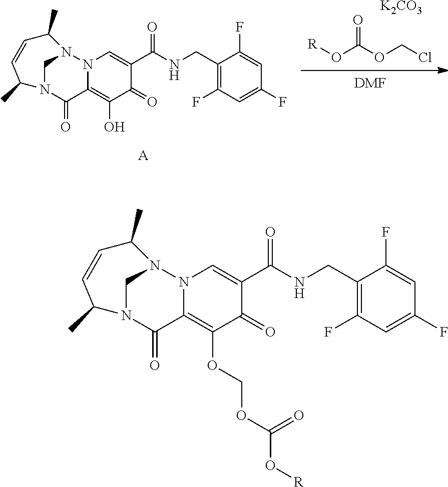

To a stirred solution of Intermediate A (1.0 eq.) in DMF (20 vol.) was added potassium carbonate (3 eq.) followed by iodomethyl carbonate (6.0 eq.). Then resulting reaction was stirred for 18 h at ambient temperature. The reaction mixture was diluted with EtOAc, and was washed with water followed by brine solution. The organic phase was dried over Na₂SO₄, filtered, and concentrated to obtain corresponding crude product.

### Purification Method-1:

The crude product was purified by silica gel column chromatography (65-70% EtOAc/hexane) and fractions containing product were pooled and concentrated. Obtained product was lyophilized from 1:1 MeCN/water to afford the desired compound.

### Purification Method-2:

The crude product was purified by silica gel column chromatography (50-70% EtOAc/hexane) and fractions containing product were pooled and concentrated. Obtained product was lyophilized from 1:1 MeCN/water to afford the desired compound.

### Purification Method-3:

The crude product was purified Prep-HPLC column chromatography in ACN: WATER, the pure fractions were collected and lyophilized to afford the desired compound.

The following compounds are prepared following the procedure of Example 5 using the chloromethyl carbonates indicated below.

Alternatively, the following compounds C75-C77, C89, C90, C97 and C98 were prepared following the procedure of Step 2 of Example 5a using the chloromethyl carbonates indicated below. The chloromethyl carbonates for compounds C90, C97 and C98 were prepared following the procedure of Step 1 of Example 5a using the alcohols indicated below. The following compounds C78-C88, and C91-C96 were prepared following the procedure of Example 5b using the alcohols indicated below. Compounds C75-C80, C89-C93, and C98 were purified by Purification Method-1. Compounds C81-C88, C94, C96, C97 were purified by Purification Method-2. Compounds C95 was purified by Purification Method-3.

| | | Chloromethyl | |
|---|---|---|---|
| Compound | | Carbonate | |
| No. | Structure | (ROCOOCH₂Cl)* | Alcohol (R—OH) |
| C75 | 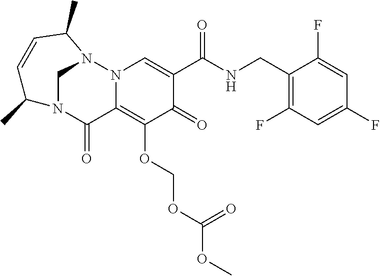 | CH₃OCOOCH₂Cl | CH₃OH |
| C76 | 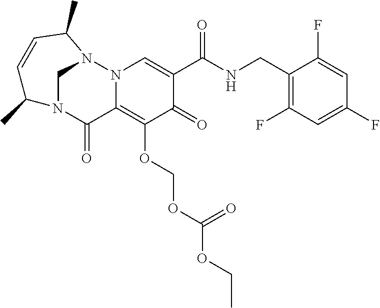 | CH₃CH₂OCOOCH₂Cl | CH₃CH₂OH |
| C77 | 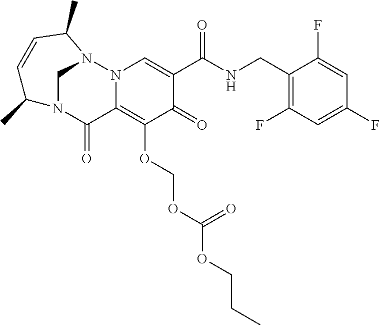 | CH₃(CH₂)₂OCOOCH₂Cl | CH₃(CH₂)₂OH |
| C78 | 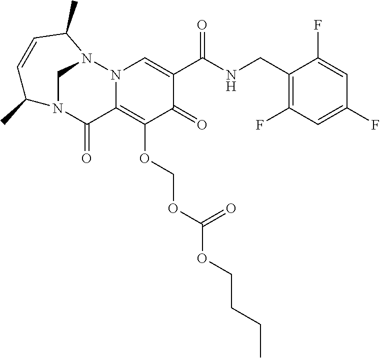 | CH₃(CH₂)₃OCOOCH₂Cl | CH₃(CH₂)₃OH |
| C79 | 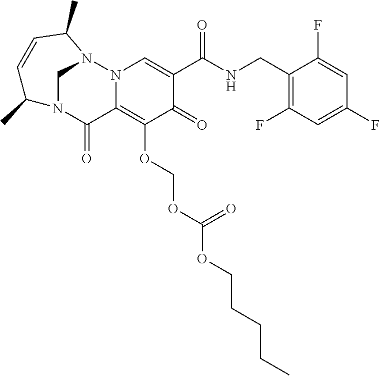 | CH₃(CH₂)₄OCOOCH₂Cl | CH₃(CH₂)₄OH |
| C80 | 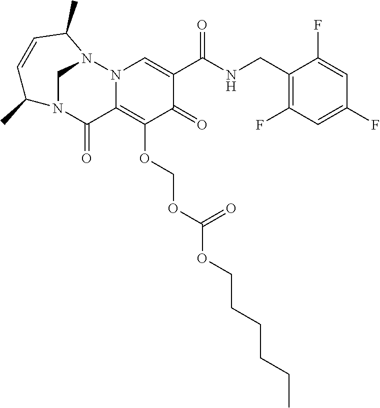 | CH₃(CH₂)₅OCOOCH₂Cl | CH₃(CH₂)₅OH |
| C81 | 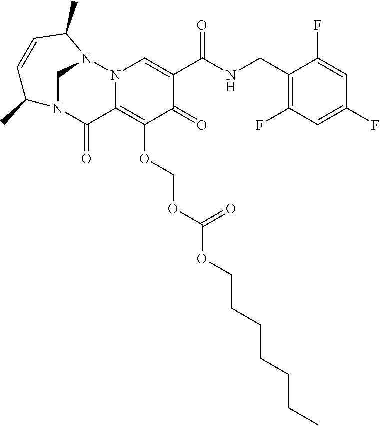 | CH₃(CH₂)₆OCOOCH₂Cl | CH₃(CH₂)₆OH |
| C82 | 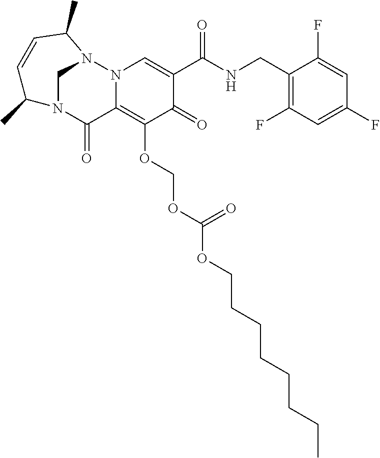 | CH₃(CH₂)₇OCOOCH₂Cl | CH₃(CH₂)₇OH |
| C83 | 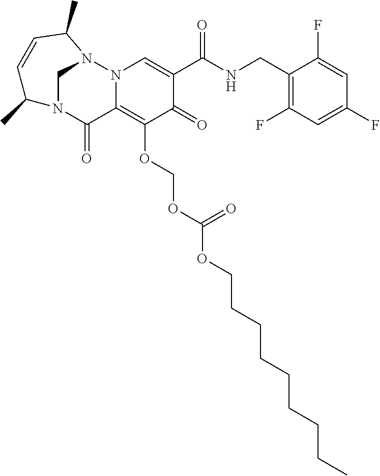 | CH₃(CH₂)₈OCOOCH₂Cl | CH₃(CH₂)₈OH |
| C84 | 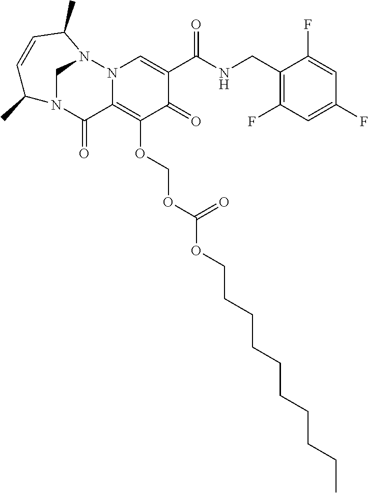 | CH₃(CH₂)₉OCOOCH₂Cl | CH₃(CH₂)₉OH |
| C85 | 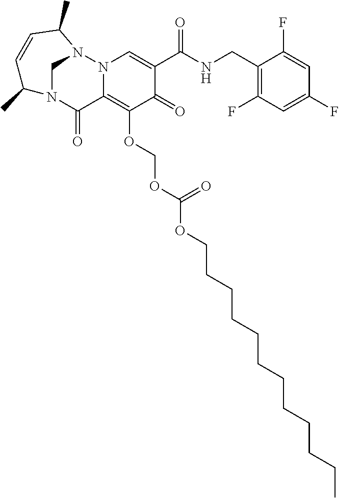 | CH₃(CH₂)₁₁OCOOCH₂Cl | CH₃(CH₂)₁₁OH |
| C86 | 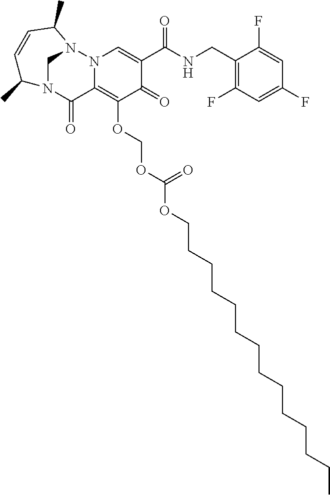 | CH₃(CH₂)₁₃OCOOCH₂Cl | CH₃(CH₂)₁₃OH |
| C87 | 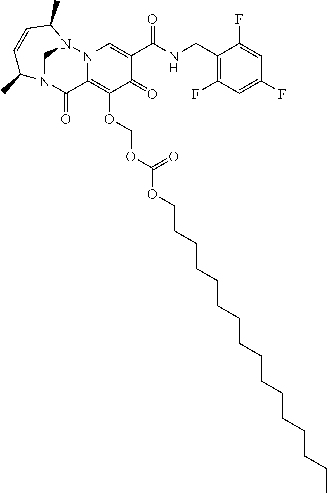 | CH₃(CH₂)₁₅OCOOCH₂Cl | CH₃(CH₂)₁₅OH |
| C88 | 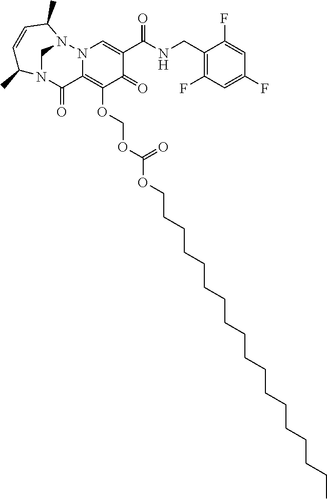 | CH₃(CH₂)₁₇OCOOCH₂Cl | CH₃(CH₂)₁₇OH |
| C89 | 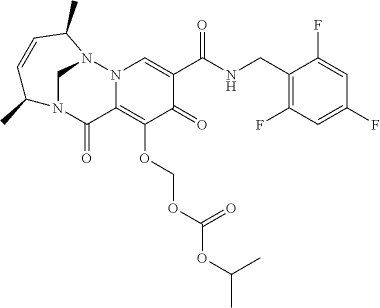 | (CH₃)₂CHOCOOCH₂Cl | (CH₃)₂CHOH |
| C90 | 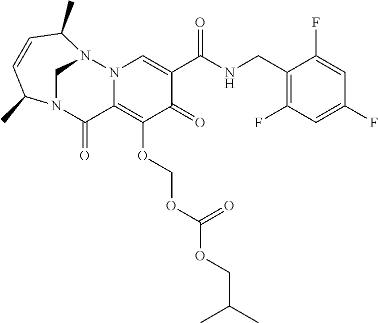 | (CH₃)₂CHCH₂OCOOCH₂Cl | (CH₃)₂CHCH₂OH |
| C91 | 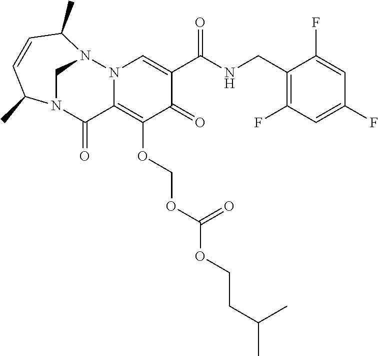 | (CH₃)₂CH(CH₂)₂OCOOCH₂Cl | (CH₃)₂CH(CH₂)₂OH |
| C92 | 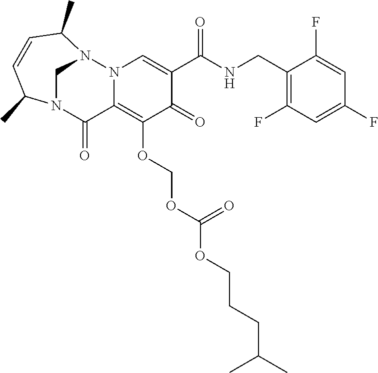 | (CH₃)₂CH(CH₂)₃OCOOCH₂Cl | (CH₃)₂CH(CH₂)₃OH |
| C93 | 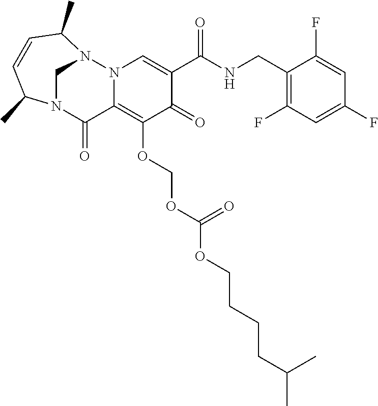 | (CH₃)₂CH(CH₂)₄OCOOCH₂Cl | (CH₃)₂CH(CH₂)₄OH |
| C94 | 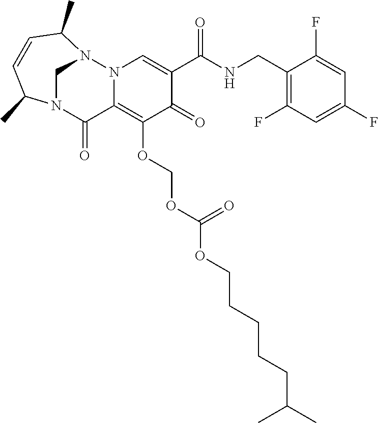 | (CH₃)₂CH(CH₂)₅OCOOCH₂Cl | (CH₃)₂CH(CH₂)₅OH |
| C95 | 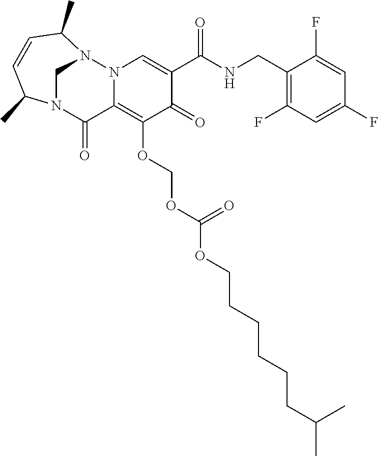 | (CH₃)₂CH(CH₂)₆OCOOCH₂Cl | (CH₃)₂CH(CH₂)₆OH |
| C96 | 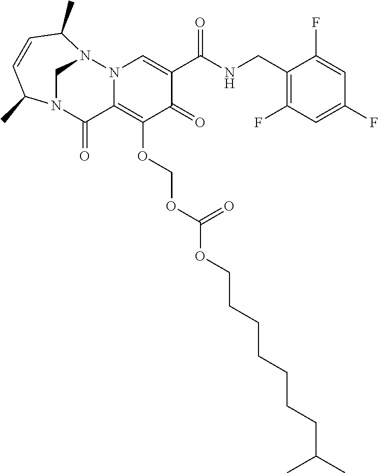 | (CH₃)2CH(CH₂)₇OCOOCH₂Cl | (CH₃)₂CH(CH₂)₇OH |
| C97 | 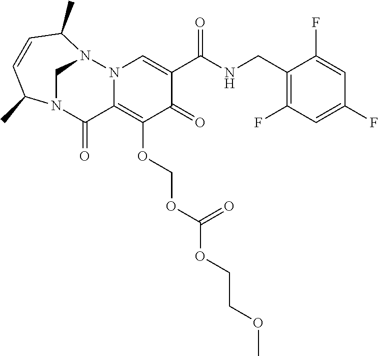 | CH₃OCH₂CH₂OCOOCH₂Cl | CH₃OCH₂CH₂OH |
| C98 | 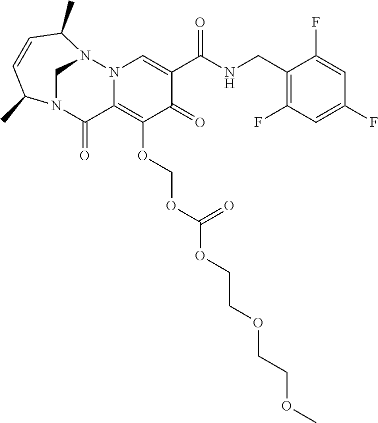 | CH₃(OCH₂CH₂)₂OCOOCH₂Cl | CH₃(OCH₂CH₂)₂OH |

*Commercially available or prepared from the corresponding alcohol.

### Example 6: Synthesis of Acetal Carbonates of Intermediate a

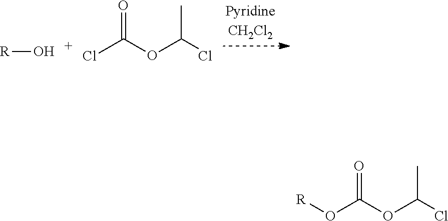

To a mixture of alcohol and 1-chloroethyl chloroformate in CH₂Cl₂ at 0 °C is added pyridine. The reaction mixture is allowed to warm to rt and stir for 2 h. The reaction mixture is washed with 0.5 N HCl (2×) and the organic phase is dried over Na₂SO₄, filtered, and concentrated to afford the desired product. The product is used directly in the next step.

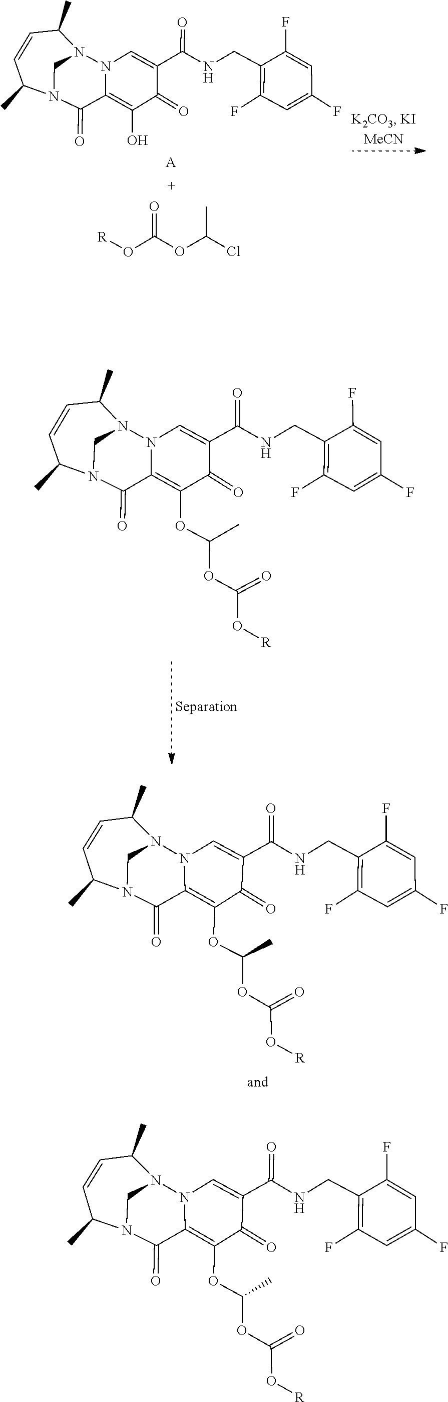

To a mixture of Intermediate A in MeCN is added potassium carbonate, potassium iodide, and the 1-chloroethyl carbonate. The mixture is heated to 70 °C for 3 h, cooled to room temperature, and filtered. The filter cake is rinsed with MeCN and the filtrate is concentrated. The residue is purified by reverse phase prep HPLC (5-100% MeCN/H₂O w/0.1% TFA) to afford the desired compound after lyophilization. The diastereomers are separated (for example by chiral SFC or chiral HPLC). Stereochemistry is arbitrarily assigned.

### Example 6a: Alternative Synthesis of Acetal Carbonates of Intermediate a

### Step 1: Synthesis of Chloromethyl Carbonate

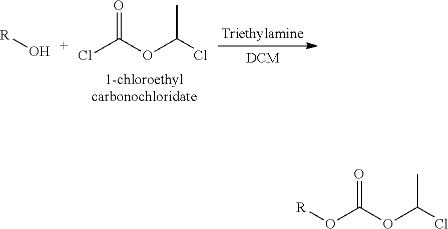

To a mixture of the corresponding alcohol (1.0 eq.) in DCM (20 vol.) was added 1-chloroethyl carbonochloridate (1.20 eq.) under vigorous stirring. The mixture was then cooled to 0 °C and triethylamine (1.50 eq.) was added. The reaction mixture was stirred at room temperature for 18 h. The reaction mixture was diluted with CH₂Cl₂, washed with water and followed by brine, dried over Na₂SO₄, filtered, and concentrated to afford the desired 1-chloroethyl carbonate intermediate.

### Step 2: Synthesis of Iodomethyl Carbonate

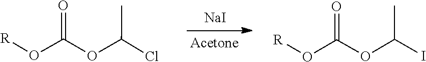

To a stirred solution of corresponding 1-chloroethyl carbonate (1.0 eq.) in acetone, were added sodium iodide (1.5 eq.) at ambient temperature and stirred for 18 h at 45 °C. The reaction mixture was diluted with EtOAc and was washed with water and brine. The organic phase was dried over Na₂SO₄, filtered, and concentrated to obtain the corresponding 1-iodoethyl carbonate.

### Step 3: Alkylation

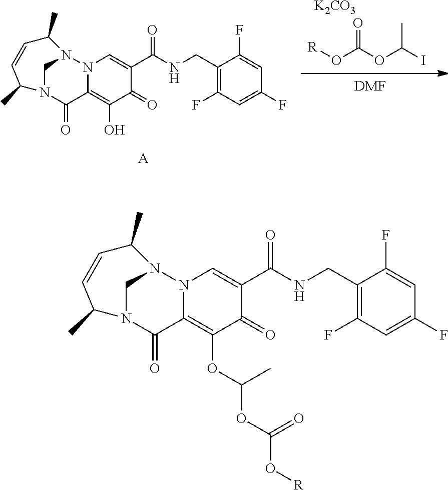

To a stirred solution of Intermediate A (1.0 eq.) in DMF (20 vol.), was added potassium carbonate (3 eq.) followed by 1-iodoethyl carbonate (6.0 eq.). Then resulting reaction was stirred for 18 h at ambient temperature. The reaction mixture was diluted with EtOAc, and was washed with water followed by brine solution. The organic phase was dried over Na₂SO₄, filtered, and concentrated to obtained crude product. The crude product was purified by silica gel column chromatography (65-70% EtOAc/hexane) and fractions containing product were pooled and concentrated. Pure products were purified by Chiral SFC separation method below. Each isomer was concentrated following lyophilized from 1:1 MeCN/water to afford the desired compound.

The following compounds are prepared following the procedure of Example 6 using the chloromethyl carbonates indicated below.

Alternatively, the following compounds were prepared following the procedure of Example 6a using the alcohol indicated below.

| | | Chloromethyl | |
|---|---|---|---|
| Compound | | Carbonate | |
| No. | Structure | (ROCOOCH(CH₃)Cl)* | Alcohol (R—OH) |
| C99 | 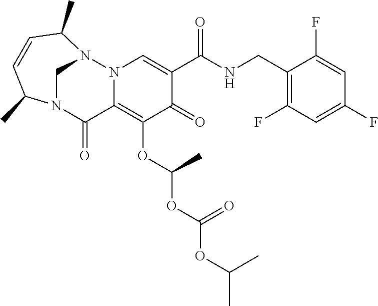 | 1-chloroethyl isopropyl carbonate | (CH₃)₂CHOH |
| C100 | 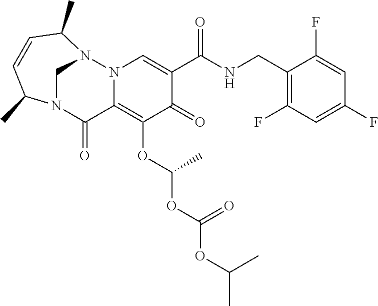 | 1-chloroethyl isopropyl carbonate | (CH₃)₂CHOH |

*Commercially available or prepared from the corresponding alcohol.

### Chiral SFC Separation Method (for Example 99 and Example 100):

Column: CHIRALPAK IG (4.6*250 mm) 5m
Co-Solvent:0.5% DEA IN METHANOL
Total Flow: 3 mL/min
% of CO2: 60
% of Co-Solvent: 40
ABPR: 1500 psi
Column Temp: 30 °C
Diluent: MEOH
Peak 1: Example 99
Peak 2: Example 100

The characterization data of the compounds C75-C100 are shown in the table below.

| | Chemical | |
|---|---|---|
| Compound | Formula | |
| No. | MS (m/z) | ¹H NMR Peak Table |
| C75 | C24H23F3N4O7 | 1H NMR (400 MHz, DMSO) δ 10.27 (t, J = 5.8 Hz, 1H), |
| | 537.25 [M + H] | 8.36 (s, 1H), 7.27-7.15 (m, 2H), 5.84 (d, J = 6.5 Hz, 1H), |
| | | 5.68-5.60 (m, 2H), 5.40-5.26 (m, 1H), 5.30-5.27 (m, |
| | | 1H), 5.01 (d, J = 14.5 Hz, 1H), 4.66 (d, J = 14.5 Hz, 1H), |
| | | 4.55 (d, J = 5.8 Hz, 2H), 3.80 (dq, J = 6.5, 3.2 Hz, 1H), |
| | | 3.70 (s, 3H), 1.29 (dd, J = 19.3, 7.0 Hz, 6H). |
| C76 | C25H25F3N4O7 | 1H NMR (400 MHz, DMSO) δ 10.26 (t, J = 5.8 Hz, 1H), |
| | 551.28 [M + H] | 8.36 (s, 1H), 7.27-7.15 (m, 2H), 5.82 (d, J = 6.4 Hz, 1H), |
| | | 5.68-5.60 (m, 2H), 5.40-5.25 (m, 2H), 5.01 (d, J = 14.5 |
| | | Hz, 1H), 4.66 (d, J = 14.5 Hz, 1H), 4.55 (d, J = 5.7 Hz, |
| | | 2H), 4.11 (q, J = 7.1 Hz, 2H), 3.80 (dq, J = 6.5, 3.1 Hz, |
| | | 1H), 1.29 (dd, J = 19.8, 7.0 Hz, 6H), 1.18 (t, J = 7.1 Hz, 3H). |
| C77 | C26H27F3N4O7 | 1H NMR (400 MHz, DMSO) δ 10.26 (t, J = 5.8 Hz, 1H), |
| | 565.27 [M + H] | 8.36 (s, 1H), 7.27-7.16 (m, 2H), 5.83 (d, J = 6.5 Hz, 1H), |
| | | 5.68-5.60 (m, 2H), 5.40-5.31 (m, 1H), 5.28 (dd, J = |
| | | 7.4, 2.7 Hz, 1H), 5.01 (d, J = 14.6 Hz, 1H), 4.66 (d, J = |
| | | 14.5 Hz, 1H), 4.55 (d, J = 5.7 Hz, 2H), 4.02 (t, J = 6.6 Hz, |
| | | 2H), 3.80 (dq, J = 6.5, 3.2 Hz, 1H), 1.58 (h, J = 7.1 Hz, |
| | | 2H), 1.32 (d, J = 6.7 Hz, 3H), 1.27 (d, J = 7.3 Hz, 3H), |
| | | 0.86 (t, J = 7.4 Hz, 3H). |
| C78 | C27H29F3N4O7 | 1H NMR (400 MHz, DMSO) δ 10.26 (t, J = 5.8 Hz, 1H), |
| | 579.31 [M + H] | 8.36 (s, 1H), 7.27-7.15 (m, 2H), 5.84 (d, J = 6.5 Hz, 1H), |
| | | 5.68-5.60 (m, 2H), 5.40-5.25 (m, 2H), 5.01 (d, J = 14.6 |
| | | Hz, 1H), 4.66 (d, J = 14.5 Hz, 1H), 4.55 (d, J = 5.7 Hz, |
| | | 2H), 4.06 (t, J = 6.5 Hz, 2H), 3.79 (dq, J = 6.5, 3.2 Hz, |
| | | 1H), 1.54 (dq, J = 8.5, 6.6 Hz, 2H), 1.38-1.24 (m, 8H), |
| | | 0.86 (t, J = 7.4 Hz, 3H). |
| C79 | C28H31F3N4O7 | 1H NMR (400 MHz, DMSO) δ 10.26 (t, J = 5.8 Hz, 1H), |
| | 593.34 [M + H] | 8.36 (s, 1H), 7.27-7.15 (m, 2H), 5.84 (d, J = 6.4 Hz, 1H), |
| | | 5.68-5.60 (m, 2H), 5.40-5.25 (m, 2H), 5.01 (d, J = 14.6 |
| | | Hz, 1H), 4.66 (d, J = 14.5 Hz, 1H), 4.55 (d, J = 5.7 Hz, |
| | | 2H), 4.05 (td, J = 6.6, 1.0 Hz, 2H), 3.79 (dq, J = 6.5, 3.1 |
| | | Hz, 1H), 1.56 (q, J = 6.8 Hz, 2H), 1.35-1.22 (m, 10H), |
| | | 0.89-0.79 (m, 3H). |
| C80 | C29H33F3N4O7 | 1H NMR (400 MHz, DMSO) δ 10.26 (t, J = 5.8 Hz, 1H), |
| | 607.33 [M + H] | 8.36 (s, 1H), 7.25-7.17 (m, 2H), 5.84 (d, J = 6.5 Hz, 1H), |
| | | 5.68-5.60 (m, 2H), 5.36 (dt, J = 11.4, 3.1 Hz, 1H), 5.31- |
| | | 5.25 (m, 1H), 5.01 (d, J = 14.6 Hz, 1H), 4.66 (d, J = 14.6 |
| | | Hz, 1H), 4.55 (d, J = 5.7 Hz, 2H), 4.09-4.01 (m, 2H), |
| | | 3.82-3.73 (m, 1H), 1.55 (q, J = 6.9 Hz, 2H), 1.35-1.22 |
| | | (m, 12H), 0.89-0.81 (m, 3H). |
| C81 | C30H35F3N4O7 | 1H NMR (400 MHz, DMSO) δ 10.26 (t, J = 5.8 Hz, 1H), |
| | 621.36 [M + H] | 8.36 (s, 1H), 7.27-7.14 (m, 2H), 5.84 (d, J = 6.5 Hz, 1H), |
| | | 5.68-5.60 (m, 2H), 5.40-5.24 (m, 2H), 5.01 (d, J = 14.6 |
| | | Hz, 1H), 4.66 (d, J = 14.6 Hz, 1H), 4.55 (d, J = 5.7 Hz, |
| | | 2H), 4.05 (td, J = 6.6, 1.1 Hz, 2H), 3.79 (dq, J = 6.5, 3.1 |
| | | Hz, 1H), 1.56 (t, J = 7.0 Hz, 2H), 1.35-1.20 (m, 14H), |
| | | 0.89-0.81 (m, 3H). |
| C82 | C31H37F3N4O7 | 1H NMR (400 MHz, DMSO) δ 10.26 (t, J = 5.8 Hz, 1H), |
| | 635.35 [M + H] | 8.36 (s, 1H), 7.27-7.16 (m, 2H), 5.84 (d, J = 6.5 Hz, 1H), |
| | | 5.68-5.59 (m, 2H), 5.36 (dt, J = 11.5, 3.2 Hz, 1H), 5.29 |
| | | (d, J = 7.8 Hz, 1H), 5.01 (d, J = 14.5 Hz, 1H), 4.66 (d, J = |
| | | 14.5 Hz, 1H), 4.55 (d, J = 5.8 Hz, 2H), 4.09-4.01 (m, |
| | | 2H), 3.78 (dt, J = 6.6, 3.2 Hz, 1H), 1.55 (q, J = 6.8 Hz, |
| | | 2H), 1.36-1.17 (m, 16H), 0.89-0.81 (m, 3H). |
| C83 | C32H39F3N4O7 | 1H NMR (400 MHz, DMSO) δ 10.26 (t, J = 5.8 Hz, 1H), |
| | 649.38 [M + H] | 8.36 (s, 1H), 7.27-7.14 (m, 2H), 5.84 (d, J = 6.5 Hz, 1H), |
| | | 5.68-5.59 (m, 2H), 5.40-5.25 (m, 2H), 5.01 (d, J = 14.6 |
| | | Hz, 1H), 4.66 (d, J = 14.5 Hz, 1H), 4.55 (d, J = 5.8 Hz, |
| | | 2H), 4.10-4.01 (m, 2H), 3.79 (dq, J = 6.4, 3.1 Hz, 1H), |
| | | 1.55 (q, J = 6.7 Hz, 2H), 1.35-1.20 (m, 18H), 0.89-0.81 |
| | | (m, 3H). |
| C84 | C33H41F3N4O7 | 1H NMR (400 MHz, DMSO) δ 10.26 (t, J = 5.8 Hz, 1H), |
| | 663.37 [M + H] | 8.36 (s, 1H), 7.27-7.14 (m, 2H), 5.84 (d, J = 6.5 Hz, 1H), |
| | | 5.68-5.59 (m, 2H), 5.40-5.25 (m, 2H), 5.01 (d, J = 14.6 |
| | | Hz, 1H), 4.66 (d, J = 14.5 Hz, 1H), 4.55 (d, J = 5.7 Hz, |
| | | 2H), 4.10-4.01 (m, 2H), 3.79 (dq, J = 6.4, 3.2 Hz, 1H), |
| | | 1.55 (q, J = 6.9 Hz, 2H), 1.37-1.16 (m, 20H), 0.89-0.81 |
| | | (m, 3H). |
| C85 | C35H45F3N4O7 | 1H NMR (400 MHz, DMSO) δ 10.26 (t, J = 5.7 Hz, 1H), |
| | 691.48 [M + H] | 8.36 (s, 1H), 7.20 (t, J = 8.6 Hz, 2H), 5.84 (d, J = 6.5 Hz, |
| | | 1H), 5.68-5.59 (m, 2H), 5.41-5.23 (m, 2H), 5.01 (d, J = |
| | | 14.6 Hz, 1H), 4.66 (d, J = 14.4 Hz, 1H), 4.55 (d, J = 5.7 |
| | | Hz, 2H), 4.05 (t, J = 6.4 Hz, 2H), 3.82-3.74 (m, 1H), 1.54 |
| | | (d, J = 7.3 Hz, 2H), 1.41-1.08 (m, 24H), 0.89-0.81 (m, 3H). |
| C86 | C37H49F3N4O7 | 1H NMR (400 MHz, DMSO) δ 10.26 (t, J = 5.8 Hz, 1H), |
| | 719.65 [M + H] | 8.36 (s, 1H), 7.27-7.15 (m, 2H), 5.84 (d, J = 6.5 Hz, 1H), |
| | | 5.68-5.59 (m, 2H), 5.36 (ddd, J = 11.4, 3.6, 2.6 Hz, 1H), |
| | | 5.28 (dd, J = 7.0, 3.1 Hz, 1H), 5.01 (d, J = 14.6 Hz, 1H), |
| | | 4.66 (d, J = 14.5 Hz, 1H), 4.55 (d, J = 5.7 Hz, 2H), 4.10- |
| | | 4.01 (m, 2H), 3.79 (dq, J = 6.5, 3.1 Hz, 1H), 1.54 (q, J = |
| | | 6.8 Hz, 2H), 1.37-1.14 (m, 28H), 0.89-0.81 (m, 3H). |
| C87 | C39H53F3N4O7 | 1H NMR (400 MHz, DMSO) δ 10.26 (t, J = 5.8 Hz, 1H), |
| | 747.48 [M + H] | 8.36 (s, 1H), 7.27-7.15 (m, 2H), 5.84 (d, J = 6.5 Hz, 1H), |
| | | 5.68-5.59 (m, 2H), 5.40-5.25 (m, 1H), 5.32-5.27 (m, |
| | | 1H), 5.01 (d, J = 14.6 Hz, 1H), 4.66 (d, J = 14.4 Hz, 1H), |
| | | 4.55 (d, J = 5.8 Hz, 2H), 4.09-4.01 (m, 2H), 3.79 (dq, J = |
| | | 6.5, 3.2 Hz, 1H), 1.54 (q, J = 6.9 Hz, 2H), 1.40-1.09 (m, |
| | | 32H), 0.89-0.81 (m, 3H). |
| C88 | C41H57F3N4O7 | 1H NMR (400 MHz, DMSO) δ 10.26 (t, J = 5.7 Hz, 1H), |
| | 775.55 [M + H] | 8.36 (s, 1H), 7.20 (dd, J = 9.2, 8.0 Hz, 2H), 5.84 (d, J = |
| | | 6.5 Hz, 1H), 5.68-5.59 (m, 2H), 5.35 (ddd, J = 11.5, 3.6, |
| | | 2.6 Hz, 1H), 5.28 (dd, J = 7.3, 2.9 Hz, 1H), 5.01 (d, J = |
| | | 14.6 Hz, 1H), 4.66 (d, J = 14.5 Hz, 1H), 4.55 (d, J = 5.8 |
| | | Hz, 2H), 4.09-4.01 (m, 2H), 3.78 (dq, J = 6.5, 3.2 Hz, |
| | | 1H), 1.54 (q, J = 6.9 Hz, 2H), 1.32 (d, J = 6.7 Hz, 3H), |
| | | 1.31-1.20 (m, 33H), 0.89-0.81 (m, 3H). |
| C89 | C26H27F3N4O7 | 1H NMR (400 MHz, DMSO) δ 10.26 (t, J = 5.8 Hz, 1H), |
| | 565.31 [M + H] | 8.36 (s, 1H), 7.28-7.16 (m, 2H), 5.81 (d, J = 6.5 Hz, 1H), |
| | | 5.68-5.59 (m, 2H), 5.40-5.25 (m, 2H), 5.01 (d, J = 14.5 |
| | | Hz, 1H), 4.75 (h, J = 6.3 Hz, 1H), 4.66 (d, J = 14.5 Hz, |
| | | 1H), 4.55 (d, J = 5.7 Hz, 2H), 3.80 (dq, J = 6.5, 3.2 Hz, |
| | | 1H), 1.29 (dd, J = 19.8, 7.0 Hz, 6H), 1.19 (dd, J = 6.2, 1.1 |
| | | Hz, 6H). |
| C90 | C27H29F3N4O7 | 1H NMR (400 MHz, DMSO) δ 10.26 (t, J = 5.8 Hz, 1H), |
| | 579.31 [M + H] | 8.36 (s, 1H), 7.25-7.17 (m, 2H), 5.83 (d, J = 6.5 Hz, 1H), |
| | | 5.70-5.59 (m, 2H), 5.40-5.25 (m, 2H), 5.01 (d, J = 14.6 |
| | | Hz, 1H), 4.66 (d, J = 14.4 Hz, 1H), 4.55 (d, J = 5.7 Hz, |
| | | 2H), 3.85 (d, J = 6.6 Hz, 2H), 3.79 (dd, J = 6.6, 3.4 Hz, |
| | | 1H), 1.86 (hept, J = 6.7 Hz, 1H), 1.29 (dd, J = 20.2, 7.0 |
| | | Hz, 6H), 0.86 (dd, J = 6.7, 2.4 Hz, 6H). |
| C91 | C28H31F3N4O7 | 1H NMR (400 MHz, DMSO) δ 10.26 (t, J = 5.8 Hz, 1H), |
| | 593.30 [M + H] | 8.36 (s, 1H), 7.21 (dd, J = 9.2, 8.0 Hz, 2H), 5.85 (d, J = |
| | | 6.5 Hz, 1H), 5.70-5.60 (m, 2H), 5.40-5.25 (m, 2H), |
| | | 5.01 (d, J = 14.6 Hz, 1H), 4.66 (d, J = 14.5 Hz, 1H), 4.55 |
| | | (d, J = 5.7 Hz, 2H), 4.09 (t, J = 6.6 Hz, 2H), 3.78 (dq, J = |
| | | 6.6, 3.3 Hz, 1H), 1.63 (dp, J = 13.5, 6.7 Hz, 1H), 1.46 (q, J = |
| | | 6.7 Hz, 2H), 1.29 (dd, J = 19.1, 7.0 Hz, 6H), 0.89-0.77 |
| | | (m, 6H). |
| C92 | C29H33F3N4O7 | 1H NMR (400 MHz, DMSO) δ 10.26 (t, J = 5.8 Hz, 1H), |
| | 607.37 [M + H] | 8.36 (s, 1H), 7.27-7.15 (m, 2H), 5.84 (d, J = 6.5 Hz, 1H), |
| | | 5.68-5.60 (m, 2H), 5.40-5.28 (m, 2H), 5.01 (d, J = 14.6 |
| | | Hz, 1H), 4.66 (d, J = 14.5 Hz, 1H), 4.55 (d, J = 5.7 Hz, |
| | | 2H), 4.04 (td, J = 6.6, 1.4 Hz, 2H), 3.78 (dq, J = 6.5, 3.2 |
| | | Hz, 1H), 1.63-1.52 (m, 2H), 1.56-1.43 (m, 1H), 1.29 |
| | | (dd, J = 19.6, 7.0 Hz, 6H), 1.22-1.12 (m, 2H), 0.84 (d, J = |
| | | 6.6 Hz, 6H). |
| C93 | C30H35F3N4O7 | 1H NMR (400 MHz, DMSO) δ 10.26 (t, J = 5.8 Hz, 1H), |
| | 621.36 [M + H] | 8.36 (s, 1H), 7.27-7.15 (m, 2H), 5.85 (d, J = 6.5 Hz, 1H), |
| | | 5.68-5.60 (m, 2H), 5.40-5.25 (m, 2H), 5.01 (d, J = 14.5 |
| | | Hz, 1H), 4.66 (d, J = 14.5 Hz, 1H), 4.55 (d, J = 5.7 Hz, |
| | | 2H), 4.05 (td, J = 6.6, 1.0 Hz, 2H), 3.78 (dq, J = 6.5, 3.1 |
| | | Hz, 1H), 1.60-1.50 (m, 2H), 1.47 (dt, J = 13.3, 6.8 Hz, |
| | | 1H), 1.29 (dd, J = 19.6, 7.0 Hz, 8H), 1.19-1.06 (m, 2H), |
| | | 0.84 (d, J = 6.6 Hz, 6H). |
| C94 | C31H37F3N4O7 | 1H NMR (400 MHz, DMSO) δ 10.26 (t, J = 5.8 Hz, 1H), |
| | 635.35 [M + H] | 8.36 (s, 1H), 7.27-7.15 (m, 2H), 5.84 (d, J = 6.5 Hz, 1H), |
| | | 5.68-5.59 (m, 2H), 5.40-5.25 (m, 2H), 5.01 (d, J = 14.6 |
| | | Hz, 1H), 4.66 (d, J = 14.4 Hz, 1H), 4.55 (d, J = 5.7 Hz, |
| | | 2H), 4.09-4.01 (m, 2H), 3.79 (dq, J = 6.4, 3.2 Hz, 1H), |
| | | 1.60-1.42 (m, 3H), 1.35-1.22 (m, 10H), 1.13 (dd, J = |
| | | 8.6, 5.2 Hz, 2H), 0.84 (d, J = 6.6 Hz, 6H). |
| C95 | C32H39F3N4O7 | 1H NMR (400 MHz, DMSO) δ 10.26 (t, J = 5.8 Hz, 1H), |
| | 649.38 [M + H] | 8.36 (s, 1H), 7.25-7.16 (m, 2H), 5.84 (d, J = 6.5 Hz, 1H), |
| | | 5.68-5.59 (m, 2H), 5.36 (dt, J = 11.4, 3.1 Hz, 1H), 5.29 |
| | | (d, J = 7.5 Hz, 1H), 5.01 (d, J = 14.6 Hz, 1H), 4.66 (d, J = |
| | | 14.5 Hz, 1H), 4.55 (d, J = 5.7 Hz, 2H), 4.05 (t, J = 6.5 Hz, |
| | | 2H), 3.79 (dt, J = 6.6, 3.2 Hz, 1H), 1.60-1.52 (m, 2H), |
| | | 1.48 (td, J = 13.3, 6.7 Hz, 1H), 1.35-1.18 (m, 12 H), 1.13 |
| | | (t, J = 6.6 Hz, 2H), 0.84 (d, J = 6.6 Hz, 6H). |
| C96 | C33H41F3N4O7 | 1H NMR (400 MHz, DMSO) δ 10.26 (t, J = 5.8 Hz, 1H), |
| | 663.37 [M + H] | 8.36 (s, 1H), 7.25-7.16 (m, 2H), 5.84 (d, J = 6.5 Hz, 1H), |
| | | 5.68-5.59 (m, 2H), 5.40-5.27 (m, 2H), 5.01 (d, J = 14.6 |
| | | Hz, 1H), 4.66 (d, J = 14.5 Hz, 1H), 4.55 (d, J = 5.7 Hz, |
| | | 2H), 4.05 (t, J = 6.4 Hz, 2H), 3.79 (dq, J = 6.4, 3.1 Hz, |
| | | 1H), 1.59-1.51 (m, 2H), 1.48 (dq, J = 13.3, 6.7 Hz, 1H), |
| | | 1.35-1.19 (m, 14H), 1.12 (d, J = 7.0 Hz, 2H), 0.84 (d, J = |
| | | 6.6 Hz, 6H). |
| C97 | C26H27F3N4O8 | 1H NMR (400 MHz, DMSO) δ 10.26 (t, J = 5.8 Hz, 1H), |
| | 581.28 [M + H] | 8.36 (s, 1H), 7.27-7.15 (m, 2H), 5.84 (d, J = 6.4 Hz, 1H), |
| | | 5.70-5.60 (m, 2H), 5.40-5.25 (m, 2H), 5.01 (d, J = 14.5 |
| | | Hz, 1H), 4.66 (d, J = 14.5 Hz, 1H), 4.55 (d, J = 5.7 Hz, |
| | | 2H), 4.18 (ddd, J = 5.8, 3.6, 1.2 Hz, 2H), 3.80 (dq, J = 6.5, |
| | | 3.2 Hz, 1H), 3.55-3.48 (m, 2H), 3.23 (s, 3H), 1.30 (dd, J = |
| | | 18.9, 7.0 Hz, 6H). |
| C98 | C28H31F3N4O9 | 1H NMR (400 MHz, DMSO) δ 10.26 (t, J = 5.8 Hz, 1H), |
| | 625.31 [M + H] | 8.36 (s, 1H), 7.25-7.16 (m, 2H), 5.85 (d, J = 6.4 Hz, 1H), |
| | | 5.70-5.60 (m, 2H), 5.40-5.26 (m, 2H), 5.01 (d, J = 14.6 |
| | | Hz, 1H), 4.66 (d, J = 14.5 Hz, 1H), 4.55 (d, J = 5.8 Hz, |
| | | 2H), 4.18 (q, J = 4.3 Hz, 2H), 3.80 (dd, J = 6.6, 3.3 Hz, |
| | | 1H), 3.60 (t, J = 4.6 Hz, 2H), 3.55-3.48 (m, 2H), 3.45- |
| | | 3.38 (m, 2H), 3.23 (s, 3H), 1.38-1.21 (m, 6H). |
| C99 | C27H29F3N4O7 | 1H NMR (400 MHz, DMSO) δ 10.26 (t, J = 5.8 Hz, 1H), |
| | 579.31 [M + H] | 8.36 (s, 1H), 7.27-7.15 (m, 2H), 6.54 (q, J = 5.2 Hz, 1H), |
| | | 5.65 (dt, J = 11.5, 2.3 Hz, 1H), 5.40-5.31 (m, 2H), 4.99 |
| | | (d, J = 14.6 Hz, 1H), 4.67 (d, J = 14.5 Hz, 1H), 4.61-4.49 |
| | | (m, 3H), 3.63 (dq, J = 6.4, 3.1 Hz, 1H), 1.58 (d, J = 5.3 |
| | | Hz, 3H), 1.30 (dd, J = 23.4, 7.0 Hz, 6H), 1.10 (dd, J = |
| | | 19.9, 6.3 Hz, 6H). |
| C100 | C27H29F3N4O7 | 1H NMR (400 MHz, DMSO) δ 10.24 (t, J = 5.8 Hz, 1H), |
| | 579.26 [M + H] | 8.35 (s, 1H), 7.28-7.16 (m, 2H), 6.23 (q, J = 5.2 Hz, 1H), |
| | | 5.64 (dt, J = 11.4, 2.4 Hz, 1H), 5.40-5.28 (m, 2H), 5.04 |
| | | (d, J = 14.6 Hz, 1H), 4.66-4.46 (m, 4H), 3.81 (dq, J = |
| | | 6.5, 3.2 Hz, 1H), 1.60 (d, J = 5.2 Hz, 3H), 1.29 (dd, J = |
| | | 21.4, 7.0 Hz, 6H), 1.12 (dd, J = 19.1, 6.2 Hz, 6H). |

All of the U.S. patents, U.S. patent application publications, U.S. patent applications, foreign patents, foreign patent applications and non-patent publications referred to in this specification are incorporated herein by reference, in their entirety to the extent non inconsistent with the present description.

From the forgoing it will be appreciated that, although specific embodiments have been described herein for purposes of illustration, various modifications may be made without deviating from the spirit and scope of the disclosure. Accordingly, the disclosure is not limited except as by the appended claims.

### X. EMBODIMENTS

Embodiment 1. A compound of Formula I:

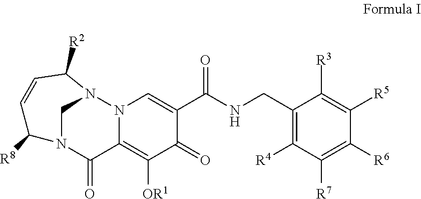

or a pharmaceutically acceptable salt thereof, wherein:
R¹ is —(CR^{1A}R^{1B}O)ₘCO(O—CR^{1C}R^{1D}—CR^{1E}R^{1F})ₙ—(O)ₚR^{1G};
   wherein m is 0 or 1;
   n is an integer between 0 and 10, inclusive;
   p is 0 or 1;
   R^{1A} is H or C₁₋₃alkyl;
   R^{1B} is H or C₁₋₃alkyl;
   each R^{1C} is independently H or C₁₋₃alkyl;
   each R^{1D} is independently H or C₁₋₃alkyl;
   each R^{1E} is independently H or C₁₋₃alkyl;
   each R^{1F} is independently H or C₁₋₃alkyl;
   R^{1G} is C₃₋₆cycloalkyl, C₁₋₂₀alkyl, C₂₋₂₀alkenyl, or a 3 to 8 membered heterocyclyl comprising 1, 2, or 3 heteroatoms selected independently from N, O, and S; wherein the C₃₋₆cycloalkyl, C₁₋₂₀alkyl, C₂₋₂₀alkenyl, or 3 to 8 membered heterocyclyl is optionally substituted with a COOH, C₃₋₆cycloalkyl, C₁₋₃alkoxy, or —NHCOOR^{1H};
   wherein R^{1H} is C₁₋₃alkyl;
R² is H or C₁₋₃alkyl;
each R³, R⁴, R⁵, R⁶ and R⁷ is independently H or halo; and
R⁸ is H or C₁₋₃alkyl.
Embodiment 2. The compound of claim 1, or a pharmaceutically acceptable salt thereof,
   wherein:
   R¹ is —(CR^{1A}R^{1B}O)ₘCO(O—CR^{1C}R^{1D}—CR^{1E}R^{1F})ₙ—(O)ₚR^{1G}G;
      wherein m is 0 or 1;
      n is an integer between 0 and 10, inclusive;
      p is 0 or 1;
      R^{1A} is H or C₁₋₃alkyl;
      R^{1B} is H or C₁₋₃alkyl;
      each R^{1C} is independently H or C₁₋₃alkyl;
      each R^{1D} is independently H or C₁₋₃alkyl;
      each R^{1E} is independently H or C₁₋₃alkyl;
      each R^{1F} is independently H or C₁₋₃alkyl;
      R^{1G} is C₃₋₆cycloalkyl, C₁₋₂₀alkyl, C₂₋₂₀alkenyl, or a 3 to 8 membered heterocyclyl comprising 1, 2, or 3 heteroatoms selected independently from N, O, and S; wherein the C₃₋₆cycloalkyl, C₁₋₂₀alkyl, C₂₋₂₀alkenyl, or 3 to 8 membered heterocyclyl is optionally substituted with a COOH, C₃₋₆cycloalkyl, or C₁₋₃alkoxy;
   R² is H or C₁₋₃alkyl;
   each R³, R⁴, R⁵, R⁶ and R⁷ is independently H or halo; and
   R⁸ is H or C₁₋₃alkyl.
Embodiment 3. The compound of claim Embodiment 1 or Embodiment 2, or the pharmaceutically acceptable salt thereof, wherein R⁵ and R⁷ are each H.
Embodiment 4. The compound of any one of claims Embodiment 1-Embodiment 3, or the pharmaceutically acceptable salt thereof, wherein R³, R⁴, and R⁶ are each independently a halo.
Embodiment 5. The compound of any one of claims Embodiment 1-Embodiment 4, or the pharmaceutically acceptable salt thereof, wherein R³, R⁴, and R⁶ are each F.
Embodiment 6. The compound of any one of claims Embodiment 1-Embodiment 5, or the pharmaceutically acceptable salt thereof, wherein R⁸ is C₁₋₃alkyl.
Embodiment 7. The compound of any one of claims Embodiment 1-Embodiment 6, or the pharmaceutically acceptable salt thereof, wherein R⁸ is methyl.
Embodiment 8. The compound of any one of claims Embodiment 1-Embodiment 7, or the pharmaceutically acceptable salt thereof, wherein R² is C₁₋₃alkyl.
Embodiment 9. The compound of any one of claims Embodiment 1-Embodiment 8, or the pharmaceutically acceptable salt thereof, wherein R² is methyl.
Embodiment 10. The compound of any one of claims Embodiment 1-Embodiment 9, or the pharmaceutically acceptable salt thereof, wherein R² is methyl and R⁸ is methyl.
Embodiment 11. The compound of any one of claims Embodiment 1-Embodiment 10, or the pharmaceutically acceptable salt thereof, wherein m is 0.
Embodiment 12. The compound of any one of claims Embodiment 1-Embodiment 10, or the pharmaceutically acceptable salt thereof, wherein m is 1.
Embodiment 13. The compound of any one of claims Embodiment 1-Embodiment 12, or the pharmaceutically acceptable salt thereof, wherein n is an integer between 0 and 7, inclusive.
Embodiment 14. The compound of any one of claims Embodiment 1-Embodiment 13, or the pharmaceutically acceptable salt thereof, wherein n is 0.
Embodiment 15. The compound of any one of claims Embodiment 1-Embodiment 13, or the pharmaceutically acceptable salt thereof, wherein n is 1, 2, or 3.
Embodiment 16. The compound of any one of claims Embodiment 1-Embodiment 15, or the pharmaceutically acceptable salt thereof, wherein p is 0.
Embodiment 17. The compound of any one of claims Embodiment 1-Embodiment 15, or the pharmaceutically acceptable salt thereof, wherein p is 1.
Embodiment 18. The compound of any one of Embodiment 1-Embodiment 11, Embodiment 13, Embodiment 14 and Embodiment 16, or the pharmaceutically acceptable salt thereof, wherein the compound is of a Formula Ia:

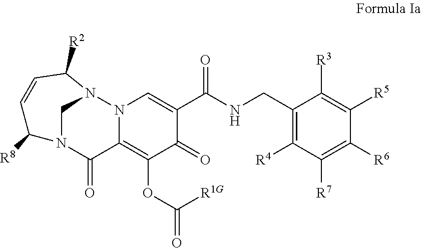

Embodiment 19. The compound of any one of Embodiment 1-Embodiment 11, Embodiment 13, Embodiment 14 and Embodiment 17, or the pharmaceutically acceptable salt thereof, wherein the compound is of a Formula Ib:

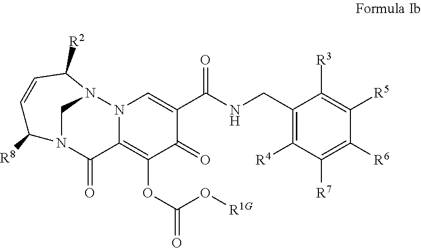

Embodiment 20. The compound of any one of Embodiment 1-Embodiment 11, Embodiment 13, Embodiment 15 and Embodiment 17, or the pharmaceutically acceptable salt thereof, wherein the compound is of a Formula Ic:

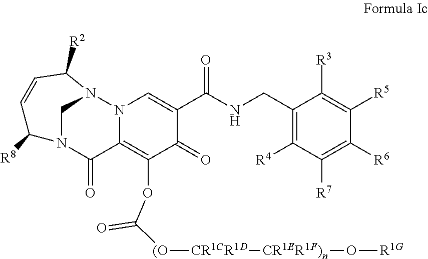

wherein n is an integer between 1 and 7, inclusive.
Embodiment 21. The compound of Embodiment 20, wherein n is 1, 2, or 3.
Embodiment 22. The compound of any one of Embodiment 1-Embodiment 10, Embodiment 12, Embodiment 13, Embodiment 15 and Embodiment 17, or the pharmaceutically acceptable salt thereof, wherein the compound is of a Formula Id:

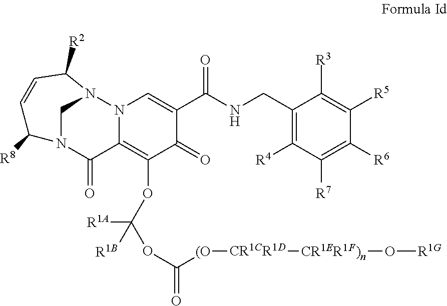

wherein n is 0, 1, 2, or 3.
Embodiment 23. The compound of Embodiment 22, or the pharmaceutically acceptable salt thereof, wherein n is 1, 2, or 3.
Embodiment 24. The compound of any one of Embodiment 1-Embodiment 10, Embodiment 12-Embodiment 14, and Embodiment 17, or the pharmaceutically acceptable salt thereof, wherein the compound is of a Formula Ie:

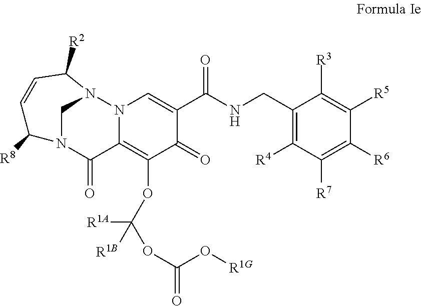

Embodiment 25. The compound of any one of claims Embodiment 1-Embodiment 10, Embodiment 12-Embodiment 14, and Embodiment 16, or the pharmaceutically acceptable salt thereof, wherein the compound is of a Formula If:

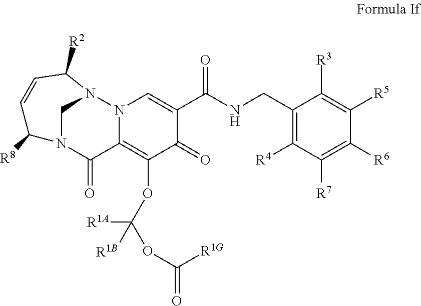

Embodiment 26. The compound of any one of Embodiment 1-Embodiment 13, Embodiment 15Embodiment 17, and Embodiment 20-Embodiment 23, or the pharmaceutically acceptable salt thereof, wherein n is 1.
Embodiment 27. The compound of any one of Embodiment 1-Embodiment 13, Embodiment 15Embodiment 17, and Embodiment 20-Embodiment 23, or the pharmaceutically acceptable salt thereof, wherein n is 2.
Embodiment 28. The compound of any one of Embodiment 1-Embodiment 10, Embodiment 12-Embodiment 17, and Embodiment 22-Embodiment 27, or the pharmaceutically acceptable salt thereof, wherein each R^{1A} is H and R^{1B} is H.
Embodiment 29. The compound of any one of Embodiment 1-Embodiment 10, Embodiment 12-Embodiment 17, and Embodiment 22-Embodiment 27, or the pharmaceutically acceptable salt thereof, wherein each R^{1A} is methyl and R^{1B} is H.
Embodiment 30. The compound of any one of Embodiment 1-Embodiment 13, Embodiment 15-Embodiment 17, Embodiment 20-Embodiment 23, and Embodiment 26-Embodiment 29, wherein each R^{1C} is H, each R^{1D} is H, each R^{1E} is H, and each R^{1F} is H.
Embodiment 31. The compound of any one of Embodiment 1-Embodiment 30, or the pharmaceutically acceptable salt thereof, wherein R^{1G} is C₅₋₆cycloalkyl, C₁₋₂₀alkyl or C₂₋₂₀alkenyl; wherein the C₅₋₆cycloalkyl, C₁₋₂₀alkyl or C₂₋₂₀alkenyl is optionally substituted with COOH, C₅₋₆cycloalkyl, C₁₋₃alkoxy, or —NHCOOR^{1H}.
Embodiment 32. The compound of any one of Embodiment 1-Embodiment 31, or the pharmaceutically acceptable salt thereof, wherein R^{1G} is C₁₋₂₀alkyl or C₂₋₂₀alkenyl; wherein the C₁₋₂₀alkyl or C₂₋₂₀alkenyl is optionally substituted with a COOH, C₅₋₆cycloalkyl or C₁₋₃alkoxy.
Embodiment 33. The compound of any one of Embodiment 1-Embodiment 32, or the pharmaceutically acceptable salt thereof, wherein R^{1G} is C₁₋₂₀alkyl or C₂₋₂₀alkenyl.
Embodiment 34. The compound of any one of Embodiment 1-Embodiment 33, wherein R^{1G} is C₁₋₂₀alkyl optionally substituted with a COOH, C₃₋₆cycloalkyl, or C₁₋₃alkoxy.
Embodiment 35. The compound of any one of Embodiment 1-Embodiment 34, or the pharmaceutically acceptable salt thereof, wherein R^{1G} is selected from the group consisting of —CH₃, —CH₂CH₃, —(CH₂)₂CH₃, —(CH₂)₃CH₃, —(CH₂)₄CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, —(CH₂)₁₃CH₃, —(CH₂)₁₄CH₃, —(CH₂)₁₅CH₃, —(CH₂)₁₆CH₃, —(CH₂)₁₇CH₃, —CH(CH₃)₂, —CH₂CH(CH₃)₂, —(CH₂)₂CH(CH₃)₂, —(CH₂)₃CH(CH₃)₂, —(CH₂)₄CH(CH₃)₂, —(CH₂)₅CH(CH₃)₂, —(CH₂)₆CH(CH₃)₂, —(CH₂)₇CH(CH₃)₂, —(CH₂)₇CH═CH(CH₂)₃CH₃, —(CH₂)₃(CH═CHCH₂)₅CH₃, —(CH₂)₇(CH═CHCH₂)₂(CH₂)₃CH₃, —CH₂CH₂-cyclopentyl, cyclopentyl, —(CH₂)₅OCH₃, —(CH₂)₃COOH, —(CH₂)₅COOH, —(CH₂)₁₆COOH, and

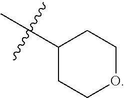 [SMILES: *C1CCOCC1]

Embodiment 36. The compound of any one of Embodiment 1-Embodiment 35, or the pharmaceutically acceptable salt thereof, wherein R¹ is selected from the group consisting of —COCH₃, —COCH₂CH₃, —CO(CH₂)₂CH₃, —CO(CH₂)₃CH₃, —CO(CH₂)₄CH₃, —CO(CH₂)₅CH₃, —CO(CH₂)₆CH₃, —CO(CH₂)₇CH₃, —CO(CH₂)₈CH₃, —CO(CH₂)₉CH₃, —CO(CH₂)₁₀CH₃, —CO(CH₂)₁₁CH₃, —CO(CH₂)₁₂CH₃, —CO(CH₂)₁₃CH₃, —CO(CH₂)₁₄CH₃, —CO(CH₂)₁₅CH₃, —CO(CH₂)₁₆CH₃, —CO(CH₂)₁₇CH₃, —COCH(CH₃)₂, —COCH₂CH(CH₃)₂, —CO(CH₂)₂CH(CH₃)₂, —CO(CH₂)₃CH(CH₃)₂, —CO(CH₂)₄CH(CH₃)₂, —CO(CH₂)₅CH(CH₃)₂, —CO(CH₂)₆CH(CH₃)₂, —CO(CH₂)₇CH(CH₃)₂, —CO(CH₂)₇CH═CH(CH₂)₃CH₃, —CO(CH₂)₃(CH═CHCH₂)₅CH₃, —CO(CH₂)₇(CH═CHCH₂)₂CH₃CH₃, —COCH₂CH₂-cyclopentyl, —CO-cyclopentyl, —CO(CH₂)₅OCH₃, —CO(CH₂)₃COOH, —CO(CH₂)₅COOH, —CO(CH₂)₁₆COOH, and

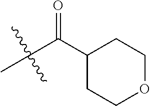 [SMILES: *C(=O)C1CCOCC1]

Embodiment 37. The compound of any one of Embodiment 36, or the pharmaceutically acceptable salt thereof, wherein R¹ is selected from the group consisting of —COCH₃, —COCH₂CH₃, —CO(CH₂)₂CH₃, —CO(CH₂)₃CH₃, —CO(CH₂)₄CH₃, —CO(CH₂)₅CH₃, —CO(CH₂)₆CH₃, —CO(CH₂)₇CH₃, —CO(CH₂)₈CH₃, —CO(CH₂)₁₀CH₃, —CO(CH₂)₁₂CH₃, —CO(CH₂)₁₄CH₃, —CO(CH₂)₁₆CH₃, —COCH(CH₃)₂, —COCH₂CH(CH₃)₂, —CO(CH₂)₂CH(CH₃)₂, —CO(CH₂)₃CH(CH₃)₂, —CO(CH₂)₄CH(CH₃)₂, —CO(CH₂)₅CH(CH₃)₂, —CO(CH₂)₆CH(CH₃)₂, —CO(CH₂)₇CH═CH(CH₂)₃CH₃, —CO(CH₂)₃(CH═CHCH₂)₅CH₃, —CO(CH₂)₇(CH═CHCH₂)₂CH₃CH₃, —COCH₂CH₂-cyclopentyl, —CO-cyclopentyl, —CO(CH₂)₅OCH₃, —CO(CH₂)₃COOH, —CO(CH₂)₅COOH, and —CO(CH₂)₁₆COOH.
Embodiment 38. The compound of any one of Embodiment 1-Embodiment 35, or the pharmaceutically acceptable salt thereof, wherein R¹ is selected from the group consisting of —CH₂OCOCH₃, —CH₂OCOCH₂CH₃, —CH₂OCO(CH₂)₂CH₃, —CH₂OCO(CH₂)₃CH₃, —CH₂OCO(CH₂)₄CH₃, —CH₂OCO(CH₂)₅CH₃, —CH₂OCO(CH₂)₆CH₃, —CH₂OCO(CH₂)₇CH₃, —CH₂OCO(CH₂)₈CH₃, —CH₂OCO(CH₂)₁₀CH₃, —CH₂OCO(CH₂)₁₂CH₃, —CH₂OCO(CH₂)₁₄CH₃, —CH₂OCO(CH₂)₁₆CH₃, —CH₂OCOCH(CH₃)₂, —CH₂OCOCH₂CH(CH₃)₂, —CH₂OCO(CH₂)₂CH(CH₃)₂, —CH₂OCO(CH₂)₃CH(CH₃)₂, —CH₂OCO(CH₂)₄CH(CH₃)₂, —CH₂OCO(CH₂)₅CH(CH₃)₂, —CH₂OCO(CH₂)₆CH(CH₃)₂, —CH₂OCO(CH₂)₇CH(CH₃)₂, —CH₂OCO(CH₂)₇CH═CH(CH₂)₃CH₃, —CH₂OCO(CH₂)₃(CH═CHCH₂)₅CH₃, —CH₂OCO(CH₂)₇(CH═CHCH₂)₂CH₃CH₃, —CH₂OCOCH₂CH₂-cyclopentyl, —CH₂OCO-cyclopentyl, —CH₂OCO(CH₂)₅OCH₃, —CH₂OCO(CH₂)₃COOH, —CH₂OCO(CH₂)₅COOH, —CH₂OCO(CH₂)₁₆COOH, and

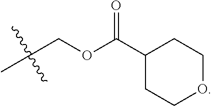 [SMILES: *COC(=O)C1CCOCC1]

Embodiment 39. The compound of Embodiment 38, or the pharmaceutically acceptable salt thereof, wherein R¹ is selected from the group consisting of —CH₂OCOCH₃, —CH₂OCOCH₂CH₃, —CH₂OCO(CH₂)₂CH₃, —CH₂OCO(CH₂)₃CH₃, —CH₂OCO(CH₂)₄CH₃, —CH₂OCO(CH₂)₅CH₃, —CH₂OCO(CH₂)₆CH₃, —CH₂OCO(CH₂)₇CH₃, —CH₂OCO(CH₂)₈CH₃, —CH₂OCO(CH₂)₁₀CH₃, —CH₂OCO(CH₂)₁₂CH₃, —CH₂OCO(CH₂)₁₄CH₃, —CH₂OCO(CH₂)₁₆CH₃, —CH₂OCOCH(CH₃)₂, —CH₂OCOCH₂CH(CH₃)₂, —CH₂OCO(CH₂)₂CH(CH₃)₂, —CH₂OCO(CH₂)₃CH(CH₃)₂, —CH₂OCO(CH₂)₄CH(CH₃)₂, —CH₂OCO(CH₂)₅CH(CH₃)₂, and —CH₂OCO(CH₂)₆CH(CH₃)₂.
Embodiment 40. The compound of any one of Embodiment 1-Embodiment 35, or the pharmaceutically acceptable salt thereof, wherein R¹ is selected from the group consisting of —COOCH₃, —COOCH₂CH₃, —COO(CH₂)₂CH₃, —COO(CH₂)₃CH₃, —COO(CH₂)₄CH₃, —COO(CH₂)₅CH₃, —COO(CH₂)₆CH₃, —COO(CH₂)₇CH₃, —COO(CH₂)₈CH₃, —COO(CH₂)₉CH₃, —COO(CH₂)₁₀CH₃, —COO(CH₂)₁₁CH₃, —COO(CH₂)₁₂CH₃, —COO(CH₂)₁₃CH₃, —COO(CH₂)₁₄CH₃, —COO(CH₂)₁₅CH₃, —COO(CH₂)₁₆CH₃, —COO(CH₂)₁₇CH₃, —COOCH(CH₃)₂, —COOCH₂CH(CH₃)₂, —COO(CH₂)₂CH(CH₃)₂, —COO(CH₂)₃CH(CH₃)₂, —COO(CH₂)₄CH(CH₃)₂, —COO(CH₂)₅CH(CH₃)₂, —COO(CH₂)₆CH(CH₃)₂, —COO(CH₂)₇CH(CH₃)₂, —COO(CH₂CH₂O)₂CH₃, —COO(CH₂)₇CH═CH(CH₂)₃CH₃, —COO(CH₂)₃(CH═CHCH₂)₅CH₃, —COO(CH₂)₇(CH═CHCH₂)₂CH₃CH₃, —COOCH₂CH₂-cyclopentyl, —COO-cyclopentyl, —COO(CH₂)₅OCH₃, —COO(CH₂)₃COOH, —COO(CH₂)₅COOH, —COO(CH₂)₁₆COOH, and

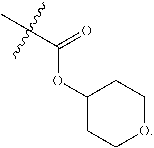 [SMILES: *C(=O)OC1CCOCC1]

Embodiment 41. The compound of Embodiment 40, or the pharmaceutically acceptable salt thereof, wherein R¹ is selected from the group consisting of —COOCH₃, —COOCH₂CH₃, —COO(CH₂)₂CH₃, —COO(CH₂)₃CH₃, —COO(CH₂)₄CH₃, —COO(CH₂)₅CH₃, —COO(CH₂)₆CH₃, —COO(CH₂)₇CH₃, —COO(CH₂)₈CH₃, —COO(CH₂)₉CH₃, —COO(CH₂)₁₁CH₃, —COO(CH₂)₁₃CH₃, —COO(CH₂)₁₅CH₃, —COO(CH₂)₁₇CH₃, —COOCH(CH₃)₂, —COOCH₂CH(CH₃)₂, —COO(CH₂)₂CH(CH₃)₂, —COO(CH₂)₃CH(CH₃)₂, —COO(CH₂)₄CH(CH₃)₂, —COO(CH₂)₅CH(CH₃)₂, —COO(CH₂)₆CH(CH₃)₂, —COO(CH₂)₇CH(CH₃)₂, —COO(CH₂CH₂O)₂CH₃, and

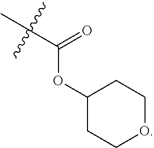 [SMILES: *C(=O)OC1CCOCC1]

Embodiment 42. The compound of any one of Embodiment 1-Embodiment 35, or the pharmaceutically acceptable salt thereof, wherein R¹ is selected from the group consisting of —CH₂OCOOCH₃, —CH₂OCOOCH₂CH₃, —CH₂OCOO(CH₂)₂CH₃, —CH₂OCOO(CH₂)₃CH₃, —CH₂OCOO(CH₂)₄CH₃, —CH₂OCOO(CH₂)₅CH₃, —CH₂OCOO(CH₂)₆CH₃, —CH₂OCOO(CH₂)₇CH₃, —CH₂OCOO(CH₂)₈CH₃, —CH₂OCOO(CH₂)₉CH₃, —CH₂OCOO(CH₂)₁₀CH₃, —CH₂OCOO(CH₂)₁₁CH₃, —CH₂OCOO(CH₂)₁₂CH₃, —CH₂OCOO(CH₂)₁₃CH₃, —CH₂OCOO(CH₂)₁₄CH₃, —CH₂OCOO(CH₂)₁₅CH₃, —CH₂OCOO(CH₂)₁₆CH₃, —CH₂OCOO(CH₂)₁₇CH₃, —CH₂OCOOCH(CH₃)₂, —CH₂OCOOCH₂CH(CH₃)₂, —CH₂OCOO(CH₂)₂CH(CH₃)₂, —CH₂OCOO(CH₂)₃CH(CH₃)₂, —CH₂OCOO(CH₂)₄CH(CH₃)₂, —CH₂OCOO(CH₂)₅CH(CH₃)₂, —CH₂OCOO(CH₂)₆CH(CH₃)₂, —CH₂OCOO(CH₂)₇CH(CH₃)₂, —CH₂OCOO(CH₂)₂OCH₃, —CH₂OCOO(CH₂CH₂O)₂CH₃, —CH(CH₃)OCOOCH(CH₃)₂, —CH₂OCOO(CH₂)₇CH═CH(CH₂)₃CH₃, —CH₂OCOO(CH₂)₃(CH═CHCH₂)₅CH₃, —CH₂OCOO(CH₂)₇(CH═CHCH₂)₂CH₃CH₃, —CH₂OCOOCH₂CH₂-cyclopentyl, —CH₂OCOO-cyclopentyl, —CH₂OCOO(CH₂)₅OCH₃, —CH₂OCOO(CH₂)₃COOH, —CH₂OCOO(CH₂)₅COOH, —CH₂OCOO(CH₂)₁₆COOH, and

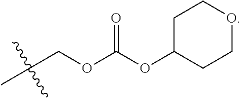 [SMILES: *COC(=O)OC1CCOCC1]

Embodiment 43. The compound of Embodiment 42, or the pharmaceutically acceptable salt thereof, wherein R¹ is selected from the group consisting of —CH₂OCOOCH₃, —CH₂OCOOCH₂CH₃, —CH₂OCOO(CH₂)₂CH₃, —CH₂OCOO(CH₂)₃CH₃, —CH₂OCOO(CH₂)₄CH₃, —CH₂OCOO(CH₂)₅CH₃, —CH₂OCOO(CH₂)₆CH₃, —CH₂OCOO(CH₂)₇CH₃, —CH₂OCOO(CH₂)₈CH₃, —CH₂OCOO(CH₂)₉CH₃, —CH₂OCOO(CH₂)₁₁CH₃, —CH₂OCOO(CH₂)₁₃CH₃, —CH₂OCOO(CH₂)₁₅CH₃, —CH₂OCOO(CH₂)₁₇CH₃, —CH₂OCOOCH(CH₃)₂, —CH₂OCOOCH₂CH(CH₃)₂, —CH₂OCOO(CH₂)₂CH(CH₃)₂, —CH₂OCOO(CH₂)₃CH(CH₃)₂, —CH₂OCOO(CH₂)₄CH(CH₃)₂, —CH₂OCOO(CH₂)₅CH(CH₃)₂, —CH₂OCOO(CH₂)₆CH(CH₃)₂, —CH₂OCOO(CH₂)₇CH(CH₃)₂, —CH₂OCOO(CH₂)₂OCH₃, —CH₂OCOO(CH₂CH₂O)₂CH₃, and —CH(CH₃)OCOOCH(CH₃)₂.
Embodiment 44. The compound of any one of Embodiment 1-Embodiment 43, wherein:
   R² is methyl;
   R³, R⁴, and R⁶ are each F;
   R⁵ and R⁷ are each H; and
   R⁸ is methyl.
Embodiment 45. The compound of Embodiment 1, or a pharmaceutically acceptable salt thereof, wherein the compound is selected from the group consisting of the compounds in Table 1, Table 2, Table 3, and Table 4.
Embodiment 46. A pharmaceutical composition comprising a therapeutically effective amount of a compound of any one of Embodiment 1-Embodiment 45, or pharmaceutically acceptable salt thereof, and a pharmaceutically acceptable excipient.
Embodiment 47. The pharmaceutical composition of Embodiment 46, further comprising one, two, three, or four additional therapeutic agents.
Embodiment 48. The pharmaceutical composition of Embodiment 47, wherein the additional therapeutic agent or agents are anti-HIV agents.
Embodiment 49. The pharmaceutical composition of Embodiment 47 or Embodiment 48, wherein the additional therapeutic agent or agents are HIV protease inhibitors, HIV non-nucleoside or non-nucleotide inhibitors of reverse transcriptase, HIV nucleoside or nucleotide inhibitors of reverse transcriptase, HIV capsid inhibitors, gp41 inhibitors, CXCR4 inhibitors, gp120 inhibitors, CCR5 inhibitors, latency reversing agents, capsid polymerization inhibitors, HIV bNAbs, TLR7 agonists, pharmacokinetic enhancers, other drugs for treating HIV, or combinations thereof.
Embodiment 50. The pharmaceutical composition of any one of Embodiment 47-Embodiment 49, wherein the additional therapeutic agent or agents are abacavir, tenofovir alafenamide, tenofovir disoproxil, N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide, or a pharmaceutically acceptable salt thereof.
Embodiment 51. The pharmaceutical composition of any one of Embodiment 46-Embodiment 50, wherein the pharmaceutical composition is for oral or parenteral administration.
Embodiment 52. A kit comprising a compound of any one of Embodiment 1-Embodiment 45, or a pharmaceutically acceptable salt thereof, and instructions for use.
Embodiment 53. The kit of Embodiment 52, further comprising one, two, three, or four additional therapeutic agent or agents.
Embodiment 54. The kit of Embodiment 53, wherein the additional therapeutic agent or agents are anti-HIV agents.
Embodiment 55. The kit of Embodiment 53 or Embodiment 54, wherein the additional therapeutic agent or agents are HIV protease inhibitors, HIV non-nucleoside or non-nucleotide inhibitors of reverse transcriptase, HIV nucleoside or nucleotide inhibitors of reverse transcriptase, HIV capsid inhibitors, gp41 inhibitors, CXCR4 inhibitors, gp120 inhibitors, CCR5 inhibitors, latency reversing agents, capsid polymerization inhibitors, HIV bNAbs, TLR7 agonists, pharmacokinetic enhancers, other drugs for treating HIV, or combinations thereof.
Embodiment 56. The kit of any one of Embodiment 53-Embodiment 55, wherein the additional therapeutic agent or agents are abacavir, tenofovir alafenamide, tenofovir disoproxil, N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide, or a pharmaceutically acceptable salt thereof.
Embodiment 57. A method of treating an HIV infection in a human having or at risk of having the infection, comprising administering to the human a therapeutically effective amount of a compound of any one of Embodiment 1-Embodiment 45, or pharmaceutically acceptable salt thereof, or a pharmaceutical composition of any one of Embodiment 46-Embodiment 51.
Embodiment 58. The method of Embodiment 57, further comprising administering to the human a therapeutically effective amount of one, two, three, or four additional therapeutic agents.
Embodiment 59. The method of Embodiment 58, wherein the additional therapeutic agent or agents are anti-HIV agents.
Embodiment 60. The method of Embodiment 58 or Embodiment 59, wherein the additional therapeutic agent or agents are HIV protease inhibitors, HIV non-nucleoside or non-nucleotide inhibitors of reverse transcriptase, HIV nucleoside or nucleotide inhibitors of reverse transcriptase, HIV capsid inhibitors, gp41 inhibitors, CXCR4 inhibitors, gp120 inhibitors, CCR5 inhibitors, latency reversing agents, capsid polymerization inhibitors, HIV bNAbs, TLR7 agonists, pharmacokinetic enhancers, other drugs for treating HIV, or combinations thereof.
Embodiment 61. The method of any one of Embodiment 58-Embodiment 60, wherein the additional therapeutic agent or agents are abacavir, tenofovir alafenamide, tenofovir disoproxil, N—((S)-1-(3-(4-chloro-3-(methylsulfonamido)-1-(2,2,2-trifluoroethyl)-1H-indazol-7-yl)-6-(3-methyl-3-(methylsulfonyl)but-1-yn-1-yl)pyridin-2-yl)-2-(3,5-difluorophenyl)ethyl)-2-((3bS,4aR)-5,5-difluoro-3-(trifluoromethyl)-3b,4,4a,5-tetrahydro-1H-cyclopropa[3,4]cyclopenta[1,2-c]pyrazol-1-yl)acetamide, or a pharmaceutically acceptable salt thereof.
Embodiment 62. The method of any one of Embodiment 57-Embodiment 61, wherein the administration is oral, intravenous, subcutaneous, or intramuscular.
Embodiment 63. Use of a compound of any one of Embodiment 1-Embodiment 45, or pharmaceutically acceptable salt thereof, or a pharmaceutical composition of any one of claims 46-51, for treating an HIV infection in a human having or at risk of having the infection.
Embodiment 64. A compound of any one of Embodiment 1-Embodiment 45, or pharmaceutically acceptable salt thereof, or a pharmaceutical composition of any one of Embodiment 46-Embodiment 51, for use in medical therapy.
Embodiment 65. A compound of any one of Embodiment 1-Embodiment 45, or pharmaceutically acceptable salt thereof, or a pharmaceutical composition of any one of Embodiment 46-Embodiment 51, for use in treating an HIV infection.
Embodiment 66. Use of a compound of any one of Embodiment 1-Embodiment 45, or pharmaceutically acceptable salt thereof, or a pharmaceutical composition of any one of Embodiment 46-Embodiment 51, in the manufacture of a medicament for treating an HIV infection in a human having or at risk of having the infection.

1. A compound of Formula I: 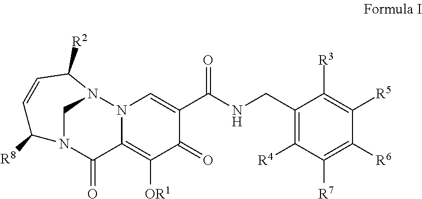
or a pharmaceutically acceptable salt thereof, wherein:
R¹ is —(CR^{1A}R^{1B}O)ₘCO(O—CR^{1C}R^{1D}—CR^{1E}R^{1F})ₙ—(O)ₚR^{1G};
wherein m is 0 or 1;
n is an integer between 0 and 10, inclusive;
p is 0 or 1;
R^{1A} is H or C₁₋₃alkyl;
R^{1B} is H or C₁₋₃alkyl;
each R^{1C} is independently H or C₁₋₃alkyl;
each R^{1D} is independently H or C₁₋₃alkyl;
each R^{1E} is independently H or C₁₋₃alkyl;
each R^{1F} is independently H or C₁₋₃alkyl;
R^{1G} is C₃₋₆cycloalkyl, C₁₋₂₀alkyl, C₂₋₂₀alkenyl, or a 3 to 8 membered heterocyclyl comprising 1, 2, or 3 heteroatoms selected independently from N, O, and S; wherein the C₃₋₆cycloalkyl, C₁₋₂₀alkyl, C₂₋₂₀alkenyl, or 3 to 8 membered heterocyclyl is optionally substituted with a COOH, C₃₋₆cycloalkyl, C₁₋₃alkoxy, or —NHCOOR^{1H};
wherein R^{1H} is C₁₋₃alkyl;
R² is H or C₁₋₃alkyl;
each R³, R⁴, R⁵, R⁶ and R⁷ is independently H or halo; and
R⁸ is H or C₁₋₃alkyl.

2. The compound of claim 1, or a pharmaceutically acceptable salt thereof, wherein:
R¹ is —(CR^{1A}R^{1B}O)ₘCO(O—CR^{1C}R^{1D}—CR^{1E}R^{1F})ₙ—(O)ₚR^{1G};
wherein m is 0 or 1;
n is an integer between 0 and 10, inclusive;
p is 0 or 1;
R^{1A} is H or C₁₋₃alkyl;
R^{1B} is H or C₁₋₃alkyl;
each R^{1C} is independently H or C₁₋₃alkyl;
each R^{1D} is independently H or C₁₋₃alkyl;
each R^{1E} is independently H or C₁₋₃alkyl;
each R^{1F} is independently H or C₁₋₃alkyl;
R^{1G} is C₃₋₆cycloalkyl, C₁₋₂₀alkyl, C₂₋₂₀alkenyl, or a 3 to 8 membered heterocyclyl comprising 1, 2, or 3 heteroatoms selected independently from N, O, and S; wherein the C₃₋₆cycloalkyl, C₁₋₂₀alkyl, C₂₋₂₀alkenyl, or 3 to 8 membered heterocyclyl is optionally substituted with a COOH, C₃₋₆cycloalkyl, or C₁₋₃alkoxy;
R² is H or C₁₋₃alkyl;
each R³, R⁴, R⁵, R⁶ and R⁷ is independently H or halo; and
R⁸ is H or C₁₋₃alkyl.

3. The compound of claim 1, or the pharmaceutically acceptable salt thereof, wherein R³, R⁴, and R⁶ are each F; and wherein R⁵ and R⁷ are each H.

4-9. (canceled)

10. The compound of claim 3, or the pharmaceutically acceptable salt thereof, wherein R² is methyl and R⁸ is methyl.

11-12. (canceled)

13. The compound of claim 10, or the pharmaceutically acceptable salt thereof, wherein n is an integer between 0 and 7, inclusive.

14-17. (canceled)

18. The compound of claim 1, or the pharmaceutically acceptable salt thereof, wherein the compound is of a Formula Ia: 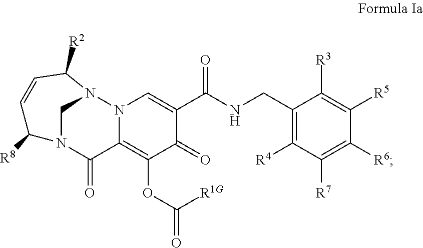
or
wherein the compound is of a Formula Ib:

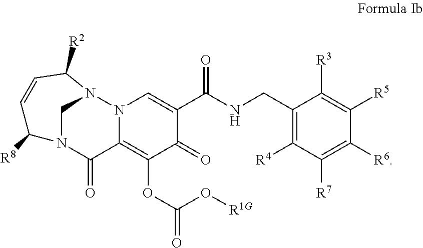

19. (canceled)

20. The compound of claim 1, or the pharmaceutically acceptable salt thereof, wherein the compound is of a Formula Ic: 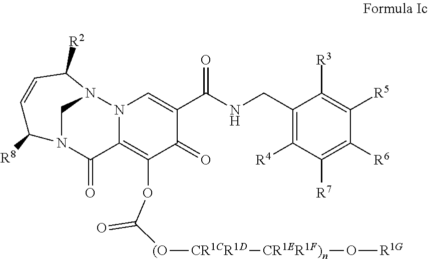
wherein n is 1, 2, or 3; or
wherein the compound is of a Formula Id: 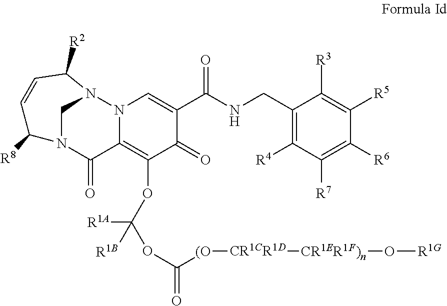
wherein n is 0, 1, 2, or 3; or n is 1, 2, or 3.

21-23. (canceled)

24. The compound of claim 1, or the pharmaceutically acceptable salt thereof, wherein the compound is of a Formula Ie: 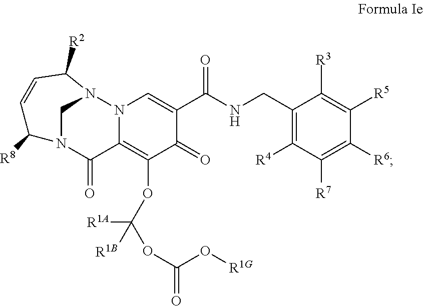
or
wherein the compound is of a Formula If:

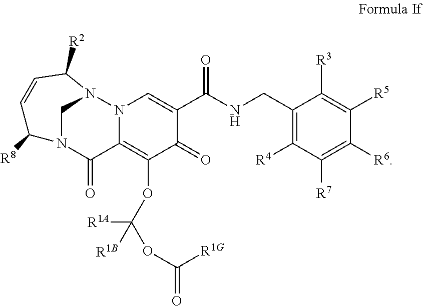

25. (canceled)

26. The compound of claim 1, or the pharmaceutically acceptable salt thereof, wherein n is 1 or 2.

27. (canceled)

28. The compound of claim 1, or the pharmaceutically acceptable salt thereof, wherein each R^{1A} is H and R^{1B} is H; or each R^{1A} is methyl and R^{1B} is H.

29. (canceled)

30. The compound of claim 1, wherein each R^{1C} is H, each R^{1D} is H, each R^{1E} is H, and each R^{1F} is H.

31. The compound of claim 1, or the pharmaceutically acceptable salt thereof, wherein R^{1G} is C₅₋₆cycloalkyl, C₁₋₂₀alkyl or C₂₋₂₀alkenyl; wherein the C₅₋₆cycloalkyl, C₁₋₂₀alkyl or C₂₋₂₀alkenyl is optionally substituted with COOH, C₅₋₆cycloalkyl, C₁₋₃alkoxy, or —NHCOOR^{1H}.

32-35. (canceled)

36. The compound of claim 1, or the pharmaceutically acceptable salt thereof, wherein R¹ is selected from the group consisting of —COCH₃, —COCH₂CH₃, —CO(CH₂)₂CH₃, —CO(CH₂)₃CH₃, —CO(CH₂)₄CH₃, —CO(CH₂)₅CH₃, —CO(CH₂)₆CH₃, —CO(CH₂)₇CH₃, —CO(CH₂)₈CH₃, —CO(CH₂)₉CH₃, —CO(CH₂)₁₀CH₃, —CO(CH₂)₁₁CH₃, —CO(CH₂)₁₂CH₃, —CO(CH₂)₁₃CH₃, —CO(CH₂)₁₄CH₃, —CO(CH₂)₁₅CH₃, —CO(CH₂)₁₆CH₃, —CO(CH₂)₁₇CH₃, —COCH(CH₃)₂, —COCH₂CH(CH₃)₂, —CO(CH₂)₂CH(CH₃)₂, —CO(CH₂)₃CH(CH₃)₂, —CO(CH₂)₄CH(CH₃)₂, —CO(CH₂)₅CH(CH₃)₂, —CO(CH₂)₆CH(CH₃)₂, —CO(CH₂)₇CH(CH₃)₂, —CO(CH₂)₇CH═CH(CH₂)₃CH₃, —CO(CH₂)₃(CH═CHCH₂)₅CH₃, —CO(CH₂)₇(CH═CHCH₂)₂CH₃CH₃, —COCH₂CH₂-cyclopentyl, —CO-cyclopentyl, —CO(CH₂)₅OCH₃, —CO(CH₂)₃COOH, —CO(CH₂)₅COOH, —CO(CH₂)₁₆COOH, and

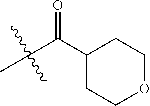 [SMILES: *C(=O)C1CCOCC1]

37. (canceled)

38. The compound of claim 1, or the pharmaceutically acceptable salt thereof, wherein R¹ is selected from the group consisting of —CH₂OCOCH₃, —CH₂OCOCH₂CH₃, —CH₂OCO(CH₂)₂CH₃, —CH₂OCO(CH₂)₃CH₃, —CH₂OCO(CH₂)₄CH₃, —CH₂OCO(CH₂)₅CH₃, —CH₂OCO(CH₂)₆CH₃, —CH₂OCO(CH₂)₇CH₃, —CH₂OCO(CH₂)₈CH₃, —CH₂OCO(CH₂)₁₀CH₃, —CH₂OCO(CH₂)₁₂CH₃, —CH₂OCO(CH₂)₁₄CH₃, —CH₂OCO(CH₂)₁₆CH₃, —CH₂OCOCH(CH₃)₂, —CH₂OCOCH₂CH(CH₃)₂, —CH₂OCO(CH₂)₂CH(CH₃)₂, —CH₂OCO(CH₂)₃CH(CH₃)₂, —CH₂OCO(CH₂)₄CH(CH₃)₂, —CH₂OCO(CH₂)₅CH(CH₃)₂, —CH₂OCO(CH₂)₆CH(CH₃)₂, —CH₂OCO(CH₂)₇CH(CH₃)₂, —CH₂OCO(CH₂)₇CH═CH(CH₂)₃CH₃, —CH₂OCO(CH₂)₃(CH═CHCH₂)₅CH₃, —CH₂OCO(CH₂)₇(CH═CHCH₂)₂CH₃CH₃, —CH₂OCOCH₂CH₂-cyclopentyl, —CH₂OCO-cyclopentyl, —CH₂OCO(CH₂)₅OCH₃, —CH₂OCO(CH₂)₃COOH, —CH₂OCO(CH₂)₅COOH, —CH₂OCO(CH₂)₁₆COOH, and

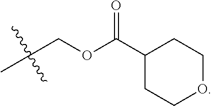 [SMILES: *COC(=O)C1CCOCC1]

39. (canceled)

40. The compound of claim 1, or the pharmaceutically acceptable salt thereof, wherein R¹ is selected from the group consisting of —COOCH₃, —COOCH₂CH₃, —COO(CH₂)₂CH₃, —COO(CH₂)₃CH₃, —COO(CH₂)₄CH₃, —COO(CH₂)₅CH₃, —COO(CH₂)₆CH₃, —COO(CH₂)₇CH₃, —COO(CH₂)₈CH₃, —COO(CH₂)₉CH₃, —COO(CH₂)₁₀CH₃, —COO(CH₂)₁₁CH₃, —COO(CH₂)₁₂CH₃, —COO(CH₂)₁₃CH₃, —COO(CH₂)₁₄CH₃, —COO(CH₂)₁₅CH₃, —COO(CH₂)₁₆CH₃, —COO(CH₂)₁₇CH₃, —COOCH(CH₃)₂, —COOCH₂CH(CH₃)₂, —COO(CH₂)₂CH(CH₃)₂, —COO(CH₂)₃CH(CH₃)₂, —COO(CH₂)₄CH(CH₃)₂, —COO(CH₂)₅CH(CH₃)₂, —COO(CH₂)₆CH(CH₃)₂, —COO(CH₂)₇CH(CH₃)₂, —COO(CH₂CH₂O)₂CH₃, —COO(CH₂)₇CH═CH(CH₂)₃CH₃, —COO(CH₂)₃(CH═CHCH₂)₅CH₃, —COO(CH₂)₇(CH═CHCH₂)₂CH₃CH₃, —COOCH₂CH₂-cyclopentyl, —COO-cyclopentyl, —COO(CH₂)₅OCH₃, —COO(CH₂)₃COOH, —COO(CH₂)₅COOH, —COO(CH₂)₁₆COOH, and

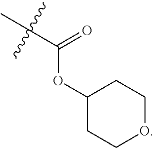 [SMILES: *C(=O)OC1CCOCC1]

41. (canceled)

42. The compound of claim 1, or the pharmaceutically acceptable salt thereof, wherein R¹ is selected from the group consisting of —CH₂OCOOCH₃, —CH₂OCOOCH₂CH₃, —CH₂OCOO(CH₂)₂CH₃, —CH₂OCOO(CH₂)₃CH₃, —CH₂OCOO(CH₂)₄CH₃, —CH₂OCOO(CH₂)₅CH₃, —CH₂OCOO(CH₂)₆CH₃, —CH₂OCOO(CH₂)₇CH₃, —CH₂OCOO(CH₂)₈CH₃, —CH₂OCOO(CH₂)₉CH₃, —CH₂OCOO(CH₂)₁₀CH₃, —CH₂OCOO(CH₂)₁₁CH₃, —CH₂OCOO(CH₂)₁₂CH₃, —CH₂OCOO(CH₂)₁₃CH₃, —CH₂OCOO(CH₂)₁₄CH₃, —CH₂OCOO(CH₂)₁₅CH₃, —CH₂OCOO(CH₂)₁₆CH₃, —CH₂OCOO(CH₂)₁₇CH₃, —CH₂OCOOCH(CH₃)₂, —CH₂OCOOCH₂CH(CH₃)₂, —CH₂OCOO(CH₂)₂CH(CH₃)₂, —CH₂OCOO(CH₂)₃CH(CH₃)₂, —CH₂OCOO(CH₂)₄CH(CH₃)₂, —CH₂OCOO(CH₂)₅CH(CH₃)₂, —CH₂OCOO(CH₂)₆CH(CH₃)₂, —CH₂OCOO(CH₂)₇CH(CH₃)₂, —CH₂OCOO(CH₂)₂OCH₃, —CH₂OCOO(CH₂CH₂O)₂CH₃, —CH(CH₃)OCOOCH(CH₃)₂, —CH₂OCOO(CH₂)₇CH═CH(CH₂)₃CH₃, —CH₂OCOO(CH₂)₃(CH═CHCH₂)₅CH₃, —CH₂OCOO(CH₂)₇(CH═CHCH₂)₂CH₃CH₃, —CH₂OCOOCH₂CH₂-cyclopentyl, —CH₂OCOO-cyclopentyl, —CH₂OCOO(CH₂)₅OCH₃, —CH₂OCOO(CH₂)₃COOH, —CH₂OCOO(CH₂)₅COOH, —CH₂OCOO(CH₂)₁₆COOH, and

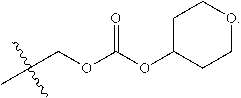 [SMILES: *COC(=O)OC1CCOCC1]

43. (canceled)

44. The compound of claim 1, wherein:
R² is methyl;
R³, R⁴, and R⁶ are each F;
R⁵ and R⁷ are each H; and
R⁸ is methyl.

45. The compound of claim 1, or a pharmaceutically acceptable salt thereof, wherein the compound is selected from the group consisting of the compounds in Table 1, Table 2, Table 3, and Table 4.

46. A pharmaceutical composition comprising a therapeutically effective amount of a compound of claim 1, or pharmaceutically acceptable salt thereof, and a pharmaceutically acceptable excipient.

47-51. (canceled)

52. A kit comprising a compound of claim 1, or a pharmaceutically acceptable salt thereof, and instructions for use.

53-56. (canceled)

57. A method of treating an HIV infection in a human having or at risk of having the infection, comprising administering to the human a therapeutically effective amount of a compound of claim 1 or pharmaceutically acceptable salt thereof, or a pharmaceutical composition of claim 46.

58-66. (canceled)
